SEQUENCE LISTING (527 sequences)

SEQ ID NO: 1 - DNA, 3315 bp, Homo Sapiens
cccctcactccccactgccattcatccagcgctgtgcagtagcccagctgcgtgtctgccgggaggggctgccaagtgccctgcctactggctgcttcccgaatccctgccattccacgcacaaacacatccacacactctctctgcctagttcacacactgagccactcgcacatgcgagcacattccttccttccttctcactctctcggcccttgacttctacaagcccatggaacatttctggaaagacgttcttgatccagcagggtaggcttgttttgatttctctctctgtagctttagcattttgagaaagcaacttacctttctggctagtgtctgtatcctagcagggagatgaggattgctgttctccatgggggtatgtgtgtgtctcctttttctttcaggacttgtaggattctttgtgccatttgcatataatttggcaggttcacattttttaagagccctatgaagtgctttttgcatgtgttttaaaaaggcatttgaaaattgaaagtgtgatttatggaaattaaatcatctgtaaaaaattgctttggaaagtaatgattgctggccataaagggaaatatctgcgatgcacctaatgtgtttttaaccctttatttgctgacaatctatagtcattaatgctaaactcgattttggcttcagctacatttgcatattgtccaacaatggtctatttttgtaagaattagataaaatgtatacttgatataaaatagtcaaaaatgtaactcttagtaacagtaagcttggcatttagatagaccatgaacacttcgtcagatactctgttgggtgtttgggatagcaattaaaacaaagtattgatagttgtatcagagtctattaggctgcagcaaaggaagtttattcaaaagtataaactatccaagattatagacgcatgatatacttcacctattttttgtctccttaatatgtatatatatatatatatatatatatatacacatatatgtgtgtgtgtatgtgcgtgtgcatgtttaacttttaattcagttaaaaacttttttctatttgtttttcatctggatatttgattctgcatatcctagcccaagtgaaccgagaagatcgagttgtaggactaaaggatagacatgcagaaatgcattttaaaaatctgttagctggaccagaccgacaatgtaacataattgccaaagctttggttcgtgacctgaggttatgtttggtatgaaaaggtcacattttatattcagttttctgaagttttggttgcataaccaacctgtggaaggcatgaacacccatgtgcgccctaaccaaaggtttttctgaatcatccttcacatgagaattcctaatgggaccaagtacagtactgtggtccaacataaacacacaagtcaggctgagagaatctcagaaggttgtggaagggtctatctactttgggagcattttgcagaggaagaaactgaggtcctggcaggttgcattctcctgatggcaaaatgcagctcttcctatatgtataccctgaatctccgcccccttcccctcagatgccccctgtcagttcccccagctgctaaatatagctgtctgtggctggctgcgtatgcaaccgcacaccccattctatctgccctatctcggttacagtgtagtcctccccagggtcatcctatgtacacactacgtatttctagccaacgaggagggggaatcaaacagaaagagagacaaacagagatatatcggagtctggcacggggcacataaggcagcacattagagaaagccggcccctggatccgtctttcgcgtttattttaagcccagtcttccctgggccacctttagcagatcctcgtgcgcccccgccccctggccgtgaaactcagcctctatccagcagcgacgacaagtaaagtaaagttcagggaagctgctctttgggatcgctccaaatcgagttgtgcctggagtgatgtttaagccaatgtcagggcaaggcaacagtccctggccgtcctccagcacctttgtaatgcatatgagctcgggagaccagtacttaaagttggaggcccgggagcccaggagctggcggagggcgttcgtcctgggactgcacttgctcccgtcgggtcgcccggcttcaccggacccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggtacccgcgcccgcgccgcccgtcggggtggccgccgcgcccggcaggagggagggagggagggagggagaagggagagcctagggagctgcgggagccgcgggacgcgcgacccgagggtgcgcgcagggagcccggggcgcgcggcccagcccgggggttctgcgtgcagcccgcgctgcgttcagagtcaagttctctcgccgggcagctgaaaaaaacgtactctccacccacttaccgtccgtgcgagaggcagacccgaaagcccgggcttcctaacaaaacacacgttggaaaaccagacaaagcagcagttatttgtgggggaaaacacctccaggcaaataaacacggggcgctttgagtcacttgggaaggtctcgctcttggcatttaaagttgggggtgtttggagttagcagagctcagcagagttttatttatccttttaatgtttttgtttaatgtgctccccaaatttcctttcatctagactat

SEQ ID NO: 2 - DNA, 7369 bp, Homo Sapiens
cagaagtcctcccttagagtgtgtctgggtacacattcaagtgcatggttgcaaacttttttttttaaagcactgaatagtactagacacttagtaggtacttaagaaatattgaatgtcgtggtggtggtgagctagaagttataaaaaaaattctttcccaaaaacaacaacaaaaagaattatttcattgtgaagctcagtaccacaaaaatttaaataattcattacaagcctttattaaaaaaaattttctccccaaagtaaacagacagacaatgtctagtctatttgaaatgcctgaaagcagaggggcttcaaggcagtgggagaaggtgcctgtcctctgctggacatttgacaaccagccctttggatggtttggatgtataggagcgaaggtgcagacagcagtggggcttagagtggggtcctgaggctgtgccgtggcctttctggggtttagccacaatcctggcctgactccagggcgaggcaggccaagggggtctgctactgtgtcctcccacccctacctgggctcccatccccacagcagaggagaaagaagcctgtcctccccgaggtcagctgcgttagaggaagaagactgggcatgtctgggcagagatttccagactctgagcagcctgagatgtcagtaattgtagctgctccaagcctgggttctgttttttagtgggatttctgttcagatgaacaatccatcctctgcaattttttaaaagcaaaactgcaaatgtttcaggcacagaaaggaggcaaaggtgaagtccaggggaggtcaggggtgtgaggtagatgggagcggatagacacatcactcatttctgtgtctgtcagaagaaccagtagacacttccagaattgtcctttatttatgtcatctccataaaccatctgcaaatgagggttatttggcatttttgtcattttggagccacagaaataaaggatgacaagcagagagccccgggcaggaggcaaaagtcctgtgttccaactatagtcatttctttgctgcatgatctgagttaggtcaccagacttctctgagccccagtttccccagcagtgtatacgggctatgtggggagtattcaggagacagacaactcactcgtcaaatcctccccttcctggccaacaaagctgctgcaaccacagggatttcttctgttcaggtgagtgtagggtgtagggagattggttcaatgtccaattcttctgtttccctggagatcaggttgcccttttttggtagtctctccaattccctccttcccggaagcatgtgacaatcaacaactttgtatacttaagttcagtggacctcaatttcctcatctgtgaaataaacgggactgaaaaatcattctggcctcaagatgctttgttggggtgtctaggtgctccaggtgcttctgggagaggtgacctagtgagggatcagtgggaatagaggtgatattgtggggcttttctggaaattgcagagaggtgcatcgtttttataatttatgaatttttatgtattaatgtcatcctcctgatcttttcagctgcattgggtaaatccttgcctgccagagtgggtcagcggtgagccagaaagggggctcattctaacagtgctgtgtcctcctggagagtgccaactcattctccaagtaaaaaaagccagatttgtggctcacttcgtggggaaatgtgtccagcgcaccaacgcaggcgagggactgggggaggagggaagtgccctcctgcagcacgcgaggttccgggaccggctggcctgctggaactcggccaggctcagctggctcggcgctgggcagccaggagcctgggccccggggagggcggtcccgggcggcgcggtgggccgagcgcgggtcccgcctccttgaggcgggcccgggcggggcggttgtatatcagggccgcgctgagctgcgccagctgaggtgtgagcagctgccgaagtcagttccttgtggagccggagctgggcgcggattcgccgaggcaccgaggcactcagaggaggtgagagagcggcggcagacaacaggggaccccgggccggcggcccagagccgagccaagcgtgcccgcgtgtgtccctgcgtgtccgcgaggatgcgtgttcgcgggtgtgtgctgcgttcacaggtgtttctgcggcaggtgaatgacgggcgtgggtcggtgcgcgctcggcttgcgcacacggtgtctctaagtgcgcgggtgacgagagtcgggatgtgccggagaccccggggcggagagcgggattacaagtacaggaatccctggtcacgctccccgcccctggaaacccagctggggcgagggagggcgtggacgggaccgttctgggagctcgcctttggctgcggttggctccaggccccaggcgcagtttgctcgcggcgtggggatgaagtccgtgtccctggaggggcccaggaagggcgaggaaagcggagtggagtaagttcgtctaggatcggtcccgggtggctctgggatccaatctgcgccgccctggcccaggtcccaggttcaggtcctttacgccactgtgtccaccacctggctgagcgctgaggtcagcgcgggctgtttcctggcccttgggaatgtgccaggacccgtcccctaaggactagcgaggaggtgactcactgtgacaaggagaccccagggaacggactgtatgaggtcagaaccccgcccgggatggggtacagcgggactccagaagccctctcccctgccccttcgcggtctccgtcctcccatcggcacagtgacctatttggctggaacagtttgttcccaaggaagccgggcactggaggtccgggacaccgcgtcgggtccccgctccgcggcgcgctgtaggggtcggggagtcacggccctgctctgggcgggctctaaccagcctgtcagtcggggaagggcaagggtctcctctacctctttcccaccgcggccgggagaatcgcggcccagcctgtcctcgggtcggggcgctggactccggggcgggagcggagcccacgcctggatgggaggcggggagggttcatgtctttgaggggtggggggtctggggggcacgacgctgctcagggcctctatcagctgcctcgggggctcagggcttcccgacctagcccagattccctctccgaaagctacagggctgagcggagcaggggggcgagtcgccccctggggcgccgccgcctggcgcggaccacagcgcgtcctctccgtcccaaacccctgggggacacttgcgccctcttcgtgaggaaaagcatcttggagctgggttaggaacttggggcgcccaggcagcttcccctctccttgcctccctccacgtcgcgtttctgggaggacttgcgagcggttttgttttcgttgctcccgtctatttttattttccagggatctgactcatcccgtgctttgggcgtggagataaggtggaggggccggctcccggcgcgcgcgcgcgtgcgtgtctgcgcgggcgtgtgtgtgtgtgtgtgtgtgtgtgtgtgtgtgtgtgtctgtgtcagagacggcacaagagcgcgcggtttcccaacagcggcgggagtttcggaagcctggccggctcagcgtgacgtgttcgcggccccccggtcccctcccattctccccctccccaccccagggtgacgcgcagccggagtggaagcagttttggcgggcgagcagcgccttgcaggaaactgactcatcactactccctccagcggtccgaggctctgcccacgcacctcccactccgcgcgtgatttcctggaggccggcgccccctcccggccctggcgggaatagcacacaggctttcccgcggagtggggctggccggcgcgaaccgccgcggctactcctgggctcatccgagatcaacccctatgccattaccaccccttcaaaggagcactccttaggttcaacagtattcactgagctcttactggaaattaaaatatggctgaagtctaaggcaggaaggccaataaaggaggctatttttaattgtttctaaaacaagggtttgcgtttctgagttttctttgggctgaaagttattatgagcatgagagcagattttgatgggggaggagaggcctatgagagccataagagaaggaggggtggtagaagaggagagggtgcctgcctagatcctagtcctgtcttgaactcccgagagccagggaatatccagcaccttgatgaagccctaggcgggcgcctcctccttgtgcctatgatgtattgagacccagaatgtccatttcaaacataccagtgtgtctccgcttggctggcaacccaagagtgcccatctgaggaattgtgccaaacacttgcttgaatcttcaatctggattaagttggtctcgggaggcagggcctcagcaatctatattttgaaaaaactccctaggtgcttttctttctttctttttttctttctttctttctttctttctttctttctttctttctttctttctttctttttctttctttctttctttttctttcttttctttctttctttctttctttctttctttctttctttctttcttcctttctctttctctctttctttctctttctctctttctttcttttctttcgacagagttgcactctgtcacccaggctggagtgcaatggcaccatcctggactcaagtagtcctcctgtttcagcctcccaagtaaccgggaccacaggcgtgatccccccgcccccatgcccagatttttttttttttttttttttttttgagatgcggtctcgctctgtcacccaggctggagtgcagtggcgtgatctcggctcactgcaagctccgcctcccgggttcacgccattctcctgcctcagcctcccgagtagctgggactacaggtgctgccatcatgcccggctaatttttttttgtatttttagtagagacggggtttcaccgtgttagccaggatggtctcaatctcctgacctcgtgctccgccctcctcggcctcccaaagtgctgggattacaggtgtgagacactgcacccaaccacccagctaatttttatttatttttatttttagtagagacagggtctcagctagttgcccaggctagtcttggacccttgggctcaaatgattctcccacctctgcctcccagagtattaggattacaggcataagccactgcccctggcctccccaagtgattgtgatgggcctctctggttaagaaacctcaaaattagagagggagtggggttcaatactacagcacaggactcagggcaaacaggcctgggttcagatcctggctgtgccacttatgaactgtgtgatgttaggcaagttacttaacttatctgagccttggttgcctcttctgtaaaaagggagctaatagatatccactttttaggaggattgatatttttaaactgcttagaacagcccccaaacataaaaatatataaataaatcccaactcatgcctagcagagggtggatagaggttatttgagggctctgtccactgtactgggtgacccctttatggggcagtggcctttggcctttttagctgtatgactcaggggcaagtctcatatctcttccatctcctgccctttaaacttggtgtgaagttaccaagagcctcctctcccaaccagctgggacgtgaaactgtgggctccactgatcacaagcagtggggtgaggtggggtggagcagatgtggcatgtgtcccgggcttcctgcctcatgaggactcagcagagctttcacccccagaaactgcaagttgggacttgtccctaggaaaatccagttgctgccaaggtcgtgcagtcactcagccctggagtcaagccagagcaggcaggtaggtgccagggctccctcatgggcaaactcactctccgttttccctctcctgaagggggaggagaggagccaggtagaccagccacctttaattttctttttgcctgcaaaacggtttccttggacacaggcaacacgaggcaggggctgccaggtgtctagacttcagatcacctgatgtgcctggcaggatgtggctcagcctgggagaaatcatcccttgcgctgccccgcccggcccctccttacccctaggccacccgcctgacgacatccttgggaaaggccctcagcctacagcacctgtcagctgctgtctgaaggaggtagttggcagggggaagtgataggggggaggctcagtaaaactgaaggcagagaggaataatcatacttctgttttcaatgcacttctctatacgaagtgctgctggcacgttacctacattaactcagttaattctcatgtctatcctctgagacagtcactattactatccccattttatagatgaggaaactagagctcagacaagttaagttgcttgcccagggtcacctagtaaaacctggactccagcccaggtgatctggctccagagccctcctgcttaaccaccaggatacagcctttcattcagctctgttctgtctgccttgctgcatggactctgtgatcaatttcttgagtatgtgtctgtagccatgctctttaaacttgtacatggccccatttatggatgaggaaactgagacctagagacattaagtggctttttaaagcttacgtagtaactggcagagctaggaccacaacccgggtgctttttgccccaaagtcccgggtacttttacttggcagagcagggttaccctacttggggatctgggtcgggggacttaggaggctggaggaactgtcagactgtttcttcttttgggaattgaccttctggccagggctgcgattaggaaactgctggactctggcaattcacacatatttggggggcattcacacccatgagggacacctctggggggaaaacaaattgattttagctgataatacctggtggcaaacaggaccctggtccttgctcttgcaatagacttgcctttgttgacattagcttgcccttcagttgcctgctctcccagtgaccttggtgtgccaggctggctgagctctgctggtgggggtcaggcctcctgtgggaaggaagcaggaagaccagctggaaggagtgagagagaccctctggtaggaagacgtcacctgaggtgacacagcaaagcccggccaggtaacatagtgtctaatctccgccgtgaccagggccttccttgtatctctgctgcagg

SEQ ID NO: 3 - DNA, 2986 bp, Homo Sapiens
gagggtcccttccagctgtcacattctggagcgtatgagatgaggtaggcacacaaagtggacaagatgtggctaagaaaacaagctacacatcaagctcatctgtagcataggtgcttaagaaaactttgctgctgtgtaatattagaacggaaggttggtttccagtaaaatgcattaactttggctcaaaccaagatgatgggtaccgggcatgggggtggggaggcagttgaagatccactgagctttgtctcagggcagccctgctcatcgtcctactttaccttccaccacggtgctcaagcccacactgagagagaaatttccagctgcaaaagggagaagagaaacgctggaatactagtatcggacgttaggacatggttgtggtgttttaaaaatcatttcatcatctggagtttgaccccgaggggagtattttcacccttcagccctctgaaagcattcactagcatctgaatattgttctgagtttgttggagcagtgaaatctggtgagagagaagggtggaggaaggaaggagctgttgtatttggcggctggactcaggtagaggaaactgctacaatcccgggaaagaacagaaaagtagaaagggacgagttcccacacgcagccaatgtccatggccttaactgtgcttgggaaggaagatcctgggccaggggtgtaccctcgtttttcaaaactaaacgtgtctgagacagctacaaagtttattaagggacttgagagactagagttttttgttttttttttttaatcttgagttcctttcttattttcattgagggagagcttgagttcatgataagtgccgcgtctactcctggctaatttctaaaagaaagacgttcgctttggcttcttccctaggcccccagcctccccagggatggcagaaacttctgggttaaggctgagcgaaccattgcccactgcctccaccagcccccagcaaaggcacgccggcgggggggcgcccagcccccccagcaaacgctccgcggcctcccccgcagaccacgaggtgggggccgctggggagggccgagctgggggcagctcgccaccccggctcctagcgagctgccggcgaccttcgcggtcctctggtccaggtcccggcttcccgggcgaggagcgggagggaggtcggggcttaggcgccgcggcgaacccgccaacgcagcgccgggccccgaacctcaggccccgccccaggttcccggccgtttggctagtttgtttgtcttaattttaatttctccgaggccagccagagcaggtttgttggcagcagtacccctccagcagtcacgcgaccagccaatctcccggcggcgctcggggaggcggcgcgctcgggaacgaggggaggtggcggaaccgcgccggggccaccttaaggccgcgctcgccagcctcggcggggcggctcccgccgccgcaaccaatggatctcctcctctgtttaaatagactcgccgtgtcaatcattttcttcttcgtcagcctcccttccaccgccatattgggccactaaaaaaagggggctcgtcttttcggggtgtttttctccccctcccctgtccccgcttgctcacggctctgcgactccgacgccggcaaggtttggagagcggctgggttcgcgggacccgcgggcttgcacccgcccagactcggacgggctttgccaccctctccgcttgcctggtcccctctcctctccgccctcccgctcgccagtccatttgatcagcggagactcggcggccgggccggggcttccccgcagcccctgcgcgctcctagagctcgggccgtggctcgtcggggtctgtgtcttttggctccgagggcagtcgctgggcttccgagaggggttcgggctgcgtaggggcgctttgttttgttcggttttgtttttttgagagtgcgagagaggcggtcgtgcagacccgggagaaagatgtcaaacgtgcgagtgtctaacgggagccctagcctggagcggatggacgccaggcaggcggagcaccccaagccctcggcctgcaggaacctcttcggcccggtggaccacgaagagttaacccgggacttggagaagcactgcagagacatggaagaggcgagccagcgcaagtggaatttcgattttcagaatcacaaacccctagagggcaagtacgagtggcaagaggtggagaagggcagcttgcccgagttctactacagacccccgcggccccccaaaggtgcctgcaaggtgccggcgcaggagagccaggatgtcagcgggagccgcccggcggcgcctttaattggggctccggctaactctgaggacacgcatttggtggacccaaagactgatccgtcggacagccagacggggttagcggagcaatgcgcaggaataaggaagcgacctgcaaccgacggtaatgaccctttcccaaccatagaatgtgtttggggccccgctttgcctgctggagggtgttaaccttagcttgcttttcggcgtattctgatttagctttgggagagctaactttattggtcttaggtgttcagtgctacctggcccactgcttgtctgtttgtgacttttaagtcagaaactggagatggtaagatccgataatttccctaacttaatacatcgcggtccctctcactagcaactcctaggtatgtgacaaagttgggatgtttatcaacggtccgcctcctggctagggaaagagctctggggcggagaatgcactttctgttttttgaaaacaacctcattttgtgcccttaaaagccactggggatgacggatccaggattgtgggtggaggtagtgggtttttcatcccctgactatggggccaacttctgccagccattgttttttctaataaagattgtgtgttctttttaaaaatttcccctgcgcttaga

SEQ ID NO: 4 - DNA, 5666 bp, Homo Sapiens
aaaattagaacttttacctccttgcgcttgttatactctttagtgctgtttaacttttctttgtaagtgagggtggtggagggtgcccataatcttttcagggagtaagttcttcttggtctttctttctttctttctttctttttttcttgagaccaagtttcgctcttgtctcccaggctggagtgcaatggcgcgatctcggctcactgcaacctccgccttctcctgggttcaagcgattctcctacatcagcctccgagtagctgggattacaggcatgcgccaccaagccccgctaattttgtattttttagtagagacagggtttcgccatgttggtcaggcttgtctcgaactcctggcctcaggtgatccgcctgtctcggcctcccagaatgctgggattatagacgtgagccaccgcatccggactttccttttatgtaatagtgataattctatccaaagcatttttttttttttttgagtcggagtctcattctgtcacccaggctggagggtggtggcgcgatctcggcttactgcaacctctgcctcccgggttcaagcgattctcctgcctcagcctcctgagtagctggaattacacacgtgcgccaccatggccagctaatttttgtatttttagtagagacggggtgtcaccattttggccaagctggcctcgaactcctgacctcaggtgatctgcccgcctcggcttcccaaagtgctgggattacaggtgtgagccaccgcgtcctgctccaaagcattttctttctatgcctcaaaacaagattgcaagccagtcctcaaagcggataattcaagagctaacaggtattagcttaggatgtgtggcactgttcttaaggcttatatgtattaatacatcatttaaactcacaacaacccctataaagcagggggcactcatattcccttccccctttataattacgaaaaatgcaaggtattttcagtaggaaagagaaatgtgagaagtgtgaaggagacaggacagtatttgaagctggtctttggatcactgtgcaactctgcttctagaacactgagcactttttctggtctaggaattatgactttgagaatggagtccgtccttccaatgactccctccccattttcctatctgcctacaggcagaattctcccccgtccgtattaaataaacctcatcttttcagagtctgctcttataccaggcaatgtacacgtctgagaaacccttgccccagacagccgttttacacgcaggaggggaaggggaggggaaggagagagcagtccgactctccaaaaggaatcctttgaactagggtttctgacttagtgaaccccgcgctcctgaaaatcaagggttgagggggtagggggacactttctagtcgtacaggtgatttcgattctcggtggggctctcacaactaggaaagaatagttttgctttttcttatgattaaaagaagaagccatactttccctatgacaccaaacaccccgattcaatttggcagttaggaaggttgtatcgcggaggaaggaaacggggcgggggcggatttctttttaacagagtgaacgcactcaaacacgcctttgctggcaggcgggggagcgcggctgggagcagggaggccggagggcggtgtggggggcaggtggggaggagcccagtcctccttccttgccaacgctggctctggcgagggctgcttccggctggtgcccccgggggagacccaacctggggcgacttcaggggtgccacattcgctaagtgctcggagttaatagcacctcctccgagcactcgctcacggcgtccccttgcctggaaagataccgcggtccctccagaggatttgagggacagggtcggagggggctcttccgccagcaccggaggaagaaagaggaggggctggctggtcaccagagggtggggcggaccgcgtgcgctcggcggctgcggagagggggagagcaggcagcgggcggcggggagcagcatggagccggcggcggggagcagcatggagccttcggctgactggctggccacggccgcggcccggggtcgggtagaggaggtgcgggcgctgctggaggcgggggcgctgcccaacgcaccgaatagttacggtcggaggccgatccaggtgggtagagggtctgcagcgggagcaggggatggcgggcgactctggaggacgaagtttgcaggggaattggaatcaggtagcgcttcgattctccggaaaaaggggaggcttcctggggagttttcagaaggggtttgtaatcacagacctcctcctggcgacgccctgggggcttgggaagccaaggaagaggaatgaggagccacgcgcgtacagatctctcgaatgctgagaagatctgaaggggggaacatatttgtattagatggaagtatgctctttatcagatacaaaatttacgaacgtttgggataaaaagggagtcttaaagaaatgtaagatgtgctgggactacttagcctccaattcacagatacctggatggagcttatctttcttactaggagggattatcagtggaaatctgtggtgtatgttggaataaatatcgaatataaattttgatcgaaattattcagaagcggccgggcgcggtgcctcacgccttgtaatcccttcactttgggagatcaaggcggggggaatcacctgaggtcgggagttcgagaccagcctggccaacaggtgaaacctcgcctctactaaaaatacaaaaagtagccgggggtggtggcaggcgcctgtaatcccagctactcgggaggttgaggcaggagaatcgcttgaacccgggaggctgaggttgtagtgaacagcgagatggagccacttcactccagcctgggtgacagagtgagactttgtcgaaagaaagaaagagagaaagagagagagaaaaattattcagaagcaactacatattgtgtttatttttaactgagtagggcaaataaatatatgtttgctgtaggaacttaggaaataatgagccacattcatgtgatcattccagaggtaatatgtagttaccattttgggaatatctgctaacatttttgctcttttactatctttagcttacttgatatagtttatttgtgataagagttttcaattcctcatttttgaacagaggtgtttctcctctccctactcctgttttgtgagggagttaggggaggatttaaaagtaattaatacatgggtaacttagcatctctaaaattttgccaacagcttgaacccgggagtttggctttgtagtcctacaatatcttagaagagaccttatttgtttaaaaacaaaaaggaaaaagaaaagtggatagttttgacaatttttaatggagaagggagaagaacatgtagaaaaggggaaatgatgttggcttagaatcctaactacattggtgtttaatataggaacatttatttatataacattttaaagtactaaattcatattagtatattatcaaatggatatattatcaaatgggtttaagcatcctacacattttaattcaattgattcattttctttttgctttggatttctatcatgatttaaatatttacatatgggttactttttagatttttcatactatgaaatataagaaaaacctttaaggctagttttatgaccaagacgaaggacttcattgaatacacaaaacaataaatatactgcaacattttgtctttctttttgtagctgcaatttggtttgcttatactttctctttgtctctttgaaaactgagtcagtttcactttctcaggacaggatttaataaccataatataatttagtataattccttgatttaggcaaattatgcaatttgtgtttagtatgaaatgtacctaaaaataagtaactcctctttaacaccaccatcctcaaactaatataacaaataacagttatcctaaaataaattgtctacttccaccatgcagcactcaaattttaaggttgctatgactgcagacagtattttaaaattcctctctggaaatggctttgtttccaagatgatttaggaaccaaagaggtgaccatctcttgtttaatgaactctcaaatcataaacctgggaagtgttttagtttcctactgctgctgttacaaattatcacaaatgtgttagctaaaacaaacacaaaattattattttacagttctagagatcagaagtcaaaaatgggtccacaaggtttcattccttttggaaactctaaggggcaatctgtttccttgtcttttccagcttctagtgaccatcaaattccttggctcatggtctctgtattttctctgtggcctgtgcttccattcttgtatcttctctctgactgtgaccctctaataaaaacacttggggttatgttgggcccaccctgaaaattctggataatctccctcaagaccattaattaaatcacatctgcaaagcctcttttgccacataagttaatgtattaaaagtttttgaggattaggacatagacattgggggtgggggggcattattcagcctaccacaggaaggaattttagggttaattaaactagccttcttattttatacttgaagaaattgaagttttggaattggagagcattatgctaaatgaaataagccaaacacagaaagacaaatatcacatgttctcacttatctgtgaaatataaaacaattacattcttagcagtaaagagtagaatggtggttactagagctggggggtgggaggaatggggagatggtaatcaagatataaagcctcagttaagatgggaggaataagtttgattgttttttttgagatgtgtttcatagcatgatgaatatagctaaatagtaaatcccaaatgctctcatttgacaaaaatgtcaaatatttgagatgatggataggttacttagcttgacttaataattccccattgtgttcaaagatcataacttcatattgtaccacataaatatatacaactgtactatcccaatatataattttaaaactaatataatgaaaaagaaattgaagttcaacattcccagaagctaagtgtaacttaaaagttttgtgagaatttgttttaacaaacaaacaagttttctctttttaacaattaccacattctgcgcttggatatacagcagtgaacaaaaaaaaaaaaaaaaaaaaaaatctccaggcctaacataatttcaggaagaaatttcagtagttgtatctcaggggaaatacaggaagttagcctggagtaaaagtcagtctgtccctgcccctttgctattttgcccgtgcctcacagtgctctctgcctgtgacgacagctccgcagaagttcggaggatataatggaattcattgtgtactgaagaatggatagagaactcaagaaggaaattggaaactggaagcaaatgtaggggtaattagacacctggggcttgtgtgggggtctgcttggcggtgagggggctctacacaagcttcctttccgtcatgccggcccccaccctggctctgaccattctgttctctctggcagg

SEQ ID NO: 5 - DNA, 5085 bp, Homo Sapiens
tgatggttgcacaactctgagtacatgaaaaatcaatgaactgatactttgagtgagctgtatgatactggaattacacctcaataaagcatggtaactgttttaagataggctggaaagagaaagcctgaaaacaacaataatgatattaataaattagtttacttctctagtctcatatacttctgtgcccacacttgctcctgttctattcataatggtccccttgcagttgccatattatatcctgccatttgatgcccggtgaacattctatacctgcttcccagaattctctttacctttcctctatctgcctaacttccacatatctaaaattaatcagagtaaactatttactagaacaaccaactccaaatcctagtaacctaacatgataaaggtttgtttctcactcatatagcccctccccagatgatcgaggggtccaggctccttacctctagtggctcccccaccttctggagtcttctgcattctttatacatggttgagataaactatgagtcattagcacagctagaccttgaggtcctacaagaaaatttgcaaatcattcactctgttttgaacaaggtatatttaagatgatgttaaaatacccaatggtcttgggtcaaatacagtttatgactgtgtatctaaaatatatattgcaatattcttccctttttctactgacttcatgaatttagcggggatccattttataagctcaaagataattacttttcagactaagaatatttagggtaaaaagtactgttcaacatctctactgaggatgttatgatgtagcacactgtataagctggagctaaaggaaactttccttaaagtgctatttactaaaaattggaacacattccttaagacaaatcgaagtgtggcacacaacatccaaacttccatcatagatacagaggtgttaccatctcccactcccaaatttctttgtcacgctgaggatactcaagaggagcaggacatgttggtcgcagcaggagaaacttgaaagcattcacttttatggaactcataagggagagaatttcttattttagtatcgtccttgatacatttattattttaaaagataatgtagccaaatgtcttcctctgtgttaaatctttacaaaactgaaatcttaaaatggtgacaaaaattctacttctgatagaatctattcatttttccaattagatagggcataattcttaatttgcaaaacaaaacgtaatatgcttatgaggttccatcccaaagaacctgctattgagagtagcattcagaataacgggtggaaatgccaactccagagtttcagatcctaccggtaattggggtagggaggggctttgggcggggcctccctagaggaggaggcgttgttagaaagctgtctggccagtccacagctgtcactaatcggggtaagccttgttgtatttgtgcgtgtgggtggcattctcaatgagaactagcttcacttgtcatttgagtgaaatctacaacccgaggcggctagtgctcccgcactactgggatctgagatcttcggagatgactgtcgcccgcagtacggagccagcagaagtccgacccttcctgggaatgggctgtaccgagaggtccgactagccccagggttttagtgagggggcagtggaactcagcgagggactgagagcttcacagcatgcacgagtttgatgccagagaaaaagtcgggagataaaggagccgcgtgtcactaaattgccgtcgcagccgcagccactcaagtgccggacttgtgagtactctgcgtctccagtcctcggacagaagttggagaactctcttggagaactccccgagttaggagacgagatctcctaacaattactactttttcttgcgctccccacttgccgctcgctgggacaaacgacagccacagttcccctgacgacaggatggaggccaagggcaggagctgaccagcgccgccctcccccgcccccgacccaggaggtggagatccctccggtccagccacattcaacacccactttctcctccctctgcccctatattcccgaaaccccctcctccttcccttttccctcctccctggagacgggggaggagaaaaggggagtccagtcgtcatgactgagctgaaggcaaagggtccccgggctccccacgtggcgggcggcccgccctcccccgaggtcggatccccactgctgtgtcgcccagccgcaggtccgttcccggggagccagacctcggacaccttgcctgaagtttcggccatacctatctccctggacgggctactcttccctcggccctgccagggacaggacccctccgacgaaaagacgcaggaccagcagtcgctgtcggacgtggagggcgcatattccagagctgaagctacaaggggtgctggaggcagcagttctagtcccccagaaaaggacagcggactgctggacagtgtcttggacactctgttggcgccctcaggtcccgggcagagccaacccagccctcccgcctgcgaggtcaccagctcttggtgcctgtttggccccgaacttcccgaagatccaccggctgcccccgccacccagcgggtgttgtccccgctcatgagccggtccgggtgcaaggttggagacagctccgggacggcagctgcccataaagtgctgccccggggcctgtcaccagcccggcagctgctgctcccggcctctgagagccctcactggtccggggccccagtgaagccgtctccgcaggccgctgcggtggaggttgaggaggaggatggctctgagtccgaggagtctgcgggtccgcttctgaagggcaaacctcgggctctgggtggcgcggcggctggaggaggagccgcggctgtcccgccggggcggcagcaggaggcgtcgccctggtccccaaggaagattcccgcttctcagcgcccagggtcgccctggtggagcaggacgcgccgatcgtccgggacgtccccgctggccaccacggtgatggatttcatccacgtgcctatcctgcctctcaatcacgccttattggcagcccgcactcggcagctgctggaagacgaaagttacgacggcggggccggggctgccagcgcctttgccccgccgcggagttcaccctgtgcctcgtccaccccggtcgctgtaggcgacttccccgactgcgcgtacccgcccgacgccgagcccaaggacgacgcgtaccctctctatagcgacttccagccgcccgctctaaagataaaggaggaggaggaaggcgcggaggcctccgcgcgctccccgcgttcctaccttgtggccggtgccaaccccgcagccttcccggatttcccgttggggccaccgcccccgctgccgccgcgagcgaccccatccagacccggggaagcggcggtgacggccgcacccgccagtgcctcagtctcgtctgcgtcctcctcggggtcgaccctggagtgcatcctgtacaaagcggagggcgcgccgccccagcagggcccgttcgcgccgccgccctgcaaggcgccgggcgcgagcggctgcctgctcccgcgggacggcctgccctccacctccgcctctgccgccgccgccggggcggcccccgcgctctaccctgcactcggcctcaacgggctcccgcagctcggctaccaggccgccgtgctcaaggagggcctgccgcaggtctacccgccctatctcaactacctgaggtgagggcccgggacggggcacgcccagcgcgtccgggagtagcggttccgttggcggcggcggccgccaaccctcagccccagccccagcgcaccgctgcgctccccggggcggccggagagggtgggcagcgggacacagcacaggggcagttgcctcccttcttcttccctcctctcctcactcttggggacacgaaggtgggcgcagaatatactatttttggggcgtgcctccctgaaagctgtttttttgtttgttttttaactttccgaatcttccagattccgaagcagaaccaaccccgatttaaaacgtgcagcgtcacactaggtccgctgtagcccagtggggcagaaagtgcgcggcgagttgggggctttatgaaatgcttctttcttagaagaaggacgtttaccaggagtgcttgtcttggagaggagttaaggcaccgttcccccgggaggggtgggacttgagaggtggccggccagaaccgaaagcagcaccatcttagggatttgaacacttcagtggctcagttttcttaagaatctcaagattaaaattaagttcacgtgggaaatgtttaaactgtggatttaaacgcctgtcactgcattgcaccgttttcttattattgcttgctattcactacaattttttttatatacaggtttaaaaaacactactttgcatactgaagtaatggaatgtaaaaaaagaatgctctgtttggaatcttatgttgtgaataggcaaaacagtgtcagtgtattggacaatactttaaaatgacaaacatatacttgcttaagtaagcaatgattacagggttgtgttttaaaaactcaaaaccaaaacattgcaaagtaccatcgaacttttaaagccaaaccatatttgttttgacccagcatacagacaggaaggacataacatttcatttgtcaaagactaaattgtttctatataaagagttttgtagaaagatttccttttaaccgactttaactttctaggacataatattatacactaattattgttcttttatattggtgctactgatgaatggctaatcatttgcaagtatggtgaatccagttacggatagtctattaccaagtttagtttgcatgtctttcaagtgtatatatacagttctgtttttaaaatctcctttcaccctgttaatactggtttaagaaacctttagtattagatagtggtgcacttaaaaataaatggagtactttgttttgcatttcaagg

SEQ ID NO: 6 - DNA, 8222 bp, Homo Sapiens
aagattagatgtaatcttgccaagcagccactttgtaaactgtatagtcttatgcagatggaaggaagggcctgtgcctaccttgatcatagcactaaacaaactgtactgtattttcattcctcttagttatctccctaaaaagactctgagttccttgaacacaggaaggtgttttatttgattttgttatcctcagcatgtagcagtgtctgacacacagtaggtgctctatcactgtgagagggatggatggatgggtggagttacagatggatagaaggatagatggagggatgggtggatgatggatggatagatggatggaggggggatgatgaatggagggataatgagtggatgaatgagggaatgggtggatggatggatggagggatggaggaacagatagatagatggagggatgggtgggtgatggatggatagatggatggagggagggatgatgaatggagggataatgaatggatgaatgaggggatgggtggatggatgaatggagggatgatgggtggatgaatgaattgagggatggatggatgaacacatggatggatggatagatggatagatggaggaactggtggattttggatggatgggtggatggatagatgaatgaatgcctggatagacaaagagatgatggatagatgaatagatgaattaagggatgtcggatagatggagggattgatagatgttggatggatgggtggtggatggatagatgagtgaatgcatggatagacaaagagatgatggatggatgaattaagggatgacagatggatggatggatgagtaactggatggacaagtggataaatggatagatggttgaatacctgaatggattgaaggaggatgcatggatgtaagataaggctaatcatcctccactctctttctttgcaaaaccatccacccatttactcaataaacatttattcagttcaaacttggcacaaagcaccatgtgaggcccaagagatacgtgggttaataaaacagagctcctgccctcctgaaaactgcaaagaaaggggcgtggcttcctgagttcaaatcccaactctgccagcgactagttgtacatcagtgatgtttccctactttctctcaattaaatagggataatgtcagtacctatcacattgggaggtcttgcggggattaaatgagttaccaaatgccaagtgtttgggacagggcctggcacccagcaaagtctcttgtgagtgctggctgctattatcctaatggagaagatggcatgaaaaccaggaaataggatgccctttgggaagcaatgcaacaggaacttacacaaagaaaggaaaggaggaagcaattagtggtgtctcaaaggagtatgtcaagaaaaacttttcagagggaaacctttgagcagggtcatgaaaacaggagttctctaagagattgtggacttgcctgggaccacctggctataagcacaaaaccatccggttcctttctgtcacttctggctgggtgaggggtctctggcaaaggggcagaaggtgcgtgagaggttgcgaatggccaggactgtcctggggccagccggggcacctggtggccaagcttagaaacatgacaggtcctcttgggagggctgaccgcagggagcgttgggtttcaggctgctggcgtcggcttctgtggtgccctttctgtcggctatgagagtccagacagtgcccaacctcctccccttctttccacacgcacaaccaccccaccccctgtggcctgagctgtcctgcctcgccacaatggcacctgccctaaaatagcttcccatgtgagggctagagaaaggaaaagattagaccctccctggatgagagagagaaagtgaaggagggcaggggagggggacagcgagccattgagcgatctttgtcaagcatcccagaaggtataaaaacgcccttgggaccaggcagcctcaaaccccagctgttggggccaggacacccagtgagcccatacttgctctttttgtcttcttcagactgcgccatggggctcagcgacggggaatggcagttggtgctgaacgtctgggggaaggtggaggctgacatcccaggccatgggcaggaagtcctcatcaggtaaaaggaagagattccattgcccctgccacccacaccctaagatcaagggtgttcagctgcaaggtggaaagtttgcacgtggggtaggtcagttggctgcattagttaagggtgttagaacggtcacttgctttttctttgcttttaagtgtcagggattggactcaggagagggaaaggagccatttcaggctgatgtcagcagctggaggaagcatgagaatcaaacctaggatgctcagagtccaccaggaagaattttagaattatagacagtcagagttaacaagggtcctgagagattttgtacagccacctctcttacaggatgaggacaaaaagggactgagaaggggaggacatttccagagtcacagctcattaaatgctcttaaagtgtcaaggttaagacatgctcttcaaggggagacagatctggttctagacttggctctgccactgagccactgggtgacctttgggaaagtactcaacctctcggagcctcaatttcctctcctgtacagtgaggggatatcctaatatctatatcctagaggagatgtgagaattaaataaaataatgcatgcaagaggcctggcatggttcctggcatatactgagtcctagaaatgttagtagctattactgatgaagcccaggctagggacctttcaaagcattgcaattagagaacagaagatagaggctcattagtgaccttcgatgttgagtatgtctctagtttgagaggtctgaatgatgtggtctgcaagtatatcctgccttctaccacaagggattccagaatacaccaaagaaaacaaaattctgaggtttgtaaatagagggtggctgtggtttgtacatagaagctcatctcctcgttgccttctatcccaaaggtgatacactcttctcttggccccttccctcaccattctgagctggttccctcagaagtctaataggttaagaatcaacgtttctgccaacgggaggaaggaagtgggcgccggggttgccacaagtgaatggattgtctttggcactttaatttgcagattaatttctccttatttgtgagccagagccaattttcctcttccactccgtcgtcctttgggtctcccttgggctttacccatttccagcttccgtctacctcgggagcatggctggtcctggaagctccgttatctaaaatttgagccgttggtgatccgtgctagaaacacttgaggaagaagagttgagttttctgttgactgtaatggcatcgtagtgtgtttatgtctttcttctcctgtccctgtcatctggctgcaacaggccctgttggattgcacaaagtgctcccccgaccaacacacacacacaaattgacatcaactcctgggccacagatatgccatgtgaccttggacaagtctctccacctctgggccttatttctctgctggttgagcgaagggattgtcttccatggtctccgagatcccgtcccacgctcatgccctagaattctctgagtccttgatgcacttttgcctttggcgaggaggcaggacagtcaggcgtggaggtaggagaccagagtttgcagagcagaggtctttccaaggtgatttgggagctgccagacctaaggcagtgacttgtacgtcttgaaagagactcagggtgttgcatcatcagggaggatcacggaggtttgccgggggccgggagccagggataagacataatgcctagggttgctggtccctgaaaggtcccagaggcctggtgccctgtcacgtcttaggttgtgggacatgcagagactgtgccagctggttctgccctctcagtatttggggtacagggaggatggcctccctgggggcagggtggaggaggctcatgcccaggactgcaaatgccacccgtgagagttggcatcttgactttgttctagatactgactctggattttctaactgaccttgaaaggttatgtctccaatcttctgattgtaaaagagggcaaattccatgaagcccagtctgtaaaacactgaggccttcactgcacgtacacaaacataaaattctagaacacagaagctggaaggtcacttggagattctggctccaggagtggggcttcggaagggagggggctgctgagtgggctccacacccctgtttttcctttctttactcaaaccttttttgttttatttactttggctgttttggggagcggggggaacaaaggatttcaaggtcataaaatgtttgaaaatcaccagtggtggtcactacttctcattacagggaggaactgggcctgagagtctccaccggttaatggcagccacaggcaggaccaaaaattcctgggtgatggtgatgataatgataatgataatgataatggtagagatgatgaggaggagaaagacagcaaccatttattcagcacatactgattgctgggctctgtttaagtgtttcactcactgaagcctgacaacaatcttatgaggtagatatgattatgatcaccacatttaacagagaggttaagtaacttacgccaagtcacacagcaagtaagaaaggagctggaattcaaattcaggcagcttggatccagaatccttgctccttttttttttttttgagatggagtcttgctgggattacaggtgcatgccaccatgcctggctaatttttgtatttttagtagagactaggtttcgccatgttggccaggctggtctcgaactcctgacctcaggtgatctgcccgccttggcctcccaaagtgctgggattacaagccaatgagcccggcccagaatccttgctcttgaccactatgctatactgccccatgaccttgccacacgcaagacgggctgccacccacgctctggttcttttattctagtagagccagaggggtgttcatggatctgacaatgtgctccatgttccatgtgagtgtccaattgggcctcagttcagagaacagcaggaaaatggcctcccttctctctgacttggactgaagatctgtctgatgaaccaggcccactgacagcagggttgaagctggaaacacaggccagcggggctttctgaaatactagagcaaacgccctccgtccatttccacaggctgaggggctttcatctgttcattttccggatcaggcaaatccaggagaatggccttctgtggctgcctggggggctcctagctgcaaggcctgggcccacaggaggcaggcagctgtgtggtgccggaatgatcttgcggggagacaaattagcccgattctgctctgctgtttaaagtaaatatggggtggggtggggtggctcaggcctataatcccagcactttgggaggcaggatcggggaatcgcttgagtccaggagttcgagaccagcctgagccacatagtgagaccccccttctctacaaaaaataaacaaaattagctgggcgtggtggtgtgtgcctgtagtcccagctacgcgggaggctgaggtaggaggatcacttgggcccaggagattgaggctccagtgagccaagatcacaccactgcactccagcctgggcaacagagcaataccgtgtctcaaaaaaatattaataaagtaaatatgagctctctccctggctgtctggtgttgagtggaaagaaaggcattggcgggtctagacccttttggtttacatatggatgagcaatgatccagagaggctaagtgacttcaacatcacagagcacattggcgccagaactgggaccggaactcagcttccgtaactgtcagtcccatgttctcttccctagatatcagagggcaggggcaggccccaaaatgtctgagacccccagctgccttgtggcccaaattcttggccctgctctgcctcagacagtctgctttcagcgtcctgacctctgaccatcgcctgtgcatgccaggtcttgcatctgtcaggcgggagttcaaggatcattgtcgccagataataatagtcaggagctcagggacccttggatgaaaggtgtcagagacaagccgtcagtgacgcacgaggatttggataaggtcctggagcgaggtggcaatgttgggcacggcggcagaccatgcccaacatgggggaagggagtgtatgtggttcaggcttgtgcgtgtctctgtgtgtgtgtgcaggtgagtatggcgtgtgctgggatatatttatgtgtacctctgtgtgagtgtgcaggcccatatgtgagtgtgcacacacatctgtgagagcctgcatgcatgtacacgtgtgaggtgttgatacatgtccatgtaggtatcagtttgcctgtaaatatctctgtgtaacaataacaaagtccgaatagtcatcaagagtatagacatgaagccagactgcctgggttaaatccccagctagttagtctttcggtgcctctgtttcttcctctgcaaaatggcgtctaccccatcaggattttaaaatcagttaatatggctgggcgcctgtaatcccagcactttgggaggctgaggcaggcggatcacaaggtcaggagatcgagaccatcctgcctaacatggagaattctgagaattgctcaaacccaggaggtggaggttgcggtgagcagagattgcaccactgcactccagcctgggcaacagagccagattccatctcaaaaaagaaaaacaaaaaacaaaaaaaccatgaactcattttcaggttgaggagctcagcatcctggttgtgaaataccctcctcataaaaccctgggatggagactacggggatcaggtgcttccttgtgacaacttctgggcatggtggctcagggcgcaaactggagtgtggccacaatacatactgtgtacttttacaaggatgtcacagagcctgggtatcataaaagaggagcttttcaaggaactgaaaccattagacaggagagagagccctgggcagacagggttgcccgtgccaaacatttcagctgtggcacaagggaaagggtgggagttatgaaactgttccattttgggtttaggtctgggctctgccgctagctagccaagtgaccttggccacttatctctgtggtcttccatgagtaaaaggcggaaactcactcctacccagagggcaggtctgactccctttaaccagcacccacctgctcacagcaggaaggactgaggtctaaagctggaggtgggcaggaaggactgaggtctaaagctggaggtgggcaggaaggaccaaggtctaaagttggaggtgggcaggaaggaccgaggtctaaagctggaggtggctgctcagagtcccagcagaggcctctggggcacctcactgagtgcctggcaggagtgggtgcctgtctcagggctgggttgagttgctcccaccaggacccttcgtcatctgcacagtgaggggactgggaggttcagagagtcacagcttgggctcaaaacaagcaagaggtttctgagtgtgaggattgctctggagtggaatggccctcacaggtaggagtgagcctcctgtagctagaggtatttaagcagctgaaggacaatccctgggcaggaagctgcagagatggtcgcagcgtggactagaactgctgttttggtcactcagacctcattccagcctggcttctctggacagcacccctgcaatagtgagctggtgactttacgcctcagaacctcggtttccacatctgtaaaatgggaattatatgacactcactatgtgccagacaccctgttggtacatagcacacactatctcacttaatccttcaagtagggacaagttatccccatcccttatatgaggaagctgaggcacagagaggtgaagtgaatggcccaaggtcacacagctgggaagacagggagctaaacttgaactctagtctggctgcccccagacctcacaccgcacctcccatgccgactccagccttccctgtgcccacagg

SEQ ID NO: 7 - DNA, 3051 bp, Homo Sapiens
acagtggctcatgcctgtaatctcacactttgggagaccgattgggaagattgcttgagcccaggagttggagaccagccagagcgacatagtgggaccttctctctacaaaaacatttaaaaattagccaggtacagtggtgcacccctgtagtcccagctatttgggaggtggcaagaggatctctcgagcctagaagttggaggtggcagtgagtagtcatcttgccactgcactccagcctgggtgtcaaagggagactcccctctctagaaaataataaaaaaaaaagtttgacagttttcttgcttgtgctacataaaataatggtacatattacaattgaaggcatcttataatagaaatgccctttcctgttcccttttaaggctgacccagtgctttaagaggctgatacagaagggtaaagttaagtctccacaaaatccagggaagagactgtgaaactcatcttagaaccttgtatggagtcacagctgaactaggaaaaaaaaaaaaaaaaaaaaaaggaaagaaaggcattccagagataaagaatagactaaggagaggagatatttgaagccataatgattgagaatatgtcagacttgaaatgaatcttcagttccctcaacaagacaaataaaactaactccatacctagatacatggtagaaaaactaaagaattctgctgaccaaggtattaaaagtaactaaagagaagtggtgtgaagaaagcaagagagaaacaacaaatcctgtccatcctgtaacaattgaaaatttctggctgggcgtggtggctcacgcctgtaatcccagcactttgagaggccgaggcaggtggatcacctcaggtcaggtgttcaagaccagcctggccaacatggtgaaaccccgtctctactaaaaaaaaataataataataatacaaaaattagccgggggtggtggtaggcacctgtaatcccagatactcgggaggctgaggcaggagactcacttgaacctgggaggcggaggttgcaatgagctgagatcgcgccactgtactccagcctggatgacagagcaggactccatctcaaaaagaaaggaaaagaaaagaaaaaatattaaatgtgtacgctctttgactcagctgtattacttcaaggagttgatatcaccaaaattgcctaagtgctcaaaggtgtttgtagttaaacaacaggagattgataaattatgttatatacatgtgatgctatgttttaaagaggtactgatatgataaaagatgtacgtggcataaaattaaatgtactttattaagtacttttccaagtgtttacggaatgagtgcatttttgaaaaaaaaaaagtgtattcgaacttttaaaaaagctttaaaagctttatacaatgaacgattgagtgattataagagctggcgggggaatgttaagaggatgatagggagctaagtttaacagaacaattcacctctttatcttgtgacacctacgagcgcatcaattctgtaattgaaaaataaagtgcatatttgcagcagctgtactctcttcaggctgcaaggaggcttttcctcccggtaggcttgatttgcatttcactttcactttcgtggctggaaactttctacccacgtagtgggaggctgaggagccaccataaagctggggcttgacgagccgggaccgggacccgatctccacatatgcccggacttgttctgcggccgggttcaggagtcaaagaggcggggagacctgcgcgacgctgccccgccctgcgcccgcttcctccaatgtatgctctagggggcgggcctcgcggggagcatggacacgattggccctaaagtcttccccgcaaggccgtgggctggacagcgtggtgacgtcgcaacgcggcgcagggtgagagcgcgcgcttgcggacgcggcggcattaaacggttgcaggcgtagcagagtggtcgttgtctttctaggtctcagccggtcgtcgcgacgttcgcccgctcgctctgaggctcctgaagccgaaaccagctagactttcctccttcccgcctgcctgtagcggcgttgttgccactccgccaccatgttcgaggcgcgcctggtccagggctccatcctcaagaaggtgttggaggcactcaaggacctcatcaacgaggcctgctgggatattagctccagcggtgtaaacctgcagagcatggactcgtcccacgtctctttggtgcagctcaccctgcggtctgagggcttcgacacctaccgctgcgaccgcaacctggccatgggcgtgaacctcaccaggtgagcctcgcggccccgggaagccggccccggcccgcctgcacctccggtgcttggcgggagcgctttcgagcctagccctcattggctggcgtgggccatccgcgccttctcattggcctgccacgcagtggggtggggcccagctgagcgcgcggttcggaaaagcccgcgctggctgctgcgcgaacctgctttttcgcgccaaagtcacaaagcgggtggtggcgggaaaatcaagggtttttccgcagtgccaggaacactgttccagggctctttgctcactaaatcctgttggccttgaatggacgctttagctgtggctttcttgtttctgagacggtctcggtgtgttgcccgggctggtctccaacttctgggctcaagcgatcctcccggctcagtcgcggttgactttaaatgctttataatgcccttgcgagaaatgtggcagcctgtcatcctacttagtggcaggagattgtttctatccagaagggacactgctggtggtattttagtataaatactgccagatgcgtcccaaaacgtctgcattaataatggcatcctccagcagtccgcttaccctccaccagttctgagacggcctgatgggtgagagtggtaaccccttctaaccgcgttcgaaatacagcccttcagcagacggcgttgattttaaagcatgtgtctcctgtcttctagt

SEQ ID NO: 8 - DNA, 3664 bp, Homo Sapiens
gaattcccaattatcatcctgctcccctaccctcaacacctccagcggtaactgtatcttattcatttttgtgtctatgaagcacagtgttgcactttggagatactacatagaagttggcccaaagggtaataaactgtatttgccaagatgatagcatggatttacaataacctctgaaactttataaagaaagatgattgagttaaattgatacacctagcatcacagctgtggaagcttgtggtcttctttatgttaaaggtaaaatggatataatgaattagttgccatttttagatgatatagaagttgaattcctcttaagcctctcaaagagaaagacatattaaaattcattaaaaccagtaccataaactgcacagaacagcctggttccctagagcacttctctggggcagtgagttttcatttaaaaaatgaagtttggattttaaacataagttttaaaagtgattaggctccagcaggagttcttaaattcctacctacaccatgactcccaaatgctctgcctctgttgcctaggaacaaaaattgtcaaggttgagccagatgcaatgtataacaccagatgatagatgcagctcattgtgttaaaaggtgttaaacacttaagtgcatgtgatatactctatatcacaagggtaaggaagttccattccatatcctgatttgggtcttctaatacatacttgtagtggatgtggtaatcagtctttgttctatgtcccattaggatcaatgcaaccagaaatccttaaaatggatttttattatgtctagctctgttttactgttctatttgtttattttaaaaagattttagaatggtgtttaggttgggctggtggctcacgcctgtaatctcagcactttgggaggccaaggcaggaggattgctggagcctaggagttggagaccagctagggcaacataaggagacattgtctccgcaaacaaacagaaaaaaaaaaagtaagaaagaaagagagaaagaaggaaagaacaagaaaaaggaaaaaaaaattaactgcagtaagtgcagaaatctctctcaaggatatgatggatggcaatgaagacagagtttttgacactggattatttaaagcataatttaagcctctttctctcccctcatagaattgtgaatttttgtgttttaattatatttaagccaggcgtggtggcgagtgcctgtagtcgcagctgcgctggaggctgaggccgattgcttgagcccaggatttggaggccagcatgcgcaacataatgagacccagtctctaaatgcatgcctctctctatatatttaaaattctgatgtgaaaatattttaaaatttaatacatttcaaatgtttttaattgtataataaacaaaatgtaaataataaaataatttaatattaaattcaaaaatgaggtagaaacaaagcacagcgatataaataataaattttcctttacatttttgaggcggtcttttgagttttccatttccttcttaaggtcactgaaatgtgctccttggagccagcccgcaaatcacgcatttagaaaaacataactatacactcctaaccctaagtattagaagtgaaagtaatggaatctcgatgtaaacacaatatcacttttttgatgagctattttgagtataataaatttgaactgtgccaatgctgggagaaaaaatttaaaagaagaacggagcgaacagtagcttcctgctccgctgactagaaacagtaggacgacactctcccgactggaggagagcgcttgcgctcgcactcagttggcgcccgccctcctgctttttctctagccgccctttcctctttctttcgcgctctagccacccgggaaggcctgcccagcgtagctgggctctgattggctgctttgaaagtctacgggctacccgattggtgaatccggggccctttagcgcggtgagtttgaaactgctcgcacttggcttcaaagctggctcttggaaattgagcggagagcgacgcggttgttgtagctgccgctgcggccgccgcggaataataagccgggtacagtggctggggtcagggtcgtgtctaggggacggccgagggcctcggagggcgagtattgaggaacggggtcctctaagaaggccggactggaggtcagggatctgcgcggggcccggctgggcctcggggggcggtgggcagggccttcgcctgggtctggccgtgtgagccacactgggctggctttagagggaggctatgggagcccagcctggcggggttaggcggcgtgggggtgggggttcggggctgcagtcatgagtcgaggtcggctgttctcaggaactgcctgaagaggctttcgggtcttgattgaagcaaaatccgctcccccacgctgagcgcctgcggagtagcaggctctttcctgggcccggcccctgcgcgccctgccatcccgcctcgggggtgggaaagtgggaccgcacctgggccaggtacaggcgcgggtccctagagccaggccgccctgagaaaccgatcctggaactcgtgggccttcttgggcttgctctgcccacggccccggcccacctggagctgttaagaacagtcagcccaggcacttaactctcatgagcagtgtagtgcagtttttttcatttaaaaagatacatctctgcttctttctggattgatttttctttgaagatgaatgtgagaaatagaacttaaaggtctattctgagtgtgctttataaatgattttatcatcagtgatgggtgttagaaaaatctaggtaattatgcctggagttctgacgtttcattggcaaacttcagaggaagtcttgaacttctggttgtcagcttttttttttaacccattcattgtgattcaagtataccgtttttctgcttagtccttgacattgtttaggttataattagaattttatctgtggaattgcacttttcattcttttgttttacggtgcttaagtgatatttcctactcttggaaaaagtaccttgaaagtcttgagcatgtttgtctgtgacctcagcaagcagaggaaaaagattaaaattgtcacgtgggcaactgtaaacacttaaaatgtttttgtcgttttaaacaaaattttcctaatgaacagtctgaacacattttagttctgtcctcataactttaaaaaataatgttttgaataaaattaggagtatcataaagcggtataaatttgttgaaatatttgggtataactttggcacacgcaggtactactttaaagtgtcacaaaaagggaacataattctggtttttgtttggaaagaatagacaagttgatacctgaatttgagggttattttgattggaagaagctaaatactacactagtatagaatatacacgtttccaatgtctcaggagtcaagaaggatggaaagttattgtaggagtccttacccttgtaaatattgtttgaatgtaattaatatttatggaatacttatgctgcagtaggcactgtggaaaatgctttacatatagtgtttcattaatgcttaaaactactcgagttagtggcatgtaattaagtcagtttttccttcactttagg

SEQ ID NO: 9 - DNA, 1729 bp, Homo Sapiens
ctgcattcttggctggggcattccaactagaactgccaaatttagcacataaaaataaggaggcccagttaaatttgaatttcagataaacaatgaataatttgttagtataaatatgtcccatgcaatatcttgttgaaattaaaaaaaaaaaaaaaagtcttccttccatccccacccctaccactaggcctaaggaatagggtcaggggctccaaatagaatgtggttgagaagtggaattaagcaggctaatagaaggcaaggggcaaagaagaaaccttgaatgcattgggtgctgggtgcctccttaaataagcaagaagggtgcattttgaagaattgagatagaagtctttttgggctgggtgcagttgctcgtggttgtaattccagcactttgggaggctgaggcgggaggatcacctgaggttgggagttcaagaccagcctcaccaacgtggagaaaccctgtctttactaaaaatacaaaaaattagctggtcatggtggcacatgcctgtaatcccagctgctcgggaggctgaggcaggagaatcacttgaaccagggaggcagaggttgtggtgagcagagatcgcgccattgctctccagcctgggcaacaagagcaaaagttcgtttaaaaaaaaaaaaaagtcctttcgatgtgactgtctcctcccaaatttgtagaccctcttaagatcatgcttttcagatacttcaaagattccagaagatatgccccgggggtcctggaagccacaaggtaaacacaacacatccccctccttgactatcaattttactagaggatgtggtgggaaaaccattatttgatattaaaacaaataggcttgggatggagtaggatgcaagctccccaggaaagtttaagataaaacctgagacttaaaagggtgttaagagtggcagcctagggaatttatcccggactccgggggagggggcagagtcaccagcctctgcatttagggattctccgaggaaaagtgtgagaacggctgcaggcaacccaggcgtcccggcgctaggagggacgcacccaggcctgcgcgaagagagggagaaagtgaagctgggagttgccactcccagacttgttggaatgcagttggagggggcgagctgggagcgcgcttgctcccaatcacaggagaaggaggaggtggaggaggagggctgcttgaggaagtataagaatgaagttgtgaagctgagattcccctccattgggaccggagaaaccaggggagccccccgggcagccgcgcgccccttcccacggggccctttactgcgccgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaaggtgggtctggtgtggggaggggacggagcagcggcgggaccctgccctgtggatgccccgccgaggtcccgcggccggcggggccagaggggcccggacgagctctcctatcccgaagttgtggacagtcgagacgctcagggcagccgggccctggggccctcgggcgggagggggcagttacacggcagcggctcgagatggcccatccaagagactggcgctttccaggc

SEQ ID NO: 10 - DNA, 12963 bp, Homo Sapiens
gtcaggagccctagaaacaggggagagttagaaagctggccagacctatgcttttcaagtgtagggctagggctgagcctgcctctggggtaggtaagcccccctgaatccttgagggaagtagaagacacaaactgctagataaaatgtaagctcagtctaaaagggctacgtgccgcttctcccagctctggggcatccctctcctagaaaactggactgttttacagtgaaaatctcgggggtggtcagctccctgccccgttgttatccttaccacttacagcctttcaagaagttctcaggttgggtgctgaactctgaccaggaaccactgagaaatcgaggcagctgggagaagctgtagttccaagcgctgaaaggaagatgggggacaataaacctgggtcgccaagcaaagggggcagaggcctggagaagtgggtctcaggaccagaggacagatcgacctcacacttcatctccccagactccacactccactgccatcaccacttacgtgtctccctcgtcctctgcagcgggttccccagaggtatcttccatggcttttccagaccccaactctggcccgttcgcttcttcttcagaaaggctcccgtttgcttcttctgcaggaaggcttgtattttcagaaagttcttgctcctcgattcgaggactcaactcactaggggaaccaaactctgtttccaggggagtggagagagaaactgggtccccctcccgtagctcctgggacacagctgagccagccacaggatctggggacaaccggggcggatcccccctttcgggaggcggtggcatcagttcagagtccgcatttttattcatcggggaagcgtggggagaaggatgggctggagctgggtcctggtctgaaggacagcagtccggagctaacggttgagtctccaaagtcttcatactgcagaggaagcacagcggagattagcctcagccaggatggcttcgaagttctcagggatccgacgcagagctaaagaaacccacctgtgcttccctcctcttctgggagtaggcagaagactcccgggaggagaggcgaacagcggacgccaattcttttgaaagcactgtgttccttagcaccgcgggtcgctacgggcctcttgctgtcgcgggatttcggtccaccttccgattgggccgccgcatcccggatcagatttcgcgggcgacccacggaacccgcggagccgggacgtgaaaggttagaaggtttcccgttcccatcaagccctagggctcctcgtggctgctgggagttgtagtctgaacgcttctatcttggcgagaagcgcctacgctccccctaccgagtcccgcggtaattcttaaagcacctgcaccgcccccccgccgcctgcagagggcgcagcaggtcttgcacctcttctgcatctcattctccaggcttcagacctgtctccctcattcaaaaaatatttattatcgagctcttacttgctacccagcactgatataggcactcaggaatacaacaatgaataagatagtagaaaaattctatatcctcataaggcttacgtttccatgtactgaaagcaatgaacaaataaatcttatcagagtgataagggttgtgaaggagattaaataagatggtgtgatataaagtatctgggagaaaacgttagggtgtgatattacggaaagccttcctaaaaaatgacattttaactgatgagaagaaaggatccagctgagagcaaacgcaaaagctttcttccttccacccttcatatttgacacaatgcaggattcctccaaaatgatttccaccaattctgccctcacagctctggcttgcagaattttccaccccaaaatgttagtatctacggcaccaggtcggcgagaatcctgactctgcaccctcctccccaactccatttcctttgcttcctccggcaggcggattacttgcccttacttgtcatggcgactgtccagctttgtgccaggagcctcgcaggggttgatgggattggggttttcccctcccatgtgctcaagactggcgctaaaagttttgagcttctcaaaagtctagagccaccgtccagggagcaggtagctgctgggctccggggacactttgcgttcgggctgggagcgtgctttccacgacggtgacacgcttccctggattgggtaagctcctgactgaacttgatgagtcctctctgagtcacgggctctcggctccgtgtattttcagctcgggaaaatcgctggggctgggggtggggcagtggggacttagcgagtttgggggtgagtgggatggaagcttggctagagggatcatcataggagttgcattgttgggagacctgggtgtagatgatggggatgttaggaccatccgaactcaaagttgaacgcctaggcagaggagtggagctttggggaaccttgagccggcctaaagcgtacttctttgcacatccacccggtgctgggcgtagggaatccctgaaataaaagatgcacaaagcattgaggtctgagacttttggatctcgaaacattgagaactcatagctgtatattttagagcccatggcatcctagtgaaaactggggctccattccgaaatgatcatttgggggtgatccggggagcccaagctgctaaggtcccacaacttccggacctttgtccttcctggagcgatctttccaggcagcccccggctccgctagatggagaaaatccaattgaaggctgtcagtcgtggaagtgagaagtgctaaaccaggggtttgcccgccaggccgaggaggaccgtcgcaatctgagaggcccggcagccctgttattgtttggctccacatttacatttctgcctcttgcagcagcatttccggtttctttttgccggagcagctcactattcacccgatgagaggggaggagagagagagaaaatgtcctttaggccggttcctcttacttggcagagggaggctgctattctccgcctgcatttctttttctggattacttagttatggcctttgcaaaggcaggggtatttgttttgatgcaaacctcaatccctccccttctttgaatggtgtgccccaccccccgggtcgcctgcaacctaggcggacgctaccatggcgtagacagggagggaaagaagtgtgcagaaggcaagcccggaggcactttcaagaatgagcatatctcatcttcccggagaaaaaaaaaaaagaatggtacgtctgagaatgaaattttgaaagagtgcaatgatgggtcgtttgataatttgtcgggaaaaacaatctacctgttatctagctttgggctaggccattccagttccagacgcaggctgaacgtcgtgaagcggaaggggcgggcccgcaggcgtccgtgtggtcctccgtgcagccctcggcccgagccggttcttcctggtaggaggcggaactcgaattcatttctcccgctgccccatctcttagctcgcggttgtttcattccgcagtttcttcccatgcacctgccgcgtaccggccactttgtgccgtacttacgtcatctttttcctaaatcgaggtggcatttacacacagcgccagtgcacacagcaagtgcacaggaagatgagttttggcccctaaccgctccgtgatgcctaccaagtcacagacccttttcatcgtcccagaaacgtttcatcacgtctcttcccagtcgattcccgaccccacctttattttgatctccataaccattttgcctgttggagaacttcatatagaatggaatcaggatgggcgctgtggctcacgcctgcactttggctcacgcctgcactttgggaggccgaggcgggcggattacttgaggataggagttccagaccagcgtggccaacgtggtgaatccccgtctctactaaaaaatacaaaaattagctgggcgtggtgggtgcctgtaatcccagctattcgggagggtgaggcaggagaatcgcttgaacccgggaggcagaggttgcagtgagccaagatcgtgccactacactccagcctgggcgacaagaacgaaactccgtctcaaaaaaaaggggggaatcatacattatgtgctcatttttgtcgggcttctgtccttcaatgtactgtctgacattcgttcatgttgtatatatcagtattttgctccttttcatttagtatagtccatcgattgtatatccgtccttttgatggccttttgagttgtttcccatttgcggttatgaaataaagctgctataaacattcttgtacaattctttttgtgatcatatgttttcgtgtttcttggagaaatacttaggaggggaattgtggaggaagtaaaaagtagctgtattttgaactttttcagaagctctgagttttccagagcggttgtaccattttacactccaactagcaaggtatgggagttattatggttgtgccacagccttccggacattaggtatgtcagtctttctaatgtggtatatccttgtggttgtaatttacagttctctattgactaaggatgttcagcattttttcatgtgcctattggccattcgtattttgtttgtaaagtagctcttcgagtcttttacctgttattttggtttttttgtttgtttttattgttcagttgtgggactgctttatactttctggatacaagtccttatcagatccatgagtcgtgaatgttttcttctgatctgttgcgggcctatttgtttgctttacagagtttacagaatcttaagaggagtggattaatcttttttatgttcagtatttgccttgtcctgtttaggacatctttttttttttttttaaccccagggtcatgaagatataatcttacattttcttttaggacctttatggtggtaagttttacagtaaggtccttaagccattaattaattcttaaaattaattgtttatggtgtgaggtgtaggagtcagtctctggtatctttcctgtatggaaatccagttattctgtctccacttgttgaaataggcttcctttctctactgaatgcttttaattttaattattttacagttggagtatagggctaccattttagtgctattttctttttttctttgttaatttttgagacagggactcacactgttgcccaggctagagtacaatggcacaatcaaggcttactgcagcctcgaacccctgggctcaagcagtcctctagcagcctcacgagtagctgggattactccaccacacccagctaactattttatttttttgtattgacaggatctcactatgttgcccaggctggtctcaaactgctggcctcaagctttcatcccatctcggcctcccaaagtgctgggattacaggtgtgagccaccatgcctgacctcttagtgctattttctatttatctcctctgttctctgctctctttaaacgttggaggaagaaacagtacccatcttacacaaactcttcagaaaacagaggaacagactgggcgcggtggctcatacctgtaatctcagcactttggtacgctgaggcaggggatcatttgaggtcgggagttcgagaccagcctggccaacacggcgaaaccccatctctactaaaatacaaaagtagctaggcgtgcaccatacctgtaatgccagttactcaggaggctgaggcacaagaatcccttgaacctgggaagcggaggttgcagtgagccgagattgcgccactgcactccagcctgggcaacagagtgagaccctgtctcagaaaaaaaaagaaagaaagaaaaaatagaggaatatttcccaacttgttttcgaagccaggataatcctggtaccaaaaccaaacaaggacattataagaaaagaaaatatagaccaatattcctgttagcatagacatgcaacagctaaccaattttagcaaaccaaacctggtaatatagaaaaaaggataaataggccagtcgcggtggctcacgcctgtaatcccagcactttgggaggctgaggcaggcagatcacttgaggtcaggagtttgagaccagcctgaccaacatggtgaaaccccgtttctaataaaaatacaaaaatcaggctgggcacggtggctcacgcctgtaatcccagcactttgggaggccgaggtgggcagatcacgaggtcaggagttcaagaccagcctgaccaatgtggtgaaacgccatctctactaaaaatacgaaaatcagccggtgtggtggcacctgcctgtaatcccagctactcaggaggctgaggcagaattgcttgaacccgggaggcagaggttgcagtgagccaagatcgtgccactgcactccagcctgggcgacagagcaagacttcatctcaaaaaaaaaaaaaattagctgggcatggtggtgggcacctgaaatcccagctactcgggagtctgaggcaggagaatcgcttgaacccaggaggcagaagttgcactgagctgggatcacaccattgcactccagcctgggcaacagagtgagactccatctcaaaaaaagaaaaagaaaaaggataaatacattctaaccaaataatgtttatctcatgattgtagctgattcaacattcaaaaattggcctggtgcagtagctcaggcctgtaatcccaacattttaggaggctgaggcaggaagatctcttgagcccaggatttcaagaccagcctgggcaacatagtcagactggtctttactggggggaaaaaaatcagtctgtgtaattcaccacattaacaaagggaaacataaaaaccctatgatcatttcaacagatgtagcaaaagcagttaatgatatcaacacatatgcatgattacaaaccaaccaacctcctagcaaactagggaaaggaaacttaactagtttgataacagggcgtccacagtcggagttccactagcagcatacataatggtagaaaactcagtgctgctgggggcggtggctcacgcctgtaatgccagcgctttgggaggcctaggcgggcggatcacgaggtcaggagatcgagactgtcctgactagcatgctgaaaccccgtctctactaaaaatacaaaaacaaaaaattagccgggcatggtggcgggcgcctatagtgccagctactcgggaggctgaggcgagagaatggcgtgaacccgggaggcggagcttgcagagcctagatcgtgccactgcactccagcctgggtgacagagtgagacttcgtctcaaaaaaaaaaaaaaaaaaaaaagaaaagaaaactcaacgctttttcctctaagatcaggaactagaaaaggatttgactctcacaacgttgataccatactggaggttttaaccaggcaagaaaaagaaataatgagggccgggtgcggtggctcaggcctgtaatcccagcactttgggaagccgagacgggtggatcacgaggtcaggagatcgagccatcctggctaacacggtgaaaccctgtctctactaaatatacaaaaaattagccgggcgtggtggcgggcgcctgtagtcccagctactcgggaggctgaggcaggagaatggcgtgaactcagggggcggagcttgcagtgagctgagatcgagccactgcactccagcctgggcgacagagcaagactgtgtctcaaaaaaaaaaaaagaaaaagaaataatgattagtggcccgatgtctcacgccagtaatcccagcactttgggaggccgaggtgggcagatcacctgaggtctggagttggagaccagcctgacaaagatggtgaaacctcgtctctattaaaatattaaaaaaatagccaggcgttggccgggtacagtggctcatgcctgtaaccccagcactttgggaggccgaggtgggtggatcacctgaggtcaggagttcaacaccagcctggccaacatggtgaaaccccatctctactaaaaatacaaaattagccgggcgtagtggcgggcgcctgtaatcccagctacttgggaggcttaggcaggagaatcgcttgaacctgggaggcggaggttgtagtgagccgagattgcaccattgcactccagcctgggtgacaaaagcaaaaactccgtctcaaaaaaaaaagaattagccaggggtagtggtgaacgcctgtagtcccagctactcaggaggcagaggcaggagaatcacttgaaccccggaggcagaggttgcagtgagccgagattgtcccattgcactccagcctaggcgagaagagcaaaattccatgtcaaaaaaaaaaaaaaaaaaggaaagaaaaaaaataacgattagaaaggaagaaatcaaacacattcacagccagtatgattctatacataccatggtcctaatggggccaggcgtggtggctcatgctgtaatcctagcacttttaggaggctgaggcaggtggcttccctgggaccagctggccaacatggtgaaaccccaactctaataaaaatacaaaaaatcagccaggcgtggtgagggcacctctaatcccagctactcaggaggctgaggcaggagaattgcttggacctgggaggcagaggttgcagtgagccgagatcgcgctattgcactccagcctgggcaacaagagtgaaactccggcagggtgtggtcttacgcctgtaatcccagcacttcgggaggctgagccaggccgatcacctgaggtcaggagtttgagaccaacctaacatggtgaaaccccgtctctactaaaaatacaagaattagctgggtgtagtggtgggcgcctgtaatcccagctacttgggaggctgagacagaagaattgcttgaacccaggaggtggaggttgcagtgagctgagatcatgccattgcacaccacgccgggcaacagagcgagattccgtctcaaaaaaaaaaaaaagatgaaactctatctcaaaaaaaaaaaaaagtcctaatggaaaatccataaaaagctaccaaaactaataaataaatatagcagggttgcaggttacagggcaatatagttatccctctatctgtaggggcttggttctgggactcctcacacaccaaacccacagatgtctaagtcccatatataagacggaatagtatttaacctacacatatcctcccatatagtttaaattatctagattacttacattacccccatacaatgaaaatgctaatgtacatgcaagtatgtatgtaagtacttgtactatattgtttagggaatcactggacagataggccttcaagactgataccagcagccactgttaagattctggtcaggcctgcccctgtttggggtctcagttgatctcattgccttcccacccagccaagggcacctgcatttctcttggctccctggccatttggaaggcctagttcagcctggcacatttgtatcctggcccactgatgctggtacccctgggaaggtcctgctctgaaaaacacggagattttagttgctactgaagatttgagagataaagacagggagacctgtctgtagacctgtgtccctccaagtgggattgagactttgggccccccatttcaggacagcacctcctggcctgttgactgaatagatccctgaaggaggtgtagttgcattttaggagtgggggtgggagcagtaccactgatccgcactaacaatcacacagttctctctagaataataatatagaacaagtgaaatagaacaattgcagaaagagctaacctttgttgagctcttactgtgtgcccagcactttcctcaactctacatttcccataatacatagagtactaggtaggcggggcttgggggctcacgcctgtaatcccagcactttaggaggccaaggggggtggatcacctgaggtcgggagttcaagaccagcctgactaacatggtgaaaccccgtctctactagaagtacaaaattagccaggtgtggtggcacatgcttgtagtcctagctactcagcaggctgaggcaggagaatcatttgaatccgggaggaggttgcagtaagcggagatagtgccactgtactccagcctgggcaataagagctgagactccgtctcaaaataaaataaaataaaataaaataaaataaaataaaataaaaaaagaaaagagcctgccattaaaggagctgtttggtaggggatgttttgtcagtgcaaacaacagaaaagtgggctgggcacagtggttcatgcctgtaatcccagcactttgggaggccaaggcgggcggatcacctgaagttgggagttcaagaccagcctgaccaatatggagaaaccccgtctctactaaaaatacaaaattagccgggcgcagtggccgatgcctgtaatcccagctactcgggaggctgaggcaggagaatcgcttgaacctgggaggcagaggttgcggtgagccgagatcgcaccattgcactccagcctggacgagagcaaaactctgtctcaaaaaaaaaaaaaaacagaaaagtgtaacaaacacttacagtaggcatgtttcttagcaaatctgatgacaaatttggcataaagaaagagagcatccctgaaaaaaaaaaaaagaaaaagaaagagagcatcctgcctgggcaacatagtgaaaccctgcctctacaaaaaaactcaaaaattggccgggtgcagtggctcacacctgtaatcccagcactttgggagtcggaggcgggaggatcacctgaggtcaggagttcgaaaccagcctggccaacatggcaaaaccccatctctactaaaaatacaaaaaattaatcaggcgcattggtgggcgcctgtaatcccagctactcaggaagttgaggcaagaggatcgcttgatactgggaggtggaggttacagtgagtcgagatcacaccactgcactctagcctgggtgacagggcgagactccgtctccaaaaaaaaaaagaaaaagaaaaagactaaaaaattagccaggcaggcctctgtggtcccagctacttgggaggctgaggcaggagaatcactgagcccaggagtggcaggctgtagtgagccatgattgcaccactgtaccctagcttgggcttcaaagcaagaccctgcctcaaaagaaaaaagaaagaaagaaagaacatggcgggccaggcacagtggctcacacctgtaatcccagcgctttgagaggccgaggcaggtggatcacaaggtcaggagttccacaccagcctggccaacatggtgaaaccctgtctctactaaaaatacaaaaaatcagcaggcagggtggtaggggcctgtaatcccagctactcgggaggctgaggcaggagaattgcttgaaaccagaaggcagaggttgcagtgagcctagactgcaccactgcactccagcctgggcgaaaagagccaaactccatctcaaaaaacaaacaaaaaaacaaaacaaaaaaaaacatggcagcctttgaaagcttgtctgggagaaggtgcgatgatggttgcataacttcgtgcaagatgctggtccacacaggggctgccccttgctctttctcgctctcttaacctctcatataacaggcttgtgtgttatgcacatttattgagcccaagcaggtgcaaggcattgtgatctaatactttggtcagcaagacaacaagatagatcactgccctgcccttaggaagtgtatatgctattagaggaaacagataaaataaacaaggaaaagtatcagacaatgtaagtgctatgagaatgcaaatgaggtgatgtgaattaaaataggatgacttaagtctgcacggaaggcccctacccccatgttcctggctagccaaggaaccaccagttgattagcagagaagggcagcccgtctagctagagcttttggggaagagggagtggttgttaagagatgagattaaagaagccgagacgggcccttcgtgagggggggttgtaatgcagggctgaggagtgtccgaagagaatgggcaggtgagcggtgagacagttgttcttccagaagctttgcagtgaaaggaatcaaagaaatggagccgtgtatcaggtggggaagggtgggggccaagggggtgtccttccccatacagagattgcaggctgagaatgactatatccttgttaacaggaggtgggagcagggcacggtagctcacacctgtaatcttggcactttaggaggcggaggcgggccgatcacctgaagtaaggagttcgagaccagcctggccaacatgcaaagccctgtctctactaaaaatacaaaaattagctgggtgtggtggtactcgcctgtaatcccagctactcgggagactgaggcaggagaatggcttgaacccggaaggtagaggttgcagtgagctgagatcatgccactgtgctccagcctaggtgacagagagagactccatctcaaaaaaaaaaaaaaatacaggaagggagttgggaatagggtgcacatttaggaagtcttggggatttagtggtgggaaggttggaagtccctctctgattgtcttttcctcaaagaagtgcatggctggtgtggggtggggcaggagtgcttgggttgtggtgaaacattggaagagagaatgtgaagcagccattcttttcctgctccacaggaagccgagctgtctcagacactggcatggtgttgggggagggggttccttctctgcaggcccaggtgacccagggttggaagtgtctcatgctggatccccacttttcctcttgcagc

SEQ ID NO: 11 - DNA, 3500 bp, Homo Sapiens
ctcagaacaaatggcagaggccagtgagcccagagatggtgacagggcaatgatccaggggcagctgcctgaaacgggagcaggtgaagccacagatgggagaagatggttcaggaagaaaaatccaggaatgggcaggagaggagaggaggacacaggctctgtggggctgcagcccaggatgggactaagtgtgaagacatctcagcaggtgaggccaggtcccatgaacagagaagcagctcccacctcccctgatgcacggacacacagagtgtgtggtgctgtgcccccagagtcgggctctcctgttctggtccccagggagtgagaagtgaggttgacttgtccctgctcctctctgctaccccaacattcaccttctcctcatgcccctctctctcaaatatgatttggatctatgtccccgcccaaatctcatgtcaaattgtaaaccccaatgttggaggtggggccttgtgagaagtgattggataatgcgggtggattttctgctttgatgctgtttctgtgatagagatctcacatgatctggttgtttaaaagtgtgtagcacctctcccctctctctctctctctcttactcatgctctgccatgtaagacgttcctgtttccccttcaccgtccagaatgattgtaagttttctgaggcctccccaggagcagaagccactatgcttcctgtacaactgcagaatgatgagcgaattaaacctcttttctttataaattacccagtctcaggtatttctttatagcaatgcgaggacagactaatacaatcttctactcccagatccccgcacacgcttagccccagacatcactgcccctgggagcatgcacagcgcagcctcctgccgacaaaagcaaagtcacaaaaggtgacaaaaatctgcatttggggacatctgattgtgaaagagggaggacagtacacttgtagccacagagactggggctcaccgagctgaaacctggtagcactttggcataacatgtgcatgacccgtgttcaatgtctagagatcagtgttgagtaaaacagcctggtctggggccgctgctgtccccacttccctcctgtccaccagagggcggcagagttcctcccaccctggagcctccccaggggctgctgacctccctcagccgggcccacagcccagcagggtccaccctcacccgggtcacctcggcccacgtcctcctcgccctccgagctcctcacacggactctgtcagctcctccctgcagcctatcggccgcccacctgaggcttgtcggccgcccacttgaggcctgtcggctgccctctgcaggcagctcctgtcccctacaccccctccttccccgggctcagctgaaagggcgtctcccagggcagctccctgtgatctccaggacagctcagtctctcacaggctccgacgccccctatgctgtcacctcacagccctgtcattaccattaactcctcagtcccatgaagttcactgagcgcctgtctcccggttacaggaaaactctgtgacagggaccacgtctgtcctgctctctgtggaatcccagggcccagcccagtgcctgacacggaacagatgctccataaatactggttaaatgtgtgggagatctctaaaaagaagcatatcacctccgtgtggcccccagcagtcagagtctgttccatgtggacacaggggcactggcaccagcatgggaggaggccagcaagtgcccgcggctgccccaggaatgaggcctcaacccccagagcttcagaagggaggacagaggcctgcagggaatagatcctccggcctgaccctgcagcctaatccagagttcagggtcagctcacaccacgtcgaccctggtcagcatccctagggcagttccagacaaggccggaggtctcctcttgccctccagggggtgacattgcacacagacatcactcaggaaacggattcccctggacaggaacctggctttgctaaggaagtggaggtggagcctggtttccatcccttgctccaacagacccttctgatctctcccacatacctgctctgttcctttctgggtcctatgaggaccctgttctgccaggggtccctgtgcaactccagactccctcctggtaccaccatggggaaggtggggtgatcacaggacagtcagcctcgcagagacagagaccacccaggactgtcagggagaacatggacaggccctgagccgcagctcagccaacagacacggagagggagggtccccctggagccttccccaaggacagcagagcccagagtcacccacctccctccaccacagtcctctctttccaggacacacaagacacctccccctccacatgcaggatctggggactcctgagacctctgggcctgggtctccatccctgggtcagtggcggggttggtggtactggagacagagggctggtccctccccagccaccacccagtgagcctttttctagcccccagagccacctctgtcaccttcctgttgggcatcatcccaccttcccagagccctggagagcatggggagacccgggaccctgctgggtttctctgtcacaaaggaaaataatccccctggtgtgacagacccaaggacagaacacagcagaggtcagcactggggaagacaggttgtcctcccaggggatgggggtccatccaccttgccgaaaagatttgtctgaggaactgaaaatagaagggaaaaaagaggagggacaaaagaggcagaaatgagaggggaggggacagaggacacctgaataaagaccacacccatgacccacgtgatgctgagaagtactcctgccctaggaagagactcagggcagagggaggaaggacagcagaccagacagtcacagcagccttgacaaaacgttcctggaactcaagctcttctccacagaggaggacagagcagacagcagagaccatggagtctccctcggcccctccccacagatggtgcatcccctggcagaggctcctgctcacaggtgaagggaggacaacctgggagagggtgggaggagggagctggggtctcctgggtaggacagggctgtgagacggacagagggctcctgttggagcctgaatagggaagaggacatcagagagggacaggagtcacaccagaaaaatcaaattgaactggaattggaaaggggcaggaaaacctcaagagttctattttcctagttaattgtcactggccactacgtttttaaaaatcataataactgcatcagatgacactttaaataaaaacataaccagggcatgaaacactgtcctcatccgcctaccgcggacattggaaaataagccccaggctgtggagggccctgggaaccctcatgaactcatccacaggaatctgcagcctgtcccaggcactggggtgcaaccaagatc

SEQ ID NO: 12 - DNA, 18 bp, Homo Sapiens
tctaacctcgggctgtgc

SEQ ID NO: 13 - DNA, 18 bp, Homo Sapiens
gcacagcccgaggttaga

SEQ ID NO: 14 - DNA, 18 bp, Homo Sapiens
ggcagggccggggccaga

SEQ ID NO: 15 - DNA, 18 bp, Homo Sapiens
tctggccccggccctgcc

SEQ ID NO: 16 - DNA, 18 bp, Homo Sapiens
agatatatcggagtctgg

SEQ ID NO: 17 - DNA, 18 bp, Homo Sapiens
ccagactccgatatatct

SEQ ID NO: 18 - DNA, 18 bp, Homo Sapiens
caggctcccggggcaggg

SEQ ID NO: 19 - DNA, 18 bp, Homo Sapiens
ccctgccccgggagcctg

SEQ ID NO: 20 - DNA, 18 bp, Homo Sapiens
taagtgctcggagttaat

SEQ ID NO: 21 - DNA, 18 bp, Homo Sapiens
attaactccgagcactta

SEQ ID NO: 22 - DNA, 18 bp, Homo Sapiens
ggagggggcgagctggga

SEQ ID NO: 23 - DNA, 18 bp, Homo Sapiens
tcccagctcgccccctcc

SEQ ID NO: 24 - DNA, 18 bp, Homo Sapiens
ccttgagccggcctaaag

SEQ ID NO: 25 - DNA, 18 bp, Homo Sapiens
ctttaggccggctcaagg

SEQ ID NO: 26 - DNA, 18 bp, Homo Sapiens
ctgtcagtcgtggaagtg

SEQ ID NO: 27 - DNA, 18 bp, Homo Sapiens
cacttccacgactgacag

SEQ ID NO: 28 - DNA, 18 bp, Homo Sapiens
aagtgcccgcggctgccc

SEQ ID NO: 29 - DNA, 18 bp, Homo Sapiens
gggcagccgcgggcactt

SEQ ID NO: 30 - DNA, 18 bp, Homo Sapiens
ccctgagccgcagctcag

SEQ ID NO: 31 - DNA, 18 bp, Homo Sapiens
ctgagctgcggctcaggg

SEQ ID NO: 32 - DNA, 3315 bp, Artificial Sequence
  chemically treated genomic DNA (Homo sapiens)
ttttttattttttattgttatttatttagcgttgtgtagtagtttagttgcgtgtttgtcgggaggggttgttaagtgttttgtttattggttgtttttcgaatttttgttattttacgtataaatatatttatatattttttttgtttagtttatatattgagttattcgtatatgcgagtatatttttttttttttttttattttttcggtttttgatttttataagtttatggaatatttttggaaagacgtttttgatttagtagggtaggtttgttttgatttttttttttgtagttttagtattttgagaaagtaatttatttttttggttagtgtttgtattttagtagggagatgaggattgttgttttttatgggggtatgtgtgtgtttttttttttttttaggatttgtaggattttttgtgttatttgtatataatttggtaggtttatattttttaagagttttatgaagtgttttttgtatgtgttttaaaaaggtatttgaaaattgaaagtgtgatttatggaaattaaattatttgtaaaaaattgttttggaaagtaatgattgttggttataaagggaaatatttgcgatgtatttaatgtgtttttaattttttatttgttgataatttatagttattaatgttaaattcgattttggttttagttatatttgtatattgtttaataatggtttatttttgtaagaattagataaaatgtatatttgatataaaatagttaaaaatgtaatttttagtaatagtaagtttggtatttagatagattatgaatatttcgttagatattttgttgggtgtttgggatagtaattaaaataaagtattgatagttgtattagagtttattaggttgtagtaaaggaagtttatttaaaagtataaattatttaagattatagacgtatgatatattttatttattttttgtttttttaatatgtatatatatatatatatatatatatatatatatatatgtgtgtgtgtatgtgcgtgtgtatgtttaatttttaatttagttaaaaatttttttttatttgttttttatttggatatttgattttgtatattttagtttaagtgaatcgagaagatcgagttgtaggattaaaggatagatatgtagaaatgtattttaaaaatttgttagttggattagatcgataatgtaatataattgttaaagttttggttcgtgatttgaggttatgtttggtatgaaaaggttatattttatatttagttttttgaagttttggttgtataattaatttgtggaaggtatgaatatttatgtgcgttttaattaaaggtttttttgaattattttttatatgagaatttttaatgggattaagtatagtattgtggtttaatataaatatataagttaggttgagagaattttagaaggttgtggaagggtttatttattttgggagtattttgtagaggaagaaattgaggttttggtaggttgtatttttttgatggtaaaatgtagttttttttatatgtatattttgaattttcgtttttttttttttagatgttttttgttagtttttttagttgttaaatatagttgtttgtggttggttgcgtatgtaatcgtatattttattttatttgttttatttcggttatagtgtagttttttttagggttattttatgtatatattacgtatttttagttaacgaggagggggaattaaatagaaagagagataaatagagatatatcggagtttggtacggggtatataaggtagtatattagagaaagtcggtttttggattcgtttttcgcgtttattttaagtttagttttttttgggttatttttagtagattttcgtgcgttttcgttttttggtcgtgaaatttagtttttatttagtagcgacgataagtaaagtaaagtttagggaagttgttttttgggatcgttttaaatcgagttgtgtttggagtgatgtttaagttaatgttagggtaaggtaatagtttttggtcgttttttagtatttttgtaatgtatatgagttcgggagattagtatttaaagttggaggttcgggagtttaggagttggcggagggcgttcgttttgggattgtatttgttttcgtcgggtcgttcggttttatcggattcgtaggttttcggggtagggtcggggttagagttcgcgtgtcggcgggatatgcgttgcgtcgtttttaatttcgggttgtgttttttttttaggtggttcgtcggtttttgagttttttgttttgcggggatacggtttgtattttgttcgcggttacggattatgattatgattttttatattaaagtatttgggatggttttattgtattagatttaagggaacgagttggagtttttgaatcgttcgtagtttaagatttttttggagcggtttttgggcgaggtgtatttggatagtagtaagttcgtcgtgtataattatttcgagggcgtcgtttacgagtttaacgtcgcggtcgtcgttaacgcgtaggtttacggttagatcggttttttttacggtttcgggtttgaggttgcggcgttcggttttaacggtttggggggtttttttttatttaatagcgtgttttcgagttcgttgatgttattgtattcgtcgtcgtagttgtcgttttttttgtagttttacggttagtaggtgttttattatttggagaacgagtttagcggttatacggtgcgcgaggtcggttcgtcggtattttataggtattcgcgttcgcgtcgttcgtcggggtggtcgtcgcgttcggtaggagggagggagggagggagggagaagggagagtttagggagttgcgggagtcgcgggacgcgcgattcgagggtgcgcgtagggagttcggggcgcgcggtttagttcgggggttttgcgtgtagttcgcgttgcgtttagagttaagttttttcgtcgggtagttgaaaaaaacgtattttttatttatttatcgttcgtgcgagaggtagattcgaaagttcgggttttttaataaaatatacgttggaaaattagataaagtagtagttatttgtgggggaaaatatttttaggtaaataaatacggggcgttttgagttatttgggaaggtttcgtttttggtatttaaagttgggggtgtttggagttagtagagtttagtagagttttatttatttttttaatgtttttgtttaatgtgttttttaaatttttttttatttagattat

SEQ ID NO: 33 - DNA, 3315 bp, Artificial Sequence
  chemically treated genomic DNA (Homo sapiens)
atagtttagatgaaaggaaatttggggagtatattaaataaaaatattaaaaggataaataaaattttgttgagttttgttaattttaaatatttttaattttaaatgttaagagcgagatttttttaagtgatttaaagcgtttcgtgtttatttgtttggaggtgtttttttttataaataattgttgttttgtttggttttttaacgtgtgttttgttaggaagttcgggttttcgggtttgtttttcgtacggacggtaagtgggtggagagtacgttttttttagttgttcggcgagagaatttgattttgaacgtagcgcgggttgtacgtagaattttcgggttgggtcgcgcgtttcgggttttttgcgcgtattttcgggtcgcgcgtttcgcggttttcgtagttttttaggttttttttttttttttttttttttttttttttttgtcgggcgcggcggttatttcgacgggcggcgcgggcgcgggtatttgtagaatgtcggcgggtcggtttcgcgtatcgtgtagtcgttgggttcgttttttaggtagtagggtatttgttggtcgtggggttgtaggaaaggcgatagttgcggcggcgggtgtagtagtattagcgggttcggagatacgttgttgagtggggggaaattttttaggtcgttggagtcgaacgtcgtagttttagattcggggtcgtaggggaggtcggtttgatcgtagatttgcgcgttggcggcggtcgcggcgttgaattcgtaggcggcgttttcggggtagttgtatacggcgggtttgttgttgtttaggtatatttcgtttaggggtcgttttagggggattttgagttgcggacggtttaggggttttagttcgtttttttggatttgatgtagtagggttattttagatgttttggtgtggagggttatggttatggttcgtggtcgcgggtagggtgtagatcgtgttttcgtagggtagaaggtttagaaatcggcgggttatttggaaaaagagtatagttcgaggttagaggcgacgtagcgtatgtttcgtcgatacgcgagttttggtttcggttttgtttcgggagtttgcgggttcggtgaagtcgggcgattcgacgggagtaagtgtagttttaggacgaacgtttttcgttagtttttgggttttcgggtttttaattttaagtattggtttttcgagtttatatgtattataaaggtgttggaggacggttagggattgttgttttgttttgatattggtttaaatattattttaggtataattcgatttggagcgattttaaagagtagtttttttgaattttattttatttgtcgtcgttgttggatagaggttgagttttacggttagggggcgggggcgtacgaggatttgttaaaggtggtttagggaagattgggtttaaaataaacgcgaaagacggatttaggggtcggttttttttaatgtgttgttttatgtgtttcgtgttagatttcgatatatttttgtttgtttttttttttgtttgatttttttttttcgttggttagaaatacgtagtgtgtatataggatgattttggggaggattatattgtaatcgagatagggtagatagaatggggtgtgcggttgtatacgtagttagttatagatagttatatttagtagttgggggaattgatagggggtatttgaggggaagggggcggagatttagggtatatatataggaagagttgtattttgttattaggagaatgtaatttgttaggattttagtttttttttttgtaaaatgtttttaaagtagatagatttttttataattttttgagatttttttagtttgatttgtgtgtttatgttggattatagtattgtatttggttttattaggaatttttatgtgaaggatgatttagaaaaatttttggttagggcgtatatgggtgtttatgttttttataggttggttatgtaattaaaattttagaaaattgaatataaaatgtgatttttttatattaaatataattttaggttacgaattaaagttttggtaattatgttatattgtcggtttggtttagttaatagatttttaaaatgtatttttgtatgtttattttttagttttataattcgattttttcggtttatttgggttaggatatgtagaattaaatatttagatgaaaaataaatagaaaaaagtttttaattgaattaaaagttaaatatgtatacgtatatatatatatatatatatgtgtatatatatatatatatatatatatatatatattaaggagataaaaaataggtgaagtatattatgcgtttataattttggatagtttatatttttgaataaattttttttgttgtagtttaatagattttgatataattattaatattttgttttaattgttattttaaatatttaatagagtatttgacgaagtgtttatggtttatttaaatgttaagtttattgttattaagagttatatttttgattattttatattaagtatatattttatttaatttttataaaaatagattattgttggataatatgtaaatgtagttgaagttaaaatcgagtttagtattaatgattatagattgttagtaaataaagggttaaaaatatattaggtgtatcgtagatatttttttttatggttagtaattattattttttaaagtaattttttatagatgatttaatttttataaattatatttttaatttttaaatgtttttttaaaatatatgtaaaaagtattttatagggtttttaaaaaatgtgaatttgttaaattatatgtaaatggtataaagaattttataagttttgaaagaaaaaggagatatatatatatttttatggagaatagtaatttttatttttttgttaggatatagatattagttagaaaggtaagttgtttttttaaaatgttaaagttatagagagagaaattaaaataagtttattttgttggattaagaacgtttttttagaaatgttttatgggtttgtagaagttaagggtcgagagagtgagaaggaaggaaggaatgtgttcgtatgtgcgagtggtttagtgtgtgaattaggtagagagagtgtgtggatgtgtttgtgcgtggaatggtagggattcgggaagtagttagtaggtagggtatttggtagtttttttcggtagatacgtagttgggttattgtatagcgttggatgaatggtagtggggagtgagggg

SEQ ID NO: 34 - DNA, 7369 bp, Artificial Sequence
  chemically treated genomic DNA (Homo sapiens)
tagaagtttttttttagagtgtgtttgggtatatatttaagtgtatggttgtaaatttttttttttaaagtattgaatagtattagatatttagtaggtatttaagaaatattgaatgtcgtggtggtggtgagttagaagttataaaaaaaatttttttttaaaaataataataaaaagaattattttattgtgaagtttagtattataaaaatttaaataatttattataagtttttattaaaaaaaattttttttttaaagtaaatagatagataatgtttagtttatttgaaatgtttgaaagtagaggggttttaaggtagtgggagaaggtgtttgttttttgttggatatttgataattagttttttggatggtttggatgtataggagcgaaggtgtagatagtagtggggtttagagtggggttttgaggttgtgtcgtggtttttttggggtttagttataattttggtttgattttagggcgaggtaggttaagggggtttgttattgtgtttttttatttttatttgggtttttatttttatagtagaggagaaagaagtttgtttttttcgaggttagttgcgttagaggaagaagattgggtatgtttgggtagagatttttagattttgagtagtttgagatgttagtaattgtagttgttttaagtttgggttttgttttttagtgggatttttgtttagatgaataatttattttttgtaattttttaaaagtaaaattgtaaatgttttaggtatagaaaggaggtaaaggtgaagtttaggggaggttaggggtgtgaggtagatgggagcggatagatatattatttatttttgtgtttgttagaagaattagtagatatttttagaattgttttttatttatgttatttttataaattatttgtaaatgagggttatttggtatttttgttattttggagttatagaaataaaggatgataagtagagagtttcgggtaggaggtaaaagttttgtgttttaattatagttatttttttgttgtatgatttgagttaggttattagatttttttgagttttagtttttttagtagtgtatacgggttatgtggggagtatttaggagatagataatttattcgttaaatttttttttttttggttaataaagttgttgtaattatagggattttttttgtttaggtgagtgtagggtgtagggagattggtttaatgtttaatttttttgtttttttggagattaggttgtttttttttggtagtttttttaattttttttttttcggaagtatgtgataattaataattttgtatatttaagtttagtggattttaatttttttatttgtgaaataaacgggattgaaaaattattttggttttaagatgttttgttggggtgtttaggtgttttaggtgtttttgggagaggtgatttagtgagggattagtgggaatagaggtgatattgtggggtttttttggaaattgtagagaggtgtatcgtttttataatttatgaatttttatgtattaatgttatttttttgatttttttagttgtattgggtaaatttttgtttgttagagtgggttagcggtgagttagaaagggggtttattttaatagtgttgtgtttttttggagagtgttaatttattttttaagtaaaaaaagttagatttgtggtttatttcgtggggaaatgtgtttagcgtattaacgtaggcgagggattgggggaggagggaagtgtttttttgtagtacgcgaggtttcgggatcggttggtttgttggaattcggttaggtttagttggttcggcgttgggtagttaggagtttgggtttcggggagggcggtttcgggcggcgcggtgggtcgagcgcgggtttcgtttttttgaggcgggttcgggcggggcggttgtatattagggtcgcgttgagttgcgttagttgaggtgtgagtagttgtcgaagttagttttttgtggagtcggagttgggcgcggattcgtcgaggtatcgaggtatttagaggaggtgagagagcggcggtagataataggggatttcgggtcggcggtttagagtcgagttaagcgtgttcgcgtgtgtttttgcgtgttcgcgaggatgcgtgttcgcgggtgtgtgttgcgtttataggtgtttttgcggtaggtgaatgacgggcgtgggtcggtgcgcgttcggtttgcgtatacggtgtttttaagtgcgcgggtgacgagagtcgggatgtgtcggagatttcggggcggagagcgggattataagtataggaatttttggttacgtttttcgtttttggaaatttagttggggcgagggagggcgtggacgggatcgttttgggagttcgtttttggttgcggttggttttaggttttaggcgtagtttgttcgcggcgtggggatgaagttcgtgtttttggaggggtttaggaagggcgaggaaagcggagtggagtaagttcgtttaggatcggtttcgggtggttttgggatttaatttgcgtcgttttggtttaggttttaggtttaggttttttacgttattgtgtttattatttggttgagcgttgaggttagcgcgggttgttttttggtttttgggaatgtgttaggattcgttttttaaggattagcgaggaggtgatttattgtgataaggagattttagggaacggattgtatgaggttagaatttcgttcgggatggggtatagcgggattttagaagtttttttttttgttttttcgcggttttcgtttttttatcggtatagtgatttatttggttggaatagtttgtttttaaggaagtcgggtattggaggttcgggatatcgcgtcgggttttcgtttcgcggcgcgttgtaggggtcggggagttacggttttgttttgggcgggttttaattagtttgttagtcggggaagggtaagggttttttttattttttttttatcgcggtcgggagaatcgcggtttagtttgttttcgggtcggggcgttggatttcggggcgggagcggagtttacgtttggatgggaggcggggagggtttatgtttttgaggggtggggggtttggggggtacgacgttgtttagggtttttattagttgtttcgggggtttagggtttttcgatttagtttagatttttttttcgaaagttatagggttgagcggagtaggggggcgagtcgttttttggggcgtcgtcgtttggcgcggattatagcgcgtttttttcgttttaaatttttgggggatatttgcgtttttttcgtgaggaaaagtattttggagttgggttaggaatttggggcgtttaggtagttttttttttttttgtttttttttacgtcgcgtttttgggaggatttgcgagcggttttgttttcgttgttttcgtttatttttattttttagggatttgatttatttcgtgttttgggcgtggagataaggtggaggggtcggttttcggcgcgcgcgcgcgtgcgtgtttgcgcgggcgtgtgtgtgtgtgtgtgtgtgtgtgtgtgtgtgtgtgtttgtgttagagacggtataagagcgcgcggttttttaatagcggcgggagtttcggaagtttggtcggtttagcgtgacgtgttcgcggtttttcggtttttttttattttttttttttttattttagggtgacgcgtagtcggagtggaagtagttttggcgggcgagtagcgttttgtaggaaattgatttattattattttttttagcggttcgaggttttgtttacgtattttttatttcgcgcgtgattttttggaggtcggcgttttttttcggttttggcgggaatagtatataggttttttcgcggagtggggttggtcggcgcgaatcgtcgcggttatttttgggtttattcgagattaatttttatgttattattatttttttaaaggagtattttttaggtttaatagtatttattgagtttttattggaaattaaaatatggttgaagtttaaggtaggaaggttaataaaggaggttatttttaattgtttttaaaataagggtttgcgtttttgagttttttttgggttgaaagttattatgagtatgagagtagattttgatgggggaggagaggtttatgagagttataagagaaggaggggtggtagaagaggagagggtgtttgtttagattttagttttgttttgaattttcgagagttagggaatatttagtattttgatgaagttttaggcgggcgttttttttttgtgtttatgatgtattgagatttagaatgtttattttaaatatattagtgtgttttcgtttggttggtaatttaagagtgtttatttgaggaattgtgttaaatatttgtttgaatttttaatttggattaagttggtttcgggaggtagggttttagtaatttatattttgaaaaaattttttaggtgtttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttcgatagagttgtattttgttatttaggttggagtgtaatggtattattttggatttaagtagtttttttgttttagttttttaagtaatcgggattataggcgtgattttttcgtttttatgtttagatttttttttttttttttttttttttgagatgcggtttcgttttgttatttaggttggagtgtagtggcgtgatttcggtttattgtaagtttcgtttttcgggtttacgttatttttttgttttagtttttcgagtagttgggattataggtgttgttattatgttcggttaatttttttttgtatttttagtagagacggggttttatcgtgttagttaggatggttttaattttttgatttcgtgtttcgtttttttcggttttttaaagtgttgggattataggtgtgagatattgtatttaattatttagttaatttttatttatttttatttttagtagagatagggttttagttagttgtttaggttagttttggatttttgggtttaaatgatttttttatttttgttttttagagtattaggattataggtataagttattgtttttggtttttttaagtgattgtgatgggtttttttggttaagaaattttaaaattagagagggagtggggtttaatattatagtataggatttagggtaaataggtttgggtttagattttggttgtgttatttatgaattgtgtgatgttaggtaagttatttaatttatttgagttttggttgttttttttgtaaaaagggagttaatagatatttattttttaggaggattgatatttttaaattgtttagaatagtttttaaatataaaaatatataaataaattttaatttatgtttagtagagggtggatagaggttatttgagggttttgtttattgtattgggtgatttttttatggggtagtggtttttggtttttttagttgtatgatttaggggtaagttttatattttttttattttttgttttttaaatttggtgtgaagttattaagagtttttttttttaattagttgggacgtgaaattgtgggttttattgattataagtagtggggtgaggtggggtggagtagatgtggtatgtgtttcgggttttttgttttatgaggatttagtagagtttttatttttagaaattgtaagttgggatttgtttttaggaaaatttagttgttgttaaggtcgtgtagttatttagttttggagttaagttagagtaggtaggtaggtgttagggtttttttatgggtaaatttatttttcgtttttttttttttgaagggggaggagaggagttaggtagattagttatttttaattttttttttgtttgtaaaacggtttttttggatataggtaatacgaggtaggggttgttaggtgtttagattttagattatttgatgtgtttggtaggatgtggtttagtttgggagaaattattttttgcgttgtttcgttcggtttttttttatttttaggttattcgtttgacgatatttttgggaaaggtttttagtttatagtatttgttagttgttgtttgaaggaggtagttggtagggggaagtgataggggggaggtttagtaaaattgaaggtagagaggaataattatatttttgtttttaatgtatttttttatacgaagtgttgttggtacgttatttatattaatttagttaatttttatgtttattttttgagatagttattattattatttttattttatagatgaggaaattagagtttagataagttaagttgtttgtttagggttatttagtaaaatttggattttagtttaggtgatttggttttagagtttttttgtttaattattaggatatagttttttatttagttttgttttgtttgttttgttgtatggattttgtgattaattttttgagtatgtgtttgtagttatgttttttaaatttgtatatggttttatttatggatgaggaaattgagatttagagatattaagtggttttttaaagtttacgtagtaattggtagagttaggattataattcgggtgttttttgttttaaagtttcgggtatttttatttggtagagtagggttattttatttggggatttgggtcgggggatttaggaggttggaggaattgttagattgtttttttttttgggaattgattttttggttagggttgcgattaggaaattgttggattttggtaatttatatatatttggggggtatttatatttatgagggatatttttggggggaaaataaattgattttagttgataatatttggtggtaaataggattttggtttttgtttttgtaatagatttgtttttgttgatattagtttgttttttagttgtttgtttttttagtgattttggtgtgttaggttggttgagttttgttggtgggggttaggttttttgtgggaaggaagtaggaagattagttggaaggagtgagagagattttttggtaggaagacgttatttgaggtgatatagtaaagttcggttaggtaatatagtgtttaattttcgtcgtgattagggttttttttgtatttttgttgtagg

SEQ ID NO: 35 - DNA, 7369 bp, Artificial Sequence
  chemically treated genomic DNA (Homo sapiens)
tttgtagtagagatataaggaaggttttggttacggcggagattagatattatgttatttggtcgggttttgttgtgttattttaggtgacgtttttttattagagggttttttttattttttttagttggtttttttgttttttttttataggaggtttgatttttattagtagagtttagttagtttggtatattaaggttattgggagagtaggtaattgaagggtaagttaatgttaataaaggtaagtttattgtaagagtaaggattagggttttgtttgttattaggtattattagttaaaattaatttgttttttttttagaggtgttttttatgggtgtgaatgttttttaaatatgtgtgaattgttagagtttagtagttttttaatcgtagttttggttagaaggttaatttttaaaagaagaaatagtttgatagtttttttagttttttaagtttttcgatttagatttttaagtagggtaattttgttttgttaagtaaaagtattcgggattttggggtaaaaagtattcgggttgtggttttagttttgttagttattacgtaagttttaaaaagttatttaatgtttttaggttttagtttttttatttataaatggggttatgtataagtttaaagagtatggttatagatatatatttaagaaattgattatagagtttatgtagtaaggtagatagaatagagttgaatgaaaggttgtattttggtggttaagtaggagggttttggagttagattatttgggttggagtttaggttttattaggtgattttgggtaagtaatttaatttgtttgagttttagtttttttatttataaaatggggatagtaatagtgattgttttagaggatagatatgagaattaattgagttaatgtaggtaacgtgttagtagtatttcgtatagagaagtgtattgaaaatagaagtatgattatttttttttgtttttagttttattgagttttttttttattatttttttttgttaattattttttttagatagtagttgataggtgttgtaggttgagggttttttttaaggatgtcgttaggcgggtggtttaggggtaaggaggggtcgggcggggtagcgtaagggatgattttttttaggttgagttatattttgttaggtatattaggtgatttgaagtttagatatttggtagtttttgtttcgtgttgtttgtgtttaaggaaatcgttttgtaggtaaaaagaaaattaaaggtggttggtttatttggttttttttttttttttttaggagagggaaaacggagagtgagtttgtttatgagggagttttggtatttatttgtttgttttggtttgattttagggttgagtgattgtacgattttggtagtaattggatttttttagggataagttttaatttgtagtttttgggggtgaaagttttgttgagtttttatgaggtaggaagttcgggatatatgttatatttgttttattttattttattttattgtttgtgattagtggagtttatagttttacgttttagttggttgggagaggaggtttttggtaattttatattaagtttaaagggtaggagatggaagagatatgagatttgtttttgagttatatagttaaaaaggttaaaggttattgttttataaaggggttatttagtatagtggatagagtttttaaataatttttatttattttttgttaggtatgagttgggatttatttatatatttttatgtttgggggttgttttaagtagtttaaaaatattaatttttttaaaaagtggatatttattagttttttttttatagaagaggtaattaaggtttagataagttaagtaatttgtttaatattatatagtttataagtggtatagttaggatttgaatttaggtttgtttgttttgagttttgtgttgtagtattgaattttattttttttttaattttgaggttttttaattagagaggtttattataattatttggggaggttaggggtagtggtttatgtttgtaattttaatattttgggaggtagaggtgggagaattatttgagtttaagggtttaagattagtttgggtaattagttgagattttgtttttattaaaaataaaaataaataaaaattagttgggtggttgggtgtagtgttttatatttgtaattttagtattttgggaggtcgaggagggcggagtacgaggttaggagattgagattattttggttaatacggtgaaatttcgtttttattaaaaatataaaaaaaaattagtcgggtatgatggtagtatttgtagttttagttattcgggaggttgaggtaggagaatggcgtgaattcgggaggcggagtttgtagtgagtcgagattacgttattgtattttagtttgggtgatagagcgagatcgtattttaaaaaaaaaaaaaaaaaaaaaaaaatttgggtatgggggcggggggattacgtttgtggtttcggttatttgggaggttgaaataggaggattatttgagtttaggatggtgttattgtattttagtttgggtgatagagtgtaattttgtcgaaagaaaagaaagaaagagagaaagagaaagaaagagagaaagagaaaggaagaaagaaagaaagaaagaaagaaagaaagaaagaaagaaagaaaagaaagaaaaagaaagaaagaaagaaaaagaaagaaagaaagaaagaaagaaagaaagaaagaaagaaagaaagaaagaaaaaaagaaagaaagaaaagtatttagggagtttttttaaaatatagattgttgaggttttgtttttcgagattaatttaatttagattgaagatttaagtaagtgtttggtataattttttagatgggtatttttgggttgttagttaagcggagatatattggtatgtttgaaatggatattttgggttttaatatattataggtataaggaggaggcgttcgtttagggttttattaaggtgttggatattttttggttttcgggagtttaagataggattaggatttaggtaggtattttttttttttttattattttttttttttttatggtttttataggtttttttttttttattaaaatttgtttttatgtttataataatttttagtttaaagaaaatttagaaacgtaaatttttgttttagaaataattaaaaatagttttttttattggtttttttgttttagattttagttatattttaatttttagtaagagtttagtgaatattgttgaatttaaggagtgtttttttgaaggggtggtaatggtataggggttgatttcggatgagtttaggagtagtcgcggcggttcgcgtcggttagttttatttcgcgggaaagtttgtgtgttattttcgttagggtcgggagggggcgtcggtttttaggaaattacgcgcggagtgggaggtgcgtgggtagagtttcggatcgttggagggagtagtgatgagttagttttttgtaaggcgttgttcgttcgttaaaattgtttttatttcggttgcgcgttattttggggtggggagggggagaatgggaggggatcggggggtcgcgaatacgttacgttgagtcggttaggttttcgaaattttcgtcgttgttgggaaatcgcgcgtttttgtgtcgtttttgatatagatatatatatatatatatatatatatatatatatatacgttcgcgtagatacgtacgcgcgcgcgcgtcgggagtcggtttttttattttatttttacgtttaaagtacgggatgagttagatttttggaaaataaaaatagacgggagtaacgaaaataaaatcgttcgtaagtttttttagaaacgcgacgtggagggaggtaaggagaggggaagttgtttgggcgttttaagtttttaatttagttttaagatgtttttttttacgaagagggcgtaagtgttttttaggggtttgggacggagaggacgcgttgtggttcgcgttaggcggcggcgttttagggggcgattcgtttttttgtttcgtttagttttgtagttttcggagagggaatttgggttaggtcgggaagttttgagttttcgaggtagttgatagaggttttgagtagcgtcgtgttttttagattttttattttttaaagatatgaatttttttcgttttttatttaggcgtgggtttcgttttcgtttcggagtttagcgtttcgattcgaggataggttgggtcgcgattttttcggtcgcggtgggaaagaggtagaggagatttttgttttttttcgattgataggttggttagagttcgtttagagtagggtcgtgatttttcgatttttatagcgcgtcgcggagcggggattcgacgcggtgtttcggatttttagtgttcggtttttttgggaataaattgttttagttaaataggttattgtgtcgatgggaggacggagatcgcgaaggggtaggggagagggtttttggagtttcgttgtattttatttcgggcggggttttgattttatatagttcgttttttggggtttttttgttatagtgagttattttttcgttagtttttaggggacgggttttggtatatttttaagggttaggaaatagttcgcgttgattttagcgtttagttaggtggtggatatagtggcgtaaaggatttgaatttgggatttgggttagggcggcgtagattggattttagagttattcgggatcgattttagacgaatttattttatttcgtttttttcgttttttttgggtttttttagggatacggattttatttttacgtcgcgagtaaattgcgtttggggtttggagttaatcgtagttaaaggcgagtttttagaacggtttcgtttacgttttttttcgttttagttgggtttttaggggcggggagcgtgattagggatttttgtatttgtaatttcgtttttcgtttcggggttttcggtatatttcgattttcgttattcgcgtatttagagatatcgtgtgcgtaagtcgagcgcgtatcgatttacgttcgttatttatttgtcgtagaaatatttgtgaacgtagtatatattcgcgaatacgtattttcgcggatacgtagggatatacgcgggtacgtttggttcggttttgggtcgtcggttcggggttttttgttgtttgtcgtcgtttttttattttttttgagtgtttcggtgtttcggcgaattcgcgtttagtttcggttttataaggaattgatttcggtagttgtttatattttagttggcgtagtttagcgcggttttgatatataatcgtttcgttcgggttcgttttaaggaggcgggattcgcgttcggtttatcgcgtcgttcgggatcgtttttttcggggtttaggtttttggttgtttagcgtcgagttagttgagtttggtcgagttttagtaggttagtcggtttcggaatttcgcgtgttgtaggagggtatttttttttttttttagtttttcgtttgcgttggtgcgttggatatatttttttacgaagtgagttataaatttggttttttttatttggagaatgagttggtattttttaggaggatatagtattgttagaatgagttttttttttggtttatcgttgatttattttggtaggtaaggatttatttaatgtagttgaaaagattaggaggatgatattaatatataaaaatttataaattataaaaacgatgtatttttttgtaatttttagaaaagttttataatattatttttatttttattgattttttattaggttattttttttagaagtatttggagtatttagatattttaataaagtattttgaggttagaatgattttttagtttcgtttattttatagatgaggaaattgaggtttattgaatttaagtatataaagttgttgattgttatatgttttcgggaaggagggaattggagagattattaaaaaagggtaatttgatttttagggaaatagaagaattggatattgaattaatttttttatattttatatttatttgaatagaagaaatttttgtggttgtagtagttttgttggttaggaaggggaggatttgacgagtgagttgtttgttttttgaatattttttatatagttcgtatatattgttggggaaattggggtttagagaagtttggtgatttaatttagattatgtagtaaagaaatgattatagttggaatataggatttttgttttttgttcggggttttttgtttgttattttttatttttgtggttttaaaatgataaaaatgttaaataatttttatttgtagatggtttatggagatgatataaataaaggataattttggaagtgtttattggtttttttgatagatatagaaatgagtgatgtgtttattcgtttttatttattttatatttttgattttttttggattttatttttgttttttttttgtgtttgaaatatttgtagttttgtttttaaaaaattgtagaggatggattgtttatttgaatagaaattttattaaaaaatagaatttaggtttggagtagttataattattgatattttaggttgtttagagtttggaaatttttgtttagatatgtttagttttttttttttaacgtagttgatttcggggaggataggttttttttttttttgttgtggggatgggagtttaggtaggggtgggaggatatagtagtagatttttttggtttgtttcgttttggagttaggttaggattgtggttaaattttagaaaggttacggtatagttttaggattttattttaagttttattgttgtttgtattttcgtttttatatatttaaattatttaaagggttggttgttaaatgtttagtagaggataggtatttttttttattgttttgaagtttttttgtttttaggtattttaaatagattagatattgtttgtttgtttattttggggagaaaattttttttaataaaggtttgtaatgaattatttaaatttttgtggtattgagttttataatgaaataattttttttgttgttgtttttgggaaagaattttttttataatttttagtttattattattacgatatttaatattttttaagtatttattaagtgtttagtattatttagtgttttaaaaaaaaaagtttgtaattatgtatttgaatgtgtatttagatatattttaagggaggatttttg

SEQ ID NO: 36 - DNA, 2986 bp, Artificial Sequence
  chemically treated genomic DNA (Homo sapiens)
gagggttttttttagttgttatattttggagcgtatgagatgaggtaggtatataaagtggataagatgtggttaagaaaataagttatatattaagtttatttgtagtataggtgtttaagaaaattttgttgttgtgtaatattagaacggaaggttggtttttagtaaaatgtattaattttggtttaaattaagatgatgggtatcgggtatgggggtggggaggtagttgaagatttattgagttttgttttagggtagttttgtttatcgttttattttattttttattacggtgtttaagtttatattgagagagaaatttttagttgtaaaagggagaagagaaacgttggaatattagtatcggacgttaggatatggttgtggtgttttaaaaattattttattatttggagtttgatttcgaggggagtatttttattttttagttttttgaaagtatttattagtatttgaatattgttttgagtttgttggagtagtgaaatttggtgagagagaagggtggaggaaggaaggagttgttgtatttggcggttggatttaggtagaggaaattgttataatttcgggaaagaatagaaaagtagaaagggacgagtttttatacgtagttaatgtttatggttttaattgtgtttgggaaggaagattttgggttaggggtgtattttcgttttttaaaattaaacgtgtttgagatagttataaagtttattaagggatttgagagattagagttttttgttttttttttttaattttgagttttttttttatttttattgagggagagtttgagtttatgataagtgtcgcgtttatttttggttaatttttaaaagaaagacgttcgttttggttttttttttaggtttttagtttttttagggatggtagaaatttttgggttaaggttgagcgaattattgtttattgtttttattagtttttagtaaaggtacgtcggcgggggggcgtttagtttttttagtaaacgtttcgcggtttttttcgtagattacgaggtgggggtcgttggggagggtcgagttgggggtagttcgttatttcggtttttagcgagttgtcggcgattttcgcggttttttggtttaggtttcggtttttcgggcgaggagcgggagggaggtcggggtttaggcgtcgcggcgaattcgttaacgtagcgtcgggtttcgaattttaggtttcgttttaggttttcggtcgtttggttagtttgtttgttttaattttaattttttcgaggttagttagagtaggtttgttggtagtagtatttttttagtagttacgcgattagttaatttttcggcggcgttcggggaggcggcgcgttcgggaacgaggggaggtggcggaatcgcgtcggggttattttaaggtcgcgttcgttagtttcggcggggcggttttcgtcgtcgtaattaatggatttttttttttgtttaaatagattcgtcgtgttaattattttttttttcgttagttttttttttatcgttatattgggttattaaaaaaagggggttcgttttttcggggtgttttttttttttttttttgttttcgtttgtttacggttttgcgatttcgacgtcggtaaggtttggagagcggttgggttcgcgggattcgcgggtttgtattcgtttagattcggacgggttttgttatttttttcgtttgtttggttttttttttttttcgttttttcgttcgttagtttatttgattagcggagattcggcggtcgggtcggggttttttcgtagtttttgcgcgtttttagagttcgggtcgtggttcgtcggggtttgtgttttttggtttcgagggtagtcgttgggttttcgagaggggttcgggttgcgtaggggcgttttgttttgttcggttttgtttttttgagagtgcgagagaggcggtcgtgtagattcgggagaaagatgttaaacgtgcgagtgtttaacgggagttttagtttggagcggatggacgttaggtaggcggagtattttaagttttcggtttgtaggaattttttcggttcggtggattacgaagagttaattcgggatttggagaagtattgtagagatatggaagaggcgagttagcgtaagtggaatttcgatttttagaattataaatttttagagggtaagtacgagtggtaagaggtggagaagggtagtttgttcgagttttattatagattttcgcggttttttaaaggtgtttgtaaggtgtcggcgtaggagagttaggatgttagcgggagtcgttcggcggcgtttttaattggggtttcggttaattttgaggatacgtatttggtggatttaaagattgattcgtcggatagttagacggggttagcggagtaatgcgtaggaataaggaagcgatttgtaatcgacggtaatgatttttttttaattatagaatgtgtttggggtttcgttttgtttgttggagggtgttaattttagtttgtttttcggcgtattttgatttagttttgggagagttaattttattggttttaggtgtttagtgttatttggtttattgtttgtttgtttgtgatttttaagttagaaattggagatggtaagattcgataatttttttaatttaatatatcgcggttttttttattagtaatttttaggtatgtgataaagttgggatgtttattaacggttcgttttttggttagggaaagagttttggggcggagaatgtattttttgttttttgaaaataattttattttgtgtttttaaaagttattggggatgacggatttaggattgtgggtggaggtagtgggttttttattttttgattatggggttaatttttgttagttattgttttttttaataaagattgtgtgttttttttaaaaattttttttgcgtttaga

SEQ ID NO: 37 - DNA, 2986 bp, Artificial Sequence
  chemically treated genomic DNA (Homo sapiens)
tttaagcgtaggggaaatttttaaaaagaatatataatttttattagaaaaaataatggttggtagaagttggttttatagttaggggatgaaaaatttattatttttatttataattttggattcgttatttttagtggtttttaagggtataaaatgaggttgtttttaaaaaatagaaagtgtatttttcgttttagagttttttttttagttaggaggcggatcgttgataaatattttaattttgttatatatttaggagttgttagtgagagggatcgcgatgtattaagttagggaaattatcggattttattatttttagtttttgatttaaaagttataaatagataagtagtgggttaggtagtattgaatatttaagattaataaagttagtttttttaaagttaaattagaatacgtcgaaaagtaagttaaggttaatattttttagtaggtaaagcggggttttaaatatattttatggttgggaaagggttattatcgtcggttgtaggtcgtttttttatttttgcgtattgtttcgttaatttcgtttggttgttcgacggattagtttttgggtttattaaatgcgtgtttttagagttagtcggagttttaattaaaggcgtcgtcgggcggttttcgttgatattttggtttttttgcgtcggtattttgtaggtatttttggggggtcgcgggggtttgtagtagaattcgggtaagttgtttttttttattttttgttattcgtatttgttttttaggggtttgtgattttgaaaatcgaaattttatttgcgttggttcgttttttttatgtttttgtagtgtttttttaagtttcgggttaatttttcgtggtttatcgggtcgaagaggtttttgtaggtcgagggtttggggtgtttcgtttgtttggcgtttattcgttttaggttagggttttcgttagatattcgtacgtttgatatttttttttcgggtttgtacgatcgtttttttcgtatttttaaaaaaataaaatcgaataaaataaagcgtttttacgtagttcgaatttttttcggaagtttagcgattgttttcggagttaaaagatatagatttcgacgagttacggttcgagttttaggagcgcgtaggggttgcggggaagtttcggttcggtcgtcgagttttcgttgattaaatggattggcgagcgggagggcggagaggagaggggattaggtaagcggagagggtggtaaagttcgttcgagtttgggcgggtgtaagttcgcgggtttcgcgaatttagtcgttttttaaattttgtcggcgtcggagtcgtagagtcgtgagtaagcggggataggggagggggagaaaaatatttcgaaaagacgagttttttttttttagtggtttaatatggcggtggaagggaggttgacgaagaagaaaatgattgatacggcgagtttatttaaatagaggaggagatttattggttgcggcggcgggagtcgtttcgtcgaggttggcgagcgcggttttaaggtggtttcggcgcggtttcgttattttttttcgttttcgagcgcgtcgttttttcgagcgtcgtcgggagattggttggtcgcgtgattgttggaggggtattgttgttaataaatttgttttggttggtttcggagaaattaaaattaagataaataaattagttaaacggtcgggaatttggggcggggtttgaggttcggggttcggcgttgcgttggcgggttcgtcgcggcgtttaagtttcgatttttttttcgtttttcgttcgggaagtcgggatttggattagaggatcgcgaaggtcgtcggtagttcgttaggagtcggggtggcgagttgtttttagttcggttttttttagcggtttttatttcgtggtttgcgggggaggtcgcggagcgtttgttgggggggttgggcgttttttcgtcggcgtgtttttgttgggggttggtggaggtagtgggtaatggttcgtttagttttaatttagaagtttttgttatttttggggaggttgggggtttagggaagaagttaaagcgaacgttttttttttagaaattagttaggagtagacgcggtatttattatgaatttaagtttttttttaatgaaaataagaaaggaatttaagattaaaaaaaaaaaataaaaaattttagttttttaagttttttaataaattttgtagttgttttagatacgtttagttttgaaaaacgagggtatatttttggtttaggatttttttttttaagtatagttaaggttatggatattggttgcgtgtgggaattcgtttttttttatttttttgttttttttcgggattgtagtagttttttttatttgagtttagtcgttaaatataatagtttttttttttttttattttttttttttattagattttattgttttaataaatttagaataatatttagatgttagtgaatgtttttagagggttgaagggtgaaaatatttttttcggggttaaattttagatgatgaaatgatttttaaaatattataattatgttttaacgttcgatattagtattttagcgtttttttttttttttttgtagttggaaattttttttttagtgtgggtttgagtatcgtggtggaaggtaaagtaggacgatgagtagggttgttttgagataaagtttagtggatttttaattgtttttttatttttatgttcggtatttattattttggtttgagttaaagttaatgtattttattggaaattaatttttcgttttaatattatatagtagtaaagtttttttaagtatttatgttatagatgagtttgatgtgtagtttgtttttttagttatattttgtttattttgtgtgtttattttattttatacgttttagaatgtgatagttggaagggattttt

SEQ ID NO: 38 - DNA, 5666 bp, Artificial Sequence
  chemically treated genomic DNA (Homo sapiens)
aaaattagaatttttatttttttgcgtttgttatattttttagtgttgtttaatttttttttgtaagtgagggtggtggagggtgtttataatttttttagggagtaagttttttttggttttttttttttttttttttttttttttttttgagattaagtttcgtttttgttttttaggttggagtgtaatggcgcgatttcggtttattgtaattttcgtttttttttgggtttaagcgatttttttatattagttttcgagtagttgggattataggtatgcgttattaagtttcgttaattttgtattttttagtagagatagggtttcgttatgttggttaggtttgtttcgaatttttggttttaggtgattcgtttgtttcggttttttagaatgttgggattatagacgtgagttatcgtattcggattttttttttatgtaatagtgataattttatttaaagtatttttttttttttttgagtcggagttttattttgttatttaggttggagggtggtggcgcgatttcggtttattgtaatttttgtttttcgggtttaagcgatttttttgttttagttttttgagtagttggaattatatacgtgcgttattatggttagttaatttttgtatttttagtagagacggggtgttattattttggttaagttggtttcgaatttttgattttaggtgatttgttcgtttcggttttttaaagtgttgggattataggtgtgagttatcgcgttttgttttaaagtattttttttttatgttttaaaataagattgtaagttagtttttaaagcggataatttaagagttaataggtattagtttaggatgtgtggtattgtttttaaggtttatatgtattaatatattatttaaatttataataatttttataaagtagggggtatttatatttttttttttttttataattacgaaaaatgtaaggtatttttagtaggaaagagaaatgtgagaagtgtgaaggagataggatagtatttgaagttggtttttggattattgtgtaattttgtttttagaatattgagtattttttttggtttaggaattatgattttgagaatggagttcgtttttttaatgattttttttttatttttttatttgtttataggtagaattttttttcgttcgtattaaataaattttatttttttagagtttgtttttatattaggtaatgtatacgtttgagaaatttttgttttagatagtcgttttatacgtaggaggggaaggggaggggaaggagagagtagttcgattttttaaaaggaattttttgaattagggtttttgatttagtgaatttcgcgtttttgaaaattaagggttgagggggtagggggatattttttagtcgtataggtgatttcgattttcggtggggtttttataattaggaaagaatagttttgtttttttttatgattaaaagaagaagttatattttttttatgatattaaatatttcgatttaatttggtagttaggaaggttgtatcgcggaggaaggaaacggggcgggggcggatttttttttaatagagtgaacgtatttaaatacgtttttgttggtaggcgggggagcgcggttgggagtagggaggtcggagggcggtgtggggggtaggtggggaggagtttagttttttttttttgttaacgttggttttggcgagggttgttttcggttggtgttttcgggggagatttaatttggggcgattttaggggtgttatattcgttaagtgttcggagttaatagtatttttttcgagtattcgtttacggcgtttttttgtttggaaagatatcgcggtttttttagaggatttgagggatagggtcggagggggttttttcgttagtatcggaggaagaaagaggaggggttggttggttattagagggtggggcggatcgcgtgcgttcggcggttgcggagagggggagagtaggtagcgggcggcggggagtagtatggagtcggcggcggggagtagtatggagttttcggttgattggttggttacggtcgcggttcggggtcgggtagaggaggtgcgggcgttgttggaggcgggggcgttgtttaacgtatcgaatagttacggtcggaggtcgatttaggtgggtagagggtttgtagcgggagtaggggatggcgggcgattttggaggacgaagtttgtaggggaattggaattaggtagcgtttcgatttttcggaaaaaggggaggttttttggggagtttttagaaggggtttgtaattatagatttttttttggcgacgttttgggggtttgggaagttaaggaagaggaatgaggagttacgcgcgtatagatttttcgaatgttgagaagatttgaaggggggaatatatttgtattagatggaagtatgttttttattagatataaaatttacgaacgtttgggataaaaagggagttttaaagaaatgtaagatgtgttgggattatttagtttttaatttatagatatttggatggagtttattttttttattaggagggattattagtggaaatttgtggtgtatgttggaataaatatcgaatataaattttgatcgaaattatttagaagcggtcgggcgcggtgttttacgttttgtaatttttttattttgggagattaaggcggggggaattatttgaggtcgggagttcgagattagtttggttaataggtgaaatttcgtttttattaaaaatataaaaagtagtcgggggtggtggtaggcgtttgtaattttagttattcgggaggttgaggtaggagaatcgtttgaattcgggaggttgaggttgtagtgaatagcgagatggagttattttattttagtttgggtgatagagtgagattttgtcgaaagaaagaaagagagaaagagagagagaaaaattatttagaagtaattatatattgtgtttatttttaattgagtagggtaaataaatatatgtttgttgtaggaatttaggaaataatgagttatatttatgtgattattttagaggtaatatgtagttattattttgggaatatttgttaatatttttgtttttttattatttttagtttatttgatatagtttatttgtgataagagtttttaattttttatttttgaatagaggtgttttttttttttttatttttgttttgtgagggagttaggggaggatttaaaagtaattaatatatgggtaatttagtatttttaaaattttgttaatagtttgaattcgggagtttggttttgtagttttataatattttagaagagattttatttgtttaaaaataaaaaggaaaaagaaaagtggatagttttgataatttttaatggagaagggagaagaatatgtagaaaaggggaaatgatgttggtttagaattttaattatattggtgtttaatataggaatatttatttatataatattttaaagtattaaatttatattagtatattattaaatggatatattattaaatgggtttaagtattttatatattttaatttaattgatttattttttttttgttttggatttttattatgatttaaatatttatatatgggttattttttagattttttatattatgaaatataagaaaaatttttaaggttagttttatgattaagacgaaggattttattgaatatataaaataataaatatattgtaatattttgtttttttttttgtagttgtaatttggtttgtttatattttttttttgtttttttgaaaattgagttagttttatttttttaggataggatttaataattataatataatttagtataattttttgatttaggtaaattatgtaatttgtgtttagtatgaaatgtatttaaaaataagtaatttttttttaatattattatttttaaattaatataataaataatagttattttaaaataaattgtttatttttattatgtagtatttaaattttaaggttgttatgattgtagatagtattttaaaatttttttttggaaatggttttgtttttaagatgatttaggaattaaagaggtgattattttttgtttaatgaatttttaaattataaatttgggaagtgttttagttttttattgttgttgttataaattattataaatgtgttagttaaaataaatataaaattattattttatagttttagagattagaagttaaaaatgggtttataaggttttattttttttggaaattttaaggggtaatttgtttttttgttttttttagtttttagtgattattaaattttttggtttatggtttttgtattttttttgtggtttgtgtttttatttttgtattttttttttgattgtgattttttaataaaaatatttggggttatgttgggtttattttgaaaattttggataattttttttaagattattaattaaattatatttgtaaagttttttttgttatataagttaatgtattaaaagtttttgaggattaggatatagatattgggggtgggggggtattatttagtttattataggaaggaattttagggttaattaaattagtttttttattttatatttgaagaaattgaagttttggaattggagagtattatgttaaatgaaataagttaaatatagaaagataaatattatatgtttttatttatttgtgaaatataaaataattatatttttagtagtaaagagtagaatggtggttattagagttggggggtgggaggaatggggagatggtaattaagatataaagttttagttaagatgggaggaataagtttgattgttttttttgagatgtgttttatagtatgatgaatatagttaaatagtaaattttaaatgtttttatttgataaaaatgttaaatatttgagatgatggataggttatttagtttgatttaataattttttattgtgtttaaagattataattttatattgtattatataaatatatataattgtattattttaatatataattttaaaattaatataatgaaaaagaaattgaagtttaatatttttagaagttaagtgtaatttaaaagttttgtgagaatttgttttaataaataaataagttttttttttttaataattattatattttgcgtttggatatatagtagtgaataaaaaaaaaaaaaaaaaaaaaaatttttaggtttaatataattttaggaagaaattttagtagttgtattttaggggaaatataggaagttagtttggagtaaaagttagtttgtttttgtttttttgttattttgttcgtgttttatagtgttttttgtttgtgacgatagtttcgtagaagttcggaggatataatggaatttattgtgtattgaagaatggatagagaatttaagaaggaaattggaaattggaagtaaatgtaggggtaattagatatttggggtttgtgtgggggtttgtttggcggtgagggggttttatataagtttttttttcgttatgtcggtttttattttggttttgattattttgttttttttggtagg

SEQ ID NO: 39 - DNA, 5666 bp, Artificial Sequence
  chemically treated genomic DNA (Homo sapiens)
tttgttagagagaatagaatggttagagttagggtgggggtcggtatgacggaaaggaagtttgtgtagagtttttttatcgttaagtagatttttatataagttttaggtgtttaattatttttatatttgtttttagtttttaattttttttttgagttttttatttattttttagtatataatgaattttattatatttttcgaatttttgcggagttgtcgttataggtagagagtattgtgaggtacgggtaaaatagtaaaggggtagggatagattgatttttattttaggttaattttttgtattttttttgagatataattattgaaatttttttttgaaattatgttaggtttggagatttttttttttttttttttttttgtttattgttgtatatttaagcgtagaatgtggtaattgttaaaaagagaaaatttgtttgtttgttaaaataaatttttataaaatttttaagttatatttagtttttgggaatgttgaattttaattttttttttattatattagttttaaaattatatattgggatagtatagttgtatatatttatgtggtataatatgaagttatgatttttgaatataatggggaattattaagttaagttaagtaatttatttattattttaaatatttgatatttttgttaaatgagagtatttgggatttattatttagttatatttattatgttatgaaatatattttaaaaaaaataattaaatttattttttttattttaattgaggttttatattttgattattatttttttattttttttattttttagttttagtaattattattttattttttattgttaagaatgtaattgttttatattttatagataagtgagaatatgtgatatttgtttttttgtgtttggtttattttatttagtataatgttttttaattttaaaattttaatttttttaagtataaaataagaaggttagtttaattaattttaaaatttttttttgtggtaggttgaataatgtttttttatttttaatgtttatgttttaatttttaaaaatttttaatatattaatttatgtggtaaaagaggttttgtagatgtgatttaattaatggttttgagggagattatttagaatttttagggtgggtttaatataattttaagtgtttttattagagggttatagttagagagaagatataagaatggaagtataggttatagagaaaatatagagattatgagttaaggaatttgatggttattagaagttggaaaagataaggaaatagattgttttttagagtttttaaaaggaatgaaattttgtggatttatttttgatttttgatttttagaattgtaaaataataattttgtgtttgttttagttaatatatttgtgataatttgtaatagtagtagtaggaaattaaaatattttttaggtttatgatttgagagtttattaaataagagatggttatttttttggtttttaaattattttggaaataaagttatttttagagaggaattttaaaatattgtttgtagttatagtaattttaaaatttgagtgttgtatggtggaagtagataatttattttaggataattgttatttgttatattagtttgaggatggtggtgttaaagaggagttatttatttttaggtatattttatattaaatataaattgtataatttgtttaaattaaggaattatattaaattatattatggttattaaattttgttttgagaaagtgaaattgatttagtttttaaagagataaagagaaagtataagtaaattaaattgtagttataaaaagaaagataaaatgttgtagtatatttattgttttgtgtatttaatgaagtttttcgttttggttataaaattagttttaaaggttttttttatattttatagtatgaaaaatttaaaaagtaatttatatgtaaatatttaaattatgatagaaatttaaagtaaaaagaaaatgaattaattgaattaaaatgtgtaggatgtttaaatttatttgataatatatttatttgataatatattaatatgaatttagtattttaaaatgttatataaataaatgtttttatattaaatattaatgtagttaggattttaagttaatattatttttttttttttatatgttttttttttttttttattaaaaattgttaaaattatttattttttttttttttttttgtttttaaataaataaggttttttttaagatattgtaggattataaagttaaattttcgggtttaagttgttggtaaaattttagagatgttaagttatttatgtattaattatttttaaatttttttttaatttttttataaaataggagtagggagaggagaaatatttttgtttaaaaatgaggaattgaaaatttttattataaataaattatattaagtaagttaaagatagtaaaagagtaaaaatgttagtagatatttttaaaatggtaattatatattatttttggaatgattatatgaatgtggtttattattttttaagtttttatagtaaatatatatttatttgttttatttagttaaaaataaatataatatgtagttgtttttgaataatttttttttttttttttttttttttttttttttcgataaagttttattttgttatttaggttggagtgaagtggttttatttcgttgtttattataattttagtttttcgggtttaagcgatttttttgttttaatttttcgagtagttgggattataggcgtttgttattattttcggttattttttgtatttttagtagaggcgaggttttatttgttggttaggttggtttcgaattttcgattttaggtgatttttttcgttttgattttttaaagtgaagggattataaggcgtgaggtatcgcgttcggtcgtttttgaataatttcgattaaaatttatattcgatatttattttaatatatattatagatttttattgataattttttttagtaagaaagataagttttatttaggtatttgtgaattggaggttaagtagttttagtatattttatatttttttaagattttttttttattttaaacgttcgtaaattttgtatttgataaagagtatatttttatttaatataaatatgtttttttttttagatttttttagtattcgagagatttgtacgcgcgtggttttttattttttttttttggttttttaagtttttagggcgtcgttaggaggaggtttgtgattataaattttttttgaaaattttttaggaagtttttttttttttcggagaatcgaagcgttatttgattttaatttttttgtaaatttcgttttttagagtcgttcgttattttttgttttcgttgtagattttttatttatttggatcggttttcgatcgtaattattcggtgcgttgggtagcgttttcgtttttagtagcgttcgtattttttttattcgatttcgggtcgcggtcgtggttagttagttagtcgaaggttttatgttgtttttcgtcgtcggttttatgttgtttttcgtcgttcgttgtttgtttttttttttttcgtagtcgtcgagcgtacgcggttcgttttattttttggtgattagttagtttttttttttttttttttcggtgttggcggaagagtttttttcgattttgttttttaaattttttggagggatcgcggtatttttttaggtaaggggacgtcgtgagcgagtgttcggaggaggtgttattaatttcgagtatttagcgaatgtggtatttttgaagtcgttttaggttgggtttttttcgggggtattagtcggaagtagttttcgttagagttagcgttggtaaggaaggaggattgggtttttttttatttgttttttatatcgtttttcggtttttttgtttttagtcgcgttttttcgtttgttagtaaaggcgtgtttgagtgcgtttattttgttaaaaagaaattcgttttcgtttcgtttttttttttcgcgatataatttttttaattgttaaattgaatcggggtgtttggtgttatagggaaagtatggtttttttttttaattataagaaaaagtaaaattatttttttttagttgtgagagttttatcgagaatcgaaattatttgtacgattagaaagtgtttttttatttttttaatttttgatttttaggagcgcggggtttattaagttagaaattttagtttaaaggattttttttggagagtcggattgtttttttttttttttttttttttttttttgcgtgtaaaacggttgtttggggtaagggttttttagacgtgtatattgtttggtataagagtagattttgaaaagatgaggtttatttaatacggacgggggagaattttgtttgtaggtagataggaaaatggggagggagttattggaaggacggattttatttttaaagttataatttttagattagaaaaagtgtttagtgttttagaagtagagttgtatagtgatttaaagattagttttaaatattgttttgttttttttatattttttatattttttttttttattgaaaatattttgtatttttcgtaattataaagggggaagggaatatgagtgttttttgttttataggggttgttgtgagtttaaatgatgtattaatatatataagttttaagaatagtgttatatattttaagttaatatttgttagtttttgaattattcgttttgaggattggtttgtaattttgttttgaggtatagaaagaaaatgttttggagtaggacgcggtggtttatatttgtaattttagtattttgggaagtcgaggcgggtagattatttgaggttaggagttcgaggttagtttggttaaaatggtgatatttcgtttttattaaaaatataaaaattagttggttatggtggcgtacgtgtgtaattttagttatttaggaggttgaggtaggagaatcgtttgaattcgggaggtagaggttgtagtaagtcgagatcgcgttattattttttagtttgggtgatagaatgagatttcgatttaaaaaaaaaaaaaaatgttttggatagaattattattattatataaaaggaaagttcggatgcggtggtttacgtttataattttagtattttgggaggtcgagataggcggattatttgaggttaggagttcgagataagtttgattaatatggcgaaattttgtttttattaaaaaatataaaattagcggggtttggtggcgtatgtttgtaattttagttattcggaggttgatgtaggagaatcgtttgaatttaggagaaggcggaggttgtagtgagtcgagatcgcgttattgtattttagtttgggagataagagcgaaatttggttttaagaaaaaaagaaagaaagaaagaaagaaagattaagaagaatttattttttgaaaagattatgggtattttttattatttttatttataaagaaaagttaaatagtattaaagagtataataagcgtaaggaggtaaaagttttaatttt

SEQ ID NO: 40 - DNA, 5085 bp, Artificial Sequence
  chemically treated genomic DNA (Homo sapiens)
tgatggttgtataattttgagtatatgaaaaattaatgaattgatattttgagtgagttgtatgatattggaattatattttaataaagtatggtaattgttttaagataggttggaaagagaaagtttgaaaataataataatgatattaataaattagtttatttttttagttttatatatttttgtgtttatatttgtttttgttttatttataatggtttttttgtagttgttatattatattttgttatttgatgttcggtgaatattttatatttgttttttagaattttttttattttttttttatttgtttaatttttatatatttaaaattaattagagtaaattatttattagaataattaattttaaattttagtaatttaatatgataaaggtttgttttttatttatatagtttttttttagatgatcgaggggtttaggttttttatttttagtggtttttttattttttggagttttttgtattttttatatatggttgagataaattatgagttattagtatagttagattttgaggttttataagaaaatttgtaaattatttattttgttttgaataaggtatatttaagatgatgttaaaatatttaatggttttgggttaaatatagtttatgattgtgtatttaaaatatatattgtaatatttttttttttttttattgattttatgaatttagcggggatttattttataagtttaaagataattattttttagattaagaatatttagggtaaaaagtattgtttaatatttttattgaggatgttatgatgtagtatattgtataagttggagttaaaggaaattttttttaaagtgttatttattaaaaattggaatatattttttaagataaatcgaagtgtggtatataatatttaaatttttattatagatatagaggtgttattattttttatttttaaatttttttgttacgttgaggatatttaagaggagtaggatatgttggtcgtagtaggagaaatttgaaagtatttatttttatggaatttataagggagagaattttttattttagtatcgtttttgatatatttattattttaaaagataatgtagttaaatgtttttttttgtgttaaatttttataaaattgaaattttaaaatggtgataaaaattttatttttgatagaatttatttatttttttaattagatagggtataatttttaatttgtaaaataaaacgtaatatgtttatgaggttttattttaaagaatttgttattgagagtagtatttagaataacgggtggaaatgttaattttagagttttagattttatcggtaattggggtagggaggggttttgggcggggtttttttagaggaggaggcgttgttagaaagttgtttggttagtttatagttgttattaatcggggtaagttttgttgtatttgtgcgtgtgggtggtatttttaatgagaattagttttatttgttatttgagtgaaatttataattcgaggcggttagtgttttcgtattattgggatttgagattttcggagatgattgtcgttcgtagtacggagttagtagaagttcgattttttttgggaatgggttgtatcgagaggttcgattagttttagggttttagtgagggggtagtggaatttagcgagggattgagagttttatagtatgtacgagtttgatgttagagaaaaagtcgggagataaaggagtcgcgtgttattaaattgtcgtcgtagtcgtagttatttaagtgtcggatttgtgagtattttgcgtttttagttttcggatagaagttggagaatttttttggagaatttttcgagttaggagacgagattttttaataattattatttttttttgcgttttttatttgtcgttcgttgggataaacgatagttatagtttttttgacgataggatggaggttaagggtaggagttgattagcgtcgttttttttcgttttcgatttaggaggtggagattttttcggtttagttatatttaatatttatttttttttttttttgtttttatattttcgaaatttttttttttttttttttttttttttttttggagacgggggaggagaaaaggggagtttagtcgttatgattgagttgaaggtaaagggttttcgggttttttacgtggcgggcggttcgttttttttcgaggtcggatttttattgttgtgtcgtttagtcgtaggttcgttttcggggagttagatttcggatattttgtttgaagtttcggttatatttatttttttggacgggttatttttttttcggttttgttagggataggattttttcgacgaaaagacgtaggattagtagtcgttgtcggacgtggagggcgtatattttagagttgaagttataaggggtgttggaggtagtagttttagttttttagaaaaggatagcggattgttggatagtgttttggatattttgttggcgtttttaggtttcgggtagagttaatttagttttttcgtttgcgaggttattagtttttggtgtttgtttggtttcgaatttttcgaagatttatcggttgttttcgttatttagcgggtgttgttttcgtttatgagtcggttcgggtgtaaggttggagatagtttcgggacggtagttgtttataaagtgttgtttcggggtttgttattagttcggtagttgttgttttcggtttttgagagtttttattggttcggggttttagtgaagtcgttttcgtaggtcgttgcggtggaggttgaggaggaggatggttttgagttcgaggagtttgcgggttcgtttttgaagggtaaatttcgggttttgggtggcgcggcggttggaggaggagtcgcggttgtttcgtcggggcggtagtaggaggcgtcgttttggtttttaaggaagattttcgttttttagcgtttagggtcgttttggtggagtaggacgcgtcgatcgttcgggacgttttcgttggttattacggtgatggattttatttacgtgtttattttgttttttaattacgttttattggtagttcgtattcggtagttgttggaagacgaaagttacgacggcggggtcggggttgttagcgtttttgtttcgtcgcggagtttattttgtgtttcgtttatttcggtcgttgtaggcgattttttcgattgcgcgtattcgttcgacgtcgagtttaaggacgacgcgtattttttttatagcgatttttagtcgttcgttttaaagataaaggaggaggaggaaggcgcggaggttttcgcgcgtttttcgcgtttttattttgtggtcggtgttaatttcgtagttttttcggatttttcgttggggttatcgttttcgttgtcgtcgcgagcgattttatttagattcggggaagcggcggtgacggtcgtattcgttagtgttttagtttcgtttgcgtttttttcggggtcgattttggagtgtattttgtataaagcggagggcgcgtcgttttagtagggttcgttcgcgtcgtcgttttgtaaggcgtcgggcgcgagcggttgtttgttttcgcgggacggtttgttttttattttcgtttttgtcgtcgtcgtcggggcggttttcgcgttttattttgtattcggttttaacgggttttcgtagttcggttattaggtcgtcgtgtttaaggagggtttgtcgtaggtttattcgttttattttaattatttgaggtgagggttcgggacggggtacgtttagcgcgttcgggagtagcggtttcgttggcggcggcggtcgttaatttttagttttagttttagcgtatcgttgcgtttttcggggcggtcggagagggtgggtagcgggatatagtataggggtagttgtttttttttttttttttttttttttttatttttggggatacgaaggtgggcgtagaatatattatttttggggcgtgtttttttgaaagttgtttttttgtttgttttttaatttttcgaattttttagatttcgaagtagaattaatttcgatttaaaacgtgtagcgttatattaggttcgttgtagtttagtggggtagaaagtgcgcggcgagttgggggttttatgaaatgttttttttttagaagaaggacgtttattaggagtgtttgttttggagaggagttaaggtatcgttttttcgggaggggtgggatttgagaggtggtcggttagaatcgaaagtagtattattttagggatttgaatattttagtggtttagtttttttaagaattttaagattaaaattaagtttacgtgggaaatgtttaaattgtggatttaaacgtttgttattgtattgtatcgtttttttattattgtttgttatttattataattttttttatatataggtttaaaaaatattattttgtatattgaagtaatggaatgtaaaaaaagaatgttttgtttggaattttatgttgtgaataggtaaaatagtgttagtgtattggataatattttaaaatgataaatatatatttgtttaagtaagtaatgattatagggttgtgttttaaaaatttaaaattaaaatattgtaaagtattatcgaatttttaaagttaaattatatttgttttgatttagtatatagataggaaggatataatattttatttgttaaagattaaattgtttttatataaagagttttgtagaaagatttttttttaatcgattttaattttttaggatataatattatatattaattattgtttttttatattggtgttattgatgaatggttaattatttgtaagtatggtgaatttagttacggatagtttattattaagtttagtttgtatgttttttaagtgtatatatatagttttgtttttaaaatttttttttattttgttaatattggtttaagaaatttttagtattagatagtggtgtatttaaaaataaatggagtattttgttttgtattttaagg

SEQ ID NO: 41 - DNA, 5085 bp, Artificial Sequence
  chemically treated genomic DNA (Homo sapiens)
ttttgaaatgtaaaataaagtattttatttatttttaagtgtattattatttaatattaaaggttttttaaattagtattaatagggtgaaaggagattttaaaaatagaattgtatatatatatttgaaagatatgtaaattaaatttggtaatagattattcgtaattggatttattatatttgtaaatgattagttatttattagtagtattaatataaaagaataataattagtgtataatattatgttttagaaagttaaagtcggttaaaaggaaatttttttataaaattttttatatagaaataatttagtttttgataaatgaaatgttatgttttttttgtttgtatgttgggttaaaataaatatggtttggttttaaaagttcgatggtattttgtaatgttttggttttgagtttttaaaatataattttgtaattattgtttatttaagtaagtatatgtttgttattttaaagtattgtttaatatattgatattgttttgtttatttataatataagattttaaatagagtattttttttttatattttattattttagtatgtaaagtagtgttttttaaatttgtatataaaaaaaattgtagtgaatagtaagtaataataagaaaacggtgtaatgtagtgataggcgtttaaatttatagtttaaatattttttacgtgaatttaattttaattttgagatttttaagaaaattgagttattgaagtgtttaaatttttaagatggtgttgttttcggttttggtcggttattttttaagttttatttttttcgggggaacggtgttttaatttttttttaagataagtatttttggtaaacgttttttttttaagaaagaagtattttataaagtttttaattcgtcgcgtattttttgttttattgggttatagcggatttagtgtgacgttgtacgttttaaatcggggttggttttgtttcggaatttggaagattcggaaagttaaaaaataaataaaaaaatagtttttagggaggtacgttttaaaaatagtatattttgcgtttattttcgtgtttttaagagtgaggagaggagggaagaagaagggaggtaattgtttttgtgttgtgtttcgttgtttatttttttcggtcgtttcggggagcgtagcggtgcgttggggttggggttgagggttggcggtcgtcgtcgttaacggaatcgttattttcggacgcgttgggcgtgtttcgtttcgggtttttattttaggtagttgagatagggcgggtagatttgcggtaggttttttttgagtacggcggtttggtagtcgagttgcgggagttcgttgaggtcgagtgtagggtagagcgcgggggtcgtttcggcggcggcggtagaggcggaggtggagggtaggtcgtttcgcgggagtaggtagtcgttcgcgttcggcgttttgtagggcggcggcgcgaacgggttttgttggggcggcgcgtttttcgttttgtataggatgtattttagggtcgatttcgaggaggacgtagacgagattgaggtattggcgggtgcggtcgttatcgtcgttttttcgggtttggatggggtcgttcgcggcggtagcgggggcggtggttttaacgggaaattcgggaaggttgcggggttggtatcggttataaggtaggaacgcggggagcgcgcggaggttttcgcgttttttttttttttttttatttttagagcgggcggttggaagtcgttatagagagggtacgcgtcgtttttgggttcggcgtcgggcgggtacgcgtagtcggggaagtcgtttatagcgatcggggtggacgaggtatagggtgaatttcgcggcggggtaaaggcgttggtagtttcggtttcgtcgtcgtaattttcgttttttagtagttgtcgagtgcgggttgttaataaggcgtgattgagaggtaggataggtacgtggatgaaatttattatcgtggtggttagcggggacgtttcggacgatcggcgcgttttgttttattagggcgattttgggcgttgagaagcgggaattttttttggggattagggcgacgttttttgttgtcgtttcggcgggatagtcgcggttttttttttagtcgtcgcgttatttagagttcgaggtttgttttttagaagcggattcgtagatttttcggatttagagttatttttttttttaatttttatcgtagcggtttgcggagacggttttattggggtttcggattagtgagggtttttagaggtcgggagtagtagttgtcgggttggtgataggtttcggggtagtattttatgggtagttgtcgtttcggagttgtttttaattttgtattcggatcggtttatgagcggggataatattcgttgggtggcgggggtagtcggtggattttcgggaagttcggggttaaataggtattaagagttggtgatttcgtaggcgggagggttgggttggttttgttcgggatttgagggcgttaatagagtgtttaagatattgtttagtagttcgttgtttttttttgggggattagaattgttgtttttagtattttttgtagttttagttttggaatatgcgttttttacgttcgatagcgattgttggttttgcgttttttcgtcggaggggttttgtttttggtagggtcgagggaagagtagttcgtttagggagataggtatggtcgaaattttaggtaaggtgttcgaggtttggtttttcgggaacggatttgcggttgggcgatatagtagtggggattcgatttcgggggagggcgggtcgttcgttacgtggggagttcggggattttttgtttttagtttagttatgacgattggatttttttttttttttttttcgtttttagggaggagggaaaagggaaggaggagggggtttcgggaatataggggtagagggaggagaaagtgggtgttgaatgtggttggatcggagggatttttattttttgggtcgggggcgggggagggcggcgttggttagtttttgtttttggtttttattttgtcgttaggggaattgtggttgtcgtttgttttagcgagcggtaagtggggagcgtaagaaaaagtagtaattgttaggagatttcgttttttaattcggggagttttttaagagagttttttaatttttgttcgaggattggagacgtagagtatttataagttcggtatttgagtggttgcggttgcgacggtaatttagtgatacgcggtttttttatttttcgattttttttttggtattaaattcgtgtatgttgtgaagtttttagtttttcgttgagttttattgtttttttattaaaattttggggttagtcggatttttcggtatagtttatttttaggaagggtcggatttttgttggtttcgtattgcgggcgatagttattttcgaagattttagattttagtagtgcgggagtattagtcgtttcgggttgtagattttatttaaatgataagtgaagttagtttttattgagaatgttatttatacgtataaatataataaggtttatttcgattagtgatagttgtggattggttagatagttttttaataacgtttttttttttagggaggtttcgtttaaagtttttttttattttaattatcggtaggatttgaaattttggagttggtatttttattcgttattttgaatgttatttttaatagtaggttttttgggatggaattttataagtatattacgttttgttttgtaaattaagaattatgttttatttaattggaaaaatgaatagattttattagaagtagaatttttgttattattttaagattttagttttgtaaagatttaatatagaggaagatatttggttatattattttttaaaataataaatgtattaaggacgatattaaaataagaaatttttttttttatgagttttataaaagtgaatgtttttaagttttttttgttgcgattaatatgttttgttttttttgagtatttttagcgtgataaagaaatttgggagtgggagatggtaatatttttgtatttatgatggaagtttggatgttgtgtgttatatttcgatttgttttaaggaatgtgttttaatttttagtaaatagtattttaaggaaagttttttttagttttagtttatatagtgtgttatattataatatttttagtagagatgttgaatagtattttttattttaaatatttttagtttgaaaagtaattatttttgagtttataaaatggattttcgttaaatttatgaagttagtagaaaaagggaagaatattgtaatatatattttagatatatagttataaattgtatttgatttaagattattgggtattttaatattattttaaatatattttgtttaaaatagagtgaatgatttgtaaatttttttgtaggattttaaggtttagttgtgttaatgatttatagtttattttaattatgtataaagaatgtagaagattttagaaggtgggggagttattagaggtaaggagtttggatttttcgattatttggggaggggttatatgagtgagaaataaatttttattatgttaggttattaggatttggagttggttgttttagtaaatagtttattttgattaattttagatatgtggaagttaggtagatagaggaaaggtaaagagaattttgggaagtaggtatagaatgtttatcgggtattaaatggtaggatataatatggtaattgtaaggggattattatgaatagaataggagtaagtgtgggtatagaagtatatgagattagagaagtaaattaatttattaatattattattgttgtttttaggttttttttttttagtttattttaaaatagttattatgttttattgaggtgtaattttagtattatatagtttatttaaagtattagtttattgattttttatgtatttagagttgtgtaattatta

SEQ ID NO: 42 - DNA, 8222 bp, Artificial Sequence
  chemically treated genomic DNA (Homo sapiens)
aagattagatgtaattttgttaagtagttattttgtaaattgtatagttttatgtagatggaaggaagggtttgtgtttattttgattatagtattaaataaattgtattgtatttttattttttttagttatttttttaaaaagattttgagttttttgaatataggaaggtgttttatttgattttgttatttttagtatgtagtagtgtttgatatatagtaggtgttttattattgtgagagggatggatggatgggtggagttatagatggatagaaggatagatggagggatgggtggatgatggatggatagatggatggaggggggatgatgaatggagggataatgagtggatgaatgagggaatgggtggatggatggatggagggatggaggaatagatagatagatggagggatgggtgggtgatggatggatagatggatggagggagggatgatgaatggagggataatgaatggatgaatgaggggatgggtggatggatgaatggagggatgatgggtggatgaatgaattgagggatggatggatgaatatatggatggatggatagatggatagatggaggaattggtggattttggatggatgggtggatggatagatgaatgaatgtttggatagataaagagatgatggatagatgaatagatgaattaagggatgtcggatagatggagggattgatagatgttggatggatgggtggtggatggatagatgagtgaatgtatggatagataaagagatgatggatggatgaattaagggatgatagatggatggatggatgagtaattggatggataagtggataaatggatagatggttgaatatttgaatggattgaaggaggatgtatggatgtaagataaggttaattattttttattttttttttttgtaaaattatttatttatttatttaataaatatttatttagtttaaatttggtataaagtattatgtgaggtttaagagatacgtgggttaataaaatagagtttttgtttttttgaaaattgtaaagaaaggggcgtggttttttgagtttaaattttaattttgttagcgattagttgtatattagtgatgtttttttatttttttttaattaaatagggataatgttagtatttattatattgggaggttttgcggggattaaatgagttattaaatgttaagtgtttgggatagggtttggtatttagtaaagttttttgtgagtgttggttgttattattttaatggagaagatggtatgaaaattaggaaataggatgttttttgggaagtaatgtaataggaatttatataaagaaaggaaaggaggaagtaattagtggtgttttaaaggagtatgttaagaaaaattttttagagggaaatttttgagtagggttatgaaaataggagttttttaagagattgtggatttgtttgggattatttggttataagtataaaattattcggtttttttttgttatttttggttgggtgaggggtttttggtaaaggggtagaaggtgcgtgagaggttgcgaatggttaggattgttttggggttagtcggggtatttggtggttaagtttagaaatatgataggtttttttgggagggttgatcgtagggagcgttgggttttaggttgttggcgtcggtttttgtggtgttttttttgtcggttatgagagtttagatagtgtttaattttttttttttttttttatacgtataattattttattttttgtggtttgagttgttttgtttcgttataatggtatttgttttaaaatagttttttatgtgagggttagagaaaggaaaagattagattttttttggatgagagagagaaagtgaaggagggtaggggagggggatagcgagttattgagcgatttttgttaagtattttagaaggtataaaaacgtttttgggattaggtagttttaaattttagttgttggggttaggatatttagtgagtttatatttgttttttttgttttttttagattgcgttatggggtttagcgacggggaatggtagttggtgttgaacgtttgggggaaggtggaggttgatattttaggttatgggtaggaagtttttattaggtaaaaggaagagattttattgtttttgttatttatattttaagattaagggtgtttagttgtaaggtggaaagtttgtacgtggggtaggttagttggttgtattagttaagggtgttagaacggttatttgttttttttttgtttttaagtgttagggattggatttaggagagggaaaggagttattttaggttgatgttagtagttggaggaagtatgagaattaaatttaggatgtttagagtttattaggaagaattttagaattatagatagttagagttaataagggttttgagagattttgtatagttattttttttataggatgaggataaaaagggattgagaaggggaggatatttttagagttatagtttattaaatgtttttaaagtgttaaggttaagatatgttttttaaggggagatagatttggttttagatttggttttgttattgagttattgggtgatttttgggaaagtatttaatttttcggagttttaatttttttttttgtatagtgaggggatattttaatatttatattttagaggagatgtgagaattaaataaaataatgtatgtaagaggtttggtatggtttttggtatatattgagttttagaaatgttagtagttattattgatgaagtttaggttagggattttttaaagtattgtaattagagaatagaagatagaggtttattagtgattttcgatgttgagtatgtttttagtttgagaggtttgaatgatgtggtttgtaagtatattttgttttttattataagggattttagaatatattaaagaaaataaaattttgaggtttgtaaatagagggtggttgtggtttgtatatagaagtttattttttcgttgttttttattttaaaggtgatatattttttttttggtttttttttttattattttgagttggtttttttagaagtttaataggttaagaattaacgtttttgttaacgggaggaaggaagtgggcgtcggggttgttataagtgaatggattgtttttggtattttaatttgtagattaatttttttttatttgtgagttagagttaattttttttttttatttcgtcgttttttgggttttttttgggttttatttatttttagttttcgtttatttcgggagtatggttggttttggaagtttcgttatttaaaatttgagtcgttggtgattcgtgttagaaatatttgaggaagaagagttgagttttttgttgattgtaatggtatcgtagtgtgtttatgtttttttttttttgtttttgttatttggttgtaataggttttgttggattgtataaagtgttttttcgattaatatatatatataaattgatattaatttttgggttatagatatgttatgtgattttggataagtttttttatttttgggttttatttttttgttggttgagcgaagggattgttttttatggttttcgagatttcgttttacgtttatgttttagaattttttgagtttttgatgtatttttgtttttggcgaggaggtaggatagttaggcgtggaggtaggagattagagtttgtagagtagaggtttttttaaggtgatttgggagttgttagatttaaggtagtgatttgtacgttttgaaagagatttagggtgttgtattattagggaggattacggaggtttgtcgggggtcgggagttagggataagatataatgtttagggttgttggtttttgaaaggttttagaggtttggtgttttgttacgttttaggttgtgggatatgtagagattgtgttagttggttttgtttttttagtatttggggtatagggaggatggtttttttgggggtagggtggaggaggtttatgtttaggattgtaaatgttattcgtgagagttggtattttgattttgttttagatattgattttggattttttaattgattttgaaaggttatgtttttaattttttgattgtaaaagagggtaaattttatgaagtttagtttgtaaaatattgaggtttttattgtacgtatataaatataaaattttagaatatagaagttggaaggttatttggagattttggttttaggagtggggtttcggaagggagggggttgttgagtgggttttatatttttgttttttttttttttatttaaattttttttgttttatttattttggttgttttggggagcggggggaataaaggattttaaggttataaaatgtttgaaaattattagtggtggttattattttttattatagggaggaattgggtttgagagtttttatcggttaatggtagttataggtaggattaaaaatttttgggtgatggtgatgataatgataatgataatgataatggtagagatgatgaggaggagaaagatagtaattatttatttagtatatattgattgttgggttttgtttaagtgttttatttattgaagtttgataataattttatgaggtagatatgattatgattattatatttaatagagaggttaagtaatttacgttaagttatatagtaagtaagaaaggagttggaatttaaatttaggtagtttggatttagaatttttgttttttttttttttttttgagatggagttttgttgggattataggtgtatgttattatgtttggttaatttttgtatttttagtagagattaggtttcgttatgttggttaggttggtttcgaatttttgattttaggtgatttgttcgttttggttttttaaagtgttgggattataagttaatgagttcggtttagaatttttgtttttgattattatgttatattgttttatgattttgttatacgtaagacgggttgttatttacgttttggtttttttattttagtagagttagaggggtgtttatggatttgataatgtgttttatgttttatgtgagtgtttaattgggttttagtttagagaatagtaggaaaatggttttttttttttttgatttggattgaagatttgtttgatgaattaggtttattgatagtagggttgaagttggaaatataggttagcggggttttttgaaatattagagtaaacgtttttcgtttatttttataggttgaggggtttttatttgtttatttttcggattaggtaaatttaggagaatggttttttgtggttgtttggggggtttttagttgtaaggtttgggtttataggaggtaggtagttgtgtggtgtcggaatgattttgcggggagataaattagttcgattttgttttgttgtttaaagtaaatatggggtggggtggggtggtttaggtttataattttagtattttgggaggtaggatcggggaatcgtttgagtttaggagttcgagattagtttgagttatatagtgagattttttttttttataaaaaataaataaaattagttgggcgtggtggtgtgtgtttgtagttttagttacgcgggaggttgaggtaggaggattatttgggtttaggagattgaggttttagtgagttaagattatattattgtattttagtttgggtaatagagtaatatcgtgttttaaaaaaatattaataaagtaaatatgagttttttttttggttgtttggtgttgagtggaaagaaaggtattggcgggtttagatttttttggtttatatatggatgagtaatgatttagagaggttaagtgattttaatattatagagtatattggcgttagaattgggatcggaatttagttttcgtaattgttagttttatgtttttttttttagatattagagggtaggggtaggttttaaaatgtttgagatttttagttgttttgtggtttaaatttttggttttgttttgttttagatagtttgtttttagcgttttgatttttgattatcgtttgtgtatgttaggttttgtatttgttaggcgggagtttaaggattattgtcgttagataataatagttaggagtttagggatttttggatgaaaggtgttagagataagtcgttagtgacgtacgaggatttggataaggttttggagcgaggtggtaatgttgggtacggcggtagattatgtttaatatgggggaagggagtgtatgtggtttaggtttgtgcgtgtttttgtgtgtgtgtgtaggtgagtatggcgtgtgttgggatatatttatgtgtatttttgtgtgagtgtgtaggtttatatgtgagtgtgtatatatatttgtgagagtttgtatgtatgtatacgtgtgaggtgttgatatatgtttatgtaggtattagtttgtttgtaaatatttttgtgtaataataataaagttcgaatagttattaagagtatagatatgaagttagattgtttgggttaaatttttagttagttagtttttcggtgtttttgtttttttttttgtaaaatggcgtttattttattaggattttaaaattagttaatatggttgggcgtttgtaattttagtattttgggaggttgaggtaggcggattataaggttaggagatcgagattattttgtttaatatggagaattttgagaattgtttaaatttaggaggtggaggttgcggtgagtagagattgtattattgtattttagtttgggtaatagagttagattttattttaaaaaagaaaaataaaaaataaaaaaattatgaatttatttttaggttgaggagtttagtattttggttgtgaaatattttttttataaaattttgggatggagattacggggattaggtgtttttttgtgataatttttgggtatggtggtttagggcgtaaattggagtgtggttataatatatattgtgtatttttataaggatgttatagagtttgggtattataaaagaggagttttttaaggaattgaaattattagataggagagagagttttgggtagatagggttgttcgtgttaaatattttagttgtggtataagggaaagggtgggagttatgaaattgttttattttgggtttaggtttgggttttgtcgttagttagttaagtgattttggttatttatttttgtggttttttatgagtaaaaggcggaaatttatttttatttagagggtaggtttgattttttttaattagtatttatttgtttatagtaggaaggattgaggtttaaagttggaggtgggtaggaaggattgaggtttaaagttggaggtgggtaggaaggattaaggtttaaagttggaggtgggtaggaaggatcgaggtttaaagttggaggtggttgtttagagttttagtagaggtttttggggtattttattgagtgtttggtaggagtgggtgtttgttttagggttgggttgagttgtttttattaggatttttcgttatttgtatagtgaggggattgggaggtttagagagttatagtttgggtttaaaataagtaagaggtttttgagtgtgaggattgttttggagtggaatggtttttataggtaggagtgagttttttgtagttagaggtatttaagtagttgaaggataatttttgggtaggaagttgtagagatggtcgtagcgtggattagaattgttgttttggttatttagattttattttagtttggtttttttggatagtatttttgtaatagtgagttggtgattttacgttttagaatttcggtttttatatttgtaaaatgggaattatatgatatttattatgtgttagatattttgttggtatatagtatatattattttatttaattttttaagtagggataagttatttttattttttatatgaggaagttgaggtatagagaggtgaagtgaatggtttaaggttatatagttgggaagatagggagttaaatttgaattttagtttggttgtttttagattttatatcgtattttttatgtcgattttagttttttttgtgtttatagg

SEQ ID NO: 43 - DNA, 8222 bp, Artificial Sequence
  chemically treated genomic DNA (Homo sapiens)
tttgtgggtatagggaaggttggagtcggtatgggaggtgcggtgtgaggtttgggggtagttagattagagtttaagtttagttttttgtttttttagttgtgtgattttgggttatttattttatttttttgtgttttagtttttttatataagggatggggataatttgtttttatttgaaggattaagtgagatagtgtgtgttatgtattaatagggtgtttggtatatagtgagtgttatataatttttattttatagatgtggaaatcgaggttttgaggcgtaaagttattagtttattattgtaggggtgttgtttagagaagttaggttggaatgaggtttgagtgattaaaatagtagttttagtttacgttgcgattatttttgtagttttttgtttagggattgttttttagttgtttaaatatttttagttataggaggtttatttttatttgtgagggttattttattttagagtaatttttatatttagaaattttttgtttgttttgagtttaagttgtgattttttgaattttttagtttttttattgtgtagatgacgaagggttttggtgggagtaatttaatttagttttgagataggtatttatttttgttaggtatttagtgaggtgttttagaggtttttgttgggattttgagtagttatttttagttttagatttcggttttttttgtttatttttaattttagattttggttttttttgtttatttttagttttagattttagttttttttgtttatttttagttttagattttagttttttttgttgtgagtaggtgggtgttggttaaagggagttagatttgttttttgggtaggagtgagttttcgttttttatttatggaagattatagagataagtggttaaggttatttggttagttagcggtagagtttagatttaaatttaaaatggaatagttttataatttttatttttttttttgtgttatagttgaaatgtttggtacgggtaattttgtttgtttagggtttttttttttgtttaatggttttagttttttgaaaagttttttttttatgatatttaggttttgtgatatttttgtaaaagtatatagtatgtattgtggttatattttagtttgcgttttgagttattatgtttagaagttgttataaggaagtatttgattttcgtagtttttattttagggttttatgaggagggtattttataattaggatgttgagttttttaatttgaaaatgagtttatggtttttttgttttttgttttttttttttgagatggaatttggttttgttgtttaggttggagtgtagtggtgtaatttttgtttatcgtaatttttattttttgggtttgagtaatttttagaattttttatgttaggtaggatggtttcgattttttgattttgtgattcgtttgttttagttttttaaagtgttgggattataggcgtttagttatattaattgattttaaaattttgatggggtagacgttattttgtagaggaagaaatagaggtatcgaaagattaattagttggggatttaatttaggtagtttggttttatgtttatatttttgatgattattcggattttgttattgttatatagagatatttataggtaaattgatatttatatggatatgtattaatattttatacgtgtatatgtatgtaggtttttatagatgtgtgtgtatatttatatatgggtttgtatatttatatagaggtatatataaatatattttagtatacgttatatttatttgtatatatatatagagatacgtataagtttgaattatatatattttttttttttatgttgggtatggtttgtcgtcgtgtttaatattgttatttcgttttaggattttatttaaattttcgtgcgttattgacggtttgtttttgatattttttatttaagggtttttgagtttttgattattattatttggcgataatgatttttgaattttcgtttgatagatgtaagatttggtatgtataggcgatggttagaggttaggacgttgaaagtagattgtttgaggtagagtagggttaagaatttgggttataaggtagttgggggttttagatattttggggtttgtttttgttttttgatatttagggaagagaatatgggattgatagttacggaagttgagtttcggttttagttttggcgttaatgtgttttgtgatgttgaagttatttagtttttttggattattgtttatttatatgtaaattaaaagggtttagattcgttaatgtttttttttttatttaatattagatagttagggagagagtttatatttattttattaatatttttttgagatacggtattgttttgttgtttaggttggagtgtagtggtgtgattttggtttattggagttttaattttttgggtttaagtgatttttttattttagtttttcgcgtagttgggattataggtatatattattacgtttagttaattttgtttattttttgtagagaaggggggttttattatgtggtttaggttggtttcgaatttttggatttaagcgatttttcgattttgttttttaaagtgttgggattataggtttgagttattttattttattttatatttattttaaatagtagagtagaatcgggttaatttgttttttcgtaagattatttcggtattatatagttgtttgttttttgtgggtttaggttttgtagttaggagttttttaggtagttatagaaggttatttttttggatttgtttgattcggaaaatgaatagatgaaagttttttagtttgtggaaatggacggagggcgtttgttttagtattttagaaagtttcgttggtttgtgtttttagttttaattttgttgttagtgggtttggtttattagatagatttttagtttaagttagagagaagggaggttatttttttgttgttttttgaattgaggtttaattggatatttatatggaatatggagtatattgttagatttatgaatatttttttggttttattagaataaaagaattagagcgtgggtggtagttcgttttgcgtgtggtaaggttatggggtagtatagtatagtggttaagagtaaggattttgggtcgggtttattggtttgtaattttagtattttgggaggttaaggcgggtagattatttgaggttaggagttcgagattagtttggttaatatggcgaaatttagtttttattaaaaatataaaaattagttaggtatggtggtatgtatttgtaattttagtaagattttattttaaaaaaaaaaaaaaggagtaaggattttggatttaagttgtttgaatttgaattttagttttttttttatttgttgtgtgatttggcgtaagttatttaatttttttgttaaatgtggtgattataattatatttattttataagattgttgttaggttttagtgagtgaaatatttaaatagagtttagtaattagtatgtgttgaataaatggttgttgttttttttttttttattatttttattattattattattattattattattattattattatttaggaatttttggttttgtttgtggttgttattaatcggtggagatttttaggtttagtttttttttgtaatgagaagtagtgattattattggtgatttttaaatattttatgattttgaaattttttgtttttttcgttttttaaaatagttaaagtaaataaaataaaaaaggtttgagtaaagaaaggaaaaataggggtgtggagtttatttagtagttttttttttttcgaagttttatttttggagttagaatttttaagtgattttttagtttttgtgttttagaattttatgtttgtgtacgtgtagtgaaggttttagtgttttatagattgggttttatggaatttgtttttttttataattagaagattggagatataattttttaaggttagttagaaaatttagagttagtatttagaataaagttaagatgttaatttttacgggtggtatttgtagttttgggtatgagttttttttattttgtttttagggaggttattttttttgtattttaaatattgagagggtagaattagttggtatagtttttgtatgttttataatttaagacgtgatagggtattaggtttttgggattttttagggattagtaattttaggtattatgttttatttttggttttcggttttcggtaaattttcgtgattttttttgatgatgtaatattttgagttttttttaagacgtataagttattgttttaggtttggtagtttttaaattattttggaaagatttttgttttgtaaattttggttttttatttttacgtttgattgttttgttttttcgttaaaggtaaaagtgtattaaggatttagagaattttagggtatgagcgtgggacgggatttcggagattatggaagataattttttcgtttaattagtagagaaataaggtttagaggtggagagatttgtttaaggttatatggtatatttgtggtttaggagttgatgttaatttgtgtgtgtgtgttggtcgggggagtattttgtgtaatttaatagggtttgttgtagttagatgatagggataggagaagaaagatataaatatattacgatgttattatagttaatagaaaatttaattttttttttttaagtgtttttagtacggattattaacggtttaaattttagataacggagtttttaggattagttatgttttcgaggtagacggaagttggaaatgggtaaagtttaagggagatttaaaggacgacggagtggaagaggaaaattggttttggtttataaataaggagaaattaatttgtaaattaaagtgttaaagataatttatttatttgtggtaatttcggcgtttattttttttttttcgttggtagaaacgttgatttttaatttattagatttttgagggaattagtttagaatggtgagggaaggggttaagagaagagtgtattatttttgggatagaaggtaacgaggagatgagtttttatgtataaattatagttattttttatttataaattttagaattttgttttttttggtgtattttggaattttttgtggtagaaggtaggatatatttgtagattatattatttagattttttaaattagagatatatttaatatcgaaggttattaatgagtttttattttttgttttttaattgtaatgttttgaaaggtttttagtttgggttttattagtaatagttattaatatttttaggatttagtatatgttaggaattatgttaggttttttgtatgtattattttatttaatttttatattttttttaggatatagatattaggatatttttttattgtataggagaggaaattgaggtttcgagaggttgagtatttttttaaaggttatttagtggtttagtggtagagttaagtttagaattagatttgtttttttttgaagagtatgttttaattttgatattttaagagtatttaatgagttgtgattttggaaatgtttttttttttttagtttttttttgtttttattttgtaagagaggtggttgtataaaattttttaggatttttgttaattttgattgtttataattttaaaattttttttggtggattttgagtattttaggtttgatttttatgttttttttagttgttgatattagtttgaaatggttttttttttttttttgagtttaatttttgatatttaaaagtaaagaaaaagtaagtgatcgttttaatatttttaattaatgtagttaattgatttattttacgtgtaaattttttattttgtagttgaatatttttgattttagggtgtgggtggtaggggtaatggaattttttttttttatttgatgaggattttttgtttatggtttgggatgttagtttttatttttttttagacgtttagtattaattgttatttttcgtcgttgagttttatggcgtagtttgaagaagataaaaagagtaagtatgggtttattgggtgttttggttttaatagttggggtttgaggttgtttggttttaagggcgtttttatattttttgggatgtttgataaagatcgtttaatggttcgttgttttttttttttgtttttttttatttttttttttttatttagggagggtttaatttttttttttttttagtttttatatgggaagttattttagggtaggtgttattgtggcgaggtaggatagtttaggttatagggggtggggtggttgtgcgtgtggaaagaaggggaggaggttgggtattgtttggatttttatagtcgatagaaagggtattatagaagtcgacgttagtagtttgaaatttaacgttttttgcggttagtttttttaagaggatttgttatgtttttaagtttggttattaggtgtttcggttggttttaggatagttttggttattcgtaattttttacgtattttttgtttttttgttagagattttttatttagttagaagtgatagaaaggaatcggatggttttgtgtttatagttaggtggttttaggtaagtttataattttttagagaatttttgtttttatgattttgtttaaaggtttttttttgaaaagttttttttgatatatttttttgagatattattaattgttttttttttttttttttttgtgtaagtttttgttgtattgttttttaaagggtattttattttttggtttttatgttattttttttattaggataatagtagttagtatttataagagattttgttgggtgttaggttttgttttaaatatttggtatttggtaatttatttaattttcgtaagattttttaatgtgataggtattgatattatttttatttaattgagagaaagtagggaaatattattgatgtataattagtcgttggtagagttgggatttgaatttaggaagttacgtttttttttttgtagtttttaggagggtaggagttttgttttattaatttacgtattttttgggttttatatggtgttttgtgttaagtttgaattgaataaatgtttattgagtaaatgggtggatggttttgtaaagaaagagagtggaggatgattagttttattttatatttatgtatttttttttaatttatttaggtatttaattatttatttatttatttatttgtttatttagttatttatttatttatttatttgttattttttaatttatttatttattatttttttgtttatttatgtatttatttatttatttatttattatttatttatttaatatttattaatttttttatttattcgatattttttaatttatttatttatttatttattatttttttgtttatttaggtatttatttatttatttatttatttatttatttaaaatttattagtttttttatttatttatttatttatttatttatgtgtttatttatttattttttaatttatttatttatttattatttttttatttatttatttatttatttttttatttatttatttattatttttttatttattatttttttttttatttatttatttatttattatttatttatttttttatttatttatttgtttttttatttttttatttatttatttatttatttttttatttatttatttattatttttttatttattattttttttttatttatttatttatttattatttatttatttttttatttatttttttatttatttgtaattttatttatttatttattttttttatagtgatagagtatttattgtgtgttagatattgttatatgttgaggataataaaattaaataaaatatttttttgtgtttaaggaatttagagtttttttagggagataattaagaggaatgaaaatatagtatagtttgtttagtgttatgattaaggtaggtataggtttttttttttatttgtataagattatatagtttataaagtggttgtttggtaagattatatttaatttt

SEQ ID NO: 44 - DNA, 3051 bp, Artificial Sequence
  chemically treated genomic DNA (Homo sapiens)
atagtggtttatgtttgtaattttatattttgggagatcgattgggaagattgtttgagtttaggagttggagattagttagagcgatatagtgggattttttttttataaaaatatttaaaaattagttaggtatagtggtgtatttttgtagttttagttatttgggaggtggtaagaggatttttcgagtttagaagttggaggtggtagtgagtagttattttgttattgtattttagtttgggtgttaaagggagatttttttttttagaaaataataaaaaaaaaagtttgatagtttttttgtttgtgttatataaaataatggtatatattataattgaaggtattttataatagaaatgtttttttttgtttttttttaaggttgatttagtgttttaagaggttgatatagaagggtaaagttaagtttttataaaatttagggaagagattgtgaaatttattttagaattttgtatggagttatagttgaattaggaaaaaaaaaaaaaaaaaaaaaaggaaagaaaggtattttagagataaagaatagattaaggagaggagatatttgaagttataatgattgagaatatgttagatttgaaatgaatttttagtttttttaataagataaataaaattaattttatatttagatatatggtagaaaaattaaagaattttgttgattaaggtattaaaagtaattaaagagaagtggtgtgaagaaagtaagagagaaataataaattttgtttattttgtaataattgaaaatttttggttgggcgtggtggtttacgtttgtaattttagtattttgagaggtcgaggtaggtggattattttaggttaggtgtttaagattagtttggttaatatggtgaaatttcgtttttattaaaaaaaaataataataataatataaaaattagtcgggggtggtggtaggtatttgtaattttagatattcgggaggttgaggtaggagatttatttgaatttgggaggcggaggttgtaatgagttgagatcgcgttattgtattttagtttggatgatagagtaggattttattttaaaaagaaaggaaaagaaaagaaaaaatattaaatgtgtacgttttttgatttagttgtattattttaaggagttgatattattaaaattgtttaagtgtttaaaggtgtttgtagttaaataataggagattgataaattatgttatatatatgtgatgttatgttttaaagaggtattgatatgataaaagatgtacgtggtataaaattaaatgtattttattaagtatttttttaagtgtttacggaatgagtgtatttttgaaaaaaaaaaagtgtattcgaatttttaaaaaagttttaaaagttttatataatgaacgattgagtgattataagagttggcgggggaatgttaagaggatgatagggagttaagtttaatagaataatttatttttttattttgtgatatttacgagcgtattaattttgtaattgaaaaataaagtgtatatttgtagtagttgtattttttttaggttgtaaggaggttttttttttcggtaggtttgatttgtattttatttttattttcgtggttggaaattttttatttacgtagtgggaggttgaggagttattataaagttggggtttgacgagtcgggatcgggattcgatttttatatatgttcggatttgttttgcggtcgggtttaggagttaaagaggcggggagatttgcgcgacgttgtttcgttttgcgttcgttttttttaatgtatgttttagggggcgggtttcgcggggagtatggatacgattggttttaaagtttttttcgtaaggtcgtgggttggatagcgtggtgacgtcgtaacgcggcgtagggtgagagcgcgcgtttgcggacgcggcggtattaaacggttgtaggcgtagtagagtggtcgttgtttttttaggttttagtcggtcgtcgcgacgttcgttcgttcgttttgaggtttttgaagtcgaaattagttagatttttttttttttcgtttgtttgtagcggcgttgttgttatttcgttattatgttcgaggcgcgtttggtttagggttttatttttaagaaggtgttggaggtatttaaggattttattaacgaggtttgttgggatattagttttagcggtgtaaatttgtagagtatggattcgttttacgtttttttggtgtagtttattttgcggtttgagggtttcgatatttatcgttgcgatcgtaatttggttatgggcgtgaattttattaggtgagtttcgcggtttcgggaagtcggtttcggttcgtttgtattttcggtgtttggcgggagcgttttcgagtttagtttttattggttggcgtgggttattcgcgtttttttattggtttgttacgtagtggggtggggtttagttgagcgcgcggttcggaaaagttcgcgttggttgttgcgcgaatttgttttttcgcgttaaagttataaagcgggtggtggcgggaaaattaagggttttttcgtagtgttaggaatattgttttagggttttttgtttattaaattttgttggttttgaatggacgttttagttgtggtttttttgtttttgagacggtttcggtgtgttgttcgggttggtttttaatttttgggtttaagcgattttttcggtttagtcgcggttgattttaaatgttttataatgtttttgcgagaaatgtggtagtttgttattttatttagtggtaggagattgtttttatttagaagggatattgttggtggtattttagtataaatattgttagatgcgttttaaaacgtttgtattaataatggtattttttagtagttcgtttattttttattagttttgagacggtttgatgggtgagagtggtaattttttttaatcgcgttcgaaatatagttttttagtagacggcgttgattttaaagtatgtgttttttgttttttagt

SEQ ID NO: 45 - DNA, 3051 bp, Artificial Sequence
  chemically treated genomic DNA (Homo sapiens)
attagaagataggagatatatgttttaaaattaacgtcgtttgttgaagggttgtatttcgaacgcggttagaaggggttattatttttatttattaggtcgttttagaattggtggagggtaagcggattgttggaggatgttattattaatgtagacgttttgggacgtatttggtagtatttatattaaaatattattagtagtgttttttttggatagaaataattttttgttattaagtaggatgataggttgttatatttttcgtaagggtattataaagtatttaaagttaatcgcgattgagtcgggaggatcgtttgagtttagaagttggagattagttcgggtaatatatcgagatcgttttagaaataagaaagttatagttaaagcgtttatttaaggttaataggatttagtgagtaaagagttttggaatagtgtttttggtattgcggaaaaatttttgattttttcgttattattcgttttgtgattttggcgcgaaaaagtaggttcgcgtagtagttagcgcgggttttttcgaatcgcgcgtttagttgggttttattttattgcgtggtaggttaatgagaaggcgcggatggtttacgttagttaatgagggttaggttcgaaagcgttttcgttaagtatcggaggtgtaggcgggtcggggtcggtttttcggggtcgcgaggtttatttggtgaggtttacgtttatggttaggttgcggtcgtagcggtaggtgtcgaagtttttagatcgtagggtgagttgtattaaagagacgtgggacgagtttatgttttgtaggtttatatcgttggagttaatattttagtaggtttcgttgatgaggtttttgagtgtttttaatatttttttgaggatggagttttggattaggcgcgtttcgaatatggtggcggagtggtaataacgtcgttataggtaggcgggaaggaggaaagtttagttggtttcggttttaggagttttagagcgagcgggcgaacgtcgcgacgatcggttgagatttagaaagataacgattattttgttacgtttgtaatcgtttaatgtcgtcgcgttcgtaagcgcgcgtttttattttgcgtcgcgttgcgacgttattacgttgtttagtttacggttttgcggggaagattttagggttaatcgtgtttatgtttttcgcgaggttcgttttttagagtatatattggaggaagcgggcgtagggcggggtagcgtcgcgtaggttttttcgtttttttgatttttgaattcggtcgtagaataagttcgggtatatgtggagatcgggtttcggtttcggttcgttaagttttagttttatggtggttttttagttttttattacgtgggtagaaagtttttagttacgaaagtgaaagtgaaatgtaaattaagtttatcgggaggaaaagtttttttgtagtttgaagagagtatagttgttgtaaatatgtattttattttttaattatagaattgatgcgttcgtaggtgttataagataaagaggtgaattgttttgttaaatttagttttttattatttttttaatattttttcgttagtttttataattatttaatcgtttattgtataaagtttttaaagtttttttaaaagttcgaatatatttttttttttttaaaaatgtatttatttcgtaaatatttggaaaagtatttaataaagtatatttaattttatgttacgtatattttttattatattagtatttttttaaaatatagtattatatgtatataatataatttattaattttttgttgtttaattataaatatttttgagtatttaggtaattttggtgatattaattttttgaagtaatatagttgagttaaagagcgtatatatttaatatttttttttttttttttttttttttttgagatggagttttgttttgttatttaggttggagtatagtggcgcgattttagtttattgtaattttcgttttttaggtttaagtgagttttttgttttagtttttcgagtatttgggattataggtgtttattattattttcggttaatttttgtattattattattatttttttttagtagagacggggttttattatgttggttaggttggttttgaatatttgatttgaggtgatttatttgtttcggttttttaaagtgttgggattataggcgtgagttattacgtttagttagaaatttttaattgttataggatggataggatttgttgttttttttttgttttttttatattatttttttttagttatttttaatattttggttagtagaattttttagtttttttattatgtatttaggtatggagttagttttatttgttttgttgagggaattgaagatttattttaagtttgatatatttttaattattatggttttaaatattttttttttttagtttattttttatttttggaatgttttttttttttttttttttttttttttttttttttagtttagttgtgattttatataaggttttaagatgagttttatagttttttttttggattttgtggagatttaattttatttttttgtattagttttttaaagtattgggttagttttaaaagggaataggaaagggtatttttattataagatgtttttaattgtaatatgtattattattttatgtagtataagtaagaaaattgttaaatttttttttttattattttttagagaggggagtttttttttgatatttaggttggagtgtagtggtaagatgattatttattgttatttttaatttttaggttcgagagatttttttgttattttttaaatagttgggattataggggtgtattattgtatttggttaatttttaaatgtttttgtagagagaaggttttattatgtcgttttggttggtttttaatttttgggtttaagtaatttttttaatcggttttttaaagtgtgagattataggtatgagttattgt

SEQ ID NO: 46 - DNA, 3664 bp, Artificial Sequence
  chemically treated genomic DNA (Homo sapiens)
gaatttttaattattattttgtttttttatttttaatatttttagcggtaattgtattttatttatttttgtgtttatgaagtatagtgttgtattttggagatattatatagaagttggtttaaagggtaataaattgtatttgttaagatgatagtatggatttataataatttttgaaattttataaagaaagatgattgagttaaattgatatatttagtattatagttgtggaagtttgtggttttttttatgttaaaggtaaaatggatataatgaattagttgttatttttagatgatatagaagttgaattttttttaagttttttaaagagaaagatatattaaaatttattaaaattagtattataaattgtatagaatagtttggttttttagagtatttttttggggtagtgagtttttatttaaaaaatgaagtttggattttaaatataagttttaaaagtgattaggttttagtaggagtttttaaatttttatttatattatgatttttaaatgttttgtttttgttgtttaggaataaaaattgttaaggttgagttagatgtaatgtataatattagatgatagatgtagtttattgtgttaaaaggtgttaaatatttaagtgtatgtgatatattttatattataagggtaaggaagttttattttatattttgatttgggttttttaatatatatttgtagtggatgtggtaattagtttttgttttatgttttattaggattaatgtaattagaaatttttaaaatggatttttattatgtttagttttgttttattgttttatttgtttattttaaaaagattttagaatggtgtttaggttgggttggtggtttacgtttgtaattttagtattttgggaggttaaggtaggaggattgttggagtttaggagttggagattagttagggtaatataaggagatattgttttcgtaaataaatagaaaaaaaaaaagtaagaaagaaagagagaaagaaggaaagaataagaaaaaggaaaaaaaaattaattgtagtaagtgtagaaattttttttaaggatatgatggatggtaatgaagatagagtttttgatattggattatttaaagtataatttaagtttttttttttttttttatagaattgtgaatttttgtgttttaattatatttaagttaggcgtggtggcgagtgtttgtagtcgtagttgcgttggaggttgaggtcgattgtttgagtttaggatttggaggttagtatgcgtaatataatgagatttagtttttaaatgtatgtttttttttatatatttaaaattttgatgtgaaaatattttaaaatttaatatattttaaatgtttttaattgtataataaataaaatgtaaataataaaataatttaatattaaatttaaaaatgaggtagaaataaagtatagcgatataaataataaatttttttttatatttttgaggcggttttttgagttttttattttttttttaaggttattgaaatgtgttttttggagttagttcgtaaattacgtatttagaaaaatataattatatatttttaattttaagtattagaagtgaaagtaatggaatttcgatgtaaatataatattatttttttgatgagttattttgagtataataaatttgaattgtgttaatgttgggagaaaaaatttaaaagaagaacggagcgaatagtagttttttgtttcgttgattagaaatagtaggacgatattttttcgattggaggagagcgtttgcgttcgtatttagttggcgttcgtttttttgttttttttttagtcgtttttttttttttttttcgcgttttagttattcgggaaggtttgtttagcgtagttgggttttgattggttgttttgaaagtttacgggttattcgattggtgaattcggggttttttagcgcggtgagtttgaaattgttcgtatttggttttaaagttggtttttggaaattgagcggagagcgacgcggttgttgtagttgtcgttgcggtcgtcgcggaataataagtcgggtatagtggttggggttagggtcgtgtttaggggacggtcgagggtttcggagggcgagtattgaggaacggggttttttaagaaggtcggattggaggttagggatttgcgcggggttcggttgggtttcggggggcggtgggtagggttttcgtttgggtttggtcgtgtgagttatattgggttggttttagagggaggttatgggagtttagtttggcggggttaggcggcgtgggggtgggggttcggggttgtagttatgagtcgaggtcggttgtttttaggaattgtttgaagaggttttcgggttttgattgaagtaaaattcgtttttttacgttgagcgtttgcggagtagtaggtttttttttgggttcggtttttgcgcgttttgttatttcgtttcgggggtgggaaagtgggatcgtatttgggttaggtataggcgcgggtttttagagttaggtcgttttgagaaatcgattttggaattcgtgggtttttttgggtttgttttgtttacggtttcggtttatttggagttgttaagaatagttagtttaggtatttaatttttatgagtagtgtagtgtagttttttttatttaaaaagatatatttttgtttttttttggattgatttttttttgaagatgaatgtgagaaatagaatttaaaggtttattttgagtgtgttttataaatgattttattattagtgatgggtgttagaaaaatttaggtaattatgtttggagttttgacgttttattggtaaattttagaggaagttttgaatttttggttgttagtttttttttttaatttatttattgtgatttaagtatatcgtttttttgtttagtttttgatattgtttaggttataattagaattttatttgtggaattgtattttttatttttttgttttacggtgtttaagtgatattttttatttttggaaaaagtattttgaaagttttgagtatgtttgtttgtgattttagtaagtagaggaaaaagattaaaattgttacgtgggtaattgtaaatatttaaaatgtttttgtcgttttaaataaaatttttttaatgaatagtttgaatatattttagttttgtttttataattttaaaaaataatgttttgaataaaattaggagtattataaagcggtataaatttgttgaaatatttgggtataattttggtatacgtaggtattattttaaagtgttataaaaagggaatataattttggtttttgtttggaaagaatagataagttgatatttgaatttgagggttattttgattggaagaagttaaatattatattagtatagaatatatacgtttttaatgttttaggagttaagaaggatggaaagttattgtaggagtttttatttttgtaaatattgtttgaatgtaattaatatttatggaatatttatgttgtagtaggtattgtggaaaatgttttatatatagtgttttattaatgtttaaaattattcgagttagtggtatgtaattaagttagttttttttttattttagg

SEQ ID NO: 47 - DNA, 3664 bp, Artificial Sequence
  chemically treated genomic DNA (Homo sapiens)
tttaaagtgaaggaaaaattgatttaattatatgttattaattcgagtagttttaagtattaatgaaatattatatgtaaagtattttttatagtgtttattgtagtataagtattttataaatattaattatatttaaataatatttataagggtaaggatttttataataattttttattttttttgatttttgagatattggaaacgtgtatattttatattagtgtagtatttagttttttttaattaaaataatttttaaatttaggtattaatttgtttattttttttaaataaaaattagaattatgttttttttttgtgatattttaaagtagtatttgcgtgtgttaaagttatatttaaatattttaataaatttatatcgttttatgatatttttaattttatttaaaatattattttttaaagttatgaggatagaattaaaatgtgtttagattgtttattaggaaaattttgtttaaaacgataaaaatattttaagtgtttatagttgtttacgtgataattttaattttttttttttgtttgttgaggttatagataaatatgtttaagatttttaaggtattttttttaagagtaggaaatattatttaagtatcgtaaaataaaagaatgaaaagtgtaattttatagataaaattttaattataatttaaataatgttaaggattaagtagaaaaacggtatatttgaattataatgaatgggttaaaaaaaaaagttgataattagaagtttaagattttttttgaagtttgttaatgaaacgttagaattttaggtataattatttagatttttttaatatttattattgatgataaaattatttataaagtatatttagaatagatttttaagttttattttttatatttatttttaaagaaaaattaatttagaaagaagtagagatgtatttttttaaatgaaaaaaattgtattatattgtttatgagagttaagtgtttgggttgattgtttttaatagttttaggtgggtcggggtcgtgggtagagtaagtttaagaaggtttacgagttttaggatcggttttttagggcggtttggttttagggattcgcgtttgtatttggtttaggtgcggttttatttttttattttcgaggcgggatggtagggcgcgtaggggtcgggtttaggaaagagtttgttatttcgtaggcgtttagcgtgggggagcggattttgttttaattaagattcgaaagtttttttaggtagtttttgagaatagtcgatttcgatttatgattgtagtttcgaatttttatttttacgtcgtttaatttcgttaggttgggtttttatagtttttttttaaagttagtttagtgtggtttatacggttagatttaggcgaaggttttgtttatcgtttttcgaggtttagtcgggtttcgcgtagatttttgatttttagttcggtttttttagaggatttcgttttttaatattcgtttttcgaggttttcggtcgttttttagatacgattttgattttagttattgtattcggtttattatttcgcggcggtcgtagcggtagttataataatcgcgtcgtttttcgtttaatttttaagagttagttttgaagttaagtgcgagtagttttaaatttatcgcgttaaagggtttcggatttattaatcgggtagttcgtagatttttaaagtagttaattagagtttagttacgttgggtaggttttttcgggtggttagagcgcgaaagaaagaggaaagggcggttagagaaaaagtaggagggcgggcgttaattgagtgcgagcgtaagcgtttttttttagtcgggagagtgtcgttttattgtttttagttagcggagtaggaagttattgttcgtttcgttttttttttaaatttttttttttagtattggtatagtttaaatttattatatttaaaatagtttattaaaaaagtgatattgtgtttatatcgagattttattatttttatttttaatatttagggttaggagtgtatagttatgtttttttaaatgcgtgatttgcgggttggttttaaggagtatattttagtgattttaagaaggaaatggaaaatttaaaagatcgttttaaaaatgtaaaggaaaatttattatttatatcgttgtgttttgtttttattttatttttgaatttaatattaaattattttattatttatattttgtttattatataattaaaaatatttgaaatgtattaaattttaaaatatttttatattagaattttaaatatatagagagaggtatgtatttagagattgggttttattatgttgcgtatgttggtttttaaattttgggtttaagtaatcggttttagtttttagcgtagttgcgattataggtattcgttattacgtttggtttaaatataattaaaatataaaaatttataattttatgaggggagagaaagaggtttaaattatgttttaaataatttagtgttaaaaattttgtttttattgttatttattatatttttgagagagatttttgtatttattgtagttaatttttttttttttttttttgtttttttttttttttttttttttttttttattttttttttttttgtttgtttgcggagataatgtttttttatgttgttttagttggtttttaatttttaggttttagtaatttttttgttttggttttttaaagtgttgagattataggcgtgagttattagtttaatttaaatattattttaaaatttttttaaaataaataaatagaatagtaaaatagagttagatataataaaaatttattttaaggatttttggttgtattgattttaatgggatatagaataaagattgattattatatttattataagtatgtattagaagatttaaattaggatatggaatggaatttttttatttttgtgatatagagtatattatatgtatttaagtgtttaatattttttaatataatgagttgtatttattatttggtgttatatattgtatttggtttaattttgataatttttgtttttaggtaatagaggtagagtatttgggagttatggtgtaggtaggaatttaagaatttttgttggagtttaattatttttaaaatttatgtttaaaatttaaattttattttttaaatgaaaatttattgttttagagaagtgttttagggaattaggttgttttgtgtagtttatggtattggttttaatgaattttaatatgtttttttttttgagaggtttaagaggaatttaatttttatattatttaaaaatggtaattaatttattatatttattttatttttaatataaagaagattataagtttttatagttgtgatgttaggtgtattaatttaatttaattatttttttttataaagttttagaggttattgtaaatttatgttattattttggtaaatatagtttattattttttgggttaatttttatgtagtatttttaaagtgtaatattgtgttttatagatataaaaatgaataagatatagttatcgttggaggtgttgagggtaggggagtaggatgataattgggaattt

SEQ ID NO: 48 - DNA, 1729 bp, Artificial Sequence
  chemically treated genomic DNA (Homo sapiens)
ttgtatttttggttggggtattttaattagaattgttaaatttagtatataaaaataaggaggtttagttaaatttgaattttagataaataatgaataatttgttagtataaatatgttttatgtaatattttgttgaaattaaaaaaaaaaaaaaaagttttttttttatttttatttttattattaggtttaaggaatagggttaggggttttaaatagaatgtggttgagaagtggaattaagtaggttaatagaaggtaaggggtaaagaagaaattttgaatgtattgggtgttgggtgtttttttaaataagtaagaagggtgtattttgaagaattgagatagaagtttttttgggttgggtgtagttgttcgtggttgtaattttagtattttgggaggttgaggcgggaggattatttgaggttgggagtttaagattagttttattaacgtggagaaattttgtttttattaaaaatataaaaaattagttggttatggtggtatatgtttgtaattttagttgttcgggaggttgaggtaggagaattatttgaattagggaggtagaggttgtggtgagtagagatcgcgttattgttttttagtttgggtaataagagtaaaagttcgtttaaaaaaaaaaaaaagttttttcgatgtgattgtttttttttaaatttgtagatttttttaagattatgttttttagatattttaaagattttagaagatatgtttcgggggttttggaagttataaggtaaatataatatatttttttttttgattattaattttattagaggatgtggtgggaaaattattatttgatattaaaataaataggtttgggatggagtaggatgtaagttttttaggaaagtttaagataaaatttgagatttaaaagggtgttaagagtggtagtttagggaatttatttcggatttcgggggagggggtagagttattagtttttgtatttagggatttttcgaggaaaagtgtgagaacggttgtaggtaatttaggcgtttcggcgttaggagggacgtatttaggtttgcgcgaagagagggagaaagtgaagttgggagttgttatttttagatttgttggaatgtagttggagggggcgagttgggagcgcgtttgtttttaattataggagaaggaggaggtggaggaggagggttgtttgaggaagtataagaatgaagttgtgaagttgagatttttttttattgggatcggagaaattaggggagtttttcgggtagtcgcgcgtttttttttacggggttttttattgcgtcgcgcgttcggtttttatttttcgtagtatttcgcgtttcgcgtttttttagtcgggtttagtcggagttatggggtcggagtcgtagtgagtattatggagttggcggttttgtgtcgttgggggttttttttcgtttttttgtttttcggagtcgcgagtatttaaggtgggtttggtgtggggaggggacggagtagcggcgggattttgttttgtggatgtttcgtcgaggtttcgcggtcggcggggttagaggggttcggacgagtttttttatttcgaagttgtggatagtcgagacgtttagggtagtcgggttttggggttttcgggcgggagggggtagttatacggtagcggttcgagatggtttatttaagagattggcgttttttaggt

SEQ ID NO: 49 - DNA, 1729 bp, Artificial Sequence
  chemically treated genomic DNA (Homo sapiens)
gtttggaaagcgttagttttttggatgggttatttcgagtcgttgtcgtgtaattgttttttttcgttcgagggttttagggttcggttgttttgagcgtttcgattgtttataatttcgggataggagagttcgttcgggtttttttggtttcgtcggtcgcgggatttcggcggggtatttatagggtagggtttcgtcgttgtttcgttttttttttatattagatttattttgggtgttcgcggtttcggggggtaagagggcgaggaggagtttttagcggtataaggtcgttagttttatggtgtttattgcggtttcggttttatggtttcggttggattcggttgggagggcgcggggcgcggggtgttgcgaggggtgggggtcgggcgcgcggcgtagtaaagggtttcgtgggaaggggcgcgcggttgttcggggggtttttttggttttttcggttttaatggaggggaattttagttttataattttatttttatatttttttaagtagttttttttttttatttttttttttttttgtgattgggagtaagcgcgtttttagttcgttttttttaattgtattttaataagtttgggagtggtaatttttagttttatttttttttttttttcgcgtaggtttgggtgcgttttttttagcgtcgggacgtttgggttgtttgtagtcgtttttatattttttttcggagaatttttaaatgtagaggttggtgattttgttttttttttcggagttcgggataaattttttaggttgttatttttaatatttttttaagttttaggttttattttaaatttttttggggagtttgtattttattttattttaagtttatttgttttaatattaaataatggtttttttattatattttttagtaaaattgatagttaaggagggggatgtgttgtgtttattttgtggtttttaggattttcggggtatattttttggaatttttgaagtatttgaaaagtatgattttaagagggtttataaatttgggaggagatagttatatcgaaaggatttttttttttttttaaacgaatttttgtttttgttgtttaggttggagagtaatggcgcgatttttgtttattataatttttgtttttttggtttaagtgatttttttgttttagtttttcgagtagttgggattataggtatgtgttattatgattagttaattttttgtatttttagtaaagatagggtttttttacgttggtgaggttggttttgaatttttaattttaggtgattttttcgttttagttttttaaagtgttggaattataattacgagtaattgtatttagtttaaaaagatttttattttaattttttaaaatgtattttttttgtttatttaaggaggtatttagtatttaatgtatttaaggttttttttttgttttttgttttttattagtttgtttaattttattttttaattatattttatttggagtttttgattttattttttaggtttagtggtaggggtggggatggaaggaagatttttttttttttttttaattttaataagatattgtatgggatatatttatattaataaattatttattgtttatttgaaatttaaatttaattgggtttttttatttttatgtgttaaatttggtagttttagttggaatgttttagttaagaatgtag

SEQ ID NO: 50 - DNA, 12963 bp, Artificial Sequence
  chemically treated genomic DNA (Homo sapiens)
gttaggagttttagaaataggggagagttagaaagttggttagatttatgttttttaagtgtagggttagggttgagtttgtttttggggtaggtaagtttttttgaatttttgagggaagtagaagatataaattgttagataaaatgtaagtttagtttaaaagggttacgtgtcgttttttttagttttggggtattttttttttagaaaattggattgttttatagtgaaaatttcgggggtggttagttttttgtttcgttgttatttttattatttatagttttttaagaagtttttaggttgggtgttgaattttgattaggaattattgagaaatcgaggtagttgggagaagttgtagttttaagcgttgaaaggaagatgggggataataaatttgggtcgttaagtaaagggggtagaggtttggagaagtgggttttaggattagaggatagatcgattttatattttatttttttagattttatattttattgttattattatttacgtgtttttttcgttttttgtagcgggttttttagaggtattttttatggtttttttagattttaattttggttcgttcgttttttttttagaaaggttttcgtttgttttttttgtaggaaggtttgtatttttagaaagtttttgtttttcgattcgaggatttaatttattaggggaattaaattttgtttttaggggagtggagagagaaattgggtttttttttcgtagtttttgggatatagttgagttagttataggatttggggataatcggggcggattttttttttcgggaggcggtggtattagtttagagttcgtatttttatttatcggggaagcgtggggagaaggatgggttggagttgggttttggtttgaaggatagtagttcggagttaacggttgagtttttaaagtttttatattgtagaggaagtatagcggagattagttttagttaggatggtttcgaagtttttagggattcgacgtagagttaaagaaatttatttgtgtttttttttttttttgggagtaggtagaagattttcgggaggagaggcgaatagcggacgttaatttttttgaaagtattgtgttttttagtatcgcgggtcgttacgggttttttgttgtcgcgggatttcggtttatttttcgattgggtcgtcgtatttcggattagatttcgcgggcgatttacggaattcgcggagtcgggacgtgaaaggttagaaggtttttcgtttttattaagttttagggtttttcgtggttgttgggagttgtagtttgaacgtttttattttggcgagaagcgtttacgttttttttatcgagtttcgcggtaatttttaaagtatttgtatcgttttttcgtcgtttgtagagggcgtagtaggttttgtattttttttgtattttattttttaggttttagatttgttttttttatttaaaaaatatttattatcgagtttttatttgttatttagtattgatataggtatttaggaatataataatgaataagatagtagaaaaattttatatttttataaggtttacgtttttatgtattgaaagtaatgaataaataaattttattagagtgataagggttgtgaaggagattaaataagatggtgtgatataaagtatttgggagaaaacgttagggtgtgatattacggaaagtttttttaaaaaatgatattttaattgatgagaagaaaggatttagttgagagtaaacgtaaaagtttttttttttttattttttatatttgatataatgtaggatttttttaaaatgatttttattaattttgtttttatagttttggtttgtagaattttttattttaaaatgttagtatttacggtattaggtcggcgagaattttgattttgtatttttttttttaattttattttttttgtttttttcggtaggcggattatttgtttttatttgttatggcgattgtttagttttgtgttaggagtttcgtaggggttgatgggattggggttttttttttttatgtgtttaagattggcgttaaaagttttgagttttttaaaagtttagagttatcgtttagggagtaggtagttgttgggtttcggggatattttgcgttcgggttgggagcgtgttttttacgacggtgatacgtttttttggattgggtaagtttttgattgaatttgatgagttttttttgagttacgggttttcggtttcgtgtatttttagttcgggaaaatcgttggggttgggggtggggtagtggggatttagcgagtttgggggtgagtgggatggaagtttggttagagggattattataggagttgtattgttgggagatttgggtgtagatgatggggatgttaggattattcgaatttaaagttgaacgtttaggtagaggagtggagttttggggaattttgagtcggtttaaagcgtatttttttgtatatttattcggtgttgggcgtagggaatttttgaaataaaagatgtataaagtattgaggtttgagatttttggatttcgaaatattgagaatttatagttgtatattttagagtttatggtattttagtgaaaattggggttttatttcgaaatgattatttgggggtgattcggggagtttaagttgttaaggttttataattttcggatttttgttttttttggagcgatttttttaggtagttttcggtttcgttagatggagaaaatttaattgaaggttgttagtcgtggaagtgagaagtgttaaattaggggtttgttcgttaggtcgaggaggatcgtcgtaatttgagaggttcggtagttttgttattgtttggttttatatttatatttttgttttttgtagtagtattttcggtttttttttgtcggagtagtttattatttattcgatgagaggggaggagagagagagaaaatgttttttaggtcggttttttttatttggtagagggaggttgttatttttcgtttgtatttttttttttggattatttagttatggtttttgtaaaggtaggggtatttgttttgatgtaaattttaatttttttttttttttgaatggtgtgttttatttttcgggtcgtttgtaatttaggcggacgttattatggcgtagatagggagggaaagaagtgtgtagaaggtaagttcggaggtatttttaagaatgagtatattttattttttcggagaaaaaaaaaaaagaatggtacgtttgagaatgaaattttgaaagagtgtaatgatgggtcgtttgataatttgtcgggaaaaataatttatttgttatttagttttgggttaggttattttagttttagacgtaggttgaacgtcgtgaagcggaaggggcgggttcgtaggcgttcgtgtggtttttcgtgtagttttcggttcgagtcggttttttttggtaggaggcggaattcgaatttatttttttcgttgttttattttttagttcgcggttgttttatttcgtagtttttttttatgtatttgtcgcgtatcggttattttgtgtcgtatttacgttattttttttttaaatcgaggtggtatttatatatagcgttagtgtatatagtaagtgtataggaagatgagttttggtttttaatcgtttcgtgatgtttattaagttatagattttttttatcgttttagaaacgttttattacgtttttttttagtcgattttcgattttatttttattttgatttttataattattttgtttgttggagaattttatatagaatggaattaggatgggcgttgtggtttacgtttgtattttggtttacgtttgtattttgggaggtcgaggcgggcggattatttgaggataggagttttagattagcgtggttaacgtggtgaattttcgtttttattaaaaaatataaaaattagttgggcgtggtgggtgtttgtaattttagttattcgggagggtgaggtaggagaatcgtttgaattcgggaggtagaggttgtagtgagttaagatcgtgttattatattttagtttgggcgataagaacgaaatttcgttttaaaaaaaaggggggaattatatattatgtgtttatttttgtcgggtttttgttttttaatgtattgtttgatattcgtttatgttgtatatattagtattttgtttttttttatttagtatagtttatcgattgtatattcgtttttttgatggttttttgagttgttttttatttgcggttatgaaataaagttgttataaatatttttgtataattttttttgtgattatatgttttcgtgttttttggagaaatatttaggaggggaattgtggaggaagtaaaaagtagttgtattttgaatttttttagaagttttgagttttttagagcggttgtattattttatattttaattagtaaggtatgggagttattatggttgtgttatagtttttcggatattaggtatgttagtttttttaatgtggtatatttttgtggttgtaatttatagttttttattgattaaggatgtttagtatttttttatgtgtttattggttattcgtattttgtttgtaaagtagtttttcgagttttttatttgttattttggtttttttgtttgtttttattgtttagttgtgggattgttttatattttttggatataagtttttattagatttatgagtcgtgaatgtttttttttgatttgttgcgggtttatttgtttgttttatagagtttatagaattttaagaggagtggattaattttttttatgtttagtatttgttttgttttgtttaggatatttttttttttttttttaattttagggttatgaagatataattttatatttttttttaggatttttatggtggtaagttttatagtaaggtttttaagttattaattaatttttaaaattaattgtttatggtgtgaggtgtaggagttagtttttggtattttttttgtatggaaatttagttattttgtttttatttgttgaaataggttttttttttttattgaatgtttttaattttaattattttatagttggagtatagggttattattttagtgttattttttttttttttttgttaatttttgagatagggatttatattgttgtttaggttagagtataatggtataattaaggtttattgtagtttcgaatttttgggtttaagtagttttttagtagttttacgagtagttgggattattttattatatttagttaattattttatttttttgtattgataggattttattatgttgtttaggttggttttaaattgttggttttaagtttttattttatttcggttttttaaagtgttgggattataggtgtgagttattatgtttgattttttagtgttattttttatttattttttttgttttttgttttttttaaacgttggaggaagaaatagtatttattttatataaattttttagaaaatagaggaatagattgggcgcggtggtttatatttgtaattttagtattttggtacgttgaggtaggggattatttgaggtcgggagttcgagattagtttggttaatacggcgaaattttatttttattaaaatataaaagtagttaggcgtgtattatatttgtaatgttagttatttaggaggttgaggtataagaattttttgaatttgggaagcggaggttgtagtgagtcgagattgcgttattgtattttagtttgggtaatagagtgagattttgttttagaaaaaaaaagaaagaaagaaaaaatagaggaatattttttaatttgttttcgaagttaggataattttggtattaaaattaaataaggatattataagaaaagaaaatatagattaatatttttgttagtatagatatgtaatagttaattaattttagtaaattaaatttggtaatatagaaaaaaggataaataggttagtcgcggtggtttacgtttgtaattttagtattttgggaggttgaggtaggtagattatttgaggttaggagtttgagattagtttgattaatatggtgaaatttcgtttttaataaaaatataaaaattaggttgggtacggtggtttacgtttgtaattttagtattttgggaggtcgaggtgggtagattacgaggttaggagtttaagattagtttgattaatgtggtgaaacgttatttttattaaaaatacgaaaattagtcggtgtggtggtatttgtttgtaattttagttatttaggaggttgaggtagaattgtttgaattcgggaggtagaggttgtagtgagttaagatcgtgttattgtattttagtttgggcgatagagtaagattttattttaaaaaaaaaaaaaattagttgggtatggtggtgggtatttgaaattttagttattcgggagtttgaggtaggagaatcgtttgaatttaggaggtagaagttgtattgagttgggattatattattgtattttagtttgggtaatagagtgagattttattttaaaaaaagaaaaagaaaaaggataaatatattttaattaaataatgtttattttatgattgtagttgatttaatatttaaaaattggtttggtgtagtagtttaggtttgtaattttaatattttaggaggttgaggtaggaagattttttgagtttaggattttaagattagtttgggtaatatagttagattggtttttattggggggaaaaaaattagtttgtgtaatttattatattaataaagggaaatataaaaattttatgattattttaatagatgtagtaaaagtagttaatgatattaatatatatgtatgattataaattaattaattttttagtaaattagggaaaggaaatttaattagtttgataatagggcgtttatagtcggagttttattagtagtatatataatggtagaaaatttagtgttgttgggggcggtggtttacgtttgtaatgttagcgttttgggaggtttaggcgggcggattacgaggttaggagatcgagattgttttgattagtatgttgaaatttcgtttttattaaaaatataaaaataaaaaattagtcgggtatggtggcgggcgtttatagtgttagttattcgggaggttgaggcgagagaatggcgtgaattcgggaggcggagtttgtagagtttagatcgtgttattgtattttagtttgggtgatagagtgagatttcgttttaaaaaaaaaaaaaaaaaaaaaagaaaagaaaatttaacgtttttttttttaagattaggaattagaaaaggatttgatttttataacgttgatattatattggaggttttaattaggtaagaaaaagaaataatgagggtcgggtgcggtggtttaggtttgtaattttagtattttgggaagtcgagacgggtggattacgaggttaggagatcgagttattttggttaatacggtgaaattttgtttttattaaatatataaaaaattagtcgggcgtggtggcgggcgtttgtagttttagttattcgggaggttgaggtaggagaatggcgtgaatttagggggcggagtttgtagtgagttgagatcgagttattgtattttagtttgggcgatagagtaagattgtgttttaaaaaaaaaaaaagaaaaagaaataatgattagtggttcgatgttttacgttagtaattttagtattttgggaggtcgaggtgggtagattatttgaggtttggagttggagattagtttgataaagatggtgaaatttcgtttttattaaaatattaaaaaaatagttaggcgttggtcgggtatagtggtttatgtttgtaattttagtattttgggaggtcgaggtgggtggattatttgaggttaggagtttaatattagtttggttaatatggtgaaattttatttttattaaaaatataaaattagtcgggcgtagtggcgggcgtttgtaattttagttatttgggaggtttaggtaggagaatcgtttgaatttgggaggcggaggttgtagtgagtcgagattgtattattgtattttagtttgggtgataaaagtaaaaatttcgttttaaaaaaaaaagaattagttaggggtagtggtgaacgtttgtagttttagttatttaggaggtagaggtaggagaattatttgaatttcggaggtagaggttgtagtgagtcgagattgttttattgtattttagtttaggcgagaagagtaaaattttatgttaaaaaaaaaaaaaaaaaaggaaagaaaaaaaataacgattagaaaggaagaaattaaatatatttatagttagtatgattttatatatattatggttttaatggggttaggcgtggtggtttatgttgtaattttagtatttttaggaggttgaggtaggtggtttttttgggattagttggttaatatggtgaaattttaattttaataaaaatataaaaaattagttaggcgtggtgagggtatttttaattttagttatttaggaggttgaggtaggagaattgtttggatttgggaggtagaggttgtagtgagtcgagatcgcgttattgtattttagtttgggtaataagagtgaaatttcggtagggtgtggttttacgtttgtaattttagtatttcgggaggttgagttaggtcgattatttgaggttaggagtttgagattaatttaatatggtgaaatttcgtttttattaaaaatataagaattagttgggtgtagtggtgggcgtttgtaattttagttatttgggaggttgagatagaagaattgtttgaatttaggaggtggaggttgtagtgagttgagattatgttattgtatattacgtcgggtaatagagcgagatttcgttttaaaaaaaaaaaaaagatgaaattttattttaaaaaaaaaaaaaagttttaatggaaaatttataaaaagttattaaaattaataaataaatatagtagggttgtaggttatagggtaatatagttatttttttatttgtaggggtttggttttgggattttttatatattaaatttatagatgtttaagttttatatataagacggaatagtatttaatttatatatatttttttatatagtttaaattatttagattatttatattatttttatataatgaaaatgttaatgtatatgtaagtatgtatgtaagtatttgtattatattgtttagggaattattggatagataggtttttaagattgatattagtagttattgttaagattttggttaggtttgtttttgtttggggttttagttgattttattgtttttttatttagttaagggtatttgtattttttttggttttttggttatttggaaggtttagtttagtttggtatatttgtattttggtttattgatgttggtatttttgggaaggttttgttttgaaaaatacggagattttagttgttattgaagatttgagagataaagatagggagatttgtttgtagatttgtgtttttttaagtgggattgagattttgggttttttattttaggatagtattttttggtttgttgattgaatagatttttgaaggaggtgtagttgtattttaggagtgggggtgggagtagtattattgattcgtattaataattatatagttttttttagaataataatatagaataagtgaaatagaataattgtagaaagagttaatttttgttgagtttttattgtgtgtttagtattttttttaattttatattttttataatatatagagtattaggtaggcggggtttgggggtttacgtttgtaattttagtattttaggaggttaaggggggtggattatttgaggtcgggagtttaagattagtttgattaatatggtgaaatttcgtttttattagaagtataaaattagttaggtgtggtggtatatgtttgtagttttagttatttagtaggttgaggtaggagaattatttgaattcgggaggaggttgtagtaagcggagatagtgttattgtattttagtttgggtaataagagttgagatttcgttttaaaataaaataaaataaaataaaataaaataaaataaaataaaaaaagaaaagagtttgttattaaaggagttgtttggtaggggatgttttgttagtgtaaataatagaaaagtgggttgggtatagtggtttatgtttgtaattttagtattttgggaggttaaggcgggcggattatttgaagttgggagtttaagattagtttgattaatatggagaaatttcgtttttattaaaaatataaaattagtcgggcgtagtggtcgatgtttgtaattttagttattcgggaggttgaggtaggagaatcgtttgaatttgggaggtagaggttgcggtgagtcgagatcgtattattgtattttagtttggacgagagtaaaattttgttttaaaaaaaaaaaaaaatagaaaagtgtaataaatatttatagtaggtatgttttttagtaaatttgatgataaatttggtataaagaaagagagtatttttgaaaaaaaaaaaaagaaaaagaaagagagtattttgtttgggtaatatagtgaaattttgtttttataaaaaaatttaaaaattggtcgggtgtagtggtttatatttgtaattttagtattttgggagtcggaggcgggaggattatttgaggttaggagttcgaaattagtttggttaatatggtaaaattttatttttattaaaaatataaaaaattaattaggcgtattggtgggcgtttgtaattttagttatttaggaagttgaggtaagaggatcgtttgatattgggaggtggaggttatagtgagtcgagattatattattgtattttagtttgggtgatagggcgagatttcgtttttaaaaaaaaaaagaaaaagaaaaagattaaaaaattagttaggtaggtttttgtggttttagttatttgggaggttgaggtaggagaattattgagtttaggagtggtaggttgtagtgagttatgattgtattattgtattttagtttgggttttaaagtaagattttgttttaaaagaaaaaagaaagaaagaaagaatatggcgggttaggtatagtggtttatatttgtaattttagcgttttgagaggtcgaggtaggtggattataaggttaggagttttatattagtttggttaatatggtgaaattttgtttttattaaaaatataaaaaattagtaggtagggtggtaggggtttgtaattttagttattcgggaggttgaggtaggagaattgtttgaaattagaaggtagaggttgtagtgagtttagattgtattattgtattttagtttgggcgaaaagagttaaattttattttaaaaaataaataaaaaaataaaataaaaaaaaatatggtagtttttgaaagtttgtttgggagaaggtgcgatgatggttgtataatttcgtgtaagatgttggtttatataggggttgttttttgttttttttcgtttttttaattttttatataataggtttgtgtgttatgtatatttattgagtttaagtaggtgtaaggtattgtgatttaatattttggttagtaagataataagatagattattgttttgtttttaggaagtgtatatgttattagaggaaatagataaaataaataaggaaaagtattagataatgtaagtgttatgagaatgtaaatgaggtgatgtgaattaaaataggatgatttaagtttgtacggaaggtttttatttttatgtttttggttagttaaggaattattagttgattagtagagaagggtagttcgtttagttagagtttttggggaagagggagtggttgttaagagatgagattaaagaagtcgagacgggtttttcgtgagggggggttgtaatgtagggttgaggagtgttcgaagagaatgggtaggtgagcggtgagatagttgtttttttagaagttttgtagtgaaaggaattaaagaaatggagtcgtgtattaggtggggaagggtgggggttaagggggtgtttttttttatatagagattgtaggttgagaatgattatatttttgttaataggaggtgggagtagggtacggtagtttatatttgtaattttggtattttaggaggcggaggcgggtcgattatttgaagtaaggagttcgagattagtttggttaatatgtaaagttttgtttttattaaaaatataaaaattagttgggtgtggtggtattcgtttgtaattttagttattcgggagattgaggtaggagaatggtttgaattcggaaggtagaggttgtagtgagttgagattatgttattgtgttttagtttaggtgatagagagagattttattttaaaaaaaaaaaaaaatataggaagggagttgggaatagggtgtatatttaggaagttttggggatttagtggtgggaaggttggaagtttttttttgattgttttttttttaaagaagtgtatggttggtgtggggtggggtaggagtgtttgggttgtggtgaaatattggaagagagaatgtgaagtagttattttttttttgttttataggaagtcgagttgttttagatattggtatggtgttgggggagggggttttttttttgtaggtttaggtgatttagggttggaagtgttttatgttggatttttatttttttttttgtagt

SEQ ID NO: 51 - DNA, 12963 bp, Artificial Sequence
  chemically treated genomic DNA (Homo sapiens)
gttgtaagaggaaaagtggggatttagtatgagatatttttaattttgggttatttgggtttgtagagaaggaatttttttttttaatattatgttagtgtttgagatagttcggttttttgtggagtaggaaaagaatggttgttttatatttttttttttaatgttttattataatttaagtatttttgttttattttatattagttatgtatttttttgaggaaaagataattagagagggatttttaatttttttattattaaatttttaagattttttaaatgtgtattttatttttaattttttttttgtatttttttttttttttgagatggagtttttttttgttatttaggttggagtatagtggtatgattttagtttattgtaatttttatttttcgggtttaagttatttttttgttttagtttttcgagtagttgggattataggcgagtattattatatttagttaatttttgtatttttagtagagatagggttttgtatgttggttaggttggtttcgaattttttattttaggtgatcggttcgttttcgttttttaaagtgttaagattataggtgtgagttatcgtgttttgtttttattttttgttaataaggatatagttatttttagtttgtaatttttgtatggggaaggatatttttttggtttttatttttttttatttgatatacggttttatttttttgattttttttattgtaaagtttttggaagaataattgttttatcgtttatttgtttatttttttcggatattttttagttttgtattataatttttttttacgaagggttcgtttcggtttttttaattttattttttaataattatttttttttttttaaaagttttagttagacgggttgtttttttttgttaattaattggtggttttttggttagttaggaatatgggggtaggggtttttcgtgtagatttaagttattttattttaatttatattattttatttgtatttttatagtatttatattgtttgatatttttttttgtttattttatttgttttttttaatagtatatatattttttaagggtagggtagtgatttattttgttgttttgttgattaaagtattagattataatgttttgtatttgtttgggtttaataaatgtgtataatatataagtttgttatatgagaggttaagagagcgagaaagagtaaggggtagtttttgtgtggattagtattttgtacgaagttatgtaattattatcgtatttttttttagataagtttttaaaggttgttatgtttttttttgttttgtttttttgtttgttttttgagatggagtttggtttttttcgtttaggttggagtgtagtggtgtagtttaggtttattgtaatttttgttttttggttttaagtaatttttttgttttagtttttcgagtagttgggattataggtttttattattttgtttgttgattttttgtatttttagtagagatagggttttattatgttggttaggttggtgtggaatttttgattttgtgatttatttgtttcggttttttaaagcgttgggattataggtgtgagttattgtgtttggttcgttatgttttttttttttttttttttttttttgaggtagggttttgttttgaagtttaagttagggtatagtggtgtaattatggtttattatagtttgttatttttgggtttagtgatttttttgttttagttttttaagtagttgggattatagaggtttgtttggttaattttttagtttttttttttttttttttttttttggagacggagtttcgttttgttatttaggttagagtgtagtggtgtgatttcgatttattgtaatttttattttttagtattaagcgatttttttgttttaattttttgagtagttgggattataggcgtttattaatgcgtttgattaattttttgtatttttagtagagatggggttttgttatgttggttaggttggtttcgaatttttgattttaggtgattttttcgttttcgatttttaaagtgttgggattataggtgtgagttattgtattcggttaatttttgagtttttttgtagaggtagggttttattatgttgtttaggtaggatgtttttttttttttttttttttttttttttagggatgttttttttttttatgttaaatttgttattagatttgttaagaaatatgtttattgtaagtgtttgttatatttttttgtttttttttttttttgagatagagttttgttttcgtttaggttggagtgtaatggtgcgatttcggtttatcgtaatttttgttttttaggtttaagcgatttttttgttttagtttttcgagtagttgggattataggtatcggttattgcgttcggttaattttgtatttttagtagagacggggtttttttatattggttaggttggttttgaatttttaattttaggtgattcgttcgttttggttttttaaagtgttgggattataggtatgaattattgtgtttagtttatttttttgttgtttgtattgataaaatattttttattaaatagtttttttaatggtaggtttttttttttttttattttattttattttattttattttattttattttattttgagacggagttttagtttttattgtttaggttggagtatagtggtattattttcgtttattgtaattttttttcggatttaaatgatttttttgttttagtttgttgagtagttaggattataagtatgtgttattatatttggttaattttgtatttttagtagagacggggttttattatgttagttaggttggttttgaattttcgattttaggtgatttattttttttggttttttaaagtgttgggattataggcgtgagtttttaagtttcgtttatttagtattttatgtattatgggaaatgtagagttgaggaaagtgttgggtatatagtaagagtttaataaaggttagttttttttgtaattgttttattttatttgttttatattattattttagagagaattgtgtgattgttagtgcggattagtggtattgtttttatttttatttttaaaatgtaattatattttttttagggatttatttagttaataggttaggaggtgttgttttgaaatggggggtttaaagttttaattttatttggagggatataggtttatagataggtttttttgtttttattttttaaatttttagtagtaattaaaattttcgtgttttttagagtaggatttttttaggggtattagtattagtgggttaggatataaatgtgttaggttgaattaggttttttaaatggttagggagttaagagaaatgtaggtgtttttggttgggtgggaaggtaatgagattaattgagattttaaataggggtaggtttgattagaattttaatagtggttgttggtattagttttgaaggtttatttgtttagtgattttttaaataatatagtataagtatttatatatatatttgtatgtatattagtatttttattgtatgggggtaatgtaagtaatttagataatttaaattatatgggaggatatgtgtaggttaaatattatttcgttttatatatgggatttagatatttgtgggtttggtgtgtgaggagttttagaattaagtttttatagatagagggataattatattgttttgtaatttgtaattttgttatatttatttattagttttggtagttttttatggattttttattaggatttttttttttttttgagatagagttttattttttttttttttttgagacggaatttcgttttgttgttcggcgtggtgtgtaatggtatgattttagtttattgtaatttttattttttgggtttaagtaatttttttgttttagttttttaagtagttgggattataggcgtttattattatatttagttaatttttgtatttttagtagagacggggttttattatgttaggttggttttaaatttttgattttaggtgatcggtttggtttagtttttcgaagtgttgggattataggcgtaagattatattttgtcggagttttatttttgttgtttaggttggagtgtaatagcgcgatttcggtttattgtaatttttgttttttaggtttaagtaatttttttgttttagttttttgagtagttgggattagaggtgtttttattacgtttggttgattttttgtatttttattagagttggggttttattatgttggttagttggttttagggaagttatttgttttagttttttaaaagtgttaggattatagtatgagttattacgtttggttttattaggattatggtatgtatagaattatattggttgtgaatgtgtttgatttttttttttttaatcgttattttttttttttttttttttttttttttttttgatatggaattttgtttttttcgtttaggttggagtgtaatgggataatttcggtttattgtaatttttgttttcggggtttaagtgatttttttgtttttgttttttgagtagttgggattataggcgtttattattatttttggttaatttttttttttttgagacggagtttttgtttttgttatttaggttggagtgtaatggtgtaatttcggtttattataattttcgttttttaggtttaagcgatttttttgtttaagttttttaagtagttgggattataggcgttcgttattacgttcggttaattttgtatttttagtagagatggggttttattatgttggttaggttggtgttgaatttttgattttaggtgatttatttatttcggttttttaaagtgttggggttataggtatgagttattgtattcggttaacgtttggttatttttttaatattttaatagagacgaggttttattatttttgttaggttggtttttaattttagattttaggtgatttgtttatttcggttttttaaagtgttgggattattggcgtgagatatcgggttattaattattatttttttttttttttttttttttgagatatagttttgttttgtcgtttaggttggagtgtagtggttcgattttagtttattgtaagtttcgttttttgagtttacgttatttttttgttttagtttttcgagtagttgggattataggcgttcgttattacgttcggttaattttttgtatatttagtagagatagggttttatcgtgttagttaggatggttcgattttttgatttcgtgatttattcgtttcggttttttaaagtgttgggattataggtttgagttatcgtattcggtttttattattttttttttttgtttggttaaaatttttagtatggtattaacgttgtgagagttaaatttttttttagtttttgattttagaggaaaaagcgttgagttttttttttttttttttttttttttttttttgagacgaagttttattttgttatttaggttggagtgtagtggtacgatttaggttttgtaagtttcgtttttcgggtttacgttattttttcgttttagtttttcgagtagttggtattataggcgttcgttattatgttcggttaattttttgtttttgtatttttagtagagacggggttttagtatgttagttaggatagtttcgattttttgatttcgtgattcgttcgtttaggttttttaaagcgttggtattataggcgtgagttatcgtttttagtagtattgagttttttattattatgtatgttgttagtggaatttcgattgtggacgttttgttattaaattagttaagttttttttttttagtttgttaggaggttggttggtttgtaattatgtatatgtgttgatattattaattgtttttgttatatttgttgaaatgattatagggtttttatgtttttttttgttaatgtggtgaattatatagattgatttttttttttttagtaaagattagtttgattatgttgtttaggttggttttgaaattttgggtttaagagatttttttgttttagttttttaaaatgttgggattataggtttgagttattgtattaggttaatttttgaatgttgaattagttataattatgagataaatattatttggttagaatgtatttattttttttttttttttttttttgagatggagttttattttgttgtttaggttggagtgtaatggtgtgattttagtttagtgtaatttttgttttttgggtttaagcgatttttttgttttagattttcgagtagttgggattttaggtgtttattattatgtttagttaattttttttttttttgagatgaagttttgttttgtcgtttaggttggagtgtagtggtacgattttggtttattgtaatttttgtttttcgggtttaagtaattttgttttagttttttgagtagttgggattataggtaggtgttattatatcggttgattttcgtatttttagtagagatggcgttttattatattggttaggttggttttgaatttttgatttcgtgatttgtttatttcggttttttaaagtgttgggattataggcgtgagttatcgtgtttagtttgatttttgtatttttattagaaacggggttttattatgttggttaggttggttttaaatttttgattttaagtgatttgtttgttttagttttttaaagtgttgggattataggcgtgagttatcgcgattggtttatttatttttttttttatattattaggtttggtttgttaaaattggttagttgttgtatgtttatgttaataggaatattggtttatattttttttttttataatgtttttgtttggttttggtattaggattattttggtttcgaaaataagttgggaaatatttttttattttttttttttttttttttttttttgagatagggttttattttgttgtttaggttggagtgtagtggcgtaatttcggtttattgtaattttcgttttttaggtttaagggatttttgtgttttagttttttgagtaattggtattataggtatggtgtacgtttagttatttttgtattttagtagagatggggtttcgtcgtgttggttaggttggtttcgaattttcgattttaaatgattttttgttttagcgtattaaagtgttgagattataggtatgagttatcgcgtttagtttgtttttttgttttttgaagagtttgtgtaagatgggtattgtttttttttttaacgtttaaagagagtagagaatagaggagataaatagaaaatagtattaagaggttaggtatggtggtttatatttgtaattttagtattttgggaggtcgagatgggatgaaagtttgaggttagtagtttgagattagtttgggtaatatagtgagattttgttaatataaaaaaataaaatagttagttgggtgtggtggagtaattttagttattcgtgaggttgttagaggattgtttgagtttaggggttcgaggttgtagtaagttttgattgtgttattgtattttagtttgggtaatagtgtgagtttttgttttaaaaattaataaagaaaaaaagaaaatagtattaaaatggtagttttatattttaattgtaaaataattaaaattaaaagtatttagtagagaaaggaagtttattttaataagtggagatagaataattggatttttatataggaaagatattagagattgatttttatattttatattataaataattaattttaagaattaattaatggtttaaggattttattgtaaaatttattattataaaggttttaaaagaaaatgtaagattatatttttatgattttggggttaaaaaaaaaaaaaaagatgttttaaataggataaggtaaatattgaatataaaaaagattaatttattttttttaagattttgtaaattttgtaaagtaaataaataggttcgtaatagattagaagaaaatatttacgatttatggatttgataaggatttgtatttagaaagtataaagtagttttataattgaataataaaaataaataaaaaaattaaaataataggtaaaagattcgaagagttattttataaataaaatacgaatggttaataggtatatgaaaaaatgttgaatatttttagttaatagagaattgtaaattataattataaggatatattatattagaaagattgatatatttaatgttcggaaggttgtggtataattataataatttttatattttgttagttggagtgtaaaatggtataatcgttttggaaaatttagagtttttgaaaaagtttaaaatatagttattttttattttttttataattttttttttaagtatttttttaagaaatacgaaaatatatgattataaaaagaattgtataagaatgtttatagtagttttattttataatcgtaaatgggaaataatttaaaaggttattaaaaggacggatatataatcgatggattatattaaatgaaaaggagtaaaatattgatatatataatatgaacgaatgttagatagtatattgaaggatagaagttcgataaaaatgagtatataatgtatgattttttttttttttttgagacggagtttcgtttttgtcgtttaggttggagtgtagtggtacgattttggtttattgtaatttttgtttttcgggtttaagcgatttttttgttttattttttcgaatagttgggattataggtatttattacgtttagttaatttttgtattttttagtagagacggggatttattacgttggttacgttggtttggaatttttatttttaagtaattcgttcgtttcggttttttaaagtgtaggcgtgagttaaagtgtaggcgtgagttatagcgtttattttgattttattttatatgaagttttttaataggtaaaatggttatggagattaaaataaaggtggggtcgggaatcgattgggaagagacgtgatgaaacgtttttgggacgatgaaaagggtttgtgatttggtaggtattacggagcggttaggggttaaaatttatttttttgtgtatttgttgtgtgtattggcgttgtgtgtaaatgttatttcgatttaggaaaaagatgacgtaagtacggtataaagtggtcggtacgcggtaggtgtatgggaagaaattgcggaatgaaataatcgcgagttaagagatggggtagcgggagaaatgaattcgagtttcgttttttattaggaagaatcggttcgggtcgagggttgtacggaggattatacggacgtttgcgggttcgtttttttcgttttacgacgtttagtttgcgtttggaattggaatggtttagtttaaagttagataataggtagattgtttttttcgataaattattaaacgatttattattgtatttttttaaaattttatttttagacgtattattttttttttttttttttcgggaagatgagatatgtttatttttgaaagtgttttcgggtttgttttttgtatatttttttttttttttgtttacgttatggtagcgttcgtttaggttgtaggcgattcggggggtggggtatattatttaaagaaggggagggattgaggtttgtattaaaataaatatttttgtttttgtaaaggttataattaagtaatttagaaaaagaaatgtaggcggagaatagtagtttttttttgttaagtaagaggaatcggtttaaaggatatttttttttttttttttttttttttatcgggtgaatagtgagttgtttcggtaaaaagaaatcggaaatgttgttgtaagaggtagaaatgtaaatgtggagttaaataataatagggttgtcgggttttttagattgcgacggtttttttcggtttggcgggtaaatttttggtttagtattttttatttttacgattgatagtttttaattggattttttttatttagcggagtcgggggttgtttggaaagatcgttttaggaaggataaaggttcggaagttgtgggattttagtagtttgggtttttcggattatttttaaatgattatttcggaatggagttttagtttttattaggatgttatgggttttaaaatatatagttatgagtttttaatgtttcgagatttaaaagttttagattttaatgttttgtgtattttttattttagggattttttacgtttagtatcgggtggatgtgtaaagaagtacgttttaggtcggtttaaggttttttaaagttttatttttttgtttaggcgtttaattttgagttcggatggttttaatatttttattatttatatttaggttttttaataatgtaatttttatgatgatttttttagttaagtttttattttatttatttttaaattcgttaagtttttattgttttatttttagttttagcgattttttcgagttgaaaatatacggagtcgagagttcgtgatttagagaggatttattaagtttagttaggagtttatttaatttagggaagcgtgttatcgtcgtggaaagtacgtttttagttcgaacgtaaagtgttttcggagtttagtagttatttgttttttggacggtggttttagatttttgagaagtttaaaatttttagcgttagttttgagtatatgggaggggaaaattttaattttattaatttttgcgaggtttttggtataaagttggatagtcgttatgataagtaagggtaagtaattcgtttgtcggaggaagtaaaggaaatggagttggggaggagggtgtagagttaggattttcgtcgatttggtgtcgtagatattaatattttggggtggaaaattttgtaagttagagttgtgagggtagaattggtggaaattattttggaggaattttgtattgtgttaaatatgaagggtggaaggaagaaagtttttgcgtttgtttttagttggattttttttttttattagttaaaatgttattttttaggaaggtttttcgtaatattatattttaacgtttttttttagatattttatattatattattttatttaattttttttataatttttattattttgataagatttatttgtttattgtttttagtatatggaaacgtaagttttatgaggatatagaatttttttattattttatttattgttgtatttttgagtgtttatattagtgttgggtagtaagtaagagttcgataataaatattttttgaatgagggagataggtttgaagtttggagaatgagatgtagaagaggtgtaagatttgttgcgttttttgtaggcggcgggggggcggtgtaggtgttttaagaattatcgcgggattcggtagggggagcgtaggcgtttttcgttaagatagaagcgtttagattataatttttagtagttacgaggagttttagggtttgatgggaacgggaaattttttaattttttacgtttcggtttcgcgggtttcgtgggtcgttcgcgaaatttgattcgggatgcggcggtttaatcggaaggtggatcgaaatttcgcgatagtaagaggttcgtagcgattcgcggtgttaaggaatatagtgtttttaaaagaattggcgttcgttgttcgttttttttttcgggagttttttgtttatttttagaagaggagggaagtataggtgggtttttttagttttgcgtcggatttttgagaatttcgaagttattttggttgaggttaattttcgttgtgttttttttgtagtatgaagattttggagatttaatcgttagtttcggattgttgttttttagattaggatttagttttagtttatttttttttttacgttttttcgatgaataaaaatgcggattttgaattgatgttatcgtttttcgaaaggggggattcgtttcggttgtttttagattttgtggttggtttagttgtgttttaggagttacgggagggggatttagtttttttttttatttttttggaaatagagtttggtttttttagtgagttgagttttcgaatcgaggagtaagaattttttgaaaatataagtttttttgtagaagaagtaaacgggagtttttttgaagaagaagcgaacgggttagagttggggtttggaaaagttatggaagatatttttggggaattcgttgtagaggacgagggagatacgtaagtggtgatggtagtggagtgtggagtttggggagatgaagtgtgaggtcgatttgttttttggttttgagatttatttttttaggtttttgttttttttgtttggcgatttaggtttattgttttttattttttttttagcgtttggaattatagttttttttagttgtttcgattttttagtggtttttggttagagtttagtatttaatttgagaattttttgaaaggttgtaagtggtaaggataataacggggtagggagttgattattttcgagatttttattgtaaaatagtttagttttttaggagagggatgttttagagttgggagaagcggtacgtagtttttttagattgagtttatattttatttagtagtttgtgttttttattttttttaaggatttaggggggtttatttattttagaggtaggtttagttttagttttatatttgaaaagtataggtttggttagttttttaatttttttttgtttttagggtttttgat

SEQ ID NO: 52 - DNA, 3500 bp, Artificial Sequence
  chemically treated genomic DNA (Homo sapiens)
tttagaataaatggtagaggttagtgagtttagagatggtgatagggtaatgatttaggggtagttgtttgaaacgggagtaggtgaagttatagatgggagaagatggtttaggaagaaaaatttaggaatgggtaggagaggagaggaggatataggttttgtggggttgtagtttaggatgggattaagtgtgaagatattttagtaggtgaggttaggttttatgaatagagaagtagtttttattttttttgatgtacggatatatagagtgtgtggtgttgtgtttttagagtcgggtttttttgttttggtttttagggagtgagaagtgaggttgatttgtttttgtttttttttgttattttaatatttattttttttttatgttttttttttttaaatatgatttggatttatgttttcgtttaaattttatgttaaattgtaaattttaatgttggaggtggggttttgtgagaagtgattggataatgcgggtggattttttgttttgatgttgtttttgtgatagagattttatatgatttggttgtttaaaagtgtgtagtatttttttttttttttttttttttttttatttatgttttgttatgtaagacgtttttgttttttttttatcgtttagaatgattgtaagttttttgaggtttttttaggagtagaagttattatgttttttgtataattgtagaatgatgagcgaattaaattttttttttttataaattatttagttttaggtatttttttatagtaatgcgaggatagattaatataattttttatttttagattttcgtatacgtttagttttagatattattgtttttgggagtatgtatagcgtagttttttgtcgataaaagtaaagttataaaaggtgataaaaatttgtatttggggatatttgattgtgaaagagggaggatagtatatttgtagttatagagattggggtttatcgagttgaaatttggtagtattttggtataatatgtgtatgattcgtgtttaatgtttagagattagtgttgagtaaaatagtttggtttggggtcgttgttgtttttattttttttttgtttattagagggcggtagagttttttttattttggagtttttttaggggttgttgattttttttagtcgggtttatagtttagtagggtttatttttattcgggttatttcggtttacgtttttttcgtttttcgagttttttatacggattttgttagtttttttttgtagtttatcggtcgtttatttgaggtttgtcggtcgtttatttgaggtttgtcggttgttttttgtaggtagtttttgttttttatatttttttttttttcgggtttagttgaaagggcgttttttagggtagttttttgtgatttttaggatagtttagttttttataggtttcgacgttttttatgttgttattttatagttttgttattattattaattttttagttttatgaagtttattgagcgtttgtttttcggttataggaaaattttgtgatagggattacgtttgttttgttttttgtggaattttagggtttagtttagtgtttgatacggaatagatgttttataaatattggttaaatgtgtgggagatttttaaaaagaagtatattattttcgtgtggtttttagtagttagagtttgttttatgtggatataggggtattggtattagtatgggaggaggttagtaagtgttcgcggttgttttaggaatgaggttttaatttttagagttttagaagggaggatagaggtttgtagggaatagatttttcggtttgattttgtagtttaatttagagtttagggttagtttatattacgtcgattttggttagtatttttagggtagttttagataaggtcggaggtttttttttgttttttagggggtgatattgtatatagatattatttaggaaacggatttttttggataggaatttggttttgttaaggaagtggaggtggagtttggtttttattttttgttttaatagatttttttgattttttttatatatttgttttgtttttttttgggttttatgaggattttgttttgttaggggtttttgtgtaattttagatttttttttggtattattatggggaaggtggggtgattataggatagttagtttcgtagagatagagattatttaggattgttagggagaatatggataggttttgagtcgtagtttagttaatagatacggagagggagggtttttttggagttttttttaaggatagtagagtttagagttatttatttttttttattatagttttttttttttaggatatataagatatttttttttttatatgtaggatttggggatttttgagatttttgggtttgggtttttatttttgggttagtggcggggttggtggtattggagatagagggttggtttttttttagttattatttagtgagtttttttttagtttttagagttatttttgttatttttttgttgggtattattttatttttttagagttttggagagtatggggagattcgggattttgttgggtttttttgttataaaggaaaataatttttttggtgtgatagatttaaggatagaatatagtagaggttagtattggggaagataggttgtttttttaggggatgggggtttatttattttgtcgaaaagatttgtttgaggaattgaaaatagaagggaaaaaagaggagggataaaagaggtagaaatgagaggggaggggatagaggatatttgaataaagattatatttatgatttacgtgatgttgagaagtatttttgttttaggaagagatttagggtagagggaggaaggatagtagattagatagttatagtagttttgataaaacgtttttggaatttaagtttttttttatagaggaggatagagtagatagtagagattatggagtttttttcggtttttttttatagatggtgtattttttggtagaggtttttgtttataggtgaagggaggataatttgggagagggtgggaggagggagttggggttttttgggtaggatagggttgtgagacggatagagggtttttgttggagtttgaatagggaagaggatattagagagggataggagttatattagaaaaattaaattgaattggaattggaaaggggtaggaaaattttaagagttttatttttttagttaattgttattggttattacgtttttaaaaattataataattgtattagatgatattttaaataaaaatataattagggtatgaaatattgtttttattcgtttatcgcggatattggaaaataagttttaggttgtggagggttttgggaatttttatgaatttatttataggaatttgtagtttgttttaggtattggggtgtaattaagatt

SEQ ID NO: 53 - DNA, 3500 bp, Artificial Sequence
  chemically treated genomic DNA (Homo sapiens)
gattttggttgtattttagtgtttgggataggttgtagatttttgtggatgagtttatgagggtttttagggttttttatagtttggggtttattttttaatgttcgcggtaggcggatgaggatagtgttttatgttttggttatgtttttatttaaagtgttatttgatgtagttattatgatttttaaaaacgtagtggttagtgataattaattaggaaaatagaatttttgaggtttttttgtttttttttaattttagtttaatttgatttttttggtgtgatttttgtttttttttgatgtttttttttttatttaggttttaataggagttttttgttcgttttatagttttgttttatttaggagattttagttttttttttttatttttttttaggttgttttttttttatttgtgagtaggagtttttgttaggggatgtattatttgtggggaggggtcgagggagattttatggtttttgttgtttgttttgtttttttttgtggagaagagtttgagttttaggaacgttttgttaaggttgttgtgattgtttggtttgttgttttttttttttttgttttgagtttttttttagggtaggagtattttttagtattacgtgggttatgggtgtggtttttatttaggtgttttttgttttttttttttttatttttgttttttttgttttttttttttttttttttttatttttagttttttagataaattttttcggtaaggtggatggatttttattttttgggaggataatttgttttttttagtgttgatttttgttgtgttttgtttttgggtttgttatattagggggattatttttttttgtgatagagaaatttagtagggtttcgggtttttttatgttttttagggttttgggaaggtgggatgatgtttaataggaaggtgatagaggtggttttgggggttagaaaaaggtttattgggtggtggttggggagggattagttttttgtttttagtattattaatttcgttattgatttagggatggagatttaggtttagaggttttaggagtttttagattttgtatgtggagggggaggtgttttgtgtgttttggaaagagaggattgtggtggagggaggtgggtgattttgggttttgttgtttttggggaaggttttagggggatttttttttttcgtgtttgttggttgagttgcggtttagggtttgtttatgttttttttgatagttttgggtggtttttgtttttgcgaggttgattgttttgtgattattttattttttttatggtggtattaggagggagtttggagttgtatagggatttttggtagaatagggtttttataggatttagaaaggaatagagtaggtatgtgggagagattagaagggtttgttggagtaagggatggaaattaggttttatttttatttttttagtaaagttaggtttttgtttaggggaattcgttttttgagtgatgtttgtgtgtaatgttattttttggagggtaagaggagattttcggttttgtttggaattgttttagggatgttgattagggtcgacgtggtgtgagttgattttgaattttggattaggttgtagggttaggtcggaggatttattttttgtaggtttttgtttttttttttgaagttttgggggttgaggttttatttttggggtagtcgcgggtatttgttggtttttttttatgttggtgttagtgtttttgtgtttatatggaatagattttgattgttgggggttatacggaggtgatatgtttttttttagagattttttatatatttaattagtatttatggagtatttgtttcgtgttaggtattgggttgggttttgggattttatagagagtaggatagacgtggtttttgttatagagtttttttgtaatcgggagataggcgtttagtgaattttatgggattgaggagttaatggtaatgatagggttgtgaggtgatagtatagggggcgtcggagtttgtgagagattgagttgttttggagattatagggagttgttttgggagacgtttttttagttgagttcggggaaggagggggtgtaggggataggagttgtttgtagagggtagtcgataggttttaagtgggcggtcgataagttttaggtgggcggtcgataggttgtagggaggagttgatagagttcgtgtgaggagttcggagggcgaggaggacgtgggtcgaggtgattcgggtgagggtggattttgttgggttgtgggttcggttgagggaggttagtagtttttggggaggttttagggtgggaggaattttgtcgttttttggtggataggagggaagtggggatagtagcggttttagattaggttgttttatttaatattgatttttagatattgaatacgggttatgtatatgttatgttaaagtgttattaggttttagttcggtgagttttagtttttgtggttataagtgtattgtttttttttttttataattagatgtttttaaatgtagatttttgttattttttgtgattttgtttttgtcggtaggaggttgcgttgtgtatgtttttaggggtagtgatgtttggggttaagcgtgtgcggggatttgggagtagaagattgtattagtttgttttcgtattgttataaagaaatatttgagattgggtaatttataaagaaaagaggtttaattcgtttattattttgtagttgtataggaagtatagtggtttttgtttttggggaggttttagaaaatttataattattttggacggtgaaggggaaataggaacgttttatatggtagagtatgagtaagagagagagagagagaggggagaggtgttatatatttttaaataattagattatgtgagatttttattatagaaatagtattaaagtagaaaatttattcgtattatttaattattttttataaggttttatttttaatattggggtttataatttgatatgagatttgggcggggatatagatttaaattatatttgagagagaggggtatgaggagaaggtgaatgttggggtagtagagaggagtagggataagttaattttattttttattttttggggattagaataggagagttcgattttgggggtatagtattatatattttgtgtgttcgtgtattaggggaggtgggagttgtttttttgtttatgggatttggttttatttgttgagatgtttttatatttagttttattttgggttgtagttttatagagtttgtgtttttttttttttttttgtttatttttggatttttttttttgaattatttttttttatttgtggttttatttgttttcgttttaggtagttgtttttggattattgttttgttattatttttgggtttattggtttttgttatttgttttgag

SEQ ID NO: 54 - DNA, 3315 bp, Artificial Sequence
  chemically treated genomic DNA (Homo sapiens)
ttttttattttttattgttatttatttagtgttgtgtagtagtttagttgtgtgtttgttgggaggggttgttaagtgttttgtttattggttgttttttgaatttttgttattttatgtataaatatatttatatattttttttgtttagtttatatattgagttatttgtatatgtgagtatatttttttttttttttttatttttttggtttttgatttttataagtttatggaatatttttggaaagatgtttttgatttagtagggtaggtttgttttgatttttttttttgtagttttagtattttgagaaagtaatttatttttttggttagtgtttgtattttagtagggagatgaggattgttgttttttatgggggtatgtgtgtgtttttttttttttttaggatttgtaggattttttgtgttatttgtatataatttggtaggtttatattttttaagagttttatgaagtgttttttgtatgtgttttaaaaaggtatttgaaaattgaaagtgtgatttatggaaattaaattatttgtaaaaaattgttttggaaagtaatgattgttggttataaagggaaatatttgtgatgtatttaatgtgtttttaattttttatttgttgataatttatagttattaatgttaaatttgattttggttttagttatatttgtatattgtttaataatggtttatttttgtaagaattagataaaatgtatatttgatataaaatagttaaaaatgtaatttttagtaatagtaagtttggtatttagatagattatgaatattttgttagatattttgttgggtgtttgggatagtaattaaaataaagtattgatagttgtattagagtttattaggttgtagtaaaggaagtttatttaaaagtataaattatttaagattatagatgtatgatatattttatttattttttgtttttttaatatgtatatatatatatatatatatatatatatatatatatgtgtgtgtgtatgtgtgtgtgtatgtttaatttttaatttagttaaaaatttttttttatttgttttttatttggatatttgattttgtatattttagtttaagtgaattgagaagattgagttgtaggattaaaggatagatatgtagaaatgtattttaaaaatttgttagttggattagattgataatgtaatataattgttaaagttttggtttgtgatttgaggttatgtttggtatgaaaaggttatattttatatttagttttttgaagttttggttgtataattaatttgtggaaggtatgaatatttatgtgtgttttaattaaaggtttttttgaattattttttatatgagaatttttaatgggattaagtatagtattgtggtttaatataaatatataagttaggttgagagaattttagaaggttgtggaagggtttatttattttgggagtattttgtagaggaagaaattgaggttttggtaggttgtatttttttgatggtaaaatgtagttttttttatatgtatattttgaatttttgtttttttttttttagatgttttttgttagtttttttagttgttaaatatagttgtttgtggttggttgtgtatgtaattgtatattttattttatttgttttattttggttatagtgtagttttttttagggttattttatgtatatattatgtatttttagttaatgaggagggggaattaaatagaaagagagataaatagagatatattggagtttggtatggggtatataaggtagtatattagagaaagttggtttttggatttgttttttgtgtttattttaagtttagttttttttgggttatttttagtagatttttgtgtgtttttgttttttggttgtgaaatttagtttttatttagtagtgatgataagtaaagtaaagtttagggaagttgttttttgggattgttttaaattgagttgtgtttggagtgatgtttaagttaatgttagggtaaggtaatagtttttggttgttttttagtatttttgtaatgtatatgagtttgggagattagtatttaaagttggaggtttgggagtttaggagttggtggagggtgtttgttttgggattgtatttgtttttgttgggttgtttggttttattggatttgtaggtttttggggtagggttggggttagagtttgtgtgttggtgggatatgtgttgtgttgtttttaattttgggttgtgttttttttttaggtggtttgttggtttttgagttttttgttttgtggggatatggtttgtattttgtttgtggttatggattatgattatgattttttatattaaagtatttgggatggttttattgtattagatttaagggaatgagttggagtttttgaattgtttgtagtttaagatttttttggagtggtttttgggtgaggtgtatttggatagtagtaagtttgttgtgtataattattttgagggtgttgtttatgagtttaatgttgtggttgttgttaatgtgtaggtttatggttagattggttttttttatggttttgggtttgaggttgtggtgtttggttttaatggtttggggggtttttttttatttaatagtgtgtttttgagtttgttgatgttattgtatttgttgttgtagttgttgttttttttgtagttttatggttagtaggtgttttattatttggagaatgagtttagtggttatatggtgtgtgaggttggtttgttggtattttataggtatttgtgtttgtgttgtttgttggggtggttgttgtgtttggtaggagggagggagggagggagggagaagggagagtttagggagttgtgggagttgtgggatgtgtgatttgagggtgtgtgtagggagtttggggtgtgtggtttagtttgggggttttgtgtgtagtttgtgttgtgtttagagttaagtttttttgttgggtagttgaaaaaaatgtattttttatttatttattgtttgtgtgagaggtagatttgaaagtttgggttttttaataaaatatatgttggaaaattagataaagtagtagttatttgtgggggaaaatatttttaggtaaataaatatggggtgttttgagttatttgggaaggttttgtttttggtatttaaagttgggggtgtttggagttagtagagtttagtagagttttatttatttttttaatgtttttgtttaatgtgttttttaaatttttttttatttagattat

SEQ ID NO: 55 - DNA, 3315 bp, Artificial Sequence
  chemically treated genomic DNA (Homo sapiens)
atagtttagatgaaaggaaatttggggagtatattaaataaaaatattaaaaggataaataaaattttgttgagttttgttaattttaaatatttttaattttaaatgttaagagtgagatttttttaagtgatttaaagtgttttgtgtttatttgtttggaggtgtttttttttataaataattgttgttttgtttggttttttaatgtgtgttttgttaggaagtttgggtttttgggtttgttttttgtatggatggtaagtgggtggagagtatgttttttttagttgtttggtgagagaatttgattttgaatgtagtgtgggttgtatgtagaatttttgggttgggttgtgtgttttgggttttttgtgtgtatttttgggttgtgtgttttgtggtttttgtagttttttaggttttttttttttttttttttttttttttttttttgttgggtgtggtggttattttgatgggtggtgtgggtgtgggtatttgtagaatgttggtgggttggttttgtgtattgtgtagttgttgggtttgttttttaggtagtagggtatttgttggttgtggggttgtaggaaaggtgatagttgtggtggtgggtgtagtagtattagtgggtttggagatatgttgttgagtggggggaaattttttaggttgttggagttgaatgttgtagttttagatttggggttgtaggggaggttggtttgattgtagatttgtgtgttggtggtggttgtggtgttgaatttgtaggtggtgtttttggggtagttgtatatggtgggtttgttgttgtttaggtatattttgtttaggggttgttttagggggattttgagttgtggatggtttaggggttttagtttgtttttttggatttgatgtagtagggttattttagatgttttggtgtggagggttatggttatggtttgtggttgtgggtagggtgtagattgtgtttttgtagggtagaaggtttagaaattggtgggttatttggaaaaagagtatagtttgaggttagaggtgatgtagtgtatgttttgttgatatgtgagttttggttttggttttgttttgggagtttgtgggtttggtgaagttgggtgatttgatgggagtaagtgtagttttaggatgaatgttttttgttagtttttgggtttttgggtttttaattttaagtattggttttttgagtttatatgtattataaaggtgttggaggatggttagggattgttgttttgttttgatattggtttaaatattattttaggtataatttgatttggagtgattttaaagagtagtttttttgaattttattttatttgttgttgttgttggatagaggttgagttttatggttagggggtgggggtgtatgaggatttgttaaaggtggtttagggaagattgggtttaaaataaatgtgaaagatggatttaggggttggttttttttaatgtgttgttttatgtgttttgtgttagattttgatatatttttgtttgtttttttttttgtttgattttttttttttgttggttagaaatatgtagtgtgtatataggatgattttggggaggattatattgtaattgagatagggtagatagaatggggtgtgtggttgtatatgtagttagttatagatagttatatttagtagttgggggaattgatagggggtatttgaggggaagggggtggagatttagggtatatatataggaagagttgtattttgttattaggagaatgtaatttgttaggattttagtttttttttttgtaaaatgtttttaaagtagatagatttttttataattttttgagatttttttagtttgatttgtgtgtttatgttggattatagtattgtatttggttttattaggaatttttatgtgaaggatgatttagaaaaatttttggttagggtgtatatgggtgtttatgttttttataggttggttatgtaattaaaattttagaaaattgaatataaaatgtgatttttttatattaaatataattttaggttatgaattaaagttttggtaattatgttatattgttggtttggtttagttaatagatttttaaaatgtatttttgtatgtttattttttagttttataatttgatttttttggtttatttgggttaggatatgtagaattaaatatttagatgaaaaataaatagaaaaaagtttttaattgaattaaaagttaaatatgtatatgtatatatatatatatatatatgtgtatatatatatatatatatatatatatatatattaaggagataaaaaataggtgaagtatattatgtgtttataattttggatagtttatatttttgaataaattttttttgttgtagtttaatagattttgatataattattaatattttgttttaattgttattttaaatatttaatagagtatttgatgaagtgtttatggtttatttaaatgttaagtttattgttattaagagttatatttttgattattttatattaagtatatattttatttaatttttataaaaatagattattgttggataatatgtaaatgtagttgaagttaaaattgagtttagtattaatgattatagattgttagtaaataaagggttaaaaatatattaggtgtattgtagatatttttttttatggttagtaattattattttttaaagtaattttttatagatgatttaatttttataaattatatttttaatttttaaatgtttttttaaaatatatgtaaaaagtattttatagggtttttaaaaaatgtgaatttgttaaattatatgtaaatggtataaagaattttataagttttgaaagaaaaaggagatatatatatatttttatggagaatagtaatttttatttttttgttaggatatagatattagttagaaaggtaagttgtttttttaaaatgttaaagttatagagagagaaattaaaataagtttattttgttggattaagaatgtttttttagaaatgttttatgggtttgtagaagttaagggttgagagagtgagaaggaaggaaggaatgtgtttgtatgtgtgagtggtttagtgtgtgaattaggtagagagagtgtgtggatgtgtttgtgtgtggaatggtagggatttgggaagtagttagtaggtagggtatttggtagttttttttggtagatatgtagttgggttattgtatagtgttggatgaatggtagtggggagtgagggg

SEQ ID NO: 56 - DNA, 7369 bp, Artificial Sequence
  chemically treated genomic DNA (Homo sapiens)
tagaagtttttttttagagtgtgtttgggtatatatttaagtgtatggttgtaaatttttttttttaaagtattgaatagtattagatatttagtaggtatttaagaaatattgaatgttgtggtggtggtgagttagaagttataaaaaaaatttttttttaaaaataataataaaaagaattattttattgtgaagtttagtattataaaaatttaaataatttattataagtttttattaaaaaaaattttttttttaaagtaaatagatagataatgtttagtttatttgaaatgtttgaaagtagaggggttttaaggtagtgggagaaggtgtttgttttttgttggatatttgataattagttttttggatggtttggatgtataggagtgaaggtgtagatagtagtggggtttagagtggggttttgaggttgtgttgtggtttttttggggtttagttataattttggtttgattttagggtgaggtaggttaagggggtttgttattgtgtttttttatttttatttgggtttttatttttatagtagaggagaaagaagtttgttttttttgaggttagttgtgttagaggaagaagattgggtatgtttgggtagagatttttagattttgagtagtttgagatgttagtaattgtagttgttttaagtttgggttttgttttttagtgggatttttgtttagatgaataatttattttttgtaattttttaaaagtaaaattgtaaatgttttaggtatagaaaggaggtaaaggtgaagtttaggggaggttaggggtgtgaggtagatgggagtggatagatatattatttatttttgtgtttgttagaagaattagtagatatttttagaattgttttttatttatgttatttttataaattatttgtaaatgagggttatttggtatttttgttattttggagttatagaaataaaggatgataagtagagagttttgggtaggaggtaaaagttttgtgttttaattatagttatttttttgttgtatgatttgagttaggttattagatttttttgagttttagtttttttagtagtgtatatgggttatgtggggagtatttaggagatagataatttatttgttaaatttttttttttttggttaataaagttgttgtaattatagggattttttttgtttaggtgagtgtagggtgtagggagattggtttaatgtttaatttttttgtttttttggagattaggttgtttttttttggtagtttttttaatttttttttttttggaagtatgtgataattaataattttgtatatttaagtttagtggattttaatttttttatttgtgaaataaatgggattgaaaaattattttggttttaagatgttttgttggggtgtttaggtgttttaggtgtttttgggagaggtgatttagtgagggattagtgggaatagaggtgatattgtggggtttttttggaaattgtagagaggtgtattgtttttataatttatgaatttttatgtattaatgttatttttttgatttttttagttgtattgggtaaatttttgtttgttagagtgggttagtggtgagttagaaagggggtttattttaatagtgttgtgtttttttggagagtgttaatttattttttaagtaaaaaaagttagatttgtggtttattttgtggggaaatgtgtttagtgtattaatgtaggtgagggattgggggaggagggaagtgtttttttgtagtatgtgaggttttgggattggttggtttgttggaatttggttaggtttagttggtttggtgttgggtagttaggagtttgggttttggggagggtggttttgggtggtgtggtgggttgagtgtgggttttgtttttttgaggtgggtttgggtggggtggttgtatattagggttgtgttgagttgtgttagttgaggtgtgagtagttgttgaagttagttttttgtggagttggagttgggtgtggatttgttgaggtattgaggtatttagaggaggtgagagagtggtggtagataataggggattttgggttggtggtttagagttgagttaagtgtgtttgtgtgtgtttttgtgtgtttgtgaggatgtgtgtttgtgggtgtgtgttgtgtttataggtgtttttgtggtaggtgaatgatgggtgtgggttggtgtgtgtttggtttgtgtatatggtgtttttaagtgtgtgggtgatgagagttgggatgtgttggagattttggggtggagagtgggattataagtataggaatttttggttatgttttttgtttttggaaatttagttggggtgagggagggtgtggatgggattgttttgggagtttgtttttggttgtggttggttttaggttttaggtgtagtttgtttgtggtgtggggatgaagtttgtgtttttggaggggtttaggaagggtgaggaaagtggagtggagtaagtttgtttaggattggttttgggtggttttgggatttaatttgtgttgttttggtttaggttttaggtttaggttttttatgttattgtgtttattatttggttgagtgttgaggttagtgtgggttgttttttggtttttgggaatgtgttaggatttgttttttaaggattagtgaggaggtgatttattgtgataaggagattttagggaatggattgtatgaggttagaattttgtttgggatggggtatagtgggattttagaagtttttttttttgtttttttgtggtttttgtttttttattggtatagtgatttatttggttggaatagtttgtttttaaggaagttgggtattggaggtttgggatattgtgttgggtttttgttttgtggtgtgttgtaggggttggggagttatggttttgttttgggtgggttttaattagtttgttagttggggaagggtaagggttttttttattttttttttattgtggttgggagaattgtggtttagtttgtttttgggttggggtgttggattttggggtgggagtggagtttatgtttggatgggaggtggggagggtttatgtttttgaggggtggggggtttggggggtatgatgttgtttagggtttttattagttgttttgggggtttagggttttttgatttagtttagattttttttttgaaagttatagggttgagtggagtaggggggtgagttgttttttggggtgttgttgtttggtgtggattatagtgtgttttttttgttttaaatttttgggggatatttgtgttttttttgtgaggaaaagtattttggagttgggttaggaatttggggtgtttaggtagttttttttttttttgtttttttttatgttgtgtttttgggaggatttgtgagtggttttgtttttgttgtttttgtttatttttattttttagggatttgatttattttgtgttttgggtgtggagataaggtggaggggttggtttttggtgtgtgtgtgtgtgtgtgtttgtgtgggtgtgtgtgtgtgtgtgtgtgtgtgtgtgtgtgtgtgtgtttgtgttagagatggtataagagtgtgtggttttttaatagtggtgggagttttggaagtttggttggtttagtgtgatgtgtttgtggttttttggtttttttttattttttttttttttattttagggtgatgtgtagttggagtggaagtagttttggtgggtgagtagtgttttgtaggaaattgatttattattattttttttagtggtttgaggttttgtttatgtattttttattttgtgtgtgattttttggaggttggtgtttttttttggttttggtgggaatagtatataggtttttttgtggagtggggttggttggtgtgaattgttgtggttatttttgggtttatttgagattaatttttatgttattattatttttttaaaggagtattttttaggtttaatagtatttattgagtttttattggaaattaaaatatggttgaagtttaaggtaggaaggttaataaaggaggttatttttaattgtttttaaaataagggtttgtgtttttgagttttttttgggttgaaagttattatgagtatgagagtagattttgatgggggaggagaggtttatgagagttataagagaaggaggggtggtagaagaggagagggtgtttgtttagattttagttttgttttgaatttttgagagttagggaatatttagtattttgatgaagttttaggtgggtgttttttttttgtgtttatgatgtattgagatttagaatgtttattttaaatatattagtgtgtttttgtttggttggtaatttaagagtgtttatttgaggaattgtgttaaatatttgtttgaatttttaatttggattaagttggttttgggaggtagggttttagtaatttatattttgaaaaaattttttaggtgttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttgatagagttgtattttgttatttaggttggagtgtaatggtattattttggatttaagtagtttttttgttttagttttttaagtaattgggattataggtgtgatttttttgtttttatgtttagatttttttttttttttttttttttttgagatgtggttttgttttgttatttaggttggagtgtagtggtgtgattttggtttattgtaagttttgttttttgggtttatgttatttttttgttttagttttttgagtagttgggattataggtgttgttattatgtttggttaatttttttttgtatttttagtagagatggggttttattgtgttagttaggatggttttaattttttgattttgtgttttgttttttttggttttttaaagtgttgggattataggtgtgagatattgtatttaattatttagttaatttttatttatttttatttttagtagagatagggttttagttagttgtttaggttagttttggatttttgggtttaaatgatttttttatttttgttttttagagtattaggattataggtataagttattgtttttggtttttttaagtgattgtgatgggtttttttggttaagaaattttaaaattagagagggagtggggtttaatattatagtataggatttagggtaaataggtttgggtttagattttggttgtgttatttatgaattgtgtgatgttaggtaagttatttaatttatttgagttttggttgttttttttgtaaaaagggagttaatagatatttattttttaggaggattgatatttttaaattgtttagaatagtttttaaatataaaaatatataaataaattttaatttatgtttagtagagggtggatagaggttatttgagggttttgtttattgtattgggtgatttttttatggggtagtggtttttggtttttttagttgtatgatttaggggtaagttttatattttttttattttttgttttttaaatttggtgtgaagttattaagagtttttttttttaattagttgggatgtgaaattgtgggttttattgattataagtagtggggtgaggtggggtggagtagatgtggtatgtgttttgggttttttgttttatgaggatttagtagagtttttatttttagaaattgtaagttgggatttgtttttaggaaaatttagttgttgttaaggttgtgtagttatttagttttggagttaagttagagtaggtaggtaggtgttagggtttttttatgggtaaatttattttttgtttttttttttttgaagggggaggagaggagttaggtagattagttatttttaattttttttttgtttgtaaaatggtttttttggatataggtaatatgaggtaggggttgttaggtgtttagattttagattatttgatgtgtttggtaggatgtggtttagtttgggagaaattattttttgtgttgttttgtttggtttttttttatttttaggttatttgtttgatgatatttttgggaaaggtttttagtttatagtatttgttagttgttgtttgaaggaggtagttggtagggggaagtgataggggggaggtttagtaaaattgaaggtagagaggaataattatatttttgtttttaatgtatttttttatatgaagtgttgttggtatgttatttatattaatttagttaatttttatgtttattttttgagatagttattattattatttttattttatagatgaggaaattagagtttagataagttaagttgtttgtttagggttatttagtaaaatttggattttagtttaggtgatttggttttagagtttttttgtttaattattaggatatagttttttatttagttttgttttgtttgttttgttgtatggattttgtgattaattttttgagtatgtgtttgtagttatgttttttaaatttgtatatggttttatttatggatgaggaaattgagatttagagatattaagtggttttttaaagtttatgtagtaattggtagagttaggattataatttgggtgttttttgttttaaagttttgggtatttttatttggtagagtagggttattttatttggggatttgggttgggggatttaggaggttggaggaattgttagattgtttttttttttgggaattgattttttggttagggttgtgattaggaaattgttggattttggtaatttatatatatttggggggtatttatatttatgagggatatttttggggggaaaataaattgattttagttgataatatttggtggtaaataggattttggtttttgtttttgtaatagatttgtttttgttgatattagtttgttttttagttgtttgtttttttagtgattttggtgtgttaggttggttgagttttgttggtgggggttaggttttttgtgggaaggaagtaggaagattagttggaaggagtgagagagattttttggtaggaagatgttatttgaggtgatatagtaaagtttggttaggtaatatagtgtttaatttttgttgtgattagggttttttttgtatttttgttgtagg

SEQ ID NO: 57 - DNA, 7369 bp, Artificial Sequence
  chemically treated genomic DNA (Homo sapiens)
tttgtagtagagatataaggaaggttttggttatggtggagattagatattatgttatttggttgggttttgttgtgttattttaggtgatgtttttttattagagggttttttttattttttttagttggtttttttgttttttttttataggaggtttgatttttattagtagagtttagttagtttggtatattaaggttattgggagagtaggtaattgaagggtaagttaatgttaataaaggtaagtttattgtaagagtaaggattagggttttgtttgttattaggtattattagttaaaattaatttgttttttttttagaggtgttttttatgggtgtgaatgttttttaaatatgtgtgaattgttagagtttagtagttttttaattgtagttttggttagaaggttaatttttaaaagaagaaatagtttgatagtttttttagttttttaagttttttgatttagatttttaagtagggtaattttgttttgttaagtaaaagtatttgggattttggggtaaaaagtatttgggttgtggttttagttttgttagttattatgtaagttttaaaaagttatttaatgtttttaggttttagtttttttatttataaatggggttatgtataagtttaaagagtatggttatagatatatatttaagaaattgattatagagtttatgtagtaaggtagatagaatagagttgaatgaaaggttgtattttggtggttaagtaggagggttttggagttagattatttgggttggagtttaggttttattaggtgattttgggtaagtaatttaatttgtttgagttttagtttttttatttataaaatggggatagtaatagtgattgttttagaggatagatatgagaattaattgagttaatgtaggtaatgtgttagtagtattttgtatagagaagtgtattgaaaatagaagtatgattatttttttttgtttttagttttattgagttttttttttattatttttttttgttaattattttttttagatagtagttgataggtgttgtaggttgagggttttttttaaggatgttgttaggtgggtggtttaggggtaaggaggggttgggtggggtagtgtaagggatgattttttttaggttgagttatattttgttaggtatattaggtgatttgaagtttagatatttggtagtttttgttttgtgttgtttgtgtttaaggaaattgttttgtaggtaaaaagaaaattaaaggtggttggtttatttggttttttttttttttttttaggagagggaaaatggagagtgagtttgtttatgagggagttttggtatttatttgtttgttttggtttgattttagggttgagtgattgtatgattttggtagtaattggatttttttagggataagttttaatttgtagtttttgggggtgaaagttttgttgagtttttatgaggtaggaagtttgggatatatgttatatttgttttattttattttattttattgtttgtgattagtggagtttatagttttatgttttagttggttgggagaggaggtttttggtaattttatattaagtttaaagggtaggagatggaagagatatgagatttgtttttgagttatatagttaaaaaggttaaaggttattgttttataaaggggttatttagtatagtggatagagtttttaaataatttttatttattttttgttaggtatgagttgggatttatttatatatttttatgtttgggggttgttttaagtagtttaaaaatattaatttttttaaaaagtggatatttattagttttttttttatagaagaggtaattaaggtttagataagttaagtaatttgtttaatattatatagtttataagtggtatagttaggatttgaatttaggtttgtttgttttgagttttgtgttgtagtattgaattttattttttttttaattttgaggttttttaattagagaggtttattataattatttggggaggttaggggtagtggtttatgtttgtaattttaatattttgggaggtagaggtgggagaattatttgagtttaagggtttaagattagtttgggtaattagttgagattttgtttttattaaaaataaaaataaataaaaattagttgggtggttgggtgtagtgttttatatttgtaattttagtattttgggaggttgaggagggtggagtatgaggttaggagattgagattattttggttaatatggtgaaattttgtttttattaaaaatataaaaaaaaattagttgggtatgatggtagtatttgtagttttagttatttgggaggttgaggtaggagaatggtgtgaatttgggaggtggagtttgtagtgagttgagattatgttattgtattttagtttgggtgatagagtgagattgtattttaaaaaaaaaaaaaaaaaaaaaaaaatttgggtatgggggtggggggattatgtttgtggttttggttatttgggaggttgaaataggaggattatttgagtttaggatggtgttattgtattttagtttgggtgatagagtgtaattttgttgaaagaaaagaaagaaagagagaaagagaaagaaagagagaaagagaaaggaagaaagaaagaaagaaagaaagaaagaaagaaagaaagaaagaaaagaaagaaaaagaaagaaagaaagaaaaagaaagaaagaaagaaagaaagaaagaaagaaagaaagaaagaaagaaagaaaaaaagaaagaaagaaaagtatttagggagtttttttaaaatatagattgttgaggttttgttttttgagattaatttaatttagattgaagatttaagtaagtgtttggtataattttttagatgggtatttttgggttgttagttaagtggagatatattggtatgtttgaaatggatattttgggttttaatatattataggtataaggaggaggtgtttgtttagggttttattaaggtgttggatattttttggtttttgggagtttaagataggattaggatttaggtaggtattttttttttttttattattttttttttttttatggtttttataggtttttttttttttattaaaatttgtttttatgtttataataatttttagtttaaagaaaatttagaaatgtaaatttttgttttagaaataattaaaaatagttttttttattggtttttttgttttagattttagttatattttaatttttagtaagagtttagtgaatattgttgaatttaaggagtgtttttttgaaggggtggtaatggtataggggttgattttggatgagtttaggagtagttgtggtggtttgtgttggttagttttattttgtgggaaagtttgtgtgttatttttgttagggttgggagggggtgttggtttttaggaaattatgtgtggagtgggaggtgtgtgggtagagttttggattgttggagggagtagtgatgagttagttttttgtaaggtgttgtttgtttgttaaaattgtttttattttggttgtgtgttattttggggtggggagggggagaatgggaggggattggggggttgtgaatatgttatgttgagttggttaggtttttgaaatttttgttgttgttgggaaattgtgtgtttttgtgttgtttttgatatagatatatatatatatatatatatatatatatatatatatgtttgtgtagatatgtatgtgtgtgtgtgttgggagttggtttttttattttatttttatgtttaaagtatgggatgagttagatttttggaaaataaaaatagatgggagtaatgaaaataaaattgtttgtaagtttttttagaaatgtgatgtggagggaggtaaggagaggggaagttgtttgggtgttttaagtttttaatttagttttaagatgtttttttttatgaagagggtgtaagtgttttttaggggtttgggatggagaggatgtgttgtggtttgtgttaggtggtggtgttttagggggtgatttgtttttttgttttgtttagttttgtagtttttggagagggaatttgggttaggttgggaagttttgagtttttgaggtagttgatagaggttttgagtagtgttgtgttttttagattttttattttttaaagatatgaattttttttgttttttatttaggtgtgggttttgtttttgttttggagtttagtgttttgatttgaggataggttgggttgtgatttttttggttgtggtgggaaagaggtagaggagatttttgtttttttttgattgataggttggttagagtttgtttagagtagggttgtgattttttgatttttatagtgtgttgtggagtggggatttgatgtggtgttttggatttttagtgtttggtttttttgggaataaattgttttagttaaataggttattgtgttgatgggaggatggagattgtgaaggggtaggggagagggtttttggagttttgttgtattttattttgggtggggttttgattttatatagtttgttttttggggtttttttgttatagtgagttatttttttgttagtttttaggggatgggttttggtatatttttaagggttaggaaatagtttgtgttgattttagtgtttagttaggtggtggatatagtggtgtaaaggatttgaatttgggatttgggttagggtggtgtagattggattttagagttatttgggattgattttagatgaatttattttattttgttttttttgttttttttgggtttttttagggatatggattttatttttatgttgtgagtaaattgtgtttggggtttggagttaattgtagttaaaggtgagtttttagaatggttttgtttatgtttttttttgttttagttgggtttttaggggtggggagtgtgattagggatttttgtatttgtaattttgttttttgttttggggtttttggtatattttgatttttgttatttgtgtatttagagatattgtgtgtgtaagttgagtgtgtattgatttatgtttgttatttatttgttgtagaaatatttgtgaatgtagtatatatttgtgaatatgtatttttgtggatatgtagggatatatgtgggtatgtttggtttggttttgggttgttggtttggggttttttgttgtttgttgttgtttttttattttttttgagtgttttggtgttttggtgaatttgtgtttagttttggttttataaggaattgattttggtagttgtttatattttagttggtgtagtttagtgtggttttgatatataattgttttgtttgggtttgttttaaggaggtgggatttgtgtttggtttattgtgttgtttgggattgttttttttggggtttaggtttttggttgtttagtgttgagttagttgagtttggttgagttttagtaggttagttggttttggaattttgtgtgttgtaggagggtatttttttttttttttagttttttgtttgtgttggtgtgttggatatatttttttatgaagtgagttataaatttggttttttttatttggagaatgagttggtattttttaggaggatatagtattgttagaatgagttttttttttggtttattgttgatttattttggtaggtaaggatttatttaatgtagttgaaaagattaggaggatgatattaatatataaaaatttataaattataaaaatgatgtatttttttgtaatttttagaaaagttttataatattatttttatttttattgattttttattaggttattttttttagaagtatttggagtatttagatattttaataaagtattttgaggttagaatgattttttagttttgtttattttatagatgaggaaattgaggtttattgaatttaagtatataaagttgttgattgttatatgtttttgggaaggagggaattggagagattattaaaaaagggtaatttgatttttagggaaatagaagaattggatattgaattaatttttttatattttatatttatttgaatagaagaaatttttgtggttgtagtagttttgttggttaggaaggggaggatttgatgagtgagttgtttgttttttgaatattttttatatagtttgtatatattgttggggaaattggggtttagagaagtttggtgatttaatttagattatgtagtaaagaaatgattatagttggaatataggatttttgttttttgtttggggttttttgtttgttattttttatttttgtggttttaaaatgataaaaatgttaaataatttttatttgtagatggtttatggagatgatataaataaaggataattttggaagtgtttattggtttttttgatagatatagaaatgagtgatgtgtttatttgtttttatttattttatatttttgattttttttggattttatttttgttttttttttgtgtttgaaatatttgtagttttgtttttaaaaaattgtagaggatggattgtttatttgaatagaaattttattaaaaaatagaatttaggtttggagtagttataattattgatattttaggttgtttagagtttggaaatttttgtttagatatgtttagttttttttttttaatgtagttgattttggggaggataggttttttttttttttgttgtggggatgggagtttaggtaggggtgggaggatatagtagtagatttttttggtttgttttgttttggagttaggttaggattgtggttaaattttagaaaggttatggtatagttttaggattttattttaagttttattgttgtttgtatttttgtttttatatatttaaattatttaaagggttggttgttaaatgtttagtagaggataggtatttttttttattgttttgaagtttttttgtttttaggtattttaaatagattagatattgtttgtttgtttattttggggagaaaattttttttaataaaggtttgtaatgaattatttaaatttttgtggtattgagttttataatgaaataattttttttgttgttgtttttgggaaagaattttttttataatttttagtttattattattatgatatttaatattttttaagtatttattaagtgtttagtattatttagtgttttaaaaaaaaaagtttgtaattatgtatttgaatgtgtatttagatatattttaagggaggatttttg

SEQ ID NO: 58 - DNA, 2986 bp, Artificial Sequence
  chemically treated genomic DNA (Homo sapiens)
gagggttttttttagttgttatattttggagtgtatgagatgaggtaggtatataaagtggataagatgtggttaagaaaataagttatatattaagtttatttgtagtataggtgtttaagaaaattttgttgttgtgtaatattagaatggaaggttggtttttagtaaaatgtattaattttggtttaaattaagatgatgggtattgggtatgggggtggggaggtagttgaagatttattgagttttgttttagggtagttttgtttattgttttattttattttttattatggtgtttaagtttatattgagagagaaatttttagttgtaaaagggagaagagaaatgttggaatattagtattggatgttaggatatggttgtggtgttttaaaaattattttattatttggagtttgattttgaggggagtatttttattttttagttttttgaaagtatttattagtatttgaatattgttttgagtttgttggagtagtgaaatttggtgagagagaagggtggaggaaggaaggagttgttgtatttggtggttggatttaggtagaggaaattgttataattttgggaaagaatagaaaagtagaaagggatgagtttttatatgtagttaatgtttatggttttaattgtgtttgggaaggaagattttgggttaggggtgtatttttgttttttaaaattaaatgtgtttgagatagttataaagtttattaagggatttgagagattagagttttttgttttttttttttaattttgagttttttttttatttttattgagggagagtttgagtttatgataagtgttgtgtttatttttggttaatttttaaaagaaagatgtttgttttggttttttttttaggtttttagtttttttagggatggtagaaatttttgggttaaggttgagtgaattattgtttattgtttttattagtttttagtaaaggtatgttggtgggggggtgtttagtttttttagtaaatgttttgtggttttttttgtagattatgaggtgggggttgttggggagggttgagttgggggtagtttgttattttggtttttagtgagttgttggtgatttttgtggttttttggtttaggttttggttttttgggtgaggagtgggagggaggttggggtttaggtgttgtggtgaatttgttaatgtagtgttgggttttgaattttaggttttgttttaggtttttggttgtttggttagtttgtttgttttaattttaatttttttgaggttagttagagtaggtttgttggtagtagtatttttttagtagttatgtgattagttaattttttggtggtgtttggggaggtggtgtgtttgggaatgaggggaggtggtggaattgtgttggggttattttaaggttgtgtttgttagttttggtggggtggtttttgttgttgtaattaatggatttttttttttgtttaaatagatttgttgtgttaattatttttttttttgttagttttttttttattgttatattgggttattaaaaaaagggggtttgtttttttggggtgttttttttttttttttttgtttttgtttgtttatggttttgtgattttgatgttggtaaggtttggagagtggttgggtttgtgggatttgtgggtttgtatttgtttagatttggatgggttttgttattttttttgtttgtttggtttttttttttttttgtttttttgtttgttagtttatttgattagtggagatttggtggttgggttggggtttttttgtagtttttgtgtgtttttagagtttgggttgtggtttgttggggtttgtgttttttggttttgagggtagttgttgggtttttgagaggggtttgggttgtgtaggggtgttttgttttgtttggttttgtttttttgagagtgtgagagaggtggttgtgtagatttgggagaaagatgttaaatgtgtgagtgtttaatgggagttttagtttggagtggatggatgttaggtaggtggagtattttaagtttttggtttgtaggaatttttttggtttggtggattatgaagagttaatttgggatttggagaagtattgtagagatatggaagaggtgagttagtgtaagtggaattttgatttttagaattataaatttttagagggtaagtatgagtggtaagaggtggagaagggtagtttgtttgagttttattatagatttttgtggttttttaaaggtgtttgtaaggtgttggtgtaggagagttaggatgttagtgggagttgtttggtggtgtttttaattggggttttggttaattttgaggatatgtatttggtggatttaaagattgatttgttggatagttagatggggttagtggagtaatgtgtaggaataaggaagtgatttgtaattgatggtaatgatttttttttaattatagaatgtgtttggggttttgttttgtttgttggagggtgttaattttagtttgttttttggtgtattttgatttagttttgggagagttaattttattggttttaggtgtttagtgttatttggtttattgtttgtttgtttgtgatttttaagttagaaattggagatggtaagatttgataatttttttaatttaatatattgtggttttttttattagtaatttttaggtatgtgataaagttgggatgtttattaatggtttgttttttggttagggaaagagttttggggtggagaatgtattttttgttttttgaaaataattttattttgtgtttttaaaagttattggggatgatggatttaggattgtgggtggaggtagtgggttttttattttttgattatggggttaatttttgttagttattgttttttttaataaagattgtgtgttttttttaaaaattttttttgtgtttaga

SEQ ID NO: 59 - DNA, 2986 bp, Artificial Sequence
  chemically treated genomic DNA (Homo sapiens)
tttaagtgtaggggaaatttttaaaaagaatatataatttttattagaaaaaataatggttggtagaagttggttttatagttaggggatgaaaaatttattatttttatttataattttggatttgttatttttagtggtttttaagggtataaaatgaggttgtttttaaaaaatagaaagtgtattttttgttttagagttttttttttagttaggaggtggattgttgataaatattttaattttgttatatatttaggagttgttagtgagagggattgtgatgtattaagttagggaaattattggattttattatttttagtttttgatttaaaagttataaatagataagtagtgggttaggtagtattgaatatttaagattaataaagttagtttttttaaagttaaattagaatatgttgaaaagtaagttaaggttaatattttttagtaggtaaagtggggttttaaatatattttatggttgggaaagggttattattgttggttgtaggttgtttttttatttttgtgtattgttttgttaattttgtttggttgtttgatggattagtttttgggtttattaaatgtgtgtttttagagttagttggagttttaattaaaggtgttgttgggtggtttttgttgatattttggtttttttgtgttggtattttgtaggtatttttggggggttgtgggggtttgtagtagaatttgggtaagttgtttttttttattttttgttatttgtatttgttttttaggggtttgtgattttgaaaattgaaattttatttgtgttggtttgttttttttatgtttttgtagtgtttttttaagttttgggttaattttttgtggtttattgggttgaagaggtttttgtaggttgagggtttggggtgttttgtttgtttggtgtttatttgttttaggttagggtttttgttagatatttgtatgtttgatattttttttttgggtttgtatgattgttttttttgtatttttaaaaaaataaaattgaataaaataaagtgtttttatgtagtttgaattttttttggaagtttagtgattgtttttggagttaaaagatatagattttgatgagttatggtttgagttttaggagtgtgtaggggttgtggggaagttttggtttggttgttgagtttttgttgattaaatggattggtgagtgggagggtggagaggagaggggattaggtaagtggagagggtggtaaagtttgtttgagtttgggtgggtgtaagtttgtgggttttgtgaatttagttgttttttaaattttgttggtgttggagttgtagagttgtgagtaagtggggataggggagggggagaaaaatattttgaaaagatgagttttttttttttagtggtttaatatggtggtggaagggaggttgatgaagaagaaaatgattgatatggtgagtttatttaaatagaggaggagatttattggttgtggtggtgggagttgttttgttgaggttggtgagtgtggttttaaggtggttttggtgtggttttgttatttttttttgtttttgagtgtgttgtttttttgagtgttgttgggagattggttggttgtgtgattgttggaggggtattgttgttaataaatttgttttggttggttttggagaaattaaaattaagataaataaattagttaaatggttgggaatttggggtggggtttgaggtttggggtttggtgttgtgttggtgggtttgttgtggtgtttaagttttgattttttttttgttttttgtttgggaagttgggatttggattagaggattgtgaaggttgttggtagtttgttaggagttggggtggtgagttgtttttagtttggttttttttagtggtttttattttgtggtttgtgggggaggttgtggagtgtttgttgggggggttgggtgtttttttgttggtgtgtttttgttgggggttggtggaggtagtgggtaatggtttgtttagttttaatttagaagtttttgttatttttggggaggttgggggtttagggaagaagttaaagtgaatgttttttttttagaaattagttaggagtagatgtggtatttattatgaatttaagtttttttttaatgaaaataagaaaggaatttaagattaaaaaaaaaaaataaaaaattttagttttttaagttttttaataaattttgtagttgttttagatatgtttagttttgaaaaatgagggtatatttttggtttaggatttttttttttaagtatagttaaggttatggatattggttgtgtgtgggaatttgtttttttttatttttttgtttttttttgggattgtagtagttttttttatttgagtttagttgttaaatataatagtttttttttttttttattttttttttttattagattttattgttttaataaatttagaataatatttagatgttagtgaatgtttttagagggttgaagggtgaaaatattttttttggggttaaattttagatgatgaaatgatttttaaaatattataattatgttttaatgtttgatattagtattttagtgtttttttttttttttttgtagttggaaattttttttttagtgtgggtttgagtattgtggtggaaggtaaagtaggatgatgagtagggttgttttgagataaagtttagtggatttttaattgtttttttatttttatgtttggtatttattattttggtttgagttaaagttaatgtattttattggaaattaattttttgttttaatattatatagtagtaaagtttttttaagtatttatgttatagatgagtttgatgtgtagtttgtttttttagttatattttgtttattttgtgtgtttattttattttatatgttttagaatgtgatagttggaagggattttt

SEQ ID NO: 60 - DNA, 5666 bp, Artificial Sequence
  chemically treated genomic DNA (Homo sapiens)
aaaattagaatttttatttttttgtgtttgttatattttttagtgttgtttaatttttttttgtaagtgagggtggtggagggtgtttataatttttttagggagtaagttttttttggttttttttttttttttttttttttttttttttgagattaagttttgtttttgttttttaggttggagtgtaatggtgtgattttggtttattgtaatttttgtttttttttgggtttaagtgatttttttatattagtttttgagtagttgggattataggtatgtgttattaagttttgttaattttgtattttttagtagagatagggttttgttatgttggttaggtttgttttgaatttttggttttaggtgatttgtttgttttggttttttagaatgttgggattatagatgtgagttattgtatttggattttttttttatgtaatagtgataattttatttaaagtatttttttttttttttgagttggagttttattttgttatttaggttggagggtggtggtgtgattttggtttattgtaatttttgttttttgggtttaagtgatttttttgttttagttttttgagtagttggaattatatatgtgtgttattatggttagttaatttttgtatttttagtagagatggggtgttattattttggttaagttggttttgaatttttgattttaggtgatttgtttgttttggttttttaaagtgttgggattataggtgtgagttattgtgttttgttttaaagtattttttttttatgttttaaaataagattgtaagttagtttttaaagtggataatttaagagttaataggtattagtttaggatgtgtggtattgtttttaaggtttatatgtattaatatattatttaaatttataataatttttataaagtagggggtatttatatttttttttttttttataattatgaaaaatgtaaggtatttttagtaggaaagagaaatgtgagaagtgtgaaggagataggatagtatttgaagttggtttttggattattgtgtaattttgtttttagaatattgagtattttttttggtttaggaattatgattttgagaatggagtttgtttttttaatgattttttttttatttttttatttgtttataggtagaatttttttttgtttgtattaaataaattttatttttttagagtttgtttttatattaggtaatgtatatgtttgagaaatttttgttttagatagttgttttatatgtaggaggggaaggggaggggaaggagagagtagtttgattttttaaaaggaattttttgaattagggtttttgatttagtgaattttgtgtttttgaaaattaagggttgagggggtagggggatattttttagttgtataggtgattttgatttttggtggggtttttataattaggaaagaatagttttgtttttttttatgattaaaagaagaagttatattttttttatgatattaaatattttgatttaatttggtagttaggaaggttgtattgtggaggaaggaaatggggtgggggtggatttttttttaatagagtgaatgtatttaaatatgtttttgttggtaggtgggggagtgtggttgggagtagggaggttggagggtggtgtggggggtaggtggggaggagtttagttttttttttttgttaatgttggttttggtgagggttgtttttggttggtgtttttgggggagatttaatttggggtgattttaggggtgttatatttgttaagtgtttggagttaatagtattttttttgagtatttgtttatggtgtttttttgtttggaaagatattgtggtttttttagaggatttgagggatagggttggagggggtttttttgttagtattggaggaagaaagaggaggggttggttggttattagagggtggggtggattgtgtgtgtttggtggttgtggagagggggagagtaggtagtgggtggtggggagtagtatggagttggtggtggggagtagtatggagtttttggttgattggttggttatggttgtggtttggggttgggtagaggaggtgtgggtgttgttggaggtgggggtgttgtttaatgtattgaatagttatggttggaggttgatttaggtgggtagagggtttgtagtgggagtaggggatggtgggtgattttggaggatgaagtttgtaggggaattggaattaggtagtgttttgattttttggaaaaaggggaggttttttggggagtttttagaaggggtttgtaattatagatttttttttggtgatgttttgggggtttgggaagttaaggaagaggaatgaggagttatgtgtgtatagattttttgaatgttgagaagatttgaaggggggaatatatttgtattagatggaagtatgttttttattagatataaaatttatgaatgtttgggataaaaagggagttttaaagaaatgtaagatgtgttgggattatttagtttttaatttatagatatttggatggagtttattttttttattaggagggattattagtggaaatttgtggtgtatgttggaataaatattgaatataaattttgattgaaattatttagaagtggttgggtgtggtgttttatgttttgtaatttttttattttgggagattaaggtggggggaattatttgaggttgggagtttgagattagtttggttaataggtgaaattttgtttttattaaaaatataaaaagtagttgggggtggtggtaggtgtttgtaattttagttatttgggaggttgaggtaggagaattgtttgaatttgggaggttgaggttgtagtgaatagtgagatggagttattttattttagtttgggtgatagagtgagattttgttgaaagaaagaaagagagaaagagagagagaaaaattatttagaagtaattatatattgtgtttatttttaattgagtagggtaaataaatatatgtttgttgtaggaatttaggaaataatgagttatatttatgtgattattttagaggtaatatgtagttattattttgggaatatttgttaatatttttgtttttttattatttttagtttatttgatatagtttatttgtgataagagtttttaattttttatttttgaatagaggtgttttttttttttttatttttgttttgtgagggagttaggggaggatttaaaagtaattaatatatgggtaatttagtatttttaaaattttgttaatagtttgaatttgggagtttggttttgtagttttataatattttagaagagattttatttgtttaaaaataaaaaggaaaaagaaaagtggatagttttgataatttttaatggagaagggagaagaatatgtagaaaaggggaaatgatgttggtttagaattttaattatattggtgtttaatataggaatatttatttatataatattttaaagtattaaatttatattagtatattattaaatggatatattattaaatgggtttaagtattttatatattttaatttaattgatttattttttttttgttttggatttttattatgatttaaatatttatatatgggttattttttagattttttatattatgaaatataagaaaaatttttaaggttagttttatgattaagatgaaggattttattgaatatataaaataataaatatattgtaatattttgtttttttttttgtagttgtaatttggtttgtttatattttttttttgtttttttgaaaattgagttagttttatttttttaggataggatttaataattataatataatttagtataattttttgatttaggtaaattatgtaatttgtgtttagtatgaaatgtatttaaaaataagtaatttttttttaatattattatttttaaattaatataataaataatagttattttaaaataaattgtttatttttattatgtagtatttaaattttaaggttgttatgattgtagatagtattttaaaatttttttttggaaatggttttgtttttaagatgatttaggaattaaagaggtgattattttttgtttaatgaatttttaaattataaatttgggaagtgttttagttttttattgttgttgttataaattattataaatgtgttagttaaaataaatataaaattattattttatagttttagagattagaagttaaaaatgggtttataaggttttattttttttggaaattttaaggggtaatttgtttttttgttttttttagtttttagtgattattaaattttttggtttatggtttttgtattttttttgtggtttgtgtttttatttttgtattttttttttgattgtgattttttaataaaaatatttggggttatgttgggtttattttgaaaattttggataattttttttaagattattaattaaattatatttgtaaagttttttttgttatataagttaatgtattaaaagtttttgaggattaggatatagatattgggggtgggggggtattatttagtttattataggaaggaattttagggttaattaaattagtttttttattttatatttgaagaaattgaagttttggaattggagagtattatgttaaatgaaataagttaaatatagaaagataaatattatatgtttttatttatttgtgaaatataaaataattatatttttagtagtaaagagtagaatggtggttattagagttggggggtgggaggaatggggagatggtaattaagatataaagttttagttaagatgggaggaataagtttgattgttttttttgagatgtgttttatagtatgatgaatatagttaaatagtaaattttaaatgtttttatttgataaaaatgttaaatatttgagatgatggataggttatttagtttgatttaataattttttattgtgtttaaagattataattttatattgtattatataaatatatataattgtattattttaatatataattttaaaattaatataatgaaaaagaaattgaagtttaatatttttagaagttaagtgtaatttaaaagttttgtgagaatttgttttaataaataaataagttttttttttttaataattattatattttgtgtttggatatatagtagtgaataaaaaaaaaaaaaaaaaaaaaaatttttaggtttaatataattttaggaagaaattttagtagttgtattttaggggaaatataggaagttagtttggagtaaaagttagtttgtttttgtttttttgttattttgtttgtgttttatagtgttttttgtttgtgatgatagttttgtagaagtttggaggatataatggaatttattgtgtattgaagaatggatagagaatttaagaaggaaattggaaattggaagtaaatgtaggggtaattagatatttggggtttgtgtgggggtttgtttggtggtgagggggttttatataagttttttttttgttatgttggtttttattttggttttgattattttgttttttttggtagg

SEQ ID NO: 61 - DNA, 5666 bp, Artificial Sequence
  chemically treated genomic DNA (Homo sapiens)
tttgttagagagaatagaatggttagagttagggtgggggttggtatgatggaaaggaagtttgtgtagagtttttttattgttaagtagatttttatataagttttaggtgtttaattatttttatatttgtttttagtttttaattttttttttgagttttttatttattttttagtatataatgaattttattatattttttgaatttttgtggagttgttgttataggtagagagtattgtgaggtatgggtaaaatagtaaaggggtagggatagattgatttttattttaggttaattttttgtattttttttgagatataattattgaaatttttttttgaaattatgttaggtttggagatttttttttttttttttttttttgtttattgttgtatatttaagtgtagaatgtggtaattgttaaaaagagaaaatttgtttgtttgttaaaataaatttttataaaatttttaagttatatttagtttttgggaatgttgaattttaattttttttttattatattagttttaaaattatatattgggatagtatagttgtatatatttatgtggtataatatgaagttatgatttttgaatataatggggaattattaagttaagttaagtaatttatttattattttaaatatttgatatttttgttaaatgagagtatttgggatttattatttagttatatttattatgttatgaaatatattttaaaaaaaataattaaatttattttttttattttaattgaggttttatattttgattattatttttttattttttttattttttagttttagtaattattattttattttttattgttaagaatgtaattgttttatattttatagataagtgagaatatgtgatatttgtttttttgtgtttggtttattttatttagtataatgttttttaattttaaaattttaatttttttaagtataaaataagaaggttagtttaattaattttaaaatttttttttgtggtaggttgaataatgtttttttatttttaatgtttatgttttaatttttaaaaatttttaatatattaatttatgtggtaaaagaggttttgtagatgtgatttaattaatggttttgagggagattatttagaatttttagggtgggtttaatataattttaagtgtttttattagagggttatagttagagagaagatataagaatggaagtataggttatagagaaaatatagagattatgagttaaggaatttgatggttattagaagttggaaaagataaggaaatagattgttttttagagtttttaaaaggaatgaaattttgtggatttatttttgatttttgatttttagaattgtaaaataataattttgtgtttgttttagttaatatatttgtgataatttgtaatagtagtagtaggaaattaaaatattttttaggtttatgatttgagagtttattaaataagagatggttatttttttggtttttaaattattttggaaataaagttatttttagagaggaattttaaaatattgtttgtagttatagtaattttaaaatttgagtgttgtatggtggaagtagataatttattttaggataattgttatttgttatattagtttgaggatggtggtgttaaagaggagttatttatttttaggtatattttatattaaatataaattgtataatttgtttaaattaaggaattatattaaattatattatggttattaaattttgttttgagaaagtgaaattgatttagtttttaaagagataaagagaaagtataagtaaattaaattgtagttataaaaagaaagataaaatgttgtagtatatttattgttttgtgtatttaatgaagttttttgttttggttataaaattagttttaaaggttttttttatattttatagtatgaaaaatttaaaaagtaatttatatgtaaatatttaaattatgatagaaatttaaagtaaaaagaaaatgaattaattgaattaaaatgtgtaggatgtttaaatttatttgataatatatttatttgataatatattaatatgaatttagtattttaaaatgttatataaataaatgtttttatattaaatattaatgtagttaggattttaagttaatattatttttttttttttatatgttttttttttttttttattaaaaattgttaaaattatttattttttttttttttttttgtttttaaataaataaggttttttttaagatattgtaggattataaagttaaatttttgggtttaagttgttggtaaaattttagagatgttaagttatttatgtattaattatttttaaatttttttttaatttttttataaaataggagtagggagaggagaaatatttttgtttaaaaatgaggaattgaaaatttttattataaataaattatattaagtaagttaaagatagtaaaagagtaaaaatgttagtagatatttttaaaatggtaattatatattatttttggaatgattatatgaatgtggtttattattttttaagtttttatagtaaatatatatttatttgttttatttagttaaaaataaatataatatgtagttgtttttgaataattttttttttttttttttttttttttttttttttgataaagttttattttgttatttaggttggagtgaagtggttttattttgttgtttattataattttagttttttgggtttaagtgatttttttgttttaattttttgagtagttgggattataggtgtttgttattatttttggttattttttgtatttttagtagaggtgaggttttatttgttggttaggttggttttgaatttttgattttaggtgattttttttgttttgattttttaaagtgaagggattataaggtgtgaggtattgtgtttggttgtttttgaataattttgattaaaatttatatttgatatttattttaatatatattatagatttttattgataattttttttagtaagaaagataagttttatttaggtatttgtgaattggaggttaagtagttttagtatattttatatttttttaagattttttttttattttaaatgtttgtaaattttgtatttgataaagagtatatttttatttaatataaatatgtttttttttttagatttttttagtatttgagagatttgtatgtgtgtggttttttattttttttttttggttttttaagtttttagggtgttgttaggaggaggtttgtgattataaattttttttgaaaattttttaggaagttttttttttttttggagaattgaagtgttatttgattttaatttttttgtaaattttgttttttagagttgtttgttattttttgtttttgttgtagattttttatttatttggattggtttttgattgtaattatttggtgtgttgggtagtgtttttgtttttagtagtgtttgtattttttttatttgattttgggttgtggttgtggttagttagttagttgaaggttttatgttgttttttgttgttggttttatgttgttttttgttgtttgttgtttgttttttttttttttgtagttgttgagtgtatgtggtttgttttattttttggtgattagttagttttttttttttttttttttggtgttggtggaagagttttttttgattttgttttttaaattttttggagggattgtggtatttttttaggtaaggggatgttgtgagtgagtgtttggaggaggtgttattaattttgagtatttagtgaatgtggtatttttgaagttgttttaggttgggttttttttgggggtattagttggaagtagtttttgttagagttagtgttggtaaggaaggaggattgggtttttttttatttgttttttatattgttttttggtttttttgtttttagttgtgtttttttgtttgttagtaaaggtgtgtttgagtgtgtttattttgttaaaaagaaatttgtttttgttttgttttttttttttgtgatataatttttttaattgttaaattgaattggggtgtttggtgttatagggaaagtatggtttttttttttaattataagaaaaagtaaaattatttttttttagttgtgagagttttattgagaattgaaattatttgtatgattagaaagtgtttttttatttttttaatttttgatttttaggagtgtggggtttattaagttagaaattttagtttaaaggattttttttggagagttggattgtttttttttttttttttttttttttttttgtgtgtaaaatggttgtttggggtaagggttttttagatgtgtatattgtttggtataagagtagattttgaaaagatgaggtttatttaatatggatgggggagaattttgtttgtaggtagataggaaaatggggagggagttattggaaggatggattttatttttaaagttataatttttagattagaaaaagtgtttagtgttttagaagtagagttgtatagtgatttaaagattagttttaaatattgttttgttttttttatattttttatattttttttttttattgaaaatattttgtattttttgtaattataaagggggaagggaatatgagtgttttttgttttataggggttgttgtgagtttaaatgatgtattaatatatataagttttaagaatagtgttatatattttaagttaatatttgttagtttttgaattatttgttttgaggattggtttgtaattttgttttgaggtatagaaagaaaatgttttggagtaggatgtggtggtttatatttgtaattttagtattttgggaagttgaggtgggtagattatttgaggttaggagtttgaggttagtttggttaaaatggtgatattttgtttttattaaaaatataaaaattagttggttatggtggtgtatgtgtgtaattttagttatttaggaggttgaggtaggagaattgtttgaatttgggaggtagaggttgtagtaagttgagattgtgttattattttttagtttgggtgatagaatgagattttgatttaaaaaaaaaaaaaaatgttttggatagaattattattattatataaaaggaaagtttggatgtggtggtttatgtttataattttagtattttgggaggttgagataggtggattatttgaggttaggagtttgagataagtttgattaatatggtgaaattttgtttttattaaaaaatataaaattagtggggtttggtggtgtatgtttgtaattttagttatttggaggttgatgtaggagaattgtttgaatttaggagaaggtggaggttgtagtgagttgagattgtgttattgtattttagtttgggagataagagtgaaatttggttttaagaaaaaaagaaagaaagaaagaaagaaagattaagaagaatttattttttgaaaagattatgggtattttttattatttttatttataaagaaaagttaaatagtattaaagagtataataagtgtaaggaggtaaaagttttaatttt

SEQ ID NO: 62 - DNA, 5085 bp, Artificial Sequence
  chemically treated genomic DNA (Homo sapiens)
tgatggttgtataattttgagtatatgaaaaattaatgaattgatattttgagtgagttgtatgatattggaattatattttaataaagtatggtaattgttttaagataggttggaaagagaaagtttgaaaataataataatgatattaataaattagtttatttttttagttttatatatttttgtgtttatatttgtttttgttttatttataatggtttttttgtagttgttatattatattttgttatttgatgtttggtgaatattttatatttgttttttagaattttttttattttttttttatttgtttaatttttatatatttaaaattaattagagtaaattatttattagaataattaattttaaattttagtaatttaatatgataaaggtttgttttttatttatatagtttttttttagatgattgaggggtttaggttttttatttttagtggtttttttattttttggagttttttgtattttttatatatggttgagataaattatgagttattagtatagttagattttgaggttttataagaaaatttgtaaattatttattttgttttgaataaggtatatttaagatgatgttaaaatatttaatggttttgggttaaatatagtttatgattgtgtatttaaaatatatattgtaatatttttttttttttttattgattttatgaatttagtggggatttattttataagtttaaagataattattttttagattaagaatatttagggtaaaaagtattgtttaatatttttattgaggatgttatgatgtagtatattgtataagttggagttaaaggaaattttttttaaagtgttatttattaaaaattggaatatattttttaagataaattgaagtgtggtatataatatttaaatttttattatagatatagaggtgttattattttttatttttaaatttttttgttatgttgaggatatttaagaggagtaggatatgttggttgtagtaggagaaatttgaaagtatttatttttatggaatttataagggagagaattttttattttagtattgtttttgatatatttattattttaaaagataatgtagttaaatgtttttttttgtgttaaatttttataaaattgaaattttaaaatggtgataaaaattttatttttgatagaatttatttatttttttaattagatagggtataatttttaatttgtaaaataaaatgtaatatgtttatgaggttttattttaaagaatttgttattgagagtagtatttagaataatgggtggaaatgttaattttagagttttagattttattggtaattggggtagggaggggttttgggtggggtttttttagaggaggaggtgttgttagaaagttgtttggttagtttatagttgttattaattggggtaagttttgttgtatttgtgtgtgtgggtggtatttttaatgagaattagttttatttgttatttgagtgaaatttataatttgaggtggttagtgtttttgtattattgggatttgagatttttggagatgattgttgtttgtagtatggagttagtagaagtttgattttttttgggaatgggttgtattgagaggtttgattagttttagggttttagtgagggggtagtggaatttagtgagggattgagagttttatagtatgtatgagtttgatgttagagaaaaagttgggagataaaggagttgtgtgttattaaattgttgttgtagttgtagttatttaagtgttggatttgtgagtattttgtgtttttagtttttggatagaagttggagaatttttttggagaattttttgagttaggagatgagattttttaataattattatttttttttgtgttttttatttgttgtttgttgggataaatgatagttatagtttttttgatgataggatggaggttaagggtaggagttgattagtgttgtttttttttgtttttgatttaggaggtggagatttttttggtttagttatatttaatatttatttttttttttttttgtttttatatttttgaaatttttttttttttttttttttttttttttttggagatgggggaggagaaaaggggagtttagttgttatgattgagttgaaggtaaagggtttttgggttttttatgtggtgggtggtttgtttttttttgaggttggatttttattgttgtgttgtttagttgtaggtttgtttttggggagttagattttggatattttgtttgaagttttggttatatttatttttttggatgggttattttttttttggttttgttagggataggatttttttgatgaaaagatgtaggattagtagttgttgttggatgtggagggtgtatattttagagttgaagttataaggggtgttggaggtagtagttttagttttttagaaaaggatagtggattgttggatagtgttttggatattttgttggtgtttttaggttttgggtagagttaatttagtttttttgtttgtgaggttattagtttttggtgtttgtttggttttgaattttttgaagatttattggttgtttttgttatttagtgggtgttgtttttgtttatgagttggtttgggtgtaaggttggagatagttttgggatggtagttgtttataaagtgttgttttggggtttgttattagtttggtagttgttgtttttggtttttgagagtttttattggtttggggttttagtgaagttgtttttgtaggttgttgtggtggaggttgaggaggaggatggttttgagtttgaggagtttgtgggtttgtttttgaagggtaaattttgggttttgggtggtgtggtggttggaggaggagttgtggttgttttgttggggtggtagtaggaggtgttgttttggtttttaaggaagatttttgttttttagtgtttagggttgttttggtggagtaggatgtgttgattgtttgggatgtttttgttggttattatggtgatggattttatttatgtgtttattttgttttttaattatgttttattggtagtttgtatttggtagttgttggaagatgaaagttatgatggtggggttggggttgttagtgtttttgttttgttgtggagtttattttgtgttttgtttattttggttgttgtaggtgatttttttgattgtgtgtatttgtttgatgttgagtttaaggatgatgtgtattttttttatagtgatttttagttgtttgttttaaagataaaggaggaggaggaaggtgtggaggtttttgtgtgttttttgtgtttttattttgtggttggtgttaattttgtagtttttttggattttttgttggggttattgtttttgttgttgttgtgagtgattttatttagatttggggaagtggtggtgatggttgtatttgttagtgttttagttttgtttgtgttttttttggggttgattttggagtgtattttgtataaagtggagggtgtgttgttttagtagggtttgtttgtgttgttgttttgtaaggtgttgggtgtgagtggttgtttgtttttgtgggatggtttgttttttatttttgtttttgttgttgttgttggggtggtttttgtgttttattttgtatttggttttaatgggtttttgtagtttggttattaggttgttgtgtttaaggagggtttgttgtaggtttatttgttttattttaattatttgaggtgagggtttgggatggggtatgtttagtgtgtttgggagtagtggttttgttggtggtggtggttgttaatttttagttttagttttagtgtattgttgtgttttttggggtggttggagagggtgggtagtgggatatagtataggggtagttgtttttttttttttttttttttttttttatttttggggatatgaaggtgggtgtagaatatattatttttggggtgtgtttttttgaaagttgtttttttgtttgttttttaattttttgaattttttagattttgaagtagaattaattttgatttaaaatgtgtagtgttatattaggtttgttgtagtttagtggggtagaaagtgtgtggtgagttgggggttttatgaaatgttttttttttagaagaaggatgtttattaggagtgtttgttttggagaggagttaaggtattgtttttttgggaggggtgggatttgagaggtggttggttagaattgaaagtagtattattttagggatttgaatattttagtggtttagtttttttaagaattttaagattaaaattaagtttatgtgggaaatgtttaaattgtggatttaaatgtttgttattgtattgtattgtttttttattattgtttgttatttattataattttttttatatataggtttaaaaaatattattttgtatattgaagtaatggaatgtaaaaaaagaatgttttgtttggaattttatgttgtgaataggtaaaatagtgttagtgtattggataatattttaaaatgataaatatatatttgtttaagtaagtaatgattatagggttgtgttttaaaaatttaaaattaaaatattgtaaagtattattgaatttttaaagttaaattatatttgttttgatttagtatatagataggaaggatataatattttatttgttaaagattaaattgtttttatataaagagttttgtagaaagatttttttttaattgattttaattttttaggatataatattatatattaattattgtttttttatattggtgttattgatgaatggttaattatttgtaagtatggtgaatttagttatggatagtttattattaagtttagtttgtatgttttttaagtgtatatatatagttttgtttttaaaatttttttttattttgttaatattggtttaagaaatttttagtattagatagtggtgtatttaaaaataaatggagtattttgttttgtattttaagg

SEQ ID NO: 63 - DNA, 5085 bp, Artificial Sequence
  chemically treated genomic DNA (Homo sapiens)
ttttgaaatgtaaaataaagtattttatttatttttaagtgtattattatttaatattaaaggttttttaaattagtattaatagggtgaaaggagattttaaaaatagaattgtatatatatatttgaaagatatgtaaattaaatttggtaatagattatttgtaattggatttattatatttgtaaatgattagttatttattagtagtattaatataaaagaataataattagtgtataatattatgttttagaaagttaaagttggttaaaaggaaatttttttataaaattttttatatagaaataatttagtttttgataaatgaaatgttatgttttttttgtttgtatgttgggttaaaataaatatggtttggttttaaaagtttgatggtattttgtaatgttttggttttgagtttttaaaatataattttgtaattattgtttatttaagtaagtatatgtttgttattttaaagtattgtttaatatattgatattgttttgtttatttataatataagattttaaatagagtattttttttttatattttattattttagtatgtaaagtagtgttttttaaatttgtatataaaaaaaattgtagtgaatagtaagtaataataagaaaatggtgtaatgtagtgataggtgtttaaatttatagtttaaatattttttatgtgaatttaattttaattttgagatttttaagaaaattgagttattgaagtgtttaaatttttaagatggtgttgtttttggttttggttggttattttttaagttttattttttttgggggaatggtgttttaatttttttttaagataagtatttttggtaaatgttttttttttaagaaagaagtattttataaagtttttaatttgttgtgtattttttgttttattgggttatagtggatttagtgtgatgttgtatgttttaaattggggttggttttgttttggaatttggaagatttggaaagttaaaaaataaataaaaaaatagtttttagggaggtatgttttaaaaatagtatattttgtgtttatttttgtgtttttaagagtgaggagaggagggaagaagaagggaggtaattgtttttgtgttgtgttttgttgtttattttttttggttgttttggggagtgtagtggtgtgttggggttggggttgagggttggtggttgttgttgttaatggaattgttatttttggatgtgttgggtgtgttttgttttgggtttttattttaggtagttgagatagggtgggtagatttgtggtaggttttttttgagtatggtggtttggtagttgagttgtgggagtttgttgaggttgagtgtagggtagagtgtgggggttgttttggtggtggtggtagaggtggaggtggagggtaggttgttttgtgggagtaggtagttgtttgtgtttggtgttttgtagggtggtggtgtgaatgggttttgttggggtggtgtgttttttgttttgtataggatgtattttagggttgattttgaggaggatgtagatgagattgaggtattggtgggtgtggttgttattgttgtttttttgggtttggatggggttgtttgtggtggtagtgggggtggtggttttaatgggaaatttgggaaggttgtggggttggtattggttataaggtaggaatgtggggagtgtgtggaggtttttgtgttttttttttttttttttatttttagagtgggtggttggaagttgttatagagagggtatgtgttgtttttgggtttggtgttgggtgggtatgtgtagttggggaagttgtttatagtgattggggtggatgaggtatagggtgaattttgtggtggggtaaaggtgttggtagttttggttttgttgttgtaatttttgttttttagtagttgttgagtgtgggttgttaataaggtgtgattgagaggtaggataggtatgtggatgaaatttattattgtggtggttagtggggatgttttggatgattggtgtgttttgttttattagggtgattttgggtgttgagaagtgggaattttttttggggattagggtgatgttttttgttgttgttttggtgggatagttgtggttttttttttagttgttgtgttatttagagtttgaggtttgttttttagaagtggatttgtagattttttggatttagagttatttttttttttaatttttattgtagtggtttgtggagatggttttattggggttttggattagtgagggtttttagaggttgggagtagtagttgttgggttggtgataggttttggggtagtattttatgggtagttgttgttttggagttgtttttaattttgtatttggattggtttatgagtggggataatatttgttgggtggtgggggtagttggtggatttttgggaagtttggggttaaataggtattaagagttggtgattttgtaggtgggagggttgggttggttttgtttgggatttgagggtgttaatagagtgtttaagatattgtttagtagtttgttgtttttttttgggggattagaattgttgtttttagtattttttgtagttttagttttggaatatgtgttttttatgtttgatagtgattgttggttttgtgtttttttgttggaggggttttgtttttggtagggttgagggaagagtagtttgtttagggagataggtatggttgaaattttaggtaaggtgtttgaggtttggttttttgggaatggatttgtggttgggtgatatagtagtggggatttgattttgggggagggtgggttgtttgttatgtggggagtttggggattttttgtttttagtttagttatgatgattggattttttttttttttttttttgtttttagggaggagggaaaagggaaggaggagggggttttgggaatataggggtagagggaggagaaagtgggtgttgaatgtggttggattggagggatttttattttttgggttgggggtgggggagggtggtgttggttagtttttgtttttggtttttattttgttgttaggggaattgtggttgttgtttgttttagtgagtggtaagtggggagtgtaagaaaaagtagtaattgttaggagattttgttttttaatttggggagttttttaagagagttttttaatttttgtttgaggattggagatgtagagtatttataagtttggtatttgagtggttgtggttgtgatggtaatttagtgatatgtggtttttttattttttgattttttttttggtattaaatttgtgtatgttgtgaagtttttagttttttgttgagttttattgtttttttattaaaattttggggttagttggattttttggtatagtttatttttaggaagggttggatttttgttggttttgtattgtgggtgatagttatttttgaagattttagattttagtagtgtgggagtattagttgttttgggttgtagattttatttaaatgataagtgaagttagtttttattgagaatgttatttatatgtataaatataataaggtttattttgattagtgatagttgtggattggttagatagttttttaataatgtttttttttttagggaggttttgtttaaagtttttttttattttaattattggtaggatttgaaattttggagttggtatttttatttgttattttgaatgttatttttaatagtaggttttttgggatggaattttataagtatattatgttttgttttgtaaattaagaattatgttttatttaattggaaaaatgaatagattttattagaagtagaatttttgttattattttaagattttagttttgtaaagatttaatatagaggaagatatttggttatattattttttaaaataataaatgtattaaggatgatattaaaataagaaatttttttttttatgagttttataaaagtgaatgtttttaagttttttttgttgtgattaatatgttttgttttttttgagtatttttagtgtgataaagaaatttgggagtgggagatggtaatatttttgtatttatgatggaagtttggatgttgtgtgttatattttgatttgttttaaggaatgtgttttaatttttagtaaatagtattttaaggaaagttttttttagttttagtttatatagtgtgttatattataatatttttagtagagatgttgaatagtattttttattttaaatatttttagtttgaaaagtaattatttttgagtttataaaatggatttttgttaaatttatgaagttagtagaaaaagggaagaatattgtaatatatattttagatatatagttataaattgtatttgatttaagattattgggtattttaatattattttaaatatattttgtttaaaatagagtgaatgatttgtaaatttttttgtaggattttaaggtttagttgtgttaatgatttatagtttattttaattatgtataaagaatgtagaagattttagaaggtgggggagttattagaggtaaggagtttggattttttgattatttggggaggggttatatgagtgagaaataaatttttattatgttaggttattaggatttggagttggttgttttagtaaatagtttattttgattaattttagatatgtggaagttaggtagatagaggaaaggtaaagagaattttgggaagtaggtatagaatgtttattgggtattaaatggtaggatataatatggtaattgtaaggggattattatgaatagaataggagtaagtgtgggtatagaagtatatgagattagagaagtaaattaatttattaatattattattgttgtttttaggttttttttttttagtttattttaaaatagttattatgttttattgaggtgtaattttagtattatatagtttatttaaagtattagtttattgattttttatgtatttagagttgtgtaattatta

SEQ ID NO: 64 - DNA, 8222 bp, Artificial Sequence
  chemically treated genomic DNA (Homo sapiens)
aagattagatgtaattttgttaagtagttattttgtaaattgtatagttttatgtagatggaaggaagggtttgtgtttattttgattatagtattaaataaattgtattgtatttttattttttttagttatttttttaaaaagattttgagttttttgaatataggaaggtgttttatttgattttgttatttttagtatgtagtagtgtttgatatatagtaggtgttttattattgtgagagggatggatggatgggtggagttatagatggatagaaggatagatggagggatgggtggatgatggatggatagatggatggaggggggatgatgaatggagggataatgagtggatgaatgagggaatgggtggatggatggatggagggatggaggaatagatagatagatggagggatgggtgggtgatggatggatagatggatggagggagggatgatgaatggagggataatgaatggatgaatgaggggatgggtggatggatgaatggagggatgatgggtggatgaatgaattgagggatggatggatgaatatatggatggatggatagatggatagatggaggaattggtggattttggatggatgggtggatggatagatgaatgaatgtttggatagataaagagatgatggatagatgaatagatgaattaagggatgttggatagatggagggattgatagatgttggatggatgggtggtggatggatagatgagtgaatgtatggatagataaagagatgatggatggatgaattaagggatgatagatggatggatggatgagtaattggatggataagtggataaatggatagatggttgaatatttgaatggattgaaggaggatgtatggatgtaagataaggttaattattttttattttttttttttgtaaaattatttatttatttatttaataaatatttatttagtttaaatttggtataaagtattatgtgaggtttaagagatatgtgggttaataaaatagagtttttgtttttttgaaaattgtaaagaaaggggtgtggttttttgagtttaaattttaattttgttagtgattagttgtatattagtgatgtttttttatttttttttaattaaatagggataatgttagtatttattatattgggaggttttgtggggattaaatgagttattaaatgttaagtgtttgggatagggtttggtatttagtaaagttttttgtgagtgttggttgttattattttaatggagaagatggtatgaaaattaggaaataggatgttttttgggaagtaatgtaataggaatttatataaagaaaggaaaggaggaagtaattagtggtgttttaaaggagtatgttaagaaaaattttttagagggaaatttttgagtagggttatgaaaataggagttttttaagagattgtggatttgtttgggattatttggttataagtataaaattatttggtttttttttgttatttttggttgggtgaggggtttttggtaaaggggtagaaggtgtgtgagaggttgtgaatggttaggattgttttggggttagttggggtatttggtggttaagtttagaaatatgataggtttttttgggagggttgattgtagggagtgttgggttttaggttgttggtgttggtttttgtggtgttttttttgttggttatgagagtttagatagtgtttaattttttttttttttttttatatgtataattattttattttttgtggtttgagttgttttgttttgttataatggtatttgttttaaaatagttttttatgtgagggttagagaaaggaaaagattagattttttttggatgagagagagaaagtgaaggagggtaggggagggggatagtgagttattgagtgatttttgttaagtattttagaaggtataaaaatgtttttgggattaggtagttttaaattttagttgttggggttaggatatttagtgagtttatatttgttttttttgttttttttagattgtgttatggggtttagtgatggggaatggtagttggtgttgaatgtttgggggaaggtggaggttgatattttaggttatgggtaggaagtttttattaggtaaaaggaagagattttattgtttttgttatttatattttaagattaagggtgtttagttgtaaggtggaaagtttgtatgtggggtaggttagttggttgtattagttaagggtgttagaatggttatttgttttttttttgtttttaagtgttagggattggatttaggagagggaaaggagttattttaggttgatgttagtagttggaggaagtatgagaattaaatttaggatgtttagagtttattaggaagaattttagaattatagatagttagagttaataagggttttgagagattttgtatagttattttttttataggatgaggataaaaagggattgagaaggggaggatatttttagagttatagtttattaaatgtttttaaagtgttaaggttaagatatgttttttaaggggagatagatttggttttagatttggttttgttattgagttattgggtgatttttgggaaagtatttaattttttggagttttaatttttttttttgtatagtgaggggatattttaatatttatattttagaggagatgtgagaattaaataaaataatgtatgtaagaggtttggtatggtttttggtatatattgagttttagaaatgttagtagttattattgatgaagtttaggttagggattttttaaagtattgtaattagagaatagaagatagaggtttattagtgatttttgatgttgagtatgtttttagtttgagaggtttgaatgatgtggtttgtaagtatattttgttttttattataagggattttagaatatattaaagaaaataaaattttgaggtttgtaaatagagggtggttgtggtttgtatatagaagtttatttttttgttgttttttattttaaaggtgatatattttttttttggtttttttttttattattttgagttggtttttttagaagtttaataggttaagaattaatgtttttgttaatgggaggaaggaagtgggtgttggggttgttataagtgaatggattgtttttggtattttaatttgtagattaatttttttttatttgtgagttagagttaattttttttttttattttgttgttttttgggttttttttgggttttatttatttttagtttttgtttattttgggagtatggttggttttggaagttttgttatttaaaatttgagttgttggtgatttgtgttagaaatatttgaggaagaagagttgagttttttgttgattgtaatggtattgtagtgtgtttatgtttttttttttttgtttttgttatttggttgtaataggttttgttggattgtataaagtgtttttttgattaatatatatatataaattgatattaatttttgggttatagatatgttatgtgattttggataagtttttttatttttgggttttatttttttgttggttgagtgaagggattgttttttatggtttttgagattttgttttatgtttatgttttagaattttttgagtttttgatgtatttttgtttttggtgaggaggtaggatagttaggtgtggaggtaggagattagagtttgtagagtagaggtttttttaaggtgatttgggagttgttagatttaaggtagtgatttgtatgttttgaaagagatttagggtgttgtattattagggaggattatggaggtttgttgggggttgggagttagggataagatataatgtttagggttgttggtttttgaaaggttttagaggtttggtgttttgttatgttttaggttgtgggatatgtagagattgtgttagttggttttgtttttttagtatttggggtatagggaggatggtttttttgggggtagggtggaggaggtttatgtttaggattgtaaatgttatttgtgagagttggtattttgattttgttttagatattgattttggattttttaattgattttgaaaggttatgtttttaattttttgattgtaaaagagggtaaattttatgaagtttagtttgtaaaatattgaggtttttattgtatgtatataaatataaaattttagaatatagaagttggaaggttatttggagattttggttttaggagtggggttttggaagggagggggttgttgagtgggttttatatttttgttttttttttttttatttaaattttttttgttttatttattttggttgttttggggagtggggggaataaaggattttaaggttataaaatgtttgaaaattattagtggtggttattattttttattatagggaggaattgggtttgagagtttttattggttaatggtagttataggtaggattaaaaatttttgggtgatggtgatgataatgataatgataatgataatggtagagatgatgaggaggagaaagatagtaattatttatttagtatatattgattgttgggttttgtttaagtgttttatttattgaagtttgataataattttatgaggtagatatgattatgattattatatttaatagagaggttaagtaatttatgttaagttatatagtaagtaagaaaggagttggaatttaaatttaggtagtttggatttagaatttttgttttttttttttttttttgagatggagttttgttgggattataggtgtatgttattatgtttggttaatttttgtatttttagtagagattaggttttgttatgttggttaggttggttttgaatttttgattttaggtgatttgtttgttttggttttttaaagtgttgggattataagttaatgagtttggtttagaatttttgtttttgattattatgttatattgttttatgattttgttatatgtaagatgggttgttatttatgttttggtttttttattttagtagagttagaggggtgtttatggatttgataatgtgttttatgttttatgtgagtgtttaattgggttttagtttagagaatagtaggaaaatggttttttttttttttgatttggattgaagatttgtttgatgaattaggtttattgatagtagggttgaagttggaaatataggttagtggggttttttgaaatattagagtaaatgttttttgtttatttttataggttgaggggtttttatttgtttattttttggattaggtaaatttaggagaatggttttttgtggttgtttggggggtttttagttgtaaggtttgggtttataggaggtaggtagttgtgtggtgttggaatgattttgtggggagataaattagtttgattttgttttgttgtttaaagtaaatatggggtggggtggggtggtttaggtttataattttagtattttgggaggtaggattggggaattgtttgagtttaggagtttgagattagtttgagttatatagtgagattttttttttttataaaaaataaataaaattagttgggtgtggtggtgtgtgtttgtagttttagttatgtgggaggttgaggtaggaggattatttgggtttaggagattgaggttttagtgagttaagattatattattgtattttagtttgggtaatagagtaatattgtgttttaaaaaaatattaataaagtaaatatgagttttttttttggttgtttggtgttgagtggaaagaaaggtattggtgggtttagatttttttggtttatatatggatgagtaatgatttagagaggttaagtgattttaatattatagagtatattggtgttagaattgggattggaatttagtttttgtaattgttagttttatgtttttttttttagatattagagggtaggggtaggttttaaaatgtttgagatttttagttgttttgtggtttaaatttttggttttgttttgttttagatagtttgtttttagtgttttgatttttgattattgtttgtgtatgttaggttttgtatttgttaggtgggagtttaaggattattgttgttagataataatagttaggagtttagggatttttggatgaaaggtgttagagataagttgttagtgatgtatgaggatttggataaggttttggagtgaggtggtaatgttgggtatggtggtagattatgtttaatatgggggaagggagtgtatgtggtttaggtttgtgtgtgtttttgtgtgtgtgtgtaggtgagtatggtgtgtgttgggatatatttatgtgtatttttgtgtgagtgtgtaggtttatatgtgagtgtgtatatatatttgtgagagtttgtatgtatgtatatgtgtgaggtgttgatatatgtttatgtaggtattagtttgtttgtaaatatttttgtgtaataataataaagtttgaatagttattaagagtatagatatgaagttagattgtttgggttaaatttttagttagttagttttttggtgtttttgtttttttttttgtaaaatggtgtttattttattaggattttaaaattagttaatatggttgggtgtttgtaattttagtattttgggaggttgaggtaggtggattataaggttaggagattgagattattttgtttaatatggagaattttgagaattgtttaaatttaggaggtggaggttgtggtgagtagagattgtattattgtattttagtttgggtaatagagttagattttattttaaaaaagaaaaataaaaaataaaaaaattatgaatttatttttaggttgaggagtttagtattttggttgtgaaatattttttttataaaattttgggatggagattatggggattaggtgtttttttgtgataatttttgggtatggtggtttagggtgtaaattggagtgtggttataatatatattgtgtatttttataaggatgttatagagtttgggtattataaaagaggagttttttaaggaattgaaattattagataggagagagagttttgggtagatagggttgtttgtgttaaatattttagttgtggtataagggaaagggtgggagttatgaaattgttttattttgggtttaggtttgggttttgttgttagttagttaagtgattttggttatttatttttgtggttttttatgagtaaaaggtggaaatttatttttatttagagggtaggtttgattttttttaattagtatttatttgtttatagtaggaaggattgaggtttaaagttggaggtgggtaggaaggattgaggtttaaagttggaggtgggtaggaaggattaaggtttaaagttggaggtgggtaggaaggattgaggtttaaagttggaggtggttgtttagagttttagtagaggtttttggggtattttattgagtgtttggtaggagtgggtgtttgttttagggttgggttgagttgtttttattaggattttttgttatttgtatagtgaggggattgggaggtttagagagttatagtttgggtttaaaataagtaagaggtttttgagtgtgaggattgttttggagtggaatggtttttataggtaggagtgagttttttgtagttagaggtatttaagtagttgaaggataatttttgggtaggaagttgtagagatggttgtagtgtggattagaattgttgttttggttatttagattttattttagtttggtttttttggatagtatttttgtaatagtgagttggtgattttatgttttagaattttggtttttatatttgtaaaatgggaattatatgatatttattatgtgttagatattttgttggtatatagtatatattattttatttaattttttaagtagggataagttatttttattttttatatgaggaagttgaggtatagagaggtgaagtgaatggtttaaggttatatagttgggaagatagggagttaaatttgaattttagtttggttgtttttagattttatattgtattttttatgttgattttagttttttttgtgtttatagg

SEQ ID NO: 65 - DNA, 8222 bp, Artificial Sequence
  chemically treated genomic DNA (Homo sapiens)
tttgtgggtatagggaaggttggagttggtatgggaggtgtggtgtgaggtttgggggtagttagattagagtttaagtttagttttttgtttttttagttgtgtgattttgggttatttattttatttttttgtgttttagtttttttatataagggatggggataatttgtttttatttgaaggattaagtgagatagtgtgtgttatgtattaatagggtgtttggtatatagtgagtgttatataatttttattttatagatgtggaaattgaggttttgaggtgtaaagttattagtttattattgtaggggtgttgtttagagaagttaggttggaatgaggtttgagtgattaaaatagtagttttagtttatgttgtgattatttttgtagttttttgtttagggattgttttttagttgtttaaatatttttagttataggaggtttatttttatttgtgagggttattttattttagagtaatttttatatttagaaattttttgtttgttttgagtttaagttgtgattttttgaattttttagtttttttattgtgtagatgatgaagggttttggtgggagtaatttaatttagttttgagataggtatttatttttgttaggtatttagtgaggtgttttagaggtttttgttgggattttgagtagttatttttagttttagattttggttttttttgtttatttttaattttagattttggttttttttgtttatttttagttttagattttagttttttttgtttatttttagttttagattttagttttttttgttgtgagtaggtgggtgttggttaaagggagttagatttgttttttgggtaggagtgagtttttgttttttatttatggaagattatagagataagtggttaaggttatttggttagttagtggtagagtttagatttaaatttaaaatggaatagttttataatttttatttttttttttgtgttatagttgaaatgtttggtatgggtaattttgtttgtttagggtttttttttttgtttaatggttttagttttttgaaaagttttttttttatgatatttaggttttgtgatatttttgtaaaagtatatagtatgtattgtggttatattttagtttgtgttttgagttattatgtttagaagttgttataaggaagtatttgatttttgtagtttttattttagggttttatgaggagggtattttataattaggatgttgagttttttaatttgaaaatgagtttatggtttttttgttttttgttttttttttttgagatggaatttggttttgttgtttaggttggagtgtagtggtgtaatttttgtttattgtaatttttattttttgggtttgagtaatttttagaattttttatgttaggtaggatggttttgattttttgattttgtgatttgtttgttttagttttttaaagtgttgggattataggtgtttagttatattaattgattttaaaattttgatggggtagatgttattttgtagaggaagaaatagaggtattgaaagattaattagttggggatttaatttaggtagtttggttttatgtttatatttttgatgattatttggattttgttattgttatatagagatatttataggtaaattgatatttatatggatatgtattaatattttatatgtgtatatgtatgtaggtttttatagatgtgtgtgtatatttatatatgggtttgtatatttatatagaggtatatataaatatattttagtatatgttatatttatttgtatatatatatagagatatgtataagtttgaattatatatattttttttttttatgttgggtatggtttgttgttgtgtttaatattgttattttgttttaggattttatttaaatttttgtgtgttattgatggtttgtttttgatattttttatttaagggtttttgagtttttgattattattatttggtgataatgatttttgaatttttgtttgatagatgtaagatttggtatgtataggtgatggttagaggttaggatgttgaaagtagattgtttgaggtagagtagggttaagaatttgggttataaggtagttgggggttttagatattttggggtttgtttttgttttttgatatttagggaagagaatatgggattgatagttatggaagttgagttttggttttagttttggtgttaatgtgttttgtgatgttgaagttatttagtttttttggattattgtttatttatatgtaaattaaaagggtttagatttgttaatgtttttttttttatttaatattagatagttagggagagagtttatatttattttattaatatttttttgagatatggtattgttttgttgtttaggttggagtgtagtggtgtgattttggtttattggagttttaattttttgggtttaagtgatttttttattttagttttttgtgtagttgggattataggtatatattattatgtttagttaattttgtttattttttgtagagaaggggggttttattatgtggtttaggttggttttgaatttttggatttaagtgattttttgattttgttttttaaagtgttgggattataggtttgagttattttattttattttatatttattttaaatagtagagtagaattgggttaatttgtttttttgtaagattattttggtattatatagttgtttgttttttgtgggtttaggttttgtagttaggagttttttaggtagttatagaaggttatttttttggatttgtttgatttggaaaatgaatagatgaaagttttttagtttgtggaaatggatggagggtgtttgttttagtattttagaaagttttgttggtttgtgtttttagttttaattttgttgttagtgggtttggtttattagatagatttttagtttaagttagagagaagggaggttatttttttgttgttttttgaattgaggtttaattggatatttatatggaatatggagtatattgttagatttatgaatatttttttggttttattagaataaaagaattagagtgtgggtggtagtttgttttgtgtgtggtaaggttatggggtagtatagtatagtggttaagagtaaggattttgggttgggtttattggtttgtaattttagtattttgggaggttaaggtgggtagattatttgaggttaggagtttgagattagtttggttaatatggtgaaatttagtttttattaaaaatataaaaattagttaggtatggtggtatgtatttgtaattttagtaagattttattttaaaaaaaaaaaaaaggagtaaggattttggatttaagttgtttgaatttgaattttagttttttttttatttgttgtgtgatttggtgtaagttatttaatttttttgttaaatgtggtgattataattatatttattttataagattgttgttaggttttagtgagtgaaatatttaaatagagtttagtaattagtatgtgttgaataaatggttgttgttttttttttttttattatttttattattattattattattattattattattattattatttaggaatttttggttttgtttgtggttgttattaattggtggagatttttaggtttagtttttttttgtaatgagaagtagtgattattattggtgatttttaaatattttatgattttgaaattttttgttttttttgttttttaaaatagttaaagtaaataaaataaaaaaggtttgagtaaagaaaggaaaaataggggtgtggagtttatttagtagtttttttttttttgaagttttatttttggagttagaatttttaagtgattttttagtttttgtgttttagaattttatgtttgtgtatgtgtagtgaaggttttagtgttttatagattgggttttatggaatttgtttttttttataattagaagattggagatataattttttaaggttagttagaaaatttagagttagtatttagaataaagttaagatgttaatttttatgggtggtatttgtagttttgggtatgagttttttttattttgtttttagggaggttattttttttgtattttaaatattgagagggtagaattagttggtatagtttttgtatgttttataatttaagatgtgatagggtattaggtttttgggattttttagggattagtaattttaggtattatgttttatttttggtttttggtttttggtaaatttttgtgattttttttgatgatgtaatattttgagttttttttaagatgtataagttattgttttaggtttggtagtttttaaattattttggaaagatttttgttttgtaaattttggttttttatttttatgtttgattgttttgtttttttgttaaaggtaaaagtgtattaaggatttagagaattttagggtatgagtgtgggatgggattttggagattatggaagataatttttttgtttaattagtagagaaataaggtttagaggtggagagatttgtttaaggttatatggtatatttgtggtttaggagttgatgttaatttgtgtgtgtgtgttggttgggggagtattttgtgtaatttaatagggtttgttgtagttagatgatagggataggagaagaaagatataaatatattatgatgttattatagttaatagaaaatttaattttttttttttaagtgtttttagtatggattattaatggtttaaattttagataatggagtttttaggattagttatgtttttgaggtagatggaagttggaaatgggtaaagtttaagggagatttaaaggatgatggagtggaagaggaaaattggttttggtttataaataaggagaaattaatttgtaaattaaagtgttaaagataatttatttatttgtggtaattttggtgtttatttttttttttttgttggtagaaatgttgatttttaatttattagatttttgagggaattagtttagaatggtgagggaaggggttaagagaagagtgtattatttttgggatagaaggtaatgaggagatgagtttttatgtataaattatagttattttttatttataaattttagaattttgttttttttggtgtattttggaattttttgtggtagaaggtaggatatatttgtagattatattatttagattttttaaattagagatatatttaatattgaaggttattaatgagtttttattttttgttttttaattgtaatgttttgaaaggtttttagtttgggttttattagtaatagttattaatatttttaggatttagtatatgttaggaattatgttaggttttttgtatgtattattttatttaatttttatattttttttaggatatagatattaggatatttttttattgtataggagaggaaattgaggttttgagaggttgagtatttttttaaaggttatttagtggtttagtggtagagttaagtttagaattagatttgtttttttttgaagagtatgttttaattttgatattttaagagtatttaatgagttgtgattttggaaatgtttttttttttttagtttttttttgtttttattttgtaagagaggtggttgtataaaattttttaggatttttgttaattttgattgtttataattttaaaattttttttggtggattttgagtattttaggtttgatttttatgttttttttagttgttgatattagtttgaaatggttttttttttttttttgagtttaatttttgatatttaaaagtaaagaaaaagtaagtgattgttttaatatttttaattaatgtagttaattgatttattttatgtgtaaattttttattttgtagttgaatatttttgattttagggtgtgggtggtaggggtaatggaattttttttttttatttgatgaggattttttgtttatggtttgggatgttagtttttatttttttttagatgtttagtattaattgttattttttgttgttgagttttatggtgtagtttgaagaagataaaaagagtaagtatgggtttattgggtgttttggttttaatagttggggtttgaggttgtttggttttaagggtgtttttatattttttgggatgtttgataaagattgtttaatggtttgttgttttttttttttgtttttttttatttttttttttttatttagggagggtttaatttttttttttttttagtttttatatgggaagttattttagggtaggtgttattgtggtgaggtaggatagtttaggttatagggggtggggtggttgtgtgtgtggaaagaaggggaggaggttgggtattgtttggatttttatagttgatagaaagggtattatagaagttgatgttagtagtttgaaatttaatgttttttgtggttagtttttttaagaggatttgttatgtttttaagtttggttattaggtgttttggttggttttaggatagttttggttatttgtaattttttatgtattttttgtttttttgttagagattttttatttagttagaagtgatagaaaggaattggatggttttgtgtttatagttaggtggttttaggtaagtttataattttttagagaatttttgtttttatgattttgtttaaaggtttttttttgaaaagttttttttgatatatttttttgagatattattaattgttttttttttttttttttttgtgtaagtttttgttgtattgttttttaaagggtattttattttttggtttttatgttattttttttattaggataatagtagttagtatttataagagattttgttgggtgttaggttttgttttaaatatttggtatttggtaatttatttaatttttgtaagattttttaatgtgataggtattgatattatttttatttaattgagagaaagtagggaaatattattgatgtataattagttgttggtagagttgggatttgaatttaggaagttatgtttttttttttgtagtttttaggagggtaggagttttgttttattaatttatgtattttttgggttttatatggtgttttgtgttaagtttgaattgaataaatgtttattgagtaaatgggtggatggttttgtaaagaaagagagtggaggatgattagttttattttatatttatgtatttttttttaatttatttaggtatttaattatttatttatttatttatttgtttatttagttatttatttatttatttatttgttattttttaatttatttatttattatttttttgtttatttatgtatttatttatttatttatttattatttatttatttaatatttattaatttttttatttatttgatattttttaatttatttatttatttatttattatttttttgtttatttaggtatttatttatttatttatttatttatttatttaaaatttattagtttttttatttatttatttatttatttatttatgtgtttatttatttattttttaatttatttatttatttattatttttttatttatttatttatttatttttttatttatttatttattatttttttatttattatttttttttttatttatttatttatttattatttatttatttttttatttatttatttgtttttttatttttttatttatttatttatttatttttttatttatttatttattatttttttatttattattttttttttatttatttatttatttattatttatttatttttttatttatttttttatttatttgtaattttatttatttatttattttttttatagtgatagagtatttattgtgtgttagatattgttatatgttgaggataataaaattaaataaaatatttttttgtgtttaaggaatttagagtttttttagggagataattaagaggaatgaaaatatagtatagtttgtttagtgttatgattaaggtaggtataggtttttttttttatttgtataagattatatagtttataaagtggttgtttggtaagattatatttaatttt

SEQ ID NO: 66 - DNA, 3051 bp, Artificial Sequence
  chemically treated genomic DNA (Homo sapiens)
atagtggtttatgtttgtaattttatattttgggagattgattgggaagattgtttgagtttaggagttggagattagttagagtgatatagtgggattttttttttataaaaatatttaaaaattagttaggtatagtggtgtatttttgtagttttagttatttgggaggtggtaagaggattttttgagtttagaagttggaggtggtagtgagtagttattttgttattgtattttagtttgggtgttaaagggagatttttttttttagaaaataataaaaaaaaaagtttgatagtttttttgtttgtgttatataaaataatggtatatattataattgaaggtattttataatagaaatgtttttttttgtttttttttaaggttgatttagtgttttaagaggttgatatagaagggtaaagttaagtttttataaaatttagggaagagattgtgaaatttattttagaattttgtatggagttatagttgaattaggaaaaaaaaaaaaaaaaaaaaaaggaaagaaaggtattttagagataaagaatagattaaggagaggagatatttgaagttataatgattgagaatatgttagatttgaaatgaatttttagtttttttaataagataaataaaattaattttatatttagatatatggtagaaaaattaaagaattttgttgattaaggtattaaaagtaattaaagagaagtggtgtgaagaaagtaagagagaaataataaattttgtttattttgtaataattgaaaatttttggttgggtgtggtggtttatgtttgtaattttagtattttgagaggttgaggtaggtggattattttaggttaggtgtttaagattagtttggttaatatggtgaaattttgtttttattaaaaaaaaataataataataatataaaaattagttgggggtggtggtaggtatttgtaattttagatatttgggaggttgaggtaggagatttatttgaatttgggaggtggaggttgtaatgagttgagattgtgttattgtattttagtttggatgatagagtaggattttattttaaaaagaaaggaaaagaaaagaaaaaatattaaatgtgtatgttttttgatttagttgtattattttaaggagttgatattattaaaattgtttaagtgtttaaaggtgtttgtagttaaataataggagattgataaattatgttatatatatgtgatgttatgttttaaagaggtattgatatgataaaagatgtatgtggtataaaattaaatgtattttattaagtatttttttaagtgtttatggaatgagtgtatttttgaaaaaaaaaaagtgtatttgaatttttaaaaaagttttaaaagttttatataatgaatgattgagtgattataagagttggtgggggaatgttaagaggatgatagggagttaagtttaatagaataatttatttttttattttgtgatatttatgagtgtattaattttgtaattgaaaaataaagtgtatatttgtagtagttgtattttttttaggttgtaaggaggtttttttttttggtaggtttgatttgtattttatttttatttttgtggttggaaattttttatttatgtagtgggaggttgaggagttattataaagttggggtttgatgagttgggattgggatttgatttttatatatgtttggatttgttttgtggttgggtttaggagttaaagaggtggggagatttgtgtgatgttgttttgttttgtgtttgttttttttaatgtatgttttagggggtgggttttgtggggagtatggatatgattggttttaaagttttttttgtaaggttgtgggttggatagtgtggtgatgttgtaatgtggtgtagggtgagagtgtgtgtttgtggatgtggtggtattaaatggttgtaggtgtagtagagtggttgttgtttttttaggttttagttggttgttgtgatgtttgtttgtttgttttgaggtttttgaagttgaaattagttagattttttttttttttgtttgtttgtagtggtgttgttgttattttgttattatgtttgaggtgtgtttggtttagggttttatttttaagaaggtgttggaggtatttaaggattttattaatgaggtttgttgggatattagttttagtggtgtaaatttgtagagtatggatttgttttatgtttttttggtgtagtttattttgtggtttgagggttttgatatttattgttgtgattgtaatttggttatgggtgtgaattttattaggtgagttttgtggttttgggaagttggttttggtttgtttgtatttttggtgtttggtgggagtgtttttgagtttagtttttattggttggtgtgggttatttgtgtttttttattggtttgttatgtagtggggtggggtttagttgagtgtgtggtttggaaaagtttgtgttggttgttgtgtgaatttgtttttttgtgttaaagttataaagtgggtggtggtgggaaaattaagggtttttttgtagtgttaggaatattgttttagggttttttgtttattaaattttgttggttttgaatggatgttttagttgtggtttttttgtttttgagatggttttggtgtgttgtttgggttggtttttaatttttgggtttaagtgatttttttggtttagttgtggttgattttaaatgttttataatgtttttgtgagaaatgtggtagtttgttattttatttagtggtaggagattgtttttatttagaagggatattgttggtggtattttagtataaatattgttagatgtgttttaaaatgtttgtattaataatggtattttttagtagtttgtttattttttattagttttgagatggtttgatgggtgagagtggtaattttttttaattgtgtttgaaatatagttttttagtagatggtgttgattttaaagtatgtgttttttgttttttagt

SEQ ID NO: 67 - DNA, 3051 bp, Artificial Sequence
  chemically treated genomic DNA (Homo sapiens)
attagaagataggagatatatgttttaaaattaatgttgtttgttgaagggttgtattttgaatgtggttagaaggggttattatttttatttattaggttgttttagaattggtggagggtaagtggattgttggaggatgttattattaatgtagatgttttgggatgtatttggtagtatttatattaaaatattattagtagtgttttttttggatagaaataattttttgttattaagtaggatgataggttgttatattttttgtaagggtattataaagtatttaaagttaattgtgattgagttgggaggattgtttgagtttagaagttggagattagtttgggtaatatattgagattgttttagaaataagaaagttatagttaaagtgtttatttaaggttaataggatttagtgagtaaagagttttggaatagtgtttttggtattgtggaaaaatttttgatttttttgttattatttgttttgtgattttggtgtgaaaaagtaggtttgtgtagtagttagtgtgggtttttttgaattgtgtgtttagttgggttttattttattgtgtggtaggttaatgagaaggtgtggatggtttatgttagttaatgagggttaggtttgaaagtgtttttgttaagtattggaggtgtaggtgggttggggttggttttttggggttgtgaggtttatttggtgaggtttatgtttatggttaggttgtggttgtagtggtaggtgttgaagtttttagattgtagggtgagttgtattaaagagatgtgggatgagtttatgttttgtaggtttatattgttggagttaatattttagtaggttttgttgatgaggtttttgagtgtttttaatatttttttgaggatggagttttggattaggtgtgttttgaatatggtggtggagtggtaataatgttgttataggtaggtgggaaggaggaaagtttagttggttttggttttaggagttttagagtgagtgggtgaatgttgtgatgattggttgagatttagaaagataatgattattttgttatgtttgtaattgtttaatgttgttgtgtttgtaagtgtgtgtttttattttgtgttgtgttgtgatgttattatgttgtttagtttatggttttgtggggaagattttagggttaattgtgtttatgttttttgtgaggtttgttttttagagtatatattggaggaagtgggtgtagggtggggtagtgttgtgtaggtttttttgtttttttgatttttgaatttggttgtagaataagtttgggtatatgtggagattgggttttggttttggtttgttaagttttagttttatggtggttttttagttttttattatgtgggtagaaagtttttagttatgaaagtgaaagtgaaatgtaaattaagtttattgggaggaaaagtttttttgtagtttgaagagagtatagttgttgtaaatatgtattttattttttaattatagaattgatgtgtttgtaggtgttataagataaagaggtgaattgttttgttaaatttagttttttattatttttttaatatttttttgttagtttttataattatttaattgtttattgtataaagtttttaaagtttttttaaaagtttgaatatatttttttttttttaaaaatgtatttattttgtaaatatttggaaaagtatttaataaagtatatttaattttatgttatgtatattttttattatattagtatttttttaaaatatagtattatatgtatataatataatttattaattttttgttgtttaattataaatatttttgagtatttaggtaattttggtgatattaattttttgaagtaatatagttgagttaaagagtgtatatatttaatatttttttttttttttttttttttttttgagatggagttttgttttgttatttaggttggagtatagtggtgtgattttagtttattgtaatttttgttttttaggtttaagtgagttttttgttttagttttttgagtatttgggattataggtgtttattattatttttggttaatttttgtattattattattatttttttttagtagagatggggttttattatgttggttaggttggttttgaatatttgatttgaggtgatttatttgttttggttttttaaagtgttgggattataggtgtgagttattatgtttagttagaaatttttaattgttataggatggataggatttgttgttttttttttgttttttttatattatttttttttagttatttttaatattttggttagtagaattttttagtttttttattatgtatttaggtatggagttagttttatttgttttgttgagggaattgaagatttattttaagtttgatatatttttaattattatggttttaaatattttttttttttagtttattttttatttttggaatgttttttttttttttttttttttttttttttttttttagtttagttgtgattttatataaggttttaagatgagttttatagttttttttttggattttgtggagatttaattttatttttttgtattagttttttaaagtattgggttagttttaaaagggaataggaaagggtatttttattataagatgtttttaattgtaatatgtattattattttatgtagtataagtaagaaaattgttaaatttttttttttattattttttagagaggggagtttttttttgatatttaggttggagtgtagtggtaagatgattatttattgttatttttaatttttaggtttgagagatttttttgttattttttaaatagttgggattataggggtgtattattgtatttggttaatttttaaatgtttttgtagagagaaggttttattatgttgttttggttggtttttaatttttgggtttaagtaatttttttaattggttttttaaagtgtgagattataggtatgagttattgt

SEQ ID NO: 68 - DNA, 3664 bp, Artificial Sequence
  chemically treated genomic DNA (Homo sapiens)
gaatttttaattattattttgtttttttatttttaatatttttagtggtaattgtattttatttatttttgtgtttatgaagtatagtgttgtattttggagatattatatagaagttggtttaaagggtaataaattgtatttgttaagatgatagtatggatttataataatttttgaaattttataaagaaagatgattgagttaaattgatatatttagtattatagttgtggaagtttgtggttttttttatgttaaaggtaaaatggatataatgaattagttgttatttttagatgatatagaagttgaattttttttaagttttttaaagagaaagatatattaaaatttattaaaattagtattataaattgtatagaatagtttggttttttagagtatttttttggggtagtgagtttttatttaaaaaatgaagtttggattttaaatataagttttaaaagtgattaggttttagtaggagtttttaaatttttatttatattatgatttttaaatgttttgtttttgttgtttaggaataaaaattgttaaggttgagttagatgtaatgtataatattagatgatagatgtagtttattgtgttaaaaggtgttaaatatttaagtgtatgtgatatattttatattataagggtaaggaagttttattttatattttgatttgggttttttaatatatatttgtagtggatgtggtaattagtttttgttttatgttttattaggattaatgtaattagaaatttttaaaatggatttttattatgtttagttttgttttattgttttatttgtttattttaaaaagattttagaatggtgtttaggttgggttggtggtttatgtttgtaattttagtattttgggaggttaaggtaggaggattgttggagtttaggagttggagattagttagggtaatataaggagatattgtttttgtaaataaatagaaaaaaaaaaagtaagaaagaaagagagaaagaaggaaagaataagaaaaaggaaaaaaaaattaattgtagtaagtgtagaaattttttttaaggatatgatggatggtaatgaagatagagtttttgatattggattatttaaagtataatttaagtttttttttttttttttatagaattgtgaatttttgtgttttaattatatttaagttaggtgtggtggtgagtgtttgtagttgtagttgtgttggaggttgaggttgattgtttgagtttaggatttggaggttagtatgtgtaatataatgagatttagtttttaaatgtatgtttttttttatatatttaaaattttgatgtgaaaatattttaaaatttaatatattttaaatgtttttaattgtataataaataaaatgtaaataataaaataatttaatattaaatttaaaaatgaggtagaaataaagtatagtgatataaataataaatttttttttatatttttgaggtggttttttgagttttttattttttttttaaggttattgaaatgtgttttttggagttagtttgtaaattatgtatttagaaaaatataattatatatttttaattttaagtattagaagtgaaagtaatggaattttgatgtaaatataatattatttttttgatgagttattttgagtataataaatttgaattgtgttaatgttgggagaaaaaatttaaaagaagaatggagtgaatagtagttttttgttttgttgattagaaatagtaggatgatatttttttgattggaggagagtgtttgtgtttgtatttagttggtgtttgtttttttgttttttttttagttgttttttttttttttttttgtgttttagttatttgggaaggtttgtttagtgtagttgggttttgattggttgttttgaaagtttatgggttatttgattggtgaatttggggttttttagtgtggtgagtttgaaattgtttgtatttggttttaaagttggtttttggaaattgagtggagagtgatgtggttgttgtagttgttgttgtggttgttgtggaataataagttgggtatagtggttggggttagggttgtgtttaggggatggttgagggttttggagggtgagtattgaggaatggggttttttaagaaggttggattggaggttagggatttgtgtggggtttggttgggttttggggggtggtgggtagggtttttgtttgggtttggttgtgtgagttatattgggttggttttagagggaggttatgggagtttagtttggtggggttaggtggtgtgggggtgggggtttggggttgtagttatgagttgaggttggttgtttttaggaattgtttgaagaggtttttgggttttgattgaagtaaaatttgtttttttatgttgagtgtttgtggagtagtaggtttttttttgggtttggtttttgtgtgttttgttattttgttttgggggtgggaaagtgggattgtatttgggttaggtataggtgtgggtttttagagttaggttgttttgagaaattgattttggaatttgtgggtttttttgggtttgttttgtttatggttttggtttatttggagttgttaagaatagttagtttaggtatttaatttttatgagtagtgtagtgtagttttttttatttaaaaagatatatttttgtttttttttggattgatttttttttgaagatgaatgtgagaaatagaatttaaaggtttattttgagtgtgttttataaatgattttattattagtgatgggtgttagaaaaatttaggtaattatgtttggagttttgatgttttattggtaaattttagaggaagttttgaatttttggttgttagtttttttttttaatttatttattgtgatttaagtatattgtttttttgtttagtttttgatattgtttaggttataattagaattttatttgtggaattgtattttttatttttttgttttatggtgtttaagtgatattttttatttttggaaaaagtattttgaaagttttgagtatgtttgtttgtgattttagtaagtagaggaaaaagattaaaattgttatgtgggtaattgtaaatatttaaaatgtttttgttgttttaaataaaatttttttaatgaatagtttgaatatattttagttttgtttttataattttaaaaaataatgttttgaataaaattaggagtattataaagtggtataaatttgttgaaatatttgggtataattttggtatatgtaggtattattttaaagtgttataaaaagggaatataattttggtttttgtttggaaagaatagataagttgatatttgaatttgagggttattttgattggaagaagttaaatattatattagtatagaatatatatgtttttaatgttttaggagttaagaaggatggaaagttattgtaggagtttttatttttgtaaatattgtttgaatgtaattaatatttatggaatatttatgttgtagtaggtattgtggaaaatgttttatatatagtgttttattaatgtttaaaattatttgagttagtggtatgtaattaagttagttttttttttattttagg

SEQ ID NO: 69 - DNA, 3664 bp, Artificial Sequence
  chemically treated genomic DNA (Homo sapiens)
tttaaagtgaaggaaaaattgatttaattatatgttattaatttgagtagttttaagtattaatgaaatattatatgtaaagtattttttatagtgtttattgtagtataagtattttataaatattaattatatttaaataatatttataagggtaaggatttttataataattttttattttttttgatttttgagatattggaaatgtgtatattttatattagtgtagtatttagttttttttaattaaaataatttttaaatttaggtattaatttgtttattttttttaaataaaaattagaattatgttttttttttgtgatattttaaagtagtatttgtgtgtgttaaagttatatttaaatattttaataaatttatattgttttatgatatttttaattttatttaaaatattattttttaaagttatgaggatagaattaaaatgtgtttagattgtttattaggaaaattttgtttaaaatgataaaaatattttaagtgtttatagttgtttatgtgataattttaattttttttttttgtttgttgaggttatagataaatatgtttaagatttttaaggtattttttttaagagtaggaaatattatttaagtattgtaaaataaaagaatgaaaagtgtaattttatagataaaattttaattataatttaaataatgttaaggattaagtagaaaaatggtatatttgaattataatgaatgggttaaaaaaaaaagttgataattagaagtttaagattttttttgaagtttgttaatgaaatgttagaattttaggtataattatttagatttttttaatatttattattgatgataaaattatttataaagtatatttagaatagatttttaagttttattttttatatttatttttaaagaaaaattaatttagaaagaagtagagatgtatttttttaaatgaaaaaaattgtattatattgtttatgagagttaagtgtttgggttgattgtttttaatagttttaggtgggttggggttgtgggtagagtaagtttaagaaggtttatgagttttaggattggttttttagggtggtttggttttagggatttgtgtttgtatttggtttaggtgtggttttatttttttatttttgaggtgggatggtagggtgtgtaggggttgggtttaggaaagagtttgttattttgtaggtgtttagtgtgggggagtggattttgttttaattaagatttgaaagtttttttaggtagtttttgagaatagttgattttgatttatgattgtagttttgaatttttatttttatgttgtttaattttgttaggttgggtttttatagtttttttttaaagttagtttagtgtggtttatatggttagatttaggtgaaggttttgtttattgttttttgaggtttagttgggttttgtgtagatttttgatttttagtttggtttttttagaggattttgttttttaatatttgttttttgaggtttttggttgttttttagatatgattttgattttagttattgtatttggtttattattttgtggtggttgtagtggtagttataataattgtgttgttttttgtttaatttttaagagttagttttgaagttaagtgtgagtagttttaaatttattgtgttaaagggttttggatttattaattgggtagtttgtagatttttaaagtagttaattagagtttagttatgttgggtaggtttttttgggtggttagagtgtgaaagaaagaggaaagggtggttagagaaaaagtaggagggtgggtgttaattgagtgtgagtgtaagtgtttttttttagttgggagagtgttgttttattgtttttagttagtggagtaggaagttattgtttgttttgttttttttttaaatttttttttttagtattggtatagtttaaatttattatatttaaaatagtttattaaaaaagtgatattgtgtttatattgagattttattatttttatttttaatatttagggttaggagtgtatagttatgtttttttaaatgtgtgatttgtgggttggttttaaggagtatattttagtgattttaagaaggaaatggaaaatttaaaagattgttttaaaaatgtaaaggaaaatttattatttatattgttgtgttttgtttttattttatttttgaatttaatattaaattattttattatttatattttgtttattatataattaaaaatatttgaaatgtattaaattttaaaatatttttatattagaattttaaatatatagagagaggtatgtatttagagattgggttttattatgttgtgtatgttggtttttaaattttgggtttaagtaattggttttagtttttagtgtagttgtgattataggtatttgttattatgtttggtttaaatataattaaaatataaaaatttataattttatgaggggagagaaagaggtttaaattatgttttaaataatttagtgttaaaaattttgtttttattgttatttattatatttttgagagagatttttgtatttattgtagttaatttttttttttttttttttgtttttttttttttttttttttttttttttattttttttttttttgtttgtttgtggagataatgtttttttatgttgttttagttggtttttaatttttaggttttagtaatttttttgttttggttttttaaagtgttgagattataggtgtgagttattagtttaatttaaatattattttaaaatttttttaaaataaataaatagaatagtaaaatagagttagatataataaaaatttattttaaggatttttggttgtattgattttaatgggatatagaataaagattgattattatatttattataagtatgtattagaagatttaaattaggatatggaatggaatttttttatttttgtgatatagagtatattatatgtatttaagtgtttaatattttttaatataatgagttgtatttattatttggtgttatatattgtatttggtttaattttgataatttttgtttttaggtaatagaggtagagtatttgggagttatggtgtaggtaggaatttaagaatttttgttggagtttaattatttttaaaatttatgtttaaaatttaaattttattttttaaatgaaaatttattgttttagagaagtgttttagggaattaggttgttttgtgtagtttatggtattggttttaatgaattttaatatgtttttttttttgagaggtttaagaggaatttaatttttatattatttaaaaatggtaattaatttattatatttattttatttttaatataaagaagattataagtttttatagttgtgatgttaggtgtattaatttaatttaattatttttttttataaagttttagaggttattgtaaatttatgttattattttggtaaatatagtttattattttttgggttaatttttatgtagtatttttaaagtgtaatattgtgttttatagatataaaaatgaataagatatagttattgttggaggtgttgagggtaggggagtaggatgataattgggaattt

SEQ ID NO: 70 - DNA, 1729 bp, Artificial Sequence
  chemically treated genomic DNA (Homo sapiens)
ttgtatttttggttggggtattttaattagaattgttaaatttagtatataaaaataaggaggtttagttaaatttgaattttagataaataatgaataatttgttagtataaatatgttttatgtaatattttgttgaaattaaaaaaaaaaaaaaaagttttttttttatttttatttttattattaggtttaaggaatagggttaggggttttaaatagaatgtggttgagaagtggaattaagtaggttaatagaaggtaaggggtaaagaagaaattttgaatgtattgggtgttgggtgtttttttaaataagtaagaagggtgtattttgaagaattgagatagaagtttttttgggttgggtgtagttgtttgtggttgtaattttagtattttgggaggttgaggtgggaggattatttgaggttgggagtttaagattagttttattaatgtggagaaattttgtttttattaaaaatataaaaaattagttggttatggtggtatatgtttgtaattttagttgtttgggaggttgaggtaggagaattatttgaattagggaggtagaggttgtggtgagtagagattgtgttattgttttttagtttgggtaataagagtaaaagtttgtttaaaaaaaaaaaaaagtttttttgatgtgattgtttttttttaaatttgtagatttttttaagattatgttttttagatattttaaagattttagaagatatgttttgggggttttggaagttataaggtaaatataatatatttttttttttgattattaattttattagaggatgtggtgggaaaattattatttgatattaaaataaataggtttgggatggagtaggatgtaagttttttaggaaagtttaagataaaatttgagatttaaaagggtgttaagagtggtagtttagggaatttattttggattttgggggagggggtagagttattagtttttgtatttagggattttttgaggaaaagtgtgagaatggttgtaggtaatttaggtgttttggtgttaggagggatgtatttaggtttgtgtgaagagagggagaaagtgaagttgggagttgttatttttagatttgttggaatgtagttggagggggtgagttgggagtgtgtttgtttttaattataggagaaggaggaggtggaggaggagggttgtttgaggaagtataagaatgaagttgtgaagttgagatttttttttattgggattggagaaattaggggagttttttgggtagttgtgtgtttttttttatggggttttttattgtgttgtgtgtttggtttttattttttgtagtattttgtgttttgtgtttttttagttgggtttagttggagttatggggttggagttgtagtgagtattatggagttggtggttttgtgttgttgggggtttttttttgtttttttgttttttggagttgtgagtatttaaggtgggtttggtgtggggaggggatggagtagtggtgggattttgttttgtggatgttttgttgaggttttgtggttggtggggttagaggggtttggatgagtttttttattttgaagttgtggatagttgagatgtttagggtagttgggttttggggtttttgggtgggagggggtagttatatggtagtggtttgagatggtttatttaagagattggtgttttttaggt

SEQ ID NO: 71 - DNA, 1729 bp, Artificial Sequence
  chemically treated genomic DNA (Homo sapiens)
gtttggaaagtgttagttttttggatgggttattttgagttgttgttgtgtaattgtttttttttgtttgagggttttagggtttggttgttttgagtgttttgattgtttataattttgggataggagagtttgtttgggtttttttggttttgttggttgtgggattttggtggggtatttatagggtagggttttgttgttgttttgttttttttttatattagatttattttgggtgtttgtggttttggggggtaagagggtgaggaggagtttttagtggtataaggttgttagttttatggtgtttattgtggttttggttttatggttttggttggatttggttgggagggtgtggggtgtggggtgttgtgaggggtgggggttgggtgtgtggtgtagtaaagggttttgtgggaaggggtgtgtggttgtttggggggtttttttggtttttttggttttaatggaggggaattttagttttataattttatttttatatttttttaagtagttttttttttttatttttttttttttttgtgattgggagtaagtgtgtttttagtttgttttttttaattgtattttaataagtttgggagtggtaatttttagttttattttttttttttttttgtgtaggtttgggtgtgttttttttagtgttgggatgtttgggttgtttgtagttgtttttatatttttttttggagaatttttaaatgtagaggttggtgattttgtttttttttttggagtttgggataaattttttaggttgttatttttaatatttttttaagttttaggttttattttaaatttttttggggagtttgtattttattttattttaagtttatttgttttaatattaaataatggtttttttattatattttttagtaaaattgatagttaaggagggggatgtgttgtgtttattttgtggtttttaggatttttggggtatattttttggaatttttgaagtatttgaaaagtatgattttaagagggtttataaatttgggaggagatagttatattgaaaggatttttttttttttttaaatgaatttttgtttttgttgtttaggttggagagtaatggtgtgatttttgtttattataatttttgtttttttggtttaagtgatttttttgttttagttttttgagtagttgggattataggtatgtgttattatgattagttaattttttgtatttttagtaaagatagggtttttttatgttggtgaggttggttttgaatttttaattttaggtgatttttttgttttagttttttaaagtgttggaattataattatgagtaattgtatttagtttaaaaagatttttattttaattttttaaaatgtattttttttgtttatttaaggaggtatttagtatttaatgtatttaaggttttttttttgttttttgttttttattagtttgtttaattttattttttaattatattttatttggagtttttgattttattttttaggtttagtggtaggggtggggatggaaggaagatttttttttttttttttaattttaataagatattgtatgggatatatttatattaataaattatttattgtttatttgaaatttaaatttaattgggtttttttatttttatgtgttaaatttggtagttttagttggaatgttttagttaagaatgtag

SEQ ID NO: 72 - DNA, 12963 bp, Artificial Sequence
  chemically treated genomic DNA (Homo sapiens)
gttaggagttttagaaataggggagagttagaaagttggttagatttatgttttttaagtgtagggttagggttgagtttgtttttggggtaggtaagtttttttgaatttttgagggaagtagaagatataaattgttagataaaatgtaagtttagtttaaaagggttatgtgttgttttttttagttttggggtattttttttttagaaaattggattgttttatagtgaaaattttgggggtggttagttttttgttttgttgttatttttattatttatagttttttaagaagtttttaggttgggtgttgaattttgattaggaattattgagaaattgaggtagttgggagaagttgtagttttaagtgttgaaaggaagatgggggataataaatttgggttgttaagtaaagggggtagaggtttggagaagtgggttttaggattagaggatagattgattttatattttatttttttagattttatattttattgttattattatttatgtgttttttttgttttttgtagtgggttttttagaggtattttttatggtttttttagattttaattttggtttgtttgttttttttttagaaaggtttttgtttgttttttttgtaggaaggtttgtatttttagaaagtttttgttttttgatttgaggatttaatttattaggggaattaaattttgtttttaggggagtggagagagaaattgggttttttttttgtagtttttgggatatagttgagttagttataggatttggggataattggggtggatttttttttttgggaggtggtggtattagtttagagtttgtatttttatttattggggaagtgtggggagaaggatgggttggagttgggttttggtttgaaggatagtagtttggagttaatggttgagtttttaaagtttttatattgtagaggaagtatagtggagattagttttagttaggatggttttgaagtttttagggatttgatgtagagttaaagaaatttatttgtgtttttttttttttttgggagtaggtagaagatttttgggaggagaggtgaatagtggatgttaatttttttgaaagtattgtgttttttagtattgtgggttgttatgggttttttgttgttgtgggattttggtttattttttgattgggttgttgtattttggattagattttgtgggtgatttatggaatttgtggagttgggatgtgaaaggttagaaggttttttgtttttattaagttttagggttttttgtggttgttgggagttgtagtttgaatgtttttattttggtgagaagtgtttatgttttttttattgagttttgtggtaatttttaaagtatttgtattgtttttttgttgtttgtagagggtgtagtaggttttgtattttttttgtattttattttttaggttttagatttgttttttttatttaaaaaatatttattattgagtttttatttgttatttagtattgatataggtatttaggaatataataatgaataagatagtagaaaaattttatatttttataaggtttatgtttttatgtattgaaagtaatgaataaataaattttattagagtgataagggttgtgaaggagattaaataagatggtgtgatataaagtatttgggagaaaatgttagggtgtgatattatggaaagtttttttaaaaaatgatattttaattgatgagaagaaaggatttagttgagagtaaatgtaaaagtttttttttttttattttttatatttgatataatgtaggatttttttaaaatgatttttattaattttgtttttatagttttggtttgtagaattttttattttaaaatgttagtatttatggtattaggttggtgagaattttgattttgtatttttttttttaattttattttttttgttttttttggtaggtggattatttgtttttatttgttatggtgattgtttagttttgtgttaggagttttgtaggggttgatgggattggggttttttttttttatgtgtttaagattggtgttaaaagttttgagttttttaaaagtttagagttattgtttagggagtaggtagttgttgggttttggggatattttgtgtttgggttgggagtgtgttttttatgatggtgatatgtttttttggattgggtaagtttttgattgaatttgatgagttttttttgagttatgggtttttggttttgtgtatttttagtttgggaaaattgttggggttgggggtggggtagtggggatttagtgagtttgggggtgagtgggatggaagtttggttagagggattattataggagttgtattgttgggagatttgggtgtagatgatggggatgttaggattatttgaatttaaagttgaatgtttaggtagaggagtggagttttggggaattttgagttggtttaaagtgtatttttttgtatatttatttggtgttgggtgtagggaatttttgaaataaaagatgtataaagtattgaggtttgagatttttggattttgaaatattgagaatttatagttgtatattttagagtttatggtattttagtgaaaattggggttttattttgaaatgattatttgggggtgatttggggagtttaagttgttaaggttttataatttttggatttttgttttttttggagtgatttttttaggtagtttttggttttgttagatggagaaaatttaattgaaggttgttagttgtggaagtgagaagtgttaaattaggggtttgtttgttaggttgaggaggattgttgtaatttgagaggtttggtagttttgttattgtttggttttatatttatatttttgttttttgtagtagtatttttggtttttttttgttggagtagtttattatttatttgatgagaggggaggagagagagagaaaatgttttttaggttggttttttttatttggtagagggaggttgttattttttgtttgtatttttttttttggattatttagttatggtttttgtaaaggtaggggtatttgttttgatgtaaattttaatttttttttttttttgaatggtgtgttttattttttgggttgtttgtaatttaggtggatgttattatggtgtagatagggagggaaagaagtgtgtagaaggtaagtttggaggtatttttaagaatgagtatattttatttttttggagaaaaaaaaaaaagaatggtatgtttgagaatgaaattttgaaagagtgtaatgatgggttgtttgataatttgttgggaaaaataatttatttgttatttagttttgggttaggttattttagttttagatgtaggttgaatgttgtgaagtggaaggggtgggtttgtaggtgtttgtgtggttttttgtgtagtttttggtttgagttggttttttttggtaggaggtggaatttgaatttattttttttgttgttttattttttagtttgtggttgttttattttgtagtttttttttatgtatttgttgtgtattggttattttgtgttgtatttatgttattttttttttaaattgaggtggtatttatatatagtgttagtgtatatagtaagtgtataggaagatgagttttggtttttaattgttttgtgatgtttattaagttatagattttttttattgttttagaaatgttttattatgtttttttttagttgatttttgattttatttttattttgatttttataattattttgtttgttggagaattttatatagaatggaattaggatgggtgttgtggtttatgtttgtattttggtttatgtttgtattttgggaggttgaggtgggtggattatttgaggataggagttttagattagtgtggttaatgtggtgaatttttgtttttattaaaaaatataaaaattagttgggtgtggtgggtgtttgtaattttagttatttgggagggtgaggtaggagaattgtttgaatttgggaggtagaggttgtagtgagttaagattgtgttattatattttagtttgggtgataagaatgaaattttgttttaaaaaaaaggggggaattatatattatgtgtttatttttgttgggtttttgttttttaatgtattgtttgatatttgtttatgttgtatatattagtattttgtttttttttatttagtatagtttattgattgtatatttgtttttttgatggttttttgagttgttttttatttgtggttatgaaataaagttgttataaatatttttgtataattttttttgtgattatatgtttttgtgttttttggagaaatatttaggaggggaattgtggaggaagtaaaaagtagttgtattttgaatttttttagaagttttgagttttttagagtggttgtattattttatattttaattagtaaggtatgggagttattatggttgtgttatagttttttggatattaggtatgttagtttttttaatgtggtatatttttgtggttgtaatttatagttttttattgattaaggatgtttagtatttttttatgtgtttattggttatttgtattttgtttgtaaagtagttttttgagttttttatttgttattttggtttttttgtttgtttttattgtttagttgtgggattgttttatattttttggatataagtttttattagatttatgagttgtgaatgtttttttttgatttgttgtgggtttatttgtttgttttatagagtttatagaattttaagaggagtggattaattttttttatgtttagtatttgttttgttttgtttaggatatttttttttttttttttaattttagggttatgaagatataattttatatttttttttaggatttttatggtggtaagttttatagtaaggtttttaagttattaattaatttttaaaattaattgtttatggtgtgaggtgtaggagttagtttttggtattttttttgtatggaaatttagttattttgtttttatttgttgaaataggttttttttttttattgaatgtttttaattttaattattttatagttggagtatagggttattattttagtgttattttttttttttttttgttaatttttgagatagggatttatattgttgtttaggttagagtataatggtataattaaggtttattgtagttttgaatttttgggtttaagtagttttttagtagttttatgagtagttgggattattttattatatttagttaattattttatttttttgtattgataggattttattatgttgtttaggttggttttaaattgttggttttaagtttttattttattttggttttttaaagtgttgggattataggtgtgagttattatgtttgattttttagtgttattttttatttattttttttgttttttgttttttttaaatgttggaggaagaaatagtatttattttatataaattttttagaaaatagaggaatagattgggtgtggtggtttatatttgtaattttagtattttggtatgttgaggtaggggattatttgaggttgggagtttgagattagtttggttaatatggtgaaattttatttttattaaaatataaaagtagttaggtgtgtattatatttgtaatgttagttatttaggaggttgaggtataagaattttttgaatttgggaagtggaggttgtagtgagttgagattgtgttattgtattttagtttgggtaatagagtgagattttgttttagaaaaaaaaagaaagaaagaaaaaatagaggaatattttttaatttgtttttgaagttaggataattttggtattaaaattaaataaggatattataagaaaagaaaatatagattaatatttttgttagtatagatatgtaatagttaattaattttagtaaattaaatttggtaatatagaaaaaaggataaataggttagttgtggtggtttatgtttgtaattttagtattttgggaggttgaggtaggtagattatttgaggttaggagtttgagattagtttgattaatatggtgaaattttgtttttaataaaaatataaaaattaggttgggtatggtggtttatgtttgtaattttagtattttgggaggttgaggtgggtagattatgaggttaggagtttaagattagtttgattaatgtggtgaaatgttatttttattaaaaatatgaaaattagttggtgtggtggtatttgtttgtaattttagttatttaggaggttgaggtagaattgtttgaatttgggaggtagaggttgtagtgagttaagattgtgttattgtattttagtttgggtgatagagtaagattttattttaaaaaaaaaaaaaattagttgggtatggtggtgggtatttgaaattttagttatttgggagtttgaggtaggagaattgtttgaatttaggaggtagaagttgtattgagttgggattatattattgtattttagtttgggtaatagagtgagattttattttaaaaaaagaaaaagaaaaaggataaatatattttaattaaataatgtttattttatgattgtagttgatttaatatttaaaaattggtttggtgtagtagtttaggtttgtaattttaatattttaggaggttgaggtaggaagattttttgagtttaggattttaagattagtttgggtaatatagttagattggtttttattggggggaaaaaaattagtttgtgtaatttattatattaataaagggaaatataaaaattttatgattattttaatagatgtagtaaaagtagttaatgatattaatatatatgtatgattataaattaattaattttttagtaaattagggaaaggaaatttaattagtttgataatagggtgtttatagttggagttttattagtagtatatataatggtagaaaatttagtgttgttgggggtggtggtttatgtttgtaatgttagtgttttgggaggtttaggtgggtggattatgaggttaggagattgagattgttttgattagtatgttgaaattttgtttttattaaaaatataaaaataaaaaattagttgggtatggtggtgggtgtttatagtgttagttatttgggaggttgaggtgagagaatggtgtgaatttgggaggtggagtttgtagagtttagattgtgttattgtattttagtttgggtgatagagtgagattttgttttaaaaaaaaaaaaaaaaaaaaaagaaaagaaaatttaatgtttttttttttaagattaggaattagaaaaggatttgatttttataatgttgatattatattggaggttttaattaggtaagaaaaagaaataatgagggttgggtgtggtggtttaggtttgtaattttagtattttgggaagttgagatgggtggattatgaggttaggagattgagttattttggttaatatggtgaaattttgtttttattaaatatataaaaaattagttgggtgtggtggtgggtgtttgtagttttagttatttgggaggttgaggtaggagaatggtgtgaatttagggggtggagtttgtagtgagttgagattgagttattgtattttagtttgggtgatagagtaagattgtgttttaaaaaaaaaaaaagaaaaagaaataatgattagtggtttgatgttttatgttagtaattttagtattttgggaggttgaggtgggtagattatttgaggtttggagttggagattagtttgataaagatggtgaaattttgtttttattaaaatattaaaaaaatagttaggtgttggttgggtatagtggtttatgtttgtaattttagtattttgggaggttgaggtgggtggattatttgaggttaggagtttaatattagtttggttaatatggtgaaattttatttttattaaaaatataaaattagttgggtgtagtggtgggtgtttgtaattttagttatttgggaggtttaggtaggagaattgtttgaatttgggaggtggaggttgtagtgagttgagattgtattattgtattttagtttgggtgataaaagtaaaaattttgttttaaaaaaaaaagaattagttaggggtagtggtgaatgtttgtagttttagttatttaggaggtagaggtaggagaattatttgaattttggaggtagaggttgtagtgagttgagattgttttattgtattttagtttaggtgagaagagtaaaattttatgttaaaaaaaaaaaaaaaaaaggaaagaaaaaaaataatgattagaaaggaagaaattaaatatatttatagttagtatgattttatatatattatggttttaatggggttaggtgtggtggtttatgttgtaattttagtatttttaggaggttgaggtaggtggtttttttgggattagttggttaatatggtgaaattttaattttaataaaaatataaaaaattagttaggtgtggtgagggtatttttaattttagttatttaggaggttgaggtaggagaattgtttggatttgggaggtagaggttgtagtgagttgagattgtgttattgtattttagtttgggtaataagagtgaaattttggtagggtgtggttttatgtttgtaattttagtattttgggaggttgagttaggttgattatttgaggttaggagtttgagattaatttaatatggtgaaattttgtttttattaaaaatataagaattagttgggtgtagtggtgggtgtttgtaattttagttatttgggaggttgagatagaagaattgtttgaatttaggaggtggaggttgtagtgagttgagattatgttattgtatattatgttgggtaatagagtgagattttgttttaaaaaaaaaaaaaagatgaaattttattttaaaaaaaaaaaaaagttttaatggaaaatttataaaaagttattaaaattaataaataaatatagtagggttgtaggttatagggtaatatagttatttttttatttgtaggggtttggttttgggattttttatatattaaatttatagatgtttaagttttatatataagatggaatagtatttaatttatatatatttttttatatagtttaaattatttagattatttatattatttttatataatgaaaatgttaatgtatatgtaagtatgtatgtaagtatttgtattatattgtttagggaattattggatagataggtttttaagattgatattagtagttattgttaagattttggttaggtttgtttttgtttggggttttagttgattttattgtttttttatttagttaagggtatttgtattttttttggttttttggttatttggaaggtttagtttagtttggtatatttgtattttggtttattgatgttggtatttttgggaaggttttgttttgaaaaatatggagattttagttgttattgaagatttgagagataaagatagggagatttgtttgtagatttgtgtttttttaagtgggattgagattttgggttttttattttaggatagtattttttggtttgttgattgaatagatttttgaaggaggtgtagttgtattttaggagtgggggtgggagtagtattattgatttgtattaataattatatagttttttttagaataataatatagaataagtgaaatagaataattgtagaaagagttaatttttgttgagtttttattgtgtgtttagtattttttttaattttatattttttataatatatagagtattaggtaggtggggtttgggggtttatgtttgtaattttagtattttaggaggttaaggggggtggattatttgaggttgggagtttaagattagtttgattaatatggtgaaattttgtttttattagaagtataaaattagttaggtgtggtggtatatgtttgtagttttagttatttagtaggttgaggtaggagaattatttgaatttgggaggaggttgtagtaagtggagatagtgttattgtattttagtttgggtaataagagttgagattttgttttaaaataaaataaaataaaataaaataaaataaaataaaataaaaaaagaaaagagtttgttattaaaggagttgtttggtaggggatgttttgttagtgtaaataatagaaaagtgggttgggtatagtggtttatgtttgtaattttagtattttgggaggttaaggtgggtggattatttgaagttgggagtttaagattagtttgattaatatggagaaattttgtttttattaaaaatataaaattagttgggtgtagtggttgatgtttgtaattttagttatttgggaggttgaggtaggagaattgtttgaatttgggaggtagaggttgtggtgagttgagattgtattattgtattttagtttggatgagagtaaaattttgttttaaaaaaaaaaaaaaatagaaaagtgtaataaatatttatagtaggtatgttttttagtaaatttgatgataaatttggtataaagaaagagagtatttttgaaaaaaaaaaaaagaaaaagaaagagagtattttgtttgggtaatatagtgaaattttgtttttataaaaaaatttaaaaattggttgggtgtagtggtttatatttgtaattttagtattttgggagttggaggtgggaggattatttgaggttaggagtttgaaattagtttggttaatatggtaaaattttatttttattaaaaatataaaaaattaattaggtgtattggtgggtgtttgtaattttagttatttaggaagttgaggtaagaggattgtttgatattgggaggtggaggttatagtgagttgagattatattattgtattttagtttgggtgatagggtgagattttgtttttaaaaaaaaaaagaaaaagaaaaagattaaaaaattagttaggtaggtttttgtggttttagttatttgggaggttgaggtaggagaattattgagtttaggagtggtaggttgtagtgagttatgattgtattattgtattttagtttgggttttaaagtaagattttgttttaaaagaaaaaagaaagaaagaaagaatatggtgggttaggtatagtggtttatatttgtaattttagtgttttgagaggttgaggtaggtggattataaggttaggagttttatattagtttggttaatatggtgaaattttgtttttattaaaaatataaaaaattagtaggtagggtggtaggggtttgtaattttagttatttgggaggttgaggtaggagaattgtttgaaattagaaggtagaggttgtagtgagtttagattgtattattgtattttagtttgggtgaaaagagttaaattttattttaaaaaataaataaaaaaataaaataaaaaaaaatatggtagtttttgaaagtttgtttgggagaaggtgtgatgatggttgtataattttgtgtaagatgttggtttatataggggttgttttttgtttttttttgtttttttaattttttatataataggtttgtgtgttatgtatatttattgagtttaagtaggtgtaaggtattgtgatttaatattttggttagtaagataataagatagattattgttttgtttttaggaagtgtatatgttattagaggaaatagataaaataaataaggaaaagtattagataatgtaagtgttatgagaatgtaaatgaggtgatgtgaattaaaataggatgatttaagtttgtatggaaggtttttatttttatgtttttggttagttaaggaattattagttgattagtagagaagggtagtttgtttagttagagtttttggggaagagggagtggttgttaagagatgagattaaagaagttgagatgggttttttgtgagggggggttgtaatgtagggttgaggagtgtttgaagagaatgggtaggtgagtggtgagatagttgtttttttagaagttttgtagtgaaaggaattaaagaaatggagttgtgtattaggtggggaagggtgggggttaagggggtgtttttttttatatagagattgtaggttgagaatgattatatttttgttaataggaggtgggagtagggtatggtagtttatatttgtaattttggtattttaggaggtggaggtgggttgattatttgaagtaaggagtttgagattagtttggttaatatgtaaagttttgtttttattaaaaatataaaaattagttgggtgtggtggtatttgtttgtaattttagttatttgggagattgaggtaggagaatggtttgaatttggaaggtagaggttgtagtgagttgagattatgttattgtgttttagtttaggtgatagagagagattttattttaaaaaaaaaaaaaaatataggaagggagttgggaatagggtgtatatttaggaagttttggggatttagtggtgggaaggttggaagtttttttttgattgttttttttttaaagaagtgtatggttggtgtggggtggggtaggagtgtttgggttgtggtgaaatattggaagagagaatgtgaagtagttattttttttttgttttataggaagttgagttgttttagatattggtatggtgttgggggagggggttttttttttgtaggtttaggtgatttagggttggaagtgttttatgttggatttttatttttttttttgtagt

SEQ ID NO: 73 - DNA, 12963 bp, Artificial Sequence
  chemically treated genomic DNA (Homo sapiens)
gttgtaagaggaaaagtggggatttagtatgagatatttttaattttgggttatttgggtttgtagagaaggaatttttttttttaatattatgttagtgtttgagatagtttggttttttgtggagtaggaaaagaatggttgttttatatttttttttttaatgttttattataatttaagtatttttgttttattttatattagttatgtatttttttgaggaaaagataattagagagggatttttaatttttttattattaaatttttaagattttttaaatgtgtattttatttttaattttttttttgtatttttttttttttttgagatggagtttttttttgttatttaggttggagtatagtggtatgattttagtttattgtaatttttattttttgggtttaagttatttttttgttttagttttttgagtagttgggattataggtgagtattattatatttagttaatttttgtatttttagtagagatagggttttgtatgttggttaggttggttttgaattttttattttaggtgattggtttgtttttgttttttaaagtgttaagattataggtgtgagttattgtgttttgtttttattttttgttaataaggatatagttatttttagtttgtaatttttgtatggggaaggatatttttttggtttttatttttttttatttgatatatggttttatttttttgattttttttattgtaaagtttttggaagaataattgttttattgtttatttgtttattttttttggatattttttagttttgtattataatttttttttatgaagggtttgttttggtttttttaattttattttttaataattatttttttttttttaaaagttttagttagatgggttgtttttttttgttaattaattggtggttttttggttagttaggaatatgggggtaggggttttttgtgtagatttaagttattttattttaatttatattattttatttgtatttttatagtatttatattgtttgatatttttttttgtttattttatttgttttttttaatagtatatatattttttaagggtagggtagtgatttattttgttgttttgttgattaaagtattagattataatgttttgtatttgtttgggtttaataaatgtgtataatatataagtttgttatatgagaggttaagagagtgagaaagagtaaggggtagtttttgtgtggattagtattttgtatgaagttatgtaattattattgtatttttttttagataagtttttaaaggttgttatgtttttttttgttttgtttttttgtttgttttttgagatggagtttggttttttttgtttaggttggagtgtagtggtgtagtttaggtttattgtaatttttgttttttggttttaagtaatttttttgttttagttttttgagtagttgggattataggtttttattattttgtttgttgattttttgtatttttagtagagatagggttttattatgttggttaggttggtgtggaatttttgattttgtgatttatttgttttggttttttaaagtgttgggattataggtgtgagttattgtgtttggtttgttatgttttttttttttttttttttttttttgaggtagggttttgttttgaagtttaagttagggtatagtggtgtaattatggtttattatagtttgttatttttgggtttagtgatttttttgttttagttttttaagtagttgggattatagaggtttgtttggttaattttttagtttttttttttttttttttttttttggagatggagttttgttttgttatttaggttagagtgtagtggtgtgattttgatttattgtaatttttattttttagtattaagtgatttttttgttttaattttttgagtagttgggattataggtgtttattaatgtgtttgattaattttttgtatttttagtagagatggggttttgttatgttggttaggttggttttgaatttttgattttaggtgatttttttgtttttgatttttaaagtgttgggattataggtgtgagttattgtatttggttaatttttgagtttttttgtagaggtagggttttattatgttgtttaggtaggatgtttttttttttttttttttttttttttttagggatgttttttttttttatgttaaatttgttattagatttgttaagaaatatgtttattgtaagtgtttgttatatttttttgtttttttttttttttgagatagagttttgtttttgtttaggttggagtgtaatggtgtgattttggtttattgtaatttttgttttttaggtttaagtgatttttttgttttagttttttgagtagttgggattataggtattggttattgtgtttggttaattttgtatttttagtagagatggggtttttttatattggttaggttggttttgaatttttaattttaggtgatttgtttgttttggttttttaaagtgttgggattataggtatgaattattgtgtttagtttatttttttgttgtttgtattgataaaatattttttattaaatagtttttttaatggtaggtttttttttttttttattttattttattttattttattttattttattttattttgagatggagttttagtttttattgtttaggttggagtatagtggtattatttttgtttattgtaatttttttttggatttaaatgatttttttgttttagtttgttgagtagttaggattataagtatgtgttattatatttggttaattttgtatttttagtagagatggggttttattatgttagttaggttggttttgaatttttgattttaggtgatttattttttttggttttttaaagtgttgggattataggtgtgagtttttaagttttgtttatttagtattttatgtattatgggaaatgtagagttgaggaaagtgttgggtatatagtaagagtttaataaaggttagttttttttgtaattgttttattttatttgttttatattattattttagagagaattgtgtgattgttagtgtggattagtggtattgtttttatttttatttttaaaatgtaattatattttttttagggatttatttagttaataggttaggaggtgttgttttgaaatggggggtttaaagttttaattttatttggagggatataggtttatagataggtttttttgtttttattttttaaatttttagtagtaattaaaatttttgtgttttttagagtaggatttttttaggggtattagtattagtgggttaggatataaatgtgttaggttgaattaggttttttaaatggttagggagttaagagaaatgtaggtgtttttggttgggtgggaaggtaatgagattaattgagattttaaataggggtaggtttgattagaattttaatagtggttgttggtattagttttgaaggtttatttgtttagtgattttttaaataatatagtataagtatttatatatatatttgtatgtatattagtatttttattgtatgggggtaatgtaagtaatttagataatttaaattatatgggaggatatgtgtaggttaaatattattttgttttatatatgggatttagatatttgtgggtttggtgtgtgaggagttttagaattaagtttttatagatagagggataattatattgttttgtaatttgtaattttgttatatttatttattagttttggtagttttttatggattttttattaggatttttttttttttttgagatagagttttattttttttttttttttgagatggaattttgttttgttgtttggtgtggtgtgtaatggtatgattttagtttattgtaatttttattttttgggtttaagtaatttttttgttttagttttttaagtagttgggattataggtgtttattattatatttagttaatttttgtatttttagtagagatggggttttattatgttaggttggttttaaatttttgattttaggtgattggtttggtttagttttttgaagtgttgggattataggtgtaagattatattttgttggagttttatttttgttgtttaggttggagtgtaatagtgtgattttggtttattgtaatttttgttttttaggtttaagtaatttttttgttttagttttttgagtagttgggattagaggtgtttttattatgtttggttgattttttgtatttttattagagttggggttttattatgttggttagttggttttagggaagttatttgttttagttttttaaaagtgttaggattatagtatgagttattatgtttggttttattaggattatggtatgtatagaattatattggttgtgaatgtgtttgatttttttttttttaattgttattttttttttttttttttttttttttttttttgatatggaattttgttttttttgtttaggttggagtgtaatgggataattttggtttattgtaatttttgtttttggggtttaagtgatttttttgtttttgttttttgagtagttgggattataggtgtttattattatttttggttaatttttttttttttgagatggagtttttgtttttgttatttaggttggagtgtaatggtgtaattttggtttattataatttttgttttttaggtttaagtgatttttttgtttaagttttttaagtagttgggattataggtgtttgttattatgtttggttaattttgtatttttagtagagatggggttttattatgttggttaggttggtgttgaatttttgattttaggtgatttatttattttggttttttaaagtgttggggttataggtatgagttattgtatttggttaatgtttggttatttttttaatattttaatagagatgaggttttattatttttgttaggttggtttttaattttagattttaggtgatttgtttattttggttttttaaagtgttgggattattggtgtgagatattgggttattaattattatttttttttttttttttttttttgagatatagttttgttttgttgtttaggttggagtgtagtggtttgattttagtttattgtaagttttgttttttgagtttatgttatttttttgttttagttttttgagtagttgggattataggtgtttgttattatgtttggttaattttttgtatatttagtagagatagggttttattgtgttagttaggatggtttgattttttgattttgtgatttatttgttttggttttttaaagtgttgggattataggtttgagttattgtatttggtttttattattttttttttttgtttggttaaaatttttagtatggtattaatgttgtgagagttaaatttttttttagtttttgattttagaggaaaaagtgttgagttttttttttttttttttttttttttttttttgagatgaagttttattttgttatttaggttggagtgtagtggtatgatttaggttttgtaagttttgttttttgggtttatgttatttttttgttttagttttttgagtagttggtattataggtgtttgttattatgtttggttaattttttgtttttgtatttttagtagagatggggttttagtatgttagttaggatagttttgattttttgattttgtgatttgtttgtttaggttttttaaagtgttggtattataggtgtgagttattgtttttagtagtattgagttttttattattatgtatgttgttagtggaattttgattgtggatgttttgttattaaattagttaagttttttttttttagtttgttaggaggttggttggtttgtaattatgtatatgtgttgatattattaattgtttttgttatatttgttgaaatgattatagggtttttatgtttttttttgttaatgtggtgaattatatagattgatttttttttttttagtaaagattagtttgattatgttgtttaggttggttttgaaattttgggtttaagagatttttttgttttagttttttaaaatgttgggattataggtttgagttattgtattaggttaatttttgaatgttgaattagttataattatgagataaatattatttggttagaatgtatttattttttttttttttttttttttgagatggagttttattttgttgtttaggttggagtgtaatggtgtgattttagtttagtgtaatttttgttttttgggtttaagtgatttttttgttttagatttttgagtagttgggattttaggtgtttattattatgtttagttaattttttttttttttgagatgaagttttgttttgttgtttaggttggagtgtagtggtatgattttggtttattgtaatttttgttttttgggtttaagtaattttgttttagttttttgagtagttgggattataggtaggtgttattatattggttgatttttgtatttttagtagagatggtgttttattatattggttaggttggttttgaatttttgattttgtgatttgtttattttggttttttaaagtgttgggattataggtgtgagttattgtgtttagtttgatttttgtatttttattagaaatggggttttattatgttggttaggttggttttaaatttttgattttaagtgatttgtttgttttagttttttaaagtgttgggattataggtgtgagttattgtgattggtttatttatttttttttttatattattaggtttggtttgttaaaattggttagttgttgtatgtttatgttaataggaatattggtttatattttttttttttataatgtttttgtttggttttggtattaggattattttggttttgaaaataagttgggaaatatttttttattttttttttttttttttttttttttgagatagggttttattttgttgtttaggttggagtgtagtggtgtaattttggtttattgtaatttttgttttttaggtttaagggatttttgtgttttagttttttgagtaattggtattataggtatggtgtatgtttagttatttttgtattttagtagagatggggttttgttgtgttggttaggttggttttgaatttttgattttaaatgattttttgttttagtgtattaaagtgttgagattataggtatgagttattgtgtttagtttgtttttttgttttttgaagagtttgtgtaagatgggtattgtttttttttttaatgtttaaagagagtagagaatagaggagataaatagaaaatagtattaagaggttaggtatggtggtttatatttgtaattttagtattttgggaggttgagatgggatgaaagtttgaggttagtagtttgagattagtttgggtaatatagtgagattttgttaatataaaaaaataaaatagttagttgggtgtggtggagtaattttagttatttgtgaggttgttagaggattgtttgagtttaggggtttgaggttgtagtaagttttgattgtgttattgtattttagtttgggtaatagtgtgagtttttgttttaaaaattaataaagaaaaaaagaaaatagtattaaaatggtagttttatattttaattgtaaaataattaaaattaaaagtatttagtagagaaaggaagtttattttaataagtggagatagaataattggatttttatataggaaagatattagagattgatttttatattttatattataaataattaattttaagaattaattaatggtttaaggattttattgtaaaatttattattataaaggttttaaaagaaaatgtaagattatatttttatgattttggggttaaaaaaaaaaaaaaagatgttttaaataggataaggtaaatattgaatataaaaaagattaatttattttttttaagattttgtaaattttgtaaagtaaataaataggtttgtaatagattagaagaaaatatttatgatttatggatttgataaggatttgtatttagaaagtataaagtagttttataattgaataataaaaataaataaaaaaattaaaataataggtaaaagatttgaagagttattttataaataaaatatgaatggttaataggtatatgaaaaaatgttgaatatttttagttaatagagaattgtaaattataattataaggatatattatattagaaagattgatatatttaatgtttggaaggttgtggtataattataataatttttatattttgttagttggagtgtaaaatggtataattgttttggaaaatttagagtttttgaaaaagtttaaaatatagttattttttattttttttataattttttttttaagtatttttttaagaaatatgaaaatatatgattataaaaagaattgtataagaatgtttatagtagttttattttataattgtaaatgggaaataatttaaaaggttattaaaaggatggatatataattgatggattatattaaatgaaaaggagtaaaatattgatatatataatatgaatgaatgttagatagtatattgaaggatagaagtttgataaaaatgagtatataatgtatgattttttttttttttttgagatggagttttgtttttgttgtttaggttggagtgtagtggtatgattttggtttattgtaatttttgttttttgggtttaagtgatttttttgttttatttttttgaatagttgggattataggtatttattatgtttagttaatttttgtattttttagtagagatggggatttattatgttggttatgttggtttggaatttttatttttaagtaatttgtttgttttggttttttaaagtgtaggtgtgagttaaagtgtaggtgtgagttatagtgtttattttgattttattttatatgaagttttttaataggtaaaatggttatggagattaaaataaaggtggggttgggaattgattgggaagagatgtgatgaaatgtttttgggatgatgaaaagggtttgtgatttggtaggtattatggagtggttaggggttaaaatttatttttttgtgtatttgttgtgtgtattggtgttgtgtgtaaatgttattttgatttaggaaaaagatgatgtaagtatggtataaagtggttggtatgtggtaggtgtatgggaagaaattgtggaatgaaataattgtgagttaagagatggggtagtgggagaaatgaatttgagttttgttttttattaggaagaattggtttgggttgagggttgtatggaggattatatggatgtttgtgggtttgttttttttgttttatgatgtttagtttgtgtttggaattggaatggtttagtttaaagttagataataggtagattgttttttttgataaattattaaatgatttattattgtatttttttaaaattttatttttagatgtattatttttttttttttttttttgggaagatgagatatgtttatttttgaaagtgtttttgggtttgttttttgtatatttttttttttttttgtttatgttatggtagtgtttgtttaggttgtaggtgatttggggggtggggtatattatttaaagaaggggagggattgaggtttgtattaaaataaatatttttgtttttgtaaaggttataattaagtaatttagaaaaagaaatgtaggtggagaatagtagtttttttttgttaagtaagaggaattggtttaaaggatatttttttttttttttttttttttttattgggtgaatagtgagttgttttggtaaaaagaaattggaaatgttgttgtaagaggtagaaatgtaaatgtggagttaaataataatagggttgttgggttttttagattgtgatggttttttttggtttggtgggtaaatttttggtttagtattttttatttttatgattgatagtttttaattggattttttttatttagtggagttgggggttgtttggaaagattgttttaggaaggataaaggtttggaagttgtgggattttagtagtttgggttttttggattatttttaaatgattattttggaatggagttttagtttttattaggatgttatgggttttaaaatatatagttatgagtttttaatgttttgagatttaaaagttttagattttaatgttttgtgtattttttattttagggattttttatgtttagtattgggtggatgtgtaaagaagtatgttttaggttggtttaaggttttttaaagttttatttttttgtttaggtgtttaattttgagtttggatggttttaatatttttattatttatatttaggttttttaataatgtaatttttatgatgatttttttagttaagtttttattttatttatttttaaatttgttaagtttttattgttttatttttagttttagtgatttttttgagttgaaaatatatggagttgagagtttgtgatttagagaggatttattaagtttagttaggagtttatttaatttagggaagtgtgttattgttgtggaaagtatgtttttagtttgaatgtaaagtgtttttggagtttagtagttatttgttttttggatggtggttttagatttttgagaagtttaaaatttttagtgttagttttgagtatatgggaggggaaaattttaattttattaatttttgtgaggtttttggtataaagttggatagttgttatgataagtaagggtaagtaatttgtttgttggaggaagtaaaggaaatggagttggggaggagggtgtagagttaggatttttgttgatttggtgttgtagatattaatattttggggtggaaaattttgtaagttagagttgtgagggtagaattggtggaaattattttggaggaattttgtattgtgttaaatatgaagggtggaaggaagaaagtttttgtgtttgtttttagttggattttttttttttattagttaaaatgttattttttaggaaggttttttgtaatattatattttaatgtttttttttagatattttatattatattattttatttaattttttttataatttttattattttgataagatttatttgtttattgtttttagtatatggaaatgtaagttttatgaggatatagaatttttttattattttatttattgttgtatttttgagtgtttatattagtgttgggtagtaagtaagagtttgataataaatattttttgaatgagggagataggtttgaagtttggagaatgagatgtagaagaggtgtaagatttgttgtgttttttgtaggtggtgggggggtggtgtaggtgttttaagaattattgtgggatttggtagggggagtgtaggtgttttttgttaagatagaagtgtttagattataatttttagtagttatgaggagttttagggtttgatgggaatgggaaattttttaattttttatgttttggttttgtgggttttgtgggttgtttgtgaaatttgatttgggatgtggtggtttaattggaaggtggattgaaattttgtgatagtaagaggtttgtagtgatttgtggtgttaaggaatatagtgtttttaaaagaattggtgtttgttgtttgtttttttttttgggagttttttgtttatttttagaagaggagggaagtataggtgggtttttttagttttgtgttggatttttgagaattttgaagttattttggttgaggttaatttttgttgtgttttttttgtagtatgaagattttggagatttaattgttagttttggattgttgttttttagattaggatttagttttagtttatttttttttttatgtttttttgatgaataaaaatgtggattttgaattgatgttattgttttttgaaaggggggatttgttttggttgtttttagattttgtggttggtttagttgtgttttaggagttatgggagggggatttagtttttttttttatttttttggaaatagagtttggtttttttagtgagttgagtttttgaattgaggagtaagaattttttgaaaatataagtttttttgtagaagaagtaaatgggagtttttttgaagaagaagtgaatgggttagagttggggtttggaaaagttatggaagatatttttggggaatttgttgtagaggatgagggagatatgtaagtggtgatggtagtggagtgtggagtttggggagatgaagtgtgaggttgatttgttttttggttttgagatttatttttttaggtttttgttttttttgtttggtgatttaggtttattgttttttattttttttttagtgtttggaattatagttttttttagttgttttgattttttagtggtttttggttagagtttagtatttaatttgagaattttttgaaaggttgtaagtggtaaggataataatggggtagggagttgattatttttgagatttttattgtaaaatagtttagttttttaggagagggatgttttagagttgggagaagtggtatgtagtttttttagattgagtttatattttatttagtagtttgtgttttttattttttttaaggatttaggggggtttatttattttagaggtaggtttagttttagttttatatttgaaaagtataggtttggttagttttttaatttttttttgtttttagggtttttgat

SEQ ID NO: 74 - DNA, 3500 bp, Artificial Sequence
  chemically treated genomic DNA (Homo sapiens)
tttagaataaatggtagaggttagtgagtttagagatggtgatagggtaatgatttaggggtagttgtttgaaatgggagtaggtgaagttatagatgggagaagatggtttaggaagaaaaatttaggaatgggtaggagaggagaggaggatataggttttgtggggttgtagtttaggatgggattaagtgtgaagatattttagtaggtgaggttaggttttatgaatagagaagtagtttttattttttttgatgtatggatatatagagtgtgtggtgttgtgtttttagagttgggtttttttgttttggtttttagggagtgagaagtgaggttgatttgtttttgtttttttttgttattttaatatttattttttttttatgttttttttttttaaatatgatttggatttatgtttttgtttaaattttatgttaaattgtaaattttaatgttggaggtggggttttgtgagaagtgattggataatgtgggtggattttttgttttgatgttgtttttgtgatagagattttatatgatttggttgtttaaaagtgtgtagtatttttttttttttttttttttttttttatttatgttttgttatgtaagatgtttttgttttttttttattgtttagaatgattgtaagttttttgaggtttttttaggagtagaagttattatgttttttgtataattgtagaatgatgagtgaattaaattttttttttttataaattatttagttttaggtatttttttatagtaatgtgaggatagattaatataattttttatttttagatttttgtatatgtttagttttagatattattgtttttgggagtatgtatagtgtagttttttgttgataaaagtaaagttataaaaggtgataaaaatttgtatttggggatatttgattgtgaaagagggaggatagtatatttgtagttatagagattggggtttattgagttgaaatttggtagtattttggtataatatgtgtatgatttgtgtttaatgtttagagattagtgttgagtaaaatagtttggtttggggttgttgttgtttttattttttttttgtttattagagggtggtagagttttttttattttggagtttttttaggggttgttgattttttttagttgggtttatagtttagtagggtttatttttatttgggttattttggtttatgttttttttgttttttgagttttttatatggattttgttagtttttttttgtagtttattggttgtttatttgaggtttgttggttgtttatttgaggtttgttggttgttttttgtaggtagtttttgttttttatattttttttttttttgggtttagttgaaagggtgttttttagggtagttttttgtgatttttaggatagtttagttttttataggttttgatgttttttatgttgttattttatagttttgttattattattaattttttagttttatgaagtttattgagtgtttgttttttggttataggaaaattttgtgatagggattatgtttgttttgttttttgtggaattttagggtttagtttagtgtttgatatggaatagatgttttataaatattggttaaatgtgtgggagatttttaaaaagaagtatattatttttgtgtggtttttagtagttagagtttgttttatgtggatataggggtattggtattagtatgggaggaggttagtaagtgtttgtggttgttttaggaatgaggttttaatttttagagttttagaagggaggatagaggtttgtagggaatagattttttggtttgattttgtagtttaatttagagtttagggttagtttatattatgttgattttggttagtatttttagggtagttttagataaggttggaggtttttttttgttttttagggggtgatattgtatatagatattatttaggaaatggatttttttggataggaatttggttttgttaaggaagtggaggtggagtttggtttttattttttgttttaatagatttttttgattttttttatatatttgttttgtttttttttgggttttatgaggattttgttttgttaggggtttttgtgtaattttagatttttttttggtattattatggggaaggtggggtgattataggatagttagttttgtagagatagagattatttaggattgttagggagaatatggataggttttgagttgtagtttagttaatagatatggagagggagggtttttttggagttttttttaaggatagtagagtttagagttatttatttttttttattatagttttttttttttaggatatataagatatttttttttttatatgtaggatttggggatttttgagatttttgggtttgggtttttatttttgggttagtggtggggttggtggtattggagatagagggttggtttttttttagttattatttagtgagtttttttttagtttttagagttatttttgttatttttttgttgggtattattttatttttttagagttttggagagtatggggagatttgggattttgttgggtttttttgttataaaggaaaataatttttttggtgtgatagatttaaggatagaatatagtagaggttagtattggggaagataggttgtttttttaggggatgggggtttatttattttgttgaaaagatttgtttgaggaattgaaaatagaagggaaaaaagaggagggataaaagaggtagaaatgagaggggaggggatagaggatatttgaataaagattatatttatgatttatgtgatgttgagaagtatttttgttttaggaagagatttagggtagagggaggaaggatagtagattagatagttatagtagttttgataaaatgtttttggaatttaagtttttttttatagaggaggatagagtagatagtagagattatggagttttttttggtttttttttatagatggtgtattttttggtagaggtttttgtttataggtgaagggaggataatttgggagagggtgggaggagggagttggggttttttgggtaggatagggttgtgagatggatagagggtttttgttggagtttgaatagggaagaggatattagagagggataggagttatattagaaaaattaaattgaattggaattggaaaggggtaggaaaattttaagagttttatttttttagttaattgttattggttattatgtttttaaaaattataataattgtattagatgatattttaaataaaaatataattagggtatgaaatattgtttttatttgtttattgtggatattggaaaataagttttaggttgtggagggttttgggaatttttatgaatttatttataggaatttgtagtttgttttaggtattggggtgtaattaagatt

SEQ ID NO: 75 - DNA, 3500 bp, Artificial Sequence
  chemically treated genomic DNA (Homo sapiens)
gattttggttgtattttagtgtttgggataggttgtagatttttgtggatgagtttatgagggtttttagggttttttatagtttggggtttattttttaatgtttgtggtaggtggatgaggatagtgttttatgttttggttatgtttttatttaaagtgttatttgatgtagttattatgatttttaaaaatgtagtggttagtgataattaattaggaaaatagaatttttgaggtttttttgtttttttttaattttagtttaatttgatttttttggtgtgatttttgtttttttttgatgtttttttttttatttaggttttaataggagttttttgtttgttttatagttttgttttatttaggagattttagttttttttttttatttttttttaggttgttttttttttatttgtgagtaggagtttttgttaggggatgtattatttgtggggaggggttgagggagattttatggtttttgttgtttgttttgtttttttttgtggagaagagtttgagttttaggaatgttttgttaaggttgttgtgattgtttggtttgttgttttttttttttttgttttgagtttttttttagggtaggagtattttttagtattatgtgggttatgggtgtggtttttatttaggtgttttttgttttttttttttttatttttgttttttttgttttttttttttttttttttttatttttagttttttagataaatttttttggtaaggtggatggatttttattttttgggaggataatttgttttttttagtgttgatttttgttgtgttttgtttttgggtttgttatattagggggattatttttttttgtgatagagaaatttagtagggttttgggtttttttatgttttttagggttttgggaaggtgggatgatgtttaataggaaggtgatagaggtggttttgggggttagaaaaaggtttattgggtggtggttggggagggattagttttttgtttttagtattattaattttgttattgatttagggatggagatttaggtttagaggttttaggagtttttagattttgtatgtggagggggaggtgttttgtgtgttttggaaagagaggattgtggtggagggaggtgggtgattttgggttttgttgtttttggggaaggttttagggggattttttttttttgtgtttgttggttgagttgtggtttagggtttgtttatgttttttttgatagttttgggtggtttttgtttttgtgaggttgattgttttgtgattattttattttttttatggtggtattaggagggagtttggagttgtatagggatttttggtagaatagggtttttataggatttagaaaggaatagagtaggtatgtgggagagattagaagggtttgttggagtaagggatggaaattaggttttatttttatttttttagtaaagttaggtttttgtttaggggaatttgttttttgagtgatgtttgtgtgtaatgttattttttggagggtaagaggagatttttggttttgtttggaattgttttagggatgttgattagggttgatgtggtgtgagttgattttgaattttggattaggttgtagggttaggttggaggatttattttttgtaggtttttgtttttttttttgaagttttgggggttgaggttttatttttggggtagttgtgggtatttgttggtttttttttatgttggtgttagtgtttttgtgtttatatggaatagattttgattgttgggggttatatggaggtgatatgtttttttttagagattttttatatatttaattagtatttatggagtatttgttttgtgttaggtattgggttgggttttgggattttatagagagtaggatagatgtggtttttgttatagagtttttttgtaattgggagataggtgtttagtgaattttatgggattgaggagttaatggtaatgatagggttgtgaggtgatagtatagggggtgttggagtttgtgagagattgagttgttttggagattatagggagttgttttgggagatgtttttttagttgagtttggggaaggagggggtgtaggggataggagttgtttgtagagggtagttgataggttttaagtgggtggttgataagttttaggtgggtggttgataggttgtagggaggagttgatagagtttgtgtgaggagtttggagggtgaggaggatgtgggttgaggtgatttgggtgagggtggattttgttgggttgtgggtttggttgagggaggttagtagtttttggggaggttttagggtgggaggaattttgttgttttttggtggataggagggaagtggggatagtagtggttttagattaggttgttttatttaatattgatttttagatattgaatatgggttatgtatatgttatgttaaagtgttattaggttttagtttggtgagttttagtttttgtggttataagtgtattgtttttttttttttataattagatgtttttaaatgtagatttttgttattttttgtgattttgtttttgttggtaggaggttgtgttgtgtatgtttttaggggtagtgatgtttggggttaagtgtgtgtggggatttgggagtagaagattgtattagtttgtttttgtattgttataaagaaatatttgagattgggtaatttataaagaaaagaggtttaatttgtttattattttgtagttgtataggaagtatagtggtttttgtttttggggaggttttagaaaatttataattattttggatggtgaaggggaaataggaatgttttatatggtagagtatgagtaagagagagagagagagaggggagaggtgttatatatttttaaataattagattatgtgagatttttattatagaaatagtattaaagtagaaaatttatttgtattatttaattattttttataaggttttatttttaatattggggtttataatttgatatgagatttgggtggggatatagatttaaattatatttgagagagaggggtatgaggagaaggtgaatgttggggtagtagagaggagtagggataagttaattttattttttattttttggggattagaataggagagtttgattttgggggtatagtattatatattttgtgtgtttgtgtattaggggaggtgggagttgtttttttgtttatgggatttggttttatttgttgagatgtttttatatttagttttattttgggttgtagttttatagagtttgtgtttttttttttttttttgtttatttttggatttttttttttgaattatttttttttatttgtggttttatttgtttttgttttaggtagttgtttttggattattgttttgttattatttttgggtttattggtttttgttatttgttttgag

SEQ ID NO: 76 - DNA, 20 bp, Artificial Sequence
  Detection primer for ESR1
aggagggggaattaaataga

SEQ ID NO: 77 - DNA, 22 bp, Artificial Sequence
  Detection primer for ESR1
acaataaaaccatcccaaatac

SEQ ID NO: 78 - DNA, 21 bp, Artificial Sequence
  Detection primer for p21
ggattagtgggaatagaggtg

SEQ ID NO: 79 - DNA, 21 bp, Artificial Sequence
  Detection primer for p21
aaacccaaactcctaactacc

SEQ ID NO: 80 - DNA, 19 bp, Artificial Sequence
  Detection primer for p27
gtggggaggtagttgaaga

SEQ ID NO: 81 - DNA, 20 bp, Artificial Sequence
  Detection primer for p27
atacacccctaacccaaaat

SEQ ID NO: 82 - DNA, 20 bp, Artificial Sequence
  Detection primer for p16
ttgaaaattaagggttgagg

SEQ ID NO: 83 - DNA, 20 bp, Artificial Sequence
  Detection primer for p16
caccctctaataaccaacca

SEQ ID NO: 84 - DNA, 20 bp, Artificial Sequence
  Detection primer for PGR
gagggggtagtggaatttag

SEQ ID NO: 85 - DNA, 21 bp, Artificial Sequence
  Detection primer for PGR
cctttaccttcaactcaatca

SEQ ID NO: 86 - DNA, 21 bp, Artificial Sequence
  Detection primer for MB
gtttttggtaaaggggtagaa

SEQ ID NO: 87 - DNA, 22 bp, Artificial Sequence
  Detection primer for MB
cctaaaatatcaacctccacct

SEQ ID NO: 88 - DNA, 23 bp, Artificial Sequence
  Detection primer for PCNA
tttttaggttgtaaggaggtttt

SEQ ID NO: 89 - DNA, 22 bp, Artificial Sequence
  Detection primer for PCNA
taaatacctccaacacctttct

SEQ ID NO: 90 - DNA, 25 bp, Artificial Sequence
  Detection primer for CDC2
attagaagtgaaagtaatggaattt

SEQ ID NO: 91 - DNA, 19 bp, Artificial Sequence
  Detection primer for CDC2
tcaatttccaaaaaccaac

SEQ ID NO: 92 - DNA, 22 bp, Artificial Sequence
  Detection primer for ERBB2
ggagggggtagagttattagtt

SEQ ID NO: 93 - DNA, 22 bp, Artificial Sequence
  Detection primer for ERBB2
tatacttcctcaaacaaccctc

SEQ ID NO: 94 - DNA, 22 bp, Artificial Sequence
  Detection primer for TP53
gattgggtaagtttttgattga

SEQ ID NO: 95 - DNA, 22 bp, Artificial Sequence
  Detection primer for TP53
aaatctcccaacaatacaactc

SEQ ID NO: 96 - DNA, 22 bp, Artificial Sequence
  Detection primer for CEA
gtttaggatgggattaagtgtg

SEQ ID NO: 97 - DNA, 22 bp, Artificial Sequence
  Detection primer for CEA
aatcaaatatccccaaatacaa

SEQ ID NO: 98 - DNA, 18 bp, Artificial Sequence
  Detection oligonucleotide for ESR1
agatatatcggagtttgg

SEQ ID NO: 99 - DNA, 18 bp, Artificial Sequence
  Detection oligonucleotide for ESR1
agatatattggagtttgg

SEQ ID NO: 100 - DNA, 18 bp, Artificial Sequence
  Detection oligonucleotide for ESR1
ccaaactccgatatatct

SEQ ID NO: 101 - DNA, 18 bp, Artificial Sequence
  Detection oligonucleotide for ESR1
ccaaactccaatatatct

SEQ ID NO: 102 - DNA, 18 bp, Artificial Sequence
  Detection oligonucleotide for ESR1
gtttggtacggggtatat

SEQ ID NO: 103 - DNA, 18 bp, Artificial Sequence
  Detection oligonucleotide for ESR1
gtttggtatggggtatat

SEQ ID NO: 104 - DNA, 18 bp, Artificial Sequence
  Detection oligonucleotide for ESR1
atataccccgtaccaaac

SEQ ID NO: 105 - DNA, 18 bp, Artificial Sequence
  Detection oligonucleotide for ESR1
atataccccataccaaac

SEQ ID NO: 106 - DNA, 18 bp, Artificial Sequence
  Detection oligonucleotide for ESR1
gagaaagtcggtttttgg

SEQ ID NO: 107 - DNA, 18 bp, Artificial Sequence
  Detection oligonucleotide for ESR1
gagaaagttggtttttgg

SEQ ID NO: 108 - DNA, 18 bp, Artificial Sequence
  Detection oligonucleotide for ESR1
ccaaaaaccgactttctc

SEQ ID NO: 109 - DNA, 18 bp, Artificial Sequence
  Detection oligonucleotide for ESR1
ccaaaaaccaactttctc

SEQ ID NO: 110 - DNA, 18 bp, Artificial Sequence
  Detection oligonucleotide for ESR1
tttttggtcgtgaaattt

SEQ ID NO: 111 - DNA, 18 bp, Artificial Sequence
  Detection oligonucleotide for ESR1
tttttggttgtgaaattt

SEQ ID NO: 112 - DNA, 18 bp, Artificial Sequence
  Detection oligonucleotide for ESR1
aaatttcacgaccaaaaa

SEQ ID NO: 113 - DNA, 18 bp, Artificial Sequence
  Detection oligonucleotide for ESR1
aaatttcacaaccaaaaa

SEQ ID NO: 114 - DNA, 18 bp, Artificial Sequence
  Detection oligonucleotide for ESR1
tttagtagcgacgataag

SEQ ID NO: 115 - DNA, 18 bp
  Detection oligonucleotide for ESR1
tttagtagtgatgataag

SEQ ID NO: 116 - DNA, 18 bp, Artificial Sequence
  Detection oligonucleotide for ESR1
cttatcgtcgctactaaa

SEQ ID NO: 117 - DNA, 18 bp, Artificial Sequence
  Detection oligonucleotide for ESR1
cttatcatcactactaaa

SEQ ID NO: 118 - DNA, 18 bp, Artificial Sequence
  Detection oligonucleotide for ESR1
gtttaaatcgagttgtgt

SEQ ID NO: 119 - DNA, 18 bp, Artificial Sequence
  Detection oligonucleotide for ESR1
gtttaaattgagttgtgt

SEQ ID NO: 120 - DNA, 18 bp, Artificial Sequence
  Detection oligonucleotide for ESR1
acacaactcgatttaaac

SEQ ID NO: 121 - DNA, 18 bp, Artificial Sequence
  Detection oligonucleotide for ESR1
acacaactcaatttaaac

SEQ ID NO: 122 - DNA, 18 bp, Artificial Sequence
  Detection oligonucleotide for ESR1
tatgagttcgggagatta

SEQ ID NO: 123 - DNA, 18 bp, Artificial Sequence
  Detection oligonucleotide for ESR1
tatgagtttgggagatta

SEQ ID NO: 124 - DNA, 18 bp, Artificial Sequence
  Detection oligonucleotide for ESR1
taatctcccgaactcata

SEQ ID NO: 125 - DNA, 18 bp, Artificial Sequence
  Detection oligonucleotide for ESR1
taatctcccaaactcata

SEQ ID NO: 126 - DNA, 18 bp, Artificial Sequence
  Detection oligonucleotide for ESR1
tggaggttcgggagttta

SEQ ID NO: 127 - DNA, 18 bp, Artificial Sequence
  Detection oligonucleotide for ESR1
tggaggtttgggagttta

SEQ ID NO: 128 - DNA, 18 bp, Artificial Sequence
  Detection oligonucleotide for ESR1
taaactcccgaacctcca

SEQ ID NO: 129 - DNA, 18 bp, Artificial Sequence
  Detection oligonucleotide for ESR1
taaactcccaaacctcca

SEQ ID NO: 130 - DNA, 18 bp, Artificial Sequence
  Detection oligonucleotide for ESR1
taggttttcggggtaggg

SEQ ID NO: 131 - DNA, 18 bp, Artificial Sequence
  Detection oligonucleotide for ESR1
taggtttttggggtaggg

SEQ ID NO: 132 - DNA, 18 bp, Artificial Sequence
  Detection oligonucleotide for ESR1
ccctaccccgaaaaccta

SEQ ID NO: 133 - DNA, 18 bp, Artificial Sequence
  Detection oligonucleotide for ESR1
ccctaccccaaaaaccta

SEQ ID NO: 134 - DNA, 18 bp, Artificial Sequence
  Detection oligonucleotide for ESR1
ggtagggtcggggttaga

SEQ ID NO: 135 - DNA, 18 bp, Artificial Sequence
  Detection oligonucleotide for ESR1
ggtagggttggggttaga

SEQ ID NO: 136 - DNA, 18 bp, Artificial Sequence
  Detection oligonucleotide for ESR1
tctaaccccgaccctacc

SEQ ID NO: 137 - DNA, 18 bp, Artificial Sequence
  Detection oligonucleotide for ESR1
tctaaccccaaccctacc

SEQ ID NO: 138 - DNA, 18 bp, Artificial Sequence
  Detection oligonucleotide for ESR1
tttaatttcgggttgtgt

SEQ ID NO: 139 - DNA, 18 bp, Artificial Sequence
  Detection oligonucleotide for ESR1
tttaattttgggttgtgt

SEQ ID NO: 140 - DNA, 18 bp, Artificial Sequence
  Detection oligonucleotide for ESR1
acacaacccgaaattaaa

SEQ ID NO: 141 - DNA, 18 bp, Artificial Sequence
  Detection oligonucleotide for ESR1
acacaacccaaaattaaa

SEQ ID NO: 142 - DNA, 18 bp, Artificial Sequence
  Detection oligonucleotide for ESR1
aggtggttcgtcggtttt

SEQ ID NO: 143 - DNA, 18 bp, Artificial Sequence
  Detection oligonucleotide for ESR1
aggtggtttgttggtttt

SEQ ID NO: 144 - DNA, 18 bp, Artificial Sequence
  Detection oligonucleotide for ESR1
aaaaccgacgaaccacct

SEQ ID NO: 145 - DNA, 18 bp, Artificial Sequence
  Detection oligonucleotide for ESR1
aaaaccaacaaaccacct

SEQ ID NO: 146 - DNA, 18 bp, Artificial Sequence
  Detection oligonucleotide for p21
aggtgtatcgtttttata

SEQ ID NO: 147 - DNA, 18 bp, Artificial Sequence
  Detection oligonucleotide for p21
aggtgtattgtttttata

SEQ ID NO: 148 - DNA, 18 bp, Artificial Sequence
  Detection oligonucleotide for p21
tataaaaacgatacacct

SEQ ID NO: 149 - DNA, 18 bp, Artificial Sequence
  Detection oligonucleotide for p21
tataaaaacaatacacct

SEQ ID NO: 150 - DNA, 18 bp, Artificial Sequence
  Detection oligonucleotide for p21
tgggttagcggtgagtta

SEQ ID NO: 151 - DNA, 18 bp, Artificial Sequence
  Detection oligonucleotide for p21
tgggttagtggtgagtta

SEQ ID NO: 152 - DNA, 18 bp, Artificial Sequence
  Detection oligonucleotide for p21
taactcaccgctaaccca

SEQ ID NO: 153 - DNA, 18 bp, Artificial Sequence
  Detection oligonucleotide for p21
taactcaccactaaccca

SEQ ID NO: 154 - DNA, 18 bp, Artificial Sequence
  Detection oligonucleotide for p21
gtttatttcgtggggaaa

SEQ ID NO: 155 - DNA, 18 bp, Artificial Sequence
  Detection oligonucleotide for p21
gtttattttgtggggaaa

SEQ ID NO: 156 - DNA, 18 bp, Artificial Sequence
  Detection oligonucleotide for p21
tttccccacgaaataaac

SEQ ID NO: 157 - DNA, 18 bp, Artificial Sequence
  Detection oligonucleotide for p21
tttccccacaaaataaac

SEQ ID NO: 158 - DNA, 18 bp, Artificial Sequence
  Detection oligonucleotide for p21
ttgtagtacgcgaggttt

SEQ ID NO: 159 - DNA, 18 bp, Artificial Sequence
  Detection oligonucleotide for p21
ttgtagtatgtgaggttt

SEQ ID NO: 160 - DNA, 18 bp, Artificial Sequence
  Detection oligonucleotide for p21
aaacctcgcgtactacaa

SEQ ID NO: 161 - DNA, 18 bp, Artificial Sequence
  Detection oligonucleotide for p21
aaacctcacatactacaa

SEQ ID NO: 162 - DNA, 18 bp, Artificial Sequence
  Detection oligonucleotide for p21
ttggaattcggttaggtt

SEQ ID NO: 163 - DNA, 18 bp, Artificial Sequence
  Detection oligonucleotide for p21
ttggaatttggttaggtt

SEQ ID NO: 164 - DNA, 18 bp, Artificial Sequence
  Detection oligonucleotide for p21
aacctaaccgaattccaa

SEQ ID NO: 165 - DNA, 18 bp, Artificial Sequence
  Detection oligonucleotide for p21
aacctaaccaaattccaa

SEQ ID NO: 166 - DNA, 18 bp, Artificial Sequence
  Detection oligonucleotide for p21
agttggttcggcgttggg

SEQ ID NO: 167 - DNA, 18 bp, Artificial Sequence
  Detection oligonucleotide for p21
agttggtttggtgttggg

SEQ ID NO: 168 - DNA, 18 bp, Artificial Sequence
  Detection oligonucleotide for p21
cccaacgccgaaccaact

SEQ ID NO: 169 - DNA, 18 bp, Artificial Sequence
  Detection oligonucleotide for p21
cccaacaccaaaccaact

SEQ ID NO: 170 - DNA, 18 bp, Artificial Sequence
  Detection oligonucleotide for p27
ttgtttatcgttttattt

SEQ ID NO: 171 - DNA, 18 bp, Artificial Sequence
  Detection oligonucleotide for p27
ttgtttattgttttattt

SEQ ID NO: 172 - DNA, 18 bp, Artificial Sequence
  Detection oligonucleotide for p27
aaataaaacgataaacaa

SEQ ID NO: 173 - DNA, 18 bp, Artificial Sequence
  Detection oligonucleotide for p27
aaataaaacaataaacaa

SEQ ID NO: 174 - DNA, 18 bp, Artificial Sequence
  Detection oligonucleotide for p27
ttttattacggtgtttaa

SEQ ID NO: 175 - DNA, 18 bp, Artificial Sequence
  Detection oligonucleotide for p27
ttttattatggtgtttaa

SEQ ID NO: 176 - DNA, 18 bp, Artificial Sequence
  Detection oligonucleotide for p27
ttaaacaccgtaataaaa

SEQ ID NO: 177 - DNA, 18 bp, Artificial Sequence
  Detection oligonucleotide for p27
ttaaacaccataataaaa

SEQ ID NO: 178 - DNA, 18 bp, Artificial Sequence
  Detection oligonucleotide for p27
aagagaaacgttggaata

SEQ ID NO: 179 - DNA, 18 bp, Artificial Sequence
  Detection oligonucleotide for p27
aagagaaatgttggaata

SEQ ID NO: 180 - DNA, 18 bp, Artificial Sequence
  Detection oligonucleotide for p27
tattccaacgtttctctt

SEQ ID NO: 181 - DNA, 18 bp, Artificial Sequence
  Detection oligonucleotide for p27
tattccaacatttctctt

SEQ ID NO: 182 - DNA, 18 bp, Artificial Sequence
  Detection oligonucleotide for p27
tttgatttcgaggggagt

SEQ ID NO: 183 - DNA, 18 bp, Artificial Sequence
  Detection oligonucleotide for p27
tttgattttgaggggagt

SEQ ID NO: 184 - DNA, 18 bp, Artificial Sequence
  Detection oligonucleotide for p27
actcccctcgaaatcaaa

SEQ ID NO: 185 - DNA, 18 bp, Artificial Sequence
  Detection oligonucleotide for p27
actcccctcaaaatcaaa

SEQ ID NO: 186 - DNA, 18 bp, Artificial Sequence
  Detection oligonucleotide for p27
gtatttggcggttggatt

SEQ ID NO: 187 - DNA, 18 bp, Artificial Sequence
  Detection oligonucleotide for p27
gtatttggtggttggatt

SEQ ID NO: 188 - DNA, 18 bp, Artificial Sequence
  Detection oligonucleotide for p27
aatccaaccgccaaatac

SEQ ID NO: 189 - DNA, 18 bp, Artificial Sequence
  Detection oligonucleotide for p27
aatccaaccaccaaatac

SEQ ID NO: 190 - DNA, 18 bp, Artificial Sequence
  Detection oligonucleotide for p27
tataatttcgggaaagaa

SEQ ID NO: 191 - DNA, 18 bp, Artificial Sequence
  Detection oligonucleotide for p27
tataattttgggaaagaa

SEQ ID NO: 192 - DNA, 18 bp, Artificial Sequence
  Detection oligonucleotide for p27
ttctttcccgaaattata

SEQ ID NO: 193 - DNA, 18 bp, Artificial Sequence
  Detection oligonucleotide for p27
ttctttcccaaaattata

SEQ ID NO: 194 - DNA, 18 bp, Artificial Sequence
  Detection oligonucleotide for p27
gaaagggacgagttttta

SEQ ID NO: 195 - DNA, 18 bp, Artificial Sequence
  Detection oligonucleotide for p27
gaaagggatgagttttta

SEQ ID NO: 196 - DNA, 18 bp, Artificial Sequence
  Detection oligonucleotide for p27
taaaaactcgtccctttc

SEQ ID NO: 197 - DNA, 18 bp, Artificial Sequence
  Detection oligonucleotide for p27
taaaaactcatccctttc

SEQ ID NO: 198 - DNA, 18 bp, Artificial Sequence
  Detection oligonucleotide for p27
tttttatacgtagttaat

SEQ ID NO: 199 - DNA, 18 bp, Artificial Sequence
  Detection oligonucleotide for p27
tttttatatgtagttaat

SEQ ID NO: 200 - DNA, 18 bp, Artificial Sequence
  Detection oligonucleotide for p27
attaactacgtataaaaa

SEQ ID NO: 201 - DNA, 18 bp, Artificial Sequence
  Detection oligonucleotide for p27
attaactacatataaaaa

SEQ ID NO: 202 - DNA, 18 bp, Artificial Sequence
  Detection oligonucleotide for p16
tttttagtcgtataggtg

SEQ ID NO: 203 - DNA, 18 bp, Artificial Sequence
  Detection oligonucleotide for p16
tttttagttgtataggtg

SEQ ID NO: 204 - DNA, 18 bp, Artificial Sequence
  Detection oligonucleotide for p16
cacctatacgactaaaaa

SEQ ID NO: 205 - DNA, 18 bp, Artificial Sequence
  Detection oligonucleotide for p16
cacctatacaactaaaaa

SEQ ID NO: 206 - DNA, 18 bp, Artificial Sequence
  Detection oligonucleotide for p16
ggttgtatcgcggaggaa

SEQ ID NO: 207 - DNA, 18 bp, Artificial Sequence
  Detection oligonucleotide for p16
ggttgtattgtggaggaa

SEQ ID NO: 208 - DNA, 18 bp, Artificial Sequence
  Detection oligonucleotide for p16
ttcctccgcgatacaacc

SEQ ID NO: 209 - DNA, 18 bp, Artificial Sequence
  Detection oligonucleotide for p16
ttcctccacaatacaacc

SEQ ID NO: 210 - DNA, 18 bp, Artificial Sequence
  Detection oligonucleotide for p16
agagtgaacgtatttaaa

SEQ ID NO: 211 - DNA, 18 bp, Artificial Sequence
  Detection oligonucleotide for p16
agagtgaatgtatttaaa

SEQ ID NO: 212 - DNA, 18 bp, Artificial Sequence
  Detection oligonucleotide for p16
tttaaatacgttcactct

SEQ ID NO: 213 - DNA, 18 bp, Artificial Sequence
  Detection oligonucleotide for p16
tttaaatacattcactct

SEQ ID NO: 214 - DNA, 18 bp, Artificial Sequence
  Detection oligonucleotide for p16
tttgttaacgttggtttt

SEQ ID NO: 215 - DNA, 18 bp, Artificial Sequence
  Detection oligonucleotide for p16
tttgttaatgttggtttt

SEQ ID NO: 216 - DNA, 18 bp, Artificial Sequence
  Detection oligonucleotide for p16
aaaaccaacgttaacaaa

SEQ ID NO: 217 - DNA, 18 bp, Artificial Sequence
  Detection oligonucleotide for p16
aaaaccaacattaacaaa

SEQ ID NO: 218 - DNA, 18 bp, Artificial Sequence
  Detection oligonucleotide for p16
ggttttggcgagggttgt

SEQ ID NO: 219 - DNA, 18 bp, Artificial Sequence
  Detection oligonucleotide for p16
ggttttggtgagggttgt

SEQ ID NO: 220 - DNA, 18 bp, Artificial Sequence
  Detection oligonucleotide for p16
acaaccctcgccaaaacc

SEQ ID NO: 221 - DNA, 18 bp, Artificial Sequence
  Detection oligonucleotide for p16
acaaccctcaccaaaacc

SEQ ID NO: 222 - DNA, 18 bp, Artificial Sequence
  Detection oligonucleotide for p16
gttgttttcggttggtgt

SEQ ID NO: 223 - DNA, 18 bp, Artificial Sequence
  Detection oligonucleotide for p16
gttgtttttggttggtgt

SEQ ID NO: 224 - DNA, 18 bp, Artificial Sequence
  Detection oligonucleotide for p16
acaccaaccgaaaacaac

SEQ ID NO: 225 - DNA, 18 bp, Artificial Sequence
  Detection oligonucleotide for p16
acaccaaccaaaaacaac

SEQ ID NO: 226 - DNA, 18 bp, Artificial Sequence
  Detection oligonucleotide for p16
ggtgttttcgggggagat

SEQ ID NO: 227 - DNA, 18 bp, Artificial Sequence
  Detection oligonucleotide for p16
ggtgtttttgggggagat

SEQ ID NO: 228 - DNA, 18 bp, Artificial Sequence
  Detection oligonucleotide for p16
atctcccccgaaaacacc

SEQ ID NO: 229 - DNA, 18 bp, Artificial Sequence
  Detection oligonucleotide for p16
atctcccccaaaaacacc

SEQ ID NO: 230 - DNA, 18 bp, Artificial Sequence
  Detection oligonucleotide for p16
atttggggcgattttagg

SEQ ID NO: 231 - DNA, 18 bp, Artificial Sequence
  Detection oligonucleotide for p16
atttggggtgattttagg

SEQ ID NO: 232 - DNA, 18 bp, Artificial Sequence
  Detection oligonucleotide for p16
cctaaaatcgccccaaat

SEQ ID NO: 233 - DNA, 18 bp, Artificial Sequence
  Detection oligonucleotide for p16
cctaaaatcaccccaaat

SEQ ID NO: 234 - DNA, 18 bp, Artificial Sequence
  Detection oligonucleotide for p16
gttatattcgttaagtgt

SEQ ID NO: 235 - DNA, 18 bp, Artificial Sequence
  Detection oligonucleotide for p16
gttatatttgttaagtgt

SEQ ID NO: 236 - DNA, 18 bp, Artificial Sequence
  Detection oligonucleotide for p16
acacttaacgaatataac

SEQ ID NO: 237 - DNA, 18 bp, Artificial Sequence
  Detection oligonucleotide for p16
acacttaacaaatataac

SEQ ID NO: 238 - DNA, 18 bp, Artificial Sequence
  Detection oligonucleotide for p16
taagtgttcggagttaat

SEQ ID NO: 239 - DNA, 18 bp, Artificial Sequence
  Detection oligonucleotide for p16
taagtgtttggagttaat

SEQ ID NO: 240 - DNA, 18 bp, Artificial Sequence
  Detection oligonucleotide for p16
attaactccgaacactta

SEQ ID NO: 241 - DNA, 18 bp, Artificial Sequence
  Detection oligonucleotide for p16
attaactccaaacactta

SEQ ID NO: 242 - DNA, 18 bp, Artificial Sequence
  Detection oligonucleotide for p16
gatagggtcggagggggt

SEQ ID NO: 243 - DNA, 18 bp, Artificial Sequence
  Detection oligonucleotide for p16
gatagggttggagggggt

SEQ ID NO: 244 - DNA, 18 bp, Artificial Sequence
  Detection oligonucleotide for p16
accccctccgaccctatc

SEQ ID NO: 245 - DNA, 18 bp, Artificial Sequence
  Detection oligonucleotide for p16
accccctccaaccctatc

SEQ ID NO: 246 - DNA, 18 bp, Artificial Sequence
  Detection oligonucleotide for p16
gttagtatcggaggaaga

SEQ ID NO: 247 - DNA, 18 bp, Artificial Sequence
  Detection oligonucleotide for p16
gttagtattggaggaaga

SEQ ID NO: 248 - DNA, 18 bp, Artificial Sequence
  Detection oligonucleotide for p16
tcttcctccgatactaac

SEQ ID NO: 249 - DNA, 18 bp, Artificial Sequence
tcttcctccaatactaac

SEQ ID NO: 250 - DNA, 18 bp, Artificial Sequence
  Detection oligonucleotide for PGR
agtatgtacgagtttgat

SEQ ID NO: 251 - DNA, 18 bp, Artificial Sequence
  Detection oligonucleotide for PGR
agtatgtatgagtttgat

SEQ ID NO: 252 - DNA, 18 bp, Artificial Sequence
  Detection oligonucleotide for PGR
atcaaactcgtacatact

SEQ ID NO: 253 - DNA, 18 bp, Artificial Sequence
  Detection oligonucleotide for PGR
atcaaactcatacatact

SEQ ID NO: 254 - DNA, 18 bp, Artificial Sequence
  Detection oligonucleotide for PGR
gaaaaagtcgggagataa

SEQ ID NO: 255 - DNA, 18 bp, Artificial Sequence
  Detection oligonucleotide for PGR
gaaaaagttgggagataa

SEQ ID NO: 256 - DNA, 18 bp, Artificial Sequence
  Detection oligonucleotide for PGR
ttatctcccgactttttc

SEQ ID NO: 257 - DNA, 18 bp, Artificial Sequence
  Detection oligonucleotide for PGR
ttatctcccaactttttc

SEQ ID NO: 258 - DNA, 18 bp, Artificial Sequence
  Detection oligonucleotide for PGR
ttaagtgtcggatttgtg

SEQ ID NO: 259 - DNA, 18 bp, Artificial Sequence
  Detection oligonucleotide for PGR
ttaagtgttggatttgtg

SEQ ID NO: 260 - DNA, 18 bp, Artificial Sequence
  Detection oligonucleotide for PGR
cacaaatccgacacttaa

SEQ ID NO: 261 - DNA, 18 bp, Artificial Sequence
  Detection oligonucleotide for PGR
cacaaatccaacacttaa

SEQ ID NO: 262 - DNA, 18 bp, Artificial Sequence
  Detection oligonucleotide for PGR
gtattttgcgtttttagt

SEQ ID NO: 263 - DNA, 18 bp, Artificial Sequence
  Detection oligonucleotide for PGR
gtattttgtgtttttagt

SEQ ID NO: 264 - DNA, 18 bp, Artificial Sequence
  Detection oligonucleotide for PGR
actaaaaacgcaaaatac

SEQ ID NO: 265 - DNA, 18 bp, Artificial Sequence
  Detection oligonucleotide for PGR
actaaaaacacaaaatac

SEQ ID NO: 266 - DNA, 18 bp, Artificial Sequence
  Detection oligonucleotide for PGR
ttagttttcggatagaag

SEQ ID NO: 267 - DNA, 18 bp, Artificial Sequence
  Detection oligonucleotide for PGR
ttagtttttggatagaag

SEQ ID NO: 268 - DNA, 18 bp, Artificial Sequence
  Detection oligonucleotide for PGR
cttctatccgaaaactaa

SEQ ID NO: 269 - DNA, 18 bp, Artificial Sequence
  Detection oligonucleotide for PGR
cttctatccaaaaactaa

SEQ ID NO: 270 - DNA, 18 bp, Artificial Sequence
  Detection oligonucleotide for PGR
gaatttttcgagttagga

SEQ ID NO: 271 - DNA, 18 bp, Artificial Sequence
  Detection oligonucleotide for PGR
gaattttttgagttagga

SEQ ID NO: 272 - DNA, 18 bp, Artificial Sequence
  Detection oligonucleotide for PGR
tcctaactcgaaaaattc

SEQ ID NO: 273 - DNA, 18 bp, Artificial Sequence
  Detection oligonucleotide for PGR
tcctaactcaaaaaattc

SEQ ID NO: 274 - DNA, 18 bp, Artificial Sequence
  Detection oligonucleotide for PGR
ttaggagacgagattttt

SEQ ID NO: 275 - DNA, 18 bp, Artificial Sequence
  Detection oligonucleotide for PGR
ttaggagatgagattttt

SEQ ID NO: 276 - DNA, 18 bp, Artificial Sequence
  Detection oligonucleotide for PGR
aaaaatctcgtctcctaa

SEQ ID NO: 277 - DNA, 18 bp, Artificial Sequence
  Detection oligonucleotide for PGR
aaaaatctcatctcctaa

SEQ ID NO: 278 - DNA, 18 bp, Artificial Sequence
  Detection oligonucleotide for PGR
gggataaacgatagttat

SEQ ID NO: 279 - DNA, 18 bp, Artificial Sequence
  Detection oligonucleotide for PGR
gggataaatgatagttat

SEQ ID NO: 280 - DNA, 18 bp, Artificial Sequence
  Detection oligonucleotide for PGR
ataactatcgtttatccc

SEQ ID NO: 281 - DNA, 18 bp, Artificial Sequence
  Detection oligonucleotide for PGR
ataactatcatttatccc

SEQ ID NO: 282 - DNA, 18 bp, Artificial Sequence
  Detection oligonucleotide for PGR
tagaataacgggtggaaa

SEQ ID NO: 283 - DNA, 18 bp, Artificial Sequence
  Detection oligonucleotide for PGR
tagaataatgggtggaaa

SEQ ID NO: 284 - DNA, 18 bp, Artificial Sequence
  Detection oligonucleotide for PGR
tttccacccgttattcta

SEQ ID NO: 285 - DNA, 18 bp, Artificial Sequence
  Detection oligonucleotide for PGR
tttccacccattattcta

SEQ ID NO: 286 - DNA, 18 bp, Artificial Sequence
  Detection oligonucleotide for PGR
gattttatcggtaattgg

SEQ ID NO: 287 - DNA, 18 bp, Artificial Sequence
  Detection oligonucleotide for PGR
gattttattggtaattgg

SEQ ID NO: 288 - DNA, 18 bp, Artificial Sequence
  Detection oligonucleotide for PGR
ccaattaccgataaaatc

SEQ ID NO: 289 - DNA, 18 bp, Artificial Sequence
  Detection oligonucleotide for PGR
ccaattaccaataaaatc

SEQ ID NO: 290 - DNA, 18 bp, Artificial Sequence
  Detection oligonucleotide for PGR
gttttgggcggggttttt

SEQ ID NO: 291 - DNA, 18 bp, Artificial Sequence
  Detection oligonucleotide for PGR
gttttgggtggggttttt

SEQ ID NO: 292 - DNA, 18 bp, Artificial Sequence
  Detection oligonucleotide for PGR
aaaaaccccgcccaaaac

SEQ ID NO: 293 - DNA, 18 bp, Artificial Sequence
  Detection oligonucleotide for PGR
aaaaaccccacccaaaac

SEQ ID NO: 294 - DNA, 18 bp, Artificial Sequence
  Detection oligonucleotide for PGR
ttattaatcggggtaagt

SEQ ID NO: 295 - DNA, 18 bp, Artificial Sequence
  Detection oligonucleotide for PGR
ttattaattggggtaagt

SEQ ID NO: 296 - DNA, 18 bp, Artificial Sequence
  Detection oligonucleotide for PGR
acttaccccgattaataa

SEQ ID NO: 297 - DNA, 18 bp, Artificial Sequence
  Detection oligonucleotide for PGR
acttaccccaattaataa

SEQ ID NO: 298 - DNA, 18 bp, Artificial Sequence
  Detection oligonucleotide for PGR
agtgttttcgtattattg

SEQ ID NO: 299 - DNA, 18 bp, Artificial Sequence
  Detection oligonucleotide for PGR
agtgtttttgtattattg

SEQ ID NO: 300 - DNA, 18 bp, Artificial Sequence
  Detection oligonucleotide for PGR
caataatacgaaaacact

SEQ ID NO: 301 - DNA, 18 bp, Artificial Sequence
  Detection oligonucleotide for PGR
caataatacaaaaacact

SEQ ID NO: 302 - DNA, 18 bp, Artificial Sequence
  Detection oligonucleotide for PGR
gagattttcggagatgat

SEQ ID NO: 303 - DNA, 18 bp, Artificial Sequence
  Detection oligonucleotide for PGR
gagatttttggagatgat

SEQ ID NO: 304 - DNA, 18 bp, Artificial Sequence
  Detection oligonucleotide for PGR
atcatctccgaaaatctc

SEQ ID NO: 305 - DNA, 18 bp, Artificial Sequence
  Detection oligonucleotide for PGR
atcatctccaaaaatctc

SEQ ID NO: 306 - DNA, 18 bp, Artificial Sequence
  Detection oligonucleotide for PGR
gagaggttcgattagttt

SEQ ID NO: 307 - DNA, 18 bp, Artificial Sequence
  Detection oligonucleotide for PGR
gagaggtttgattagttt

SEQ ID NO: 308 - DNA, 18 bp, Artificial Sequence
  Detection oligonucleotide for PGR
aaactaatcgaacctctc

SEQ ID NO: 309 - DNA, 18 bp, Artificial Sequence
  Detection oligonucleotide for PGR
aaactaatcaaacctctc

SEQ ID NO: 310 - DNA, 18 bp, Artificial Sequence
  Detection oligonucleotide for PGR
gaatttagcgagggattg

SEQ ID NO: 311 - DNA, 18 bp, Artificial Sequence
  Detection oligonucleotide for PGR
gaatttagtgagggattg

SEQ ID NO: 312 - DNA, 18 bp, Artificial Sequence
  Detection oligonucleotide for PGR
caatccctcgctaaattc

SEQ ID NO: 313 - DNA, 18 bp, Artificial Sequence
  Detection oligonucleotide for PGR
caatccctcactaaattc

SEQ ID NO: 314 - DNA, 18 bp, Artificial Sequence
  Detection oligonucleotide for MB
agaaggtgcgtgagaggt

SEQ ID NO: 315 - DNA, 18 bp, Artificial Sequence
  Detection oligonucleotide for MB
agaaggtgtgtgagaggt

SEQ ID NO: 316 - DNA, 18 bp, Artificial Sequence
  Detection oligonucleotide for MB
acctctcacgcaccttct

SEQ ID NO: 317 - DNA, 18 bp, Artificial Sequence
  Detection oligonucleotide for MB
acctctcacacaccttct

SEQ ID NO: 318 - DNA, 18 bp, Artificial Sequence
  Detection oligonucleotide for MB
agaggttgcgaatggtta

SEQ ID NO: 319 - DNA, 18 bp, Artificial Sequence
  Detection oligonucleotide for MB
agaggttgtgaatggtta

SEQ ID NO: 320 - DNA, 18 bp, Artificial Sequence
taaccattcgcaacctct

SEQ ID NO: 321 - DNA, 18 bp, Artificial Sequence
  Detection oligonucleotide for MB
taaccattcacaacctct

SEQ ID NO: 322 - DNA, 18 bp, Artificial Sequence
  Detection oligonucleotide for MB
gggttagtcggggtattt

SEQ ID NO: 323 - DNA, 18 bp, Artificial Sequence
  Detection oligonucleotide for MB
gggttagttggggtattt

SEQ ID NO: 324 - DNA, 18 bp, Artificial Sequence
  Detection oligonucleotide for MB
aaataccccgactaaccc

SEQ ID NO: 325 - DNA, 18 bp, Artificial Sequence
  Detection oligonucleotide for MB
aaataccccaactaaccc

SEQ ID NO: 326 - DNA, 18 bp, Artificial Sequence
  Detection oligonucleotide for MB
gttgttggcgtcggtttt

SEQ ID NO: 327 - DNA, 18 bp, Artificial Sequence
  Detection oligonucleotide for MB
gttgttggtgtcggtttt

SEQ ID NO: 328 - DNA, 18 bp, Artificial Sequence
  Detection oligonucleotide for MB
aaaaccgacgccaacaac

SEQ ID NO: 329 - DNA, 18 bp, Artificial Sequence
  Detection oligonucleotide for MB
aaaaccgacaccaacaac

SEQ ID NO: 330 - DNA, 18 bp, Artificial Sequence
  Detection oligonucleotide for MB
tttttatacgtataatta

SEQ ID NO: 331 - DNA, 18 bp, Artificial Sequence
  Detection oligonucleotide for MB
tttttatatgtataatta

SEQ ID NO: 332 - DNA, 18 bp, Artificial Sequence
  Detection oligonucleotide for MB
taattatacgtataaaaa

SEQ ID NO: 333 - DNA, 18 bp, Artificial Sequence
  Detection oligonucleotide for MB
taattatacatataaaaa

SEQ ID NO: 334 - DNA, 18 bp, Artificial Sequence
  Detection oligonucleotide for MB
ttttgtttcgttataatg

SEQ ID NO: 335 - DNA, 18 bp, Artificial Sequence
  Detection oligonucleotide for MB
ttttgttttgttataatg

SEQ ID NO: 336 - DNA, 18 bp, Artificial Sequence
  Detection oligonucleotide for MB
cattataacgaaacaaaa

SEQ ID NO: 337 - DNA, 18 bp, Artificial Sequence
  Detection oligonucleotide for MB
cattataacaaaacaaaa

SEQ ID NO: 338 - DNA, 18 bp, Artificial Sequence
  Detection oligonucleotide for MB
ggggatagcgagttattg

SEQ ID NO: 339 - DNA, 18 bp, Artificial Sequence
  Detection oligonucleotide for MB
ggggatagtgagttattg

SEQ ID NO: 340 - DNA, 18 bp, Artificial Sequence
  Detection oligonucleotide for MB
caataactcgctatcccc

SEQ ID NO: 341 - DNA, 18 bp, Artificial Sequence
  Detection oligonucleotide for MB
caataactcactatcccc

SEQ ID NO: 342 - DNA, 18 bp, Artificial Sequence
  Detection oligonucleotide for MB
ttattgagcgatttttgt

SEQ ID NO: 343 - DNA, 18 bp, Artificial Sequence
  Detection oligonucleotide for MB
ttattgagtgatttttgt

SEQ ID NO: 344 - DNA, 18 bp, Artificial Sequence
  Detection oligonucleotide for MB
acaaaaatcgctcaataa

SEQ ID NO: 345 - DNA, 18 bp, Artificial Sequence
  Detection oligonucleotide for MB
acaaaaatcactcaataa

SEQ ID NO: 346 - DNA, 18 bp, Artificial Sequence
  Detection oligonucleotide for MB
ttagattgcgttatgggg

SEQ ID NO: 347 - DNA, 18 bp, Artificial Sequence
  Detection oligonucleotide for MB
ttagattgtgttatgggg

SEQ ID NO: 348 - DNA, 18 bp, Artificial Sequence
  Detection oligonucleotide for MB
ccccataacgcaatctaa

SEQ ID NO: 349 - DNA, 18 bp, Artificial Sequence
  Detection oligonucleotide for MB
ccccataacacaatctaa

SEQ ID NO: 350 - DNA, 18 bp, Artificial Sequence
  Detection oligonucleotide for MB
gggtttagcgacggggaa

SEQ ID NO: 351 - DNA, 18 bp, Artificial Sequence
  Detection oligonucleotide for MB
gggtttagtgacggggaa

SEQ ID NO: 352 - DNA, 18 bp, Artificial Sequence
  Detection oligonucleotide for MB
ttccccgtcgctaaaccc

SEQ ID NO: 353 - DNA, 18 bp, Artificial Sequence
  Detection oligonucleotide for MB
ttccccgtcactaaaccc

SEQ ID NO: 354 - DNA, 18 bp, Artificial Sequence
  Detection oligonucleotide for MB
gtgttgaacgtttggggg

SEQ ID NO: 355 - DNA, 18 bp, Artificial Sequence
  Detection oligonucleotide for MB
gtgttgaatgtttggggg

SEQ ID NO: 356 - DNA, 18 bp, Artificial Sequence
  Detection oligonucleotide for MB
cccccaaacgttcaacac

SEQ ID NO: 357 - DNA, 18 bp, Artificial Sequence
  Detection oligonucleotide for MB
cccccaaacattcaacac

SEQ ID NO: 358 - DNA, 18 bp, Artificial Sequence
  Detection oligonucleotide for PCNA
gggtttgacgagtcggga

SEQ ID NO: 359 - DNA, 18 bp, Artificial Sequence
  Detection oligonucleotide for PCNA
gggtttgatgagttggga

SEQ ID NO: 360 - DNA, 18 bp, Artificial Sequence
  Detection oligonucleotide for PCNA
tcccgactcgtcaaaccc

SEQ ID NO: 361 - DNA, 18 bp, Artificial Sequence
  Detection oligonucleotide for PCNA
tcccaactcatcaaaccc

SEQ ID NO: 362 - DNA, 18 bp, Artificial Sequence
  Detection oligonucleotide for PCNA
tatatgttcggatttgtt

SEQ ID NO: 363 - DNA, 18 bp, Artificial Sequence
  Detection oligonucleotide for PCNA
tatatgtttggatttgtt

SEQ ID NO: 364 - DNA, 18 bp, Artificial Sequence
  Detection oligonucleotide for PCNA
aacaaatccgaacatata

SEQ ID NO: 365 - DNA, 18 bp, Artificial Sequence
  Detection oligonucleotide for PCNA
aacaaatccaaacatata

SEQ ID NO: 366 - DNA, 18 bp, Artificial Sequence
  Detection oligonucleotide for PCNA
ttgttttgcggtcgggtt

SEQ ID NO: 367 - DNA, 18 bp, Artificial Sequence
  Detection oligonucleotide for PCNA
ttgttttgtggttgggtt

SEQ ID NO: 368 - DNA, 18 bp, Artificial Sequence
  Detection oligonucleotide for PCNA
aacccgaccgcaaaacaa

SEQ ID NO: 369 - DNA, 18 bp, Artificial Sequence
  Detection oligonucleotide for PCNA
aacccaaccacaaaacaa

SEQ ID NO: 370 - DNA, 18 bp, Artificial Sequence
  Detection oligonucleotide for PCNA
taaagaggcggggagatt

SEQ ID NO: 371 - DNA, 18 bp, Artificial Sequence
  Detection oligonucleotide for PCNA
taaagaggtggggagatt

SEQ ID NO: 372 - DNA, 18 bp, Artificial Sequence
  Detection oligonucleotide for PCNA
aatctccccgcctcttta

SEQ ID NO: 373 - DNA, 18 bp, Artificial Sequence
  Detection oligonucleotide for PCNA
aatctccccacctcttta

SEQ ID NO: 374 - DNA, 18 bp, Artificial Sequence
  Detection oligonucleotide for PCNA
tatggatacgattggttt

SEQ ID NO: 375 - DNA, 18 bp, Artificial Sequence
  Detection oligonucleotide for PCNA
tatggatatgattggttt

SEQ ID NO: 376 - DNA, 18 bp, Artificial Sequence
  Detection oligonucleotide for PCNA
aaaccaatcgtatccata

SEQ ID NO: 377 - DNA, 18 bp, Artificial Sequence
  Detection oligonucleotide for PCNA
aaaccaatcatatccata

SEQ ID NO: 378 - DNA, 18 bp, Artificial Sequence
  Detection oligonucleotide for PCNA
gtattaaacggttgtagg

SEQ ID NO: 379 - DNA, 18 bp, Artificial Sequence
  Detection oligonucleotide for PCNA
gtattaaatggttgtagg

SEQ ID NO: 380 - DNA, 18 bp, Artificial Sequence
  Detection oligonucleotide for PCNA
cctacaaccgtttaatac

SEQ ID NO: 381 - DNA, 18 bp, Artificial Sequence
  Detection oligonucleotide for PCNA
cctacaaccatttaatac

SEQ ID NO: 382 - DNA, 18 bp, Artificial Sequence
  Detection oligonucleotide for PCNA
gttgtaggcgtagtagag

SEQ ID NO: 383 - DNA, 18 bp, Artificial Sequence
  Detection oligonucleotide for PCNA
gttgtaggtgtagtagag

SEQ ID NO: 384 - DNA, 18 bp, Artificial Sequence
  Detection oligonucleotide for PCNA
ctctactacgcctacaac

SEQ ID NO: 385 - DNA, 18 bp, Artificial Sequence
  Detection oligonucleotide for PCNA
ctctactacacctacaac

SEQ ID NO: 386 - DNA, 18 bp, Artificial Sequence
  Detection oligonucleotide for PCNA
agagtggtcgttgttttt

SEQ ID NO: 387 - DNA, 18 bp, Artificial Sequence
  Detection oligonucleotide for PCNA
agagtggttgttgttttt

SEQ ID NO: 388 - DNA, 18 bp, Artificial Sequence
  Detection oligonucleotide for PCNA
aaaaacaacgaccactct

SEQ ID NO: 389 - DNA, 18 bp, Artificial Sequence
  Detection oligonucleotide for PCNA
aaaaacaacaaccactct

SEQ ID NO: 390 - DNA, 18 bp, Artificial Sequence
  Detection oligonucleotide for PCNA
tttgaagtcgaaattagt

SEQ ID NO: 391 - DNA, 18 bp, Artificial Sequence
  Detection oligonucleotide for PCNA
tttgaagttgaaattagt

SEQ ID NO: 392 - DNA, 18 bp, Artificial Sequence
  Detection oligonucleotide for PCNA
actaatttcgacttcaaa

SEQ ID NO: 393 - DNA, 18 bp, Artificial Sequence
  Detection oligonucleotide for PCNA
actaatttcaacttcaaa

SEQ ID NO: 394 - DNA, 18 bp, Artificial Sequence
  Detection oligonucleotide for PCNA
gtttgtagcggcgttgtt

SEQ ID NO: 395 - DNA, 18 bp, Artificial Sequence
  Detection oligonucleotide for PCNA
gtttgtagtggtgttgtt

SEQ ID NO: 396 - DNA, 18 bp, Artificial Sequence
  Detection oligonucleotide for PCNA
aacaacgccgctacaaac

SEQ ID NO: 397 - DNA, 18 bp, Artificial Sequence
  Detection oligonucleotide for PCNA
aacaacaccactacaaac

SEQ ID NO: 398 - DNA, 18 bp, Artificial Sequence
  Detection oligonucleotide for PCNA
tgttatttcgttattatg

SEQ ID NO: 399 - DNA, 18 bp, Artificial Sequence
  Detection oligonucleotide for PCNA
tgttattttgttattatg

SEQ ID NO: 400 - DNA, 18 bp, Artificial Sequence
  Detection oligonucleotide for PCNA
cataataacgaaataaca

SEQ ID NO: 401 - DNA, 18 bp, Artificial Sequence
  Detection oligonucleotide for PCNA
cataataacaaaataaca

SEQ ID NO: 402 - DNA, 18 bp, Artificial Sequence
  Detection oligonucleotide for PCNA
tttatttacgtagtggga

SEQ ID NO: 403 - DNA, 18 bp, Artificial Sequence
  Detection oligonucleotide for PCNA
tttatttatgtagtggga

SEQ ID NO: 404 - DNA, 18 bp, Artificial Sequence
  Detection oligonucleotide for PCNA
tcccactacgtaaataaa

SEQ ID NO: 405 - DNA, 18 bp, Artificial Sequence
  Detection oligonucleotide for PCNA
tcccactacataaataaa

SEQ ID NO: 406 - DNA, 18 bp, Artificial Sequence
  Detection oligonucleotide for CDC2
tagtaggacgatattttt

SEQ ID NO: 407 - DNA, 18 bp, Artificial Sequence
  Detection oligonucleotide for CDC2
tagtaggatgatattttt

SEQ ID NO: 408 - DNA, 18 bp, Artificial Sequence
  Detection oligonucleotide for CDC2
aaaaatatcgtcctacta

SEQ ID NO: 409 - DNA, 18 bp, Artificial Sequence
  Detection oligonucleotide for CDC2
aaaaatatcatcctacta

SEQ ID NO: 410 - DNA, 18 bp, Artificial Sequence
  Detection oligonucleotide for CDC2
tattttttcgattggagg

SEQ ID NO: 411 - DNA, 18 bp, Artificial Sequence
  Detection oligonucleotide for CDC2
tatttttttgattggagg

SEQ ID NO: 412 - DNA, 18 bp, Artificial Sequence
  Detection oligonucleotide for CDC2
cctccaatcgaaaaaata

SEQ ID NO: 413 - DNA, 18 bp, Artificial Sequence
  Detection oligonucleotide for CDC2
cctccaatcaaaaaaata

SEQ ID NO: 414 - DNA, 18 bp, Artificial Sequence
  Detection oligonucleotide for CDC2
taccttcccgaataacta

SEQ ID NO: 415 - DNA, 18 bp, Artificial Sequence
  Detection oligonucleotide for CDC2
taccttcccaaataacta

SEQ ID NO: 416 - DNA, 18 bp, Artificial Sequence
  Detection oligonucleotide for CDC2
aggtattgcggtagttgg

SEQ ID NO: 417 - DNA, 18 bp, Artificial Sequence
  Detection oligonucleotide for CDC2
aggtattgtggtagttgg

SEQ ID NO: 418 - DNA, 18 bp, Artificial Sequence
  Detection oligonucleotide for CDC2
ccaactaccgcaatacct

SEQ ID NO: 419 - DNA, 18 bp, Artificial Sequence
  Detection oligonucleotide for CDC2
ccaactaccacaatacct

SEQ ID NO: 420 - DNA, 18 bp, Artificial Sequence
gggttattcgattggtga

SEQ ID NO: 421 - DNA, 18 bp, Artificial Sequence
  Detection oligonucleotide for CDC2
gggttatttgattggtga

SEQ ID NO: 422 - DNA, 18 bp, Artificial Sequence
  Detection oligonucleotide for CDC2
tcaccaatcgaataaccc

SEQ ID NO: 423 - DNA, 18 bp, Artificial Sequence
  Detection oligonucleotide for CDC2
tcaccaatcaaataaccc

SEQ ID NO: 424 - DNA, 18 bp, Artificial Sequence
  Detection oligonucleotide for CDC2
ggtgaattcggggttttt

SEQ ID NO: 425 - DNA, 18 bp, Artificial Sequence
  Detection oligonucleotide for CDC2
ggtgaatttggggttttt

SEQ ID NO: 426 - DNA, 18 bp, Artificial Sequence
  Detection oligonucleotide for CDC2
aaaaaccccgaattcacc

SEQ ID NO: 427 - DNA, 18 bp, Artificial Sequence
  Detection oligonucleotide for CDC2
aaaaaccccaaattcacc

SEQ ID NO: 428 - DNA, 18 bp, Artificial Sequence
  Detection oligonucleotide for CDC2
ttttttagcgcggtgagt

SEQ ID NO: 429 - DNA, 18 bp, Artificial Sequence
  Detection oligonucleotide for CDC2
ttttttagtgtggtgagt

SEQ ID NO: 430 - DNA, 18 bp, Artificial Sequence
  Detection oligonucleotide for CDC2
actcaccgcgctaaaaaa

SEQ ID NO: 431 - DNA, 18 bp, Artificial Sequence
  Detection oligonucleotide for CDC2
actcaccacactaaaaaa

SEQ ID NO: 432 - DNA, 18 bp, Artificial Sequence
  Detection oligonucleotide for CDC2
aaattgttcgtatttggt

SEQ ID NO: 433 - DNA, 18 bp, Artificial Sequence
  Detection oligonucleotide for CDC2
aaattgtttgtatttggt

SEQ ID NO: 434 - DNA, 18 bp, Artificial Sequence
  Detection oligonucleotide for CDC2
accaaatacgaacaattt

SEQ ID NO: 435 - DNA, 18 bp, Artificial Sequence
  Detection oligonucleotide for CDC2
accaaatacaaacaattt

SEQ ID NO: 436 - DNA, 18 bp, Artificial Sequence
  Detection oligonucleotide for ERBB2
ggatttttcgaggaaaag

SEQ ID NO: 437 - DNA, 18 bp, Artificial Sequence
  Detection oligonucleotide for ERBB2
ggattttttgaggaaaag

SEQ ID NO: 438 - DNA, 18 bp, Artificial Sequence
  Detection oligonucleotide for ERBB2
tgtgagaacggttgtagg

SEQ ID NO: 439 - DNA, 18 bp, Artificial Sequence
  Detection oligonucleotide for ERBB2
tgtgagaatggttgtagg

SEQ ID NO: 440 - DNA, 18 bp, Artificial Sequence
  Detection oligonucleotide for ERBB2
aggagggacgatttaggt

SEQ ID NO: 441 - DNA, 18 bp, Artificial Sequence
  Detection oligonucleotide for ERBB2
aggagggatgatttaggt

SEQ ID NO: 442 - DNA, 18 bp, Artificial Sequence
  Detection oligonucleotide for ERBB2
aggtttgcgcgaagagag

SEQ ID NO: 443 - DNA, 18 bp, Artificial Sequence
  Detection oligonucleotide for ERBB2
aggtttgtgtgaagagag

SEQ ID NO: 444 - DNA, 18 bp, Artificial Sequence
  Detection oligonucleotide for ERBB2
ggagttgtcgatttttag

SEQ ID NO: 445 - DNA, 18 bp, Artificial Sequence
  Detection oligonucleotide for ERBB2
ggagttgttgatttttag

SEQ ID NO: 446 - DNA, 18 bp, Artificial Sequence
  Detection oligonucleotide for ERBB2
ttagatttcgttggaatg

SEQ ID NO: 447 - DNA, 18 bp, Artificial Sequence
  Detection oligonucleotide for ERBB2
ttagattttgttggaatg

SEQ ID NO: 448 - DNA, 18 bp, Artificial Sequence
  Detection oligonucleotide for ERBB2
ggagggggcgagttggga

SEQ ID NO: 449 - DNA, 18 bp, Artificial Sequence
  Detection oligonucleotide for ERBB2
ggagggggtgagttggga

SEQ ID NO: 450 - DNA, 18 bp, Artificial Sequence
  Detection oligonucleotide for ERBB2
ttgggagcgcgtttgttt

SEQ ID NO: 451 - DNA, 18 bp, Artificial Sequence
  Detection oligonucleotide for ERBB2
ttgggagtgtgtttgttt

SEQ ID NO: 452 - DNA, 18 bp, Artificial Sequence
  Detection oligonucleotide for TP53
tttttagttcgggaaaat

SEQ ID NO: 453 - DNA, 18 bp, Artificial Sequence
  Detection oligonucleotide for TP53
tttttagtttgggaaaat

SEQ ID NO: 454 - DNA, 18 bp, Artificial Sequence
  Detection oligonucleotide for TP53
ggaaaatcgttggggttg

SEQ ID NO: 455 - DNA, 18 bp, Artificial Sequence
  Detection oligonucleotide for TP53
ggaaaattgttggggttg

SEQ ID NO: 456 - DNA, 18 bp, Artificial Sequence
  Detection oligonucleotide for TP53
ggatttagcgagtttggg

SEQ ID NO: 457 - DNA, 18 bp, Artificial Sequence
  Detection oligonucleotide for TP53
ggatttagtgagtttggg

SEQ ID NO: 458 - DNA, 18 bp, Artificial Sequence
  Detection oligonucleotide for TP53
ggattattcgaatttaaa

SEQ ID NO: 459 - DNA, 18 bp, Artificial Sequence
  Detection oligonucleotide for TP53
ggattatttgaatttaaa

SEQ ID NO: 460 - DNA, 18 bp, Artificial Sequence
  Detection oligonucleotide for TP53
aagttgaacgtttaggta

SEQ ID NO: 461 - DNA, 18 bp, Artificial Sequence
  Detection oligonucleotide for TP53
aagttgaatgtttaggta

SEQ ID NO: 462 - DNA, 18 bp, Artificial Sequence
  Detection oligonucleotide for TP53
ttttgagtcggtttaaag

SEQ ID NO: 463 - DNA, 18 bp, Artificial Sequence
  Detection oligonucleotide for TP53
ttttgagttggtttaaag

SEQ ID NO: 464 - DNA, 18 bp, Artificial Sequence
  Detection oligonucleotide for TP53
gtttaaagcgtatttttt

SEQ ID NO: 465 - DNA, 18 bp, Artificial Sequence
  Detection oligonucleotide for TP53
gtttaaagtgtatttttt

SEQ ID NO: 466 - DNA, 18 bp, Artificial Sequence
  Detection oligonucleotide for TP53
tatttattcggtgttggg

SEQ ID NO: 467 - DNA, 18 bp, Artificial Sequence
  Detection oligonucleotide for TP53
tatttatttggtgttggg

SEQ ID NO: 468 - DNA, 18 bp, Artificial Sequence
  Detection oligonucleotide for TP53
gtgttgggcgtagggaat

SEQ ID NO: 469 - DNA, 18 bp, Artificial Sequence
  Detection oligonucleotide for TP53
gtgttgggtgtagggaat

SEQ ID NO: 470 - DNA, 18 bp, Artificial Sequence
  Detection oligonucleotide for TP53
ttggatttcgaaatattg

SEQ ID NO: 471 - DNA, 18 bp, Artificial Sequence
  Detection oligonucleotide for TP53
ttggattttgaaatattg

SEQ ID NO: 472 - DNA, 18 bp, Artificial Sequence
  Detection oligonucleotide for TP53
ttttatttcgaaatgatt

SEQ ID NO: 473 - DNA, 18 bp, Artificial Sequence
  Detection oligonucleotide for TP53
ttttattttgaaatgatt

SEQ ID NO: 474 - DNA, 18 bp, Artificial Sequence
  Detection oligonucleotide for TP53
gggtgattcggggagttt

SEQ ID NO: 475 - DNA, 18 bp, Artificial Sequence
  Detection oligonucleotide for TP53
gggtgatttggggagttt

SEQ ID NO: 476 - DNA, 18 bp, Artificial Sequence
  Detection oligonucleotide for TP53
ttgttagtcgtggaagtg

SEQ ID NO: 477 - DNA, 18 bp, Artificial Sequence
  Detection oligonucleotide for TP53
ttgttagttgtggaagtg

SEQ ID NO: 478 - DNA, 18 bp, Artificial Sequence
  Detection oligonucleotide for TP53
ggggtttgttcgttaggt

SEQ ID NO: 479 - DNA, 18 bp, Artificial Sequence
  Detection oligonucleotide for TP53
ggggtttgtttgttaggt

SEQ ID NO: 480 - DNA, 18 bp, Artificial Sequence
  Detection oligonucleotide for TP53
gaggatcgtcgtaatttg

SEQ ID NO: 481 - DNA, 18 bp, Artificial Sequence
  Detection oligonucleotide for TP53
gaggattgttgtaatttg

SEQ ID NO: 482 - DNA, 18 bp, Artificial Sequence
  Detection oligonucleotide for TP53
gagaggttcggtagtttt

SEQ ID NO: 483 - DNA, 18 bp, Artificial Sequence
  Detection oligonucleotide for TP53
gagaggtttggtagtttt

SEQ ID NO: 484 - DNA, 18 bp, Artificial Sequence
  Detection oligonucleotide for TP53
ttttttgtcggagtagtt

SEQ ID NO: 485 - DNA, 18 bp, Artificial Sequence
  Detection oligonucleotide for TP53
ttttttgttggagtagtt

SEQ ID NO: 486 - DNA, 18 bp, Artificial Sequence
  Detection oligonucleotide for TP53
tatttattcgatgagagg

SEQ ID NO: 487 - DNA, 18 bp, Artificial Sequence
  Detection oligonucleotide for TP53
tatttatttgatgagagg

SEQ ID NO: 488 - DNA, 18 bp, Artificial Sequence
  Detection oligonucleotide for TP53
ttttgagttacgggtttt

SEQ ID NO: 489 - DNA, 18 bp, Artificial Sequence
  Detection oligonucleotide for TP53
ttttgagttatgggtttt

SEQ ID NO: 490 - DNA, 18 bp, Artificial Sequence
  Detection oligonucleotide for CEA
ttgatgtacggatatata

SEQ ID NO: 491 - DNA, 18 bp, Artificial Sequence
  Detection oligonucleotide for CEA
ttgatgtatggatatata

SEQ ID NO: 492 - DNA, 18 bp, Artificial Sequence
  Detection oligonucleotide for CEA
tttagagtcgggtttttt

SEQ ID NO: 493 - DNA, 18 bp, Artificial Sequence
  Detection oligonucleotide for CEA
tttagagttgggtttttt

SEQ ID NO: 494 - DNA, 18 bp, Artificial Sequence
  Detection oligonucleotide for CEA
tatgttttcgtttaaatt

SEQ ID NO: 495 - DNA, 18 bp, Artificial Sequence
  Detection oligonucleotide for CEA
tatgtttttgtttaaatt

SEQ ID NO: 496 - DNA, 18 bp, Artificial Sequence
  Detection oligonucleotide for CEA
ggataatgcgggtggatt

SEQ ID NO: 497 - DNA, 18 bp, Artificial Sequence
  Detection oligonucleotide for CEA
ggataatgtgggtggatt

SEQ ID NO: 498 - DNA, 18 bp, Artificial Sequence
  Detection oligonucleotide for CEA
atgtaagacgtttttgtt

SEQ ID NO: 499 - DNA, 18 bp, Artificial Sequence
  Detection oligonucleotide for CEA
atgtaagatgtttttgtt

SEQ ID NO: 500 - DNA, 18 bp, Artificial Sequence
  Detection oligonucleotide for CEA
atgatgagcgaattaaat

SEQ ID NO: 501 - DNA, 18 bp, Artificial Sequence
  Detection oligonucleotide for CEA
atgatgagtgaattaaat

SEQ ID NO: 502 - DNA, 18 bp, Artificial Sequence
  Detection oligonucleotide for CEA
tagtaatgcgaggataga

SEQ ID NO: 503 - DNA, 18 bp, Artificial Sequence
  Detection oligonucleotide for CEA
tagtaatgtgaggataga

SEQ ID NO: 504 - DNA, 18 bp, Artificial Sequence
  Detection oligonucleotide for CEA
atgtatagcgtagttttt

SEQ ID NO: 505 - DNA, 18 bp, Artificial Sequence
  Detection oligonucleotide for CEA
atgtatagtgtagttttt

SEQ ID NO: 506 - DNA, 18 bp, Artificial Sequence
  Detection oligonucleotide for CEA
ttttttgtcgataaaagt

SEQ ID NO: 507 - DNA, 18 bp, Artificial Sequence
  Detection oligonucleotide for CEA
ttttttgttgataaaagt

SEQ ID NO: 508 - DNA, 18 bp, Artificial Sequence
  Detection oligonucleotide for CEA
aagtgttcgcggttgttt

SEQ ID NO: 509 - DNA, 18 bp, Artificial Sequence
  Detection oligonucleotide for CEA
aagtgtttgtggttgttt

SEQ ID NO: 510 - DNA, 18 bp, Artificial Sequence
  Detection oligonucleotide for CEA
agatttttcggtttgatt

SEQ ID NO: 511 - DNA, 18 bp, Artificial Sequence
  Detection oligonucleotide for CEA
agattttttggtttgatt

SEQ ID NO: 512 - DNA, 18 bp, Artificial Sequence
  Detection oligonucleotide for CEA
atattacgtcgattttgg

SEQ ID NO: 513 - DNA, 18 bp, Artificial Sequence
  Detection oligonucleotide for CEA
atattatgttgattttgg

SEQ ID NO: 514 - DNA, 18 bp, Artificial Sequence
  Detection oligonucleotide for CEA
gataaggtcggaggtttt

SEQ ID NO: 515 - DNA, 18 bp, Artificial Sequence
  Detection oligonucleotide for CEA
gataaggttggaggtttt

SEQ ID NO: 516 - DNA, 18 bp, Artificial Sequence
  Detection oligonucleotide for CEA
ttaggaaacggatttttt

SEQ ID NO: 517 - DNA, 18 bp, Artificial Sequence
  Detection oligonucleotide for CEA
ttaggaaatggatttttt

SEQ ID NO: 518 - DNA, 18 bp, Artificial Sequence
  Detection oligonucleotide for CEA
gttagtttcgtagaggat

SEQ ID NO: 519 - DNA, 18 bp, Artificial Sequence
  Detection oligonucleotide for CEA
gttagttttgtagaggat

SEQ ID NO: 520 - DNA, 18 bp, Artificial Sequence
  Detection oligonucleotide for CEA
ttttgagtcgtagtttag

SEQ ID NO: 521 - DNA, 18 bp, Artificial Sequence
  Detection oligonucleotide for CEA
ttttgagttgtagtttag

SEQ ID NO: 522 - DNA, 18 bp, Artificial Sequence
  Detection oligonucleotide for CEA
aatagatacggagaggga

SEQ ID NO: 523 - DNA, 18 bp, Artificial Sequence
  Detection oligonucleotide for CEA
aatagatatggagaggga

SEQ ID NO: 524 - DNA, 20 bp, Artificial Sequence
  Detection oligonucleotide for CEA
tggttaaatgtgtgggagat

SEQ ID NO: 525 - DNA, 20 bp, Artificial Sequence
  Detection primer for CEA
tcctgagtgatgtctgtgtg

SEQ ID NO: 526 - DNA, 20 bp, Artificial Sequence
  Detection primer for p16
atgacaccaaacaccccgat

SEQ ID NO: 527 - DNA, 19 bp, Artificial Sequence
  Detection primer for p16
ctgtccctcaaatcctctg

(19) United States
(12) Patent Application Publication
(10) Pub. No.: US 2005/0064410 A1
(43) Pub. Date: Mar. 24, 2005

(54) Method and nucleic acids for the analysis of colon cancer

(76) Inventors: Jurgen Distler, Berlin (DE); Fabian Model, Berlin (DE); Heike Taubert, Berlin (DE)

Correspondence Address: KRIEGSMAN & KRIEGSMAN, 665 FRANKLIN STREET, FRAMINGHAM, MA, 01702, US

(21) Appl. No.: 10/486,319

(22) PCT Filed: Aug. 9, 2002
(86) PCT No.: PCT/EP02/08939

(30) Foreign Application Priority Data
Aug. 9, 2001 (DE) 101 39 283.4

(51) Int. Cl. C12Q 1/68; C07H 21/04

(52) U.S. Cl. 435/6; 536/25.3

(57) ABSTRACT

The present invention relates to chemically modified genomic sequences, oligonucleotides and/or PNA-oligomers for detecting the cytosine methylation state of genomic DNA, as well as to methods for ascertaining genetic and/or epigenetic parameters of genes for use in the characterisation, grading, staging, and/or diagnosis of colon cancer, or the predisposition to colon cancer.

### FIELD OF THE INVENTION

The levels of observation that have been studied by the methodological developments of recent years in molecular biology, are the genes themselves, the translation of these genes into RNA, and the resulting proteins. The question of which gene is switched on at which point in the course of the development of an individual, and how the activation and inhibition of specific genes in specific cells and tissues are controlled is correlatable to the degree and character of the methylation of the genes or of the genome. In this respect, pathogenic conditions may manifest themselves in a changed methylation pattern of individual genes or of the genome.

The present invention relates to nucleic acids, oligonucleotides, PNA-oligomers, and to a method for the characterisation, grading, staging, treatment and/or diagnosis of colon cancer, or the predisposition to colon cancer, by analysis of the genetic and/or epigenetic parameters of genomic DNA and, in particular, with the cytosine methylation status thereof.

### PRIOR ART

Colon cancer is the second most common cause of cancer death in the United States. It describes any cancer in the colon (large intestine), from the beginning of the colon (cecum) to the end of the colon (rectum). Colon cancer is a malignant tumor in the lining of the large intestine. It starts with a single cell that mutates and grows into a visible polyp. If a polyp is allowed to remain in the colon it can grow into a cancerous tumor that can invade other organs. The mechanism behind the progression to malignancy are not comletely understood, however most polyps take 3-7 years to become cancerous. Prevention of colon cancer means stopping this process by removing the polyp before it becomes cancerous. Colon cancer represents an interaction between the genome of the colorectal epithelial cell and the host environment. Both factors are essential for the development of tumors. Colon cancers can be differentiated into nonhereditary types, which rarely occur before age 40 and hereditary colon cancers which often occur in younger people.

Human colon cancers undergo a multistage carcinogenesis pathway from adenomatous polyps to carcinoma. A number of genetic events have been characterized and include alterations in “tumor suppressor” and susceptibility genes that normally encode for proteins regulating cell cycle progression and programmed cell death (Kinzler K W, Vogelstein B. Landscaping the cancer terrain. Science. 1998 May 15;280(5366):1036-7). Given the high incidence of colon cancer in the aging population and high mortality rates for advanced disease, new prevention strategies are needed. After the diagnosis of cancer has been made it is important to determine the extent or ‘stage’ of the cancer before deciding on the treatment plan. Staging is a method of evaluating the progress of the cancer in a patient and defines the extent to which the cancer has spread to other parts of the body. There are several systems for classifying the extent or stage of cancer. One of the the two most common systems is the Stage ‘I, II, III, IV’ system, which defines four stages of cancer. Stage I represents early cancer, with a small tumor and no spread to the lymph nodes. In stages II and III, the tumor is progressively more advanced, while stage IV refers to metastatic disease that has spread to other areas of the body. One very important point to realize about these staging systems is that they only provide rough estimates of the stage of disease and chances of survival. The numbers are just averages. They do not say anything about the outcome or prognosis of any one particular patient.

Genes which are associated with colon cancer include the following.
p16 (Dai C Y, Furth E E, Mick R, Koh J, Takayama T, Niitsu Y, Enders G H. p16(INK4a) expression begins early in human colon neoplasia and correlates inversely with markers of cell proliferation. Gastroenterology. 2000 October; 119(4):929-42).
p27 (Liu D F, Ferguson K, Cooper G S, Grady W M, Willis J. p27 cell-cycle inhibitor is inversely correlated with lymph node metastases in right-sided colon cancer. J Clin Lab Anal. 1999;13(6):291-5).
p53 (Arango D, Corner G A, Wadler S, Catalano P J, Augenlicht L H. c-myc/p53 interaction determines sensitivity of human colon carcinoma cells to 5-fluorouracil in vitro and in vivo. Cancer Res. 2001 Jun. 15;61(12):4910-5).
cdc2 (Moragoda L, Jaszewski R, Majumdar A P. Curcumin induced modulation of cell cycle and apoptosis in gastric and colon cancer cells. Anticancer Res. 2001 March-April; 21(2A):873-8).
PCNA (Zhang Y, Iwama T, Sugihara K. Histochemical study of apoptosis and cell proliferation in hereditary intestinal diseases. J Med Dent Sci. 1998 June;45(2):77-84).
CEA (Vogel I, Francksen H, Soeth E, Henne-Bruns D, Kremer B, Juhl H. The carcinoembryonic antigen and its prognostic impact on immunocytologically detected intraperitoneal colorectal cancer cells. Am J. Surg. 2001 February; 181(2):188-93).
c-erbB2 (Fric P, Sovova V, Sloncova E, Lojda Z, Jirasek A, Cermak J. Different expression of some molecular markers in sporadic cancer of the left and right colon. Eur J Cancer Prev. 2000 August; 9(4):265-8).
Estrogen receptor (Campbell-Thompson M, Lynch I J, Bhardwaj B. Expression of estrogen receptor (ER) subtypes and ERbeta isoforms in colon cancer. Cancer Res. 2001 Jan. 15;61(2):632-40).
Progesterone receptor (Reich O, Regauer S, Urdl W, Lahousen M, Winter R. Expression of oestrogen and progesterone receptors in low-grade endometrial stromal sarcomas. Br J Cancer. 2000 March;82(5):1030-4) and myoglobin (Nakao A, Sakagami K, Uda M, Mitsuoka S, Ito H. Carcinosarcoma of the colon: report of a case and review of the literature. J Gastroenterol. 1998 April;33(2):276-9).

5-methylcytosine is the most frequent covalent base modification in the DNA of eukaryotic cells. It plays a role, for example, in the regulation of the transcription, in genetic imprinting, and in tumorigenesis. Therefore, the identification of 5-methylcytosine as a component of genetic information is of considerable interest. However,

5-methylcytosine positions cannot be identified by sequencing since 5-methylcytosine has the same base pairing behavior as cytosine. Moreover, the epigenetic information carried by 5-methylcytosine is completely lost during PCR amplification.

A relatively new and currently the most frequently used method for analyzing DNA for 5-methylcytosine is based upon the specific reaction of bisulfite with cytosine which, upon subsequent alkaline hydrolysis, is converted to uracil which corresponds to thymidine in its base pairing behavior. However, 5-methylcytosine remains unmodified under these conditions. Consequently, the original DNA is converted in such a manner that methylcytosine, which originally-could not be distinguished from cytosine by its hybridization behavior, can now be detected as the only remaining cytosine using “normal” molecular biological techniques, for example, by amplification and hybridization or sequencing. All of these techniques are based on base pairing which can now be fully exploited. In terms of sensitivity, the prior art is defined by a method which encloses the DNA to be analyzed in an agarose matrix, thus preventing the diffusion and renaturation of the DNA (bisulfite only reacts with single-stranded DNA), and which replaces all precipitation and purification steps with fast dialysis (Olek A, Oswald J, Walter J. A modified and improved method for bisulphite based cytosine methylation analysis. Nucleic Acids Res. 1996 Dec. 15;24(24):5064-6). Using this method, it is possible to analyze individual cells, which illustrates the potential of the method. However, currently only individual regions of a length of up to approximately 3000 base pairs are analyzed, a global analysis of cells for thousands of possible methylation events is not possible. However, this method cannot reliably analyze very small fragments from small sample quantities either. These are lost through the matrix in spite of the diffusion protection.

An overview of the further known methods of detecting 5-methylcytosine may be gathered from the following review article: Rein, T., DePamphilis, M. L., Zorbas, H., Nucleic Acids Res. 1998, 26, 2255.

To date, barring few exceptions (e.g., Zeschnigk M, Lich C, Buiting K, Doerfler W, Horsthemke B. A single-tube PCR test for the diagnosis of Angelman and Prader-Willi syndrome based on allelic methylation differences at the SNRPN locus. Eur J Hum Genet. 1997 March-April;5(2):94-8) the bisulfite technique is only used in research. Always, however, short, specific fragments of a known gene are amplified subsequent to a bisulfite treatment and either completely sequenced (Olek A, Walter J. The pre-implantation ontogeny of the H19 methylation imprint. Nat Genet. 1997 November;17(3):275-6) or individual cytosine positions are detected by a primer extension reaction (Gonzalgo M L, Jones P A. Rapid quantitation of methylation differences at specific sites using methylation-sensitive single nucleotide primer extension (Ms-SNuPE). Nucleic Acids Res. 1997 Jun. 15;25(12):2529-31, WO Patent 9500669) or by enzymatic digestion (Xiong Z, Laird P W. COBRA: a sensitive and quantitative DNA methylation assay. Nucleic Acids Res. 1997 Jun. 15;25(12):2532-4). In addition, detection by hybridization has also been described (Olek et al., WO 99 28498).

Further publications dealing with the use of the bisulfite technique for methylation detection in individual genes are: Grigg G, Clark S. Sequencing 5-methylcytosine residues in genomic DNA. Bioessays. 1994 June;16(6):431-6, 431; Zeschnigk M, Schmitz B, Dittrich B, Buiting K, Horsthemke B, Doerfler W. Imprinted segments in the human genome: different DNA methylation patterns in the Prader-Willi/Angelman syndrome region as determined by the genomic sequencing method. Hum Mol Genet. 1997 March;6(3):387-95; Feil R, Charlton J, Bird A P, Walter J, Reik W. Methylation analysis on individual chromosomes: improved protocol for bisulphite genomic sequencing. Nucleic Acids Res. 1994 Feb. 25;22(4):695-6; Martin V, Ribieras S, Song-Wang X, Rio M C, Dante R. Genomic sequencing indicates a correlation between DNA hypomethylation in the 5′ region of the pS2 gene and its expression in human breast cancer cell lines. Gene. 1995 May 19;157(1-2):261-4; WO 97/46705, WO 95/15373 and WO 95/45560.

An overview of the Prior Art in oligomer array manufacturing can be gathered from a special edition of Nature Genetics (Nature Genetics Supplement, Volume 21, January 1999), published in January 1999, and from the literature cited therein.

Fluorescently labeled probes are often used for the scanning of immobilized DNA arrays. The simple attachment of Cy3 and Cy5 dyes to the 5′-OH of the specific probe are particularly suitable for fluorescence labels. The detection of the fluorescence of the hybridized probes may be carried out, for example via a confocal microscope. Cy3 and Cy5 dyes, besides many others, are commercially available.

Matrix Assisted Laser Desorption Ionization Mass Spectrometry (MALDI-TOF) is a very efficient development for the analysis of biomolecules (Karas M, Hillenkamp F. Laser desorption ionization of proteins with molecular masses exceeding 10,000 daltons. Anal Chem. 1988 Oct. 15;60(20):2299-301). An analyte is embedded in a light-absorbing matrix. The matrix is evaporated by a short laser pulse thus transporting the analyte molecule into the vapor phase in an unfragmented manner. The analyte is ionized by collisions with matrix molecules. An applied voltage accelerates the ions into a field-free flight tube. Due to their different masses, the ions are accelerated at different rates. Smaller ions reach the detector sooner than bigger ones.

MALDI-TOF spectrometry is excellently suited to the analysis of peptides and proteins. The analysis of nucleic acids is somewhat more difficult (Gut I G, Beck S. DNA and Matrix Assisted Laser Desorption Ionization Mass Spectrometry. Current Innovations and Future Trends. 1995, 1; 147-57). The sensitivity to nucleic acids is approximately 100 times worse than to peptides and decreases disproportionally with increasing fragment size. For nucleic acids having a multiply negatively charged backbone, the ionization process via the matrix is considerably less efficient. In MALDI-TOF spectrometry, the selection of the matrix plays an eminently important role. For the desorption of peptides, several very efficient matrixes have been found which produce a very fine crystallization. There are now several responsive matrixes for DNA, however, the difference in sensitivity has not been reduced. The difference in sensitivity can be reduced by chemically modifying the DNA in such a manner that it becomes more similar to a peptide. Phosphorothioate nucleic acids in which the usual phosphates of the backbone are substituted with thiophosphates can be converted into a charge-neutral DNA using simple alkylation chemistry (Gut I G, Beck S. A procedure for selective DNA alkylation and detection by mass spectrometry. Nucleic Acids Res. 1995 Apr. 25;23(8):1367-73). The coupling of a charge tag to this modified DNA results in an increase in sensitivity to the same level as that found for peptides. A further advantage of charge tagging is the increased stability of the analysis against impurities which make the detection of unmodified substrates considerably more difficult.

Genomic DNA is obtained from DNA of cell, tissue or other test samples using standard methods. This standard methodology is found in references such as Fritsch and Maniatis eds., Molecular Cloning: A Laboratory Manual, 1989.

### DESCRIPTION OF THE INVENTION

The present invention discloses that atypical methylation in the genes estrogen receptor, p21, p27, p16, progesteron receptor, myoglobin, pcna, cdc2, c-erbB2, p53 and CEA, can be positively correlated with colon carcinogenesis. This allows the detection of colon carcinoma, or the predisposition to colon cancer by an assay that detects methylation in the genes by restriction enzyme analysis, or using a nucleic acid based method.

The disclosed invention provides a method and nucleic acids for the analysis of colon carcinomas. It discloses a means of distinguishing between healthy and cancerous colon tissue. This provides a means for the improved diagnosis, prognosis, staging and grading of colon cancer, at a molecular level, as opposed to currently used methods of a relatively subjective nature such as histological analysis. Furthermore, the disclosed invention presents improvements over the state of the art in that current methods of histological and cytological analysis require that the biopsy contain a sufficient amount of tissue. The method according to the present invention can be used for classification of minute samples.

The invention provides a method for detecting a colon cell proliferative disorder characterised in that the target nucleic acid of one or more genes taken from the group comprising estrogen receptor, p21, p27, p16, progesteron receptor, myoglobin, pcna, cdc2, c-erbB2, p53 and CEA are contacted with a reagent or series of reagents capable of distinguishing between methylated and non methylated CpG dinucleotides within the target sequence.

The present invention makes available a method for ascertaining genetic and/or epigenetic parameters of genomic DNA. The method is for use in the grading, staging, treatment and/or diagnosis of colon cancer. The method enables the analysis of cytosine methylations and single nucleotide polymorphisms.

In one embodiment of the method the genomic DNA sample is first isolated from tissue or cellular sources. Such sources may include cell lines, histological slides, body fluids, or tissue embedded in paraffin. Extraction may be by means that are standard to one skilled in the art, these include the use of detergent lysates, sonification and vortexing with glass beads. Once the nucleic acids have been extracted the genomic double stranded DNA is used in the analysis.

In a preferred embodiment the DNA may be cleaved prior to the chemical treatment, this may be any means standard in the state of the art, in particular with restriction endonucleases.

In the third step of the method, the genomic DNA sample is treated in such a manner that cytosine bases which are unmethylated at the 5′-position are converted to uracil, thymine, or another base which is dissimilar to cytosine in terms of hybridization behavior. This will be understood as ‘pretreatment’ hereinafter.

The above described treatment of genomic DNA is preferably carried out with bisulfite (sulfite, disulfite) and subsequent alkaline hydrolysis which results in the conversion of non-methylated cytosine nucleobases to uracil or to another base which is dissimilar to cytosine in terms of base pairing behavior.

In the fourth step of the method the bisulfite treated DNA is analysed using one or a combination of several methods which are known in the art namely real time PCR (Methyl Light assay), blocking oligonucleotides, methylation specific single nucleotide polymorphism extension (hereinafter referred to as MsSNuPE), methylation specific PCR (hereinafter referred to as MSP), and nucleic acid sequencing.

Fluorescence-based Real Time Quantitative PCR (Heid et al., Genome Res. 6:986-994, 1996) employs a dual-labeled fluorescent oligonucleotide probe (e.g. TaqMan™ PCR, using an ABI Prism 7700 Sequence Detection System, Perkin Elmer Applied Biosystems, Foster City, Calif.) that is hybridized concurrently with oligonucleotide primers during a continuosly monitered polymerase chain reaction. The TaqMan™ PCR reaction employs the use of a nonextendible interrogating oligonucleotide, called a TaqMan™ probe, which is designed to hybridize to a GpC-rich sequence located between the forward and reverse amplification primers. The TaqMan™ probe further comprises a fluorescent “reporter moiety” and a “quencher moiety” covalently bound to linker moieties (e.g., phosphoramidites) attached to the nucleotides of the TaqMan™ oligonucleotide. For analysis of methylation within nucleic acids subsequent to bisulphite treatment it is required that the probe be methylation specific, as described in U.S. Pat. No. 6,331,393, also known as the Methyl Light assay. Variations on the TaqMan™ detection methodology that are also suitable for use with the described invention include the use of dual probe technology (Lightcycler™) or fluorescent amplification primers (Sunrise™ technology). Both these techniques may be adapted in a manner suitable for use with bisulphite treated DNA, and moreover for methylation analysis within CpG dinucleotides.

A further suitable method for the for the assessment of methylation by analysis of bisulphite treated nucleic acids is the use of blocker oligonucleotides. The use of such oligonucleotides has been described in BioTechniques 23(4), 1997, 714-720 D. Yu, M. Mukai, Q. Liu, C. Steinman. Blocking probe oligonucleotides are hybridised to the bisulphite treated nucleic acid concurrently with the PCR primers. PCR amplification of the nucleic acid is terminated at the 5′ position of the blocking probe, thereby amplification of a nucleic acid is suppressed wherein the complementary sequence to the blocking probe is present. The probes may be designed to hybridise to the bisulphite treated nucleic acid in a methylation status specific manner. For example, for detection of methylated nucleic acids within a population of unmethylated nucleic acids suppression of the amplification of nucleic acids which are unmethylated at the position in question would be carried out by the use of blocking probes comprising a ‘CG’ at the position in question, as opposed to a ‘CA’.

In a further preferred embodiment of the method the analysis is carried out by the use of template directed oligonucleotide extension, such as MS SNuPE as described by Gonzalgo and Jones (Nucleic Acids Res. 25:2529-2531).

In an alternative embodiment of the method the assessment of the methylation state fo the CpG dinucleotides may be carried out by PCR analysis of the treated nucleic acid(s) using methylation specific PCR. Methylation specific primers (MSP) have been described, for example in U.S. Pat. No. 6,265,171 to Herman et al. MSP primers consist of an oligonucleotide specific for annealing to a nucleotide sequence containing at least one bisulphite treated CpG dinucleotide. Therefore the sequence of said primers includes at least one CG, TG or CA dinucleotide. MSP primers specific for non methylated DNA contain a ‘T’ at the 3′ position of the C position in the CpG. MSP primers generally contain relatively few cytosines as these are converted by the bisulphite reaction. However when the primers are specifc for methylated cytosine dinucleotides said cytosine positions are conserved within the primer oligonucleotides.

The primers are extended by means of a polymerase and the resultant double stranded nucleic is denatured, preferably by means of heat treatment. Successive cycles of primer annealing, extension and denaturation are carried out according to the polymerase chain reaction as described in U.S. Pat. No. 4,582,788 to Mullis.

In a further embodiment of the method the analysis is enabled by sequencing and subsequent sequence analysis of the amplificate generated in the third step of the method (Sanger F., et al., 1977 PNAS USA 74: 5463-5467).

In a particularly preferred embodiment, the method comprises the following steps:

In the first step of the method the genomic DNA sample must be isolated from tissue or cellular sources. Such sources may include cell lines, histological slides, body fluids, or tissue embedded in paraffin. Extraction may be by means that are standard to one skilled in the art, these include the use of detergent lysates, sonification and vortexing with glass beads. Once the nucleic acids have been extracted the genomic double stranded DNA is used in the analysis.

In a preferred embodiment the DNA may be cleaved prior to the chemical treatment, this may be any means standard in the state of the art, in particular with restriction endonucleases.

In the second step of the method, the genomic DNA sample is treated in such a manner that cytosine bases which are unmethylated at the 5′-position are converted to uracil, thymine, or another base which is dissimilar to cytosine in terms of hybridization behavior. This will be understood as ‘pretreatment’ hereinafter.

The above described treatment of genomic DNA is preferably carried out with bisulfite (sulfite, disulfite) and subsequent alkaline hydrolysis which results in the conversion of non-methylated cytosine nucleobases to uracil or to another base which is dissimilar to cytosine in terms of base pairing behavior.

In the third step fragments of the pretreated DNA are amplified, using sets of primer oligonucleotides according to Seq ID 76 to 97, and a, preferably heat-stable polymerase. Because of statistical and practical considerations, preferably more than ten different fragments having a length of 100-2000 base pairs are amplified. The amplification of several DNA segments can be carried out simultaneously in one and the same reaction vessel. Usually, the amplification is carried out by means of a polymerase chain reaction (PCR).

The method may also be enabled by the use of alternative primers, the design of such primers is obvious to one skilled in the art. These should include at least two oligonucleotides whose sequences are each reverse complementary or identical to an at least 18 base-pair long segment of the base sequences specified in the appendix (Seq. ID No.32 through Seq. ID No.75). Said primer oligonucleotides are preferably characterized in that they do not contain any CpG dinucleotides. In a particularly preferred embodiment of the method, the sequence of said primer oligonucleotides are designed so as to selectively anneal to and amplify, only the colon tissue specific DNA of interest, thereby minimizing the amplification of background or non relevant DNA. In the context of the present invention, background DNA is taken to mean genomic DNA which does not have a relevant tissue specific methylation pattern, in this case, the relevant tissue being colon tissue, both healthy and diseased.

According to the present invention, it is preferred that at least one primer oligonucleotide is bound to a solid phase during amplification. The different oligonucleotide and/or PNA-oligomer sequences can be arranged on a plane solid phase in the form of a rectangular or hexagonal lattice, the solid phase surface preferably being composed of silicon, glass, polystyrene, aluminum, steel, iron, copper, nickel, silver, or gold, it being possible for other materials such as nitrocellulose or plastics to be used as well.

The fragments obtained by means of the amplification can carry a directly or indirectly detectable label. Preferred are labels in the form of fluorescence labels, radionuclides, or detachable molecule fragments having a typical mass which can be detected in a mass spectrometer, it being preferred that the fragments that are produced have a single positive or negative net charge for better detectability in the mass spectrometer. The detection may be carried out and visualized by means of matrix assisted laser desorption/ionization mass spectrometry (MALDI) or using electron spray mass spectrometry (ESI).

The amplificates obtained in the third step of the method are subsequently hybridized to an array or a set of oligonucleotides and/or PNA probes. In this context, the hybridization takes place in the manner described in the following. The set of probes used during the hybridization is preferably composed of at least 10 oligonucleotides or PNA-oligomers. In the process, the amplificates serve as probes which hybridize to oligonucleotides previously bonded to a solid phase. Ina particularly preferred embodiment, the oligonucleotides are taken from the group comprising Seq IDs 98 to 523. The non-hybridized fragments are subsequently removed. Said oligonucleotides contain at least one base sequence having a length of 10 nucleotides which is reverse complementary or identical to a segment of the base sequences specified in the appendix, the segment containing at least one CpG dinucleotide. The cytosine of the CpG dinucleotide is the 5^{th} to 9^{th} nucleotide from the 5′-end of the 10-mer. One oligonucleotide exists for each CpG dinucleotide.

In the next step of the method, the non-hybridized amplificates are removed.

In the final step of the method, the hybridized amplificates are detected. In this context, it is preferred that labels attached to the amplificates are identifiable at each position of the solid phase at which an oligonucleotide sequence is located.

According to the present invention, it is preferred that the labels of the amplificates are fluorescence labels, radionuclides, or detachable molecule fragments having a typical mass which can be detected in a mass spectrometer. The mass spectrometer is preferred for the detection of the amplificates, fragments of the amplificates or of probes which are complementary to the amplificates, it being possible for the detection to be carried out and visualized by means of matrix assisted laser desorption/ionization mass spectrometry (MALDI) or using electron spray mass spectrometry (ESI). The produced fragments may have a single positive or negative net charge for better detectability in the mass spectrometer.

The aforementioned method is preferably used for ascertaining genetic and/or epigenetic parameters of genomic DNA.

In order to enable this method, the invention further provides the chemically modified DNA of the genes estrogen receptor, p21, p27, p16, progesteron receptor, myoglobin, pcna, cdc2, c-erbB2, p53 and CEA as well as oligonucleotides and/or PNA-oligomers for detecting cytosine methylations. The present invention is based on the discovery that genetic and epigenetic parameters and, in particular, the cytosine methylation patterns of genomic DNA are particularly suitable for characterisation, grading, staging, and/or diagnosis of colon cancer.

The nucleic acids according to the present invention of Seq. ID No.12 through Seq. ID No. 523 can be used for characterisation, grading, staging and/or diagnosis of genetic and/or epigenetic parameters of genomic DNA.

This objective is achieved according to the present invention using a nucleic acid containing a sequence of at least 18 bases in length of the chemically pretreated genomic DNA according to one of Seq. ID No.32 through Seq. ID No.75 and sequences complementary thereto.

The chemically modified nucleic acid could heretofore not be connected with the ascertainment of disease relevant genetic and epigenetic parameters.

The object of the present invention is further achieved by an oligonucleotide or oligomer for the analysis of pretreated DNA, for detecting the genomic cytosine methylation state, said oligonucleotide containing at least one base sequence having a length of at least 10 nucleotides which hybridizes to a pretreated genomic DNA according to Seq. ID No.32 through Seq. ID No. 75. The oligomer probes according to the present invention constitute important and effective tools which, for the first time, make it possible to ascertain specific genetic and epigenetic parameters of colon cancers, in particular, for use in characterisation, grading, staging, and/or diagnosis of colon cancer. The base sequence of the oligomers preferably contains at least one CpG dinucleotide. The probes may also exist in the form of a PNA (peptide nucleic acid) which has particularly preferred pairing properties. Particularly preferred are oligonucleotides according to the present invention in which the cytosine of the CpG dinucleotide is the 5^{th}-9^{th} nucleotide from the 5′-end of the 13-mer; in the case of PNA-oligomers, it is preferred for the cytosine of the CpG dinucleotide to be the 4^{th}-6^{th} nucleotide from the 5′-end of the 9-mer.

The oligomers according to the present invention are normally used in so called “sets” which contain at least one oligomer for each of the CpG dinucleotides of the sequences of Seq. ID No. 32 to Seq. ID No. 75. Preferred is a set which contains at least one oligomer for each of the CpG dinucleotides from one of Seq. ID No. 32 to Seq. ID No. 75.

In the case of the sets of oligonucleotides according to the present invention, it is preferred that at least one oligonucleotide is bound to a solid phase. It is further preferred that all the oligonucleotides of one set are bound to a solid phase.

The present invention moreover relates to a set of at least 10 n (oligonucleotides and/or PNA-oligomers) used for detecting the cytosine methylation state in chemically pretreated genomic DNA (Seq. ID No.32 to Seq. ID No.75 No.75 and sequences complementary thereto). These probes enable characterisation, grading, staging and/or diagnosis of genetic and epigenetic parameters of colon cancer. Furthermore, the probes enable the diagnosis of predisposition to colon cancer. The set of oligomers may also be used for detecting single nucleotide polymorphisms (SNPs) in pretreated genomic DNA according to one of Seq. ID No. 32 to Seq. ID No. 75.

According to the present invention, it is preferred that an arrangement of different oligonucleotides and/or PNA-oligomers (a so-called “array”) made available by the present invention is present in a manner that it is likewise bound to a solid phase. This array of different oligonucleotide- and/or PNA-oligomer sequences can be characterized in that it is arranged on the solid phase in the form of a rectangular or hexagonal lattice. The solid phase surface is preferably composed of silicon, glass, polystyrene, aluminum, steel, iron, copper, nickel, silver, or gold. However, nitrocellulose as well as plastics such as nylon which can exist in the form of pellets or also as resin matrices are possible as well.

Therefore, a further subject matter of the present invention is a method for manufacturing an array fixed to a carrier material for the grading, staging, and/or diagnosis of colon cancer, in which method at least one oligomer according to the present invention is coupled to a solid phase. Methods for manufacturing such arrays are known, for example, from U.S. Pat. No. 5,744,305 by means of solid-phase chemistry and photolabile protecting groups.

A further subject matter of the present invention relates to a DNA chip for the characterisation, grading, staging, and/or diagnosis of colon cancer. Furthermore the DNA chip enables the diagnosis of predisposition to colon cancer. The DNA chip contains at least one nucleic acid according to the present invention. DNA chips are known, for example, in U.S. Pat. No. 5,837,832.

Moreover, a subject matter of the present invention is a kit which may be composed, for example, of a bisulfite-containing reagent, a set of primer oligonucleotides containing at least two oligonucleotides whose sequences in each case correspond or are complementary to a 18 base long segment of the base sequences specified in the appendix (Seq. ID No.32 through Seq. ID No.75), oligonucleotides and/or PNA-oligomers as well as instructions for carrying out and evaluating the described method. However, a kit along the lines of the present invention can also contain only part of the aforementioned components.

The oligomers according to the present invention or arrays thereof as well as a kit according to the present invention are intended to be used for the characterisation, grading, staging and/or diagnosis of colon cancer, or diagnosis of predisposition to colon cancer. According to the present invention, the method is preferably used for the analysis of important genetic and/or epigenetic parameters within genomic DNA, in particular for use in characterisation, grading, staging and/or diagnosis of colon cancer, and predisposition to colon cancer.

The methods according to the present invention are used, for example, for characterisation, grading, staging and/or diagnosis of colon cancer.

A further embodiment of the invention is a method for the analysis of the methylation status of genomic DNA without the need for chemical pretreatment. In the first step of the method the genomic DNA sample must be isolated from tissue or cellular sources. Such sources may include cell lines, histological slides, body fluids, or tissue embedded in paraffin; for example, brain, central nervous system or lymphatic tissue. Extraction may be by means that are standard to one skilled in the art, these include the use of detergent lysates, sonification and vortexing with glass beads. Once the nucleic acids have been extracted the genomic double stranded DNA is used in the analysis.

In a preferred embodiment the DNA may be cleaved prior to the chemical treatment, this may be any means standard in the state of the art, in particular with restriction endonucleases. In the second step, the DNA is then digested with methylation sensitive restriction enzymes. The digestion is carried out such that hydrolysis of the DNA at the restriction site is informative of the methylation status of a specific CpG dinucleotide.

In the third step the restriction fragments are amplified. In a preferred embodiment this is carried out using a polymerase chain reaction.

In the final step the amplificates are detected. The detection may be by any means standard in the art, for example, but not limited to, gel electrophoresis analysis, hybridisation analysis, incorporation of detectable tags within the PCR products, DNA array analysis, MALDI or ESI analysis.

The present invention moreover relates to the diagnosis and/or prognosis of events which are disadvantageous or relevant to patients or individuals in which important genetic and/or epigenetic parameters within genomic DNA, said parameters obtained by means of the present invention may be compared to another set of genetic and/or epigenetic parameters, the differences serving as the basis for a diagnosis and/or prognosis of events which are disadvantageous or relevant to patients or individuals.

In the context of the present invention the term “hybridization” is to be understood as a bond of an oligonucleotide to a completely complementary sequence along the lines of the Watson-Crick base pairings in the sample DNA, forming a duplex structure.

The term “functional variants” denotes all DNA sequences which are complementary to a DNA sequence, and which hybridize to the reference sequence under stringent conditions.

In the context of the present invention, “genetic parameters” are mutations and polymorphisms of genomic DNA and sequences further required for their regulation. To be designated as mutations are, in particular, insertions, deletions, point mutations, inversions and polymorphisms and, particularly preferred, SNPs (single nucleotide polymorphisms).

In the context of the present invention, “epigenetic parameters” are, in particular, cytosine methylations and further chemical modifications of DNA bases of genomic DNA and sequences further required for their regulation. Further epigenetic parameters include, for example, the acetylation of histones which, cannot be directly analyzed using the described method but which, in turn, correlates with the DNA methylation.

In the following, the present invention will be explained in greater detail on the basis of the sequences and examples without being limited thereto.

Seq. ID 1 to 11 represent the genomic DNA of genes estrogen receptor, p21, p27, p16, progesteron receptor, myoglobin, pcna, cdc2, c-erbB2, p53 and CEA. These sequences are derived from Genbank and will be taken to include all minor variations of the sequence material which are currently unforseen, for example, but not limited to, minor deletions and SNPs.

Sequence ID 12 to 31 represent segments of genomic DNA which are particularly useful for the determination of colon cell proliferative disorder.

Sequence ID 32 to 75 exhibit the chemically pretreated sequence of genes estrogen receptor, p21, p27, p16, progesteron receptor, myoglobin, pcna, cdc2, c-erbB2, p53 and CEA. These sequences will be taken to include all minor variations of the sequence material which are currently unforseen, for example, but not limited to, minor deletions and SNPs.

Sequences having even sequence numbers (e.g., Seq. ID No. 32, 34, 36, . . . ) exhibit in each case sequences of chemically pretreated genomic DNAs.

Sequences having odd sequence numbers (e.g., Seq. ID No. 33, 35, 37 . . . ) exhibit in each case the sequences of chemically pretreated genomic DNAs. Said genomic DNAs are complementary to the genomic DNAs from which the preceeding sequence was derived (e.g., the complementary sequence to the genomic DNA from which Seq. ID No.32 is derived is the genomic sequence from which Seq. ID No.33 is derived, the complementary sequence to the genomic DNA from which Seq. ID No.33 is derived is the sequence from which Seq. ID No.34 is derived, etc.)

Sequence ID 76 to 97 exhibit the sequence of primer oligonucleotides for the amplification of chemically pretreated DNA according to Sequence IDs 32 to 75.

Sequence IDs 98 to 523 exhibit the sequence of oligomers which are particularly useful for the analysis of CpG positions within chemically pretreated DNA according to Sequence IDs 32 to 75.

The following examples describe the invention in detail without limiting the scope of the invention.

### EXAMPLE 1

### Description of PCR

The single gene PCR reaction was performed using a thermocycler (Epperdorf GmbH) using 10 ng of bisulfite treated DNA, 6 pmole of each primer, 200 μM of each dNTP, 1.5 mM MgCl₂ and 1 U of HotstartTaq (Qiagen AG). The other conditions were as recommended by the Taq polymerase manufacturer. Single genes were amplified by PCR performing a first denaturation step for 14 min at 96° C., followed by 39 cycles (60 sec at 96° C., 45 sec at 55° C. 75 sec at 72° C.) and a subsequent final elongation of 10 min at 72° C. The bisulfite DNA was prepared according to a published procedure from genomic DNA individually isolated from 12 matched samples of adenocarzinoma of the colon and healthy colon tissue. The genomic DNA was isolated using the wizzard DNA isolation kit (Promega, Madison).

### EXAMPLE 2

### Methylation Analysis of Gene p16

The following example relates to a fragment of the gene p16 in which a specific CG dinucleotide is to be analyzed for methylation.

In the first step, a genomic sequence is treated using bisulfite (hydrogen sulfite, disulfite) in such a manner that all cytosines which are not methylated at the 5-position of the base are modified in such a manner that a different base is substituted with regard to the base pairing behavior while the cytosines methylated at the 5-position remain unchanged.

If bisulfite solution is used for the reaction, then an addition takes place at the non-methylated cytosine bases. Moreover, a denaturating reagent or solvent as well as a radical interceptor must be present. A subsequent alkaline hydrolysis then gives rise to the conversion of non-methylated cytosine nucleobases to uracil. The chemically converted DNA is then used for the detection of methylated cytosines. In the second method step, the treated DNA sample is diluted with water or an aqueous solution. Preferably, the DNA is subsequently desulfonated. In the third step of the method, the DNA sample is amplified in a polymerase chain reaction, preferably using a heat-resistant DNA polymerase. In the present case, cytosines of the gene p16 are analyzed. To this end, a defined fragment having a length of 598 bp is amplified with the specific primer oligonucleotides

| | |
|---|---|
| TTGAAAATTAAGGGTTGAGG (Sequence ID 82) | |
| and | |
| CACCCTCTAATAACCAACCA. | (Sequence ID No. 83) |

The amplificate serves as a sample which hybridizes to an oligonucleotide previously bound to a solid phase, forming a duplex structure, for example TAAGTGTTCGGAGTTAAT (SEQ ID NO: 238), the cytosine to be detected being located at position 439 of the amplificate. The detection of the hybridization product is based on Cy3 and Cy5 fluorescently labelled primer oligonucleotides which have been used for the amplification. A hybridization reaction of the amplified DNA with the oligonucleotide takes place only if a methylated cytosine was present at this location in the bisulfite-treated DNA as shown for healthy tissue in FIG. 1A. Thus, the methylation status of the specific cytosine to be analyzed is inferred from the hybridization product.

In order to verify the methylation status of the position, a sample of the amplificate is further hybridized to another oligonucleotide previously bonded to a solid phase. Said olignonucleotide is identical to the oligonucleotide previously used to analyze the methylation status of the sample, with the exception of the position in question. At the position to be analysed said oligonucleotide comprises a thymine base as opposed to a cytosine base i.e TAAGTGTTTGGAGTTAAT (SEQ ID NO: 239). Therefore, the hybridisation reaction only takes place if an unmethylated cytosine was present at the position to be analysed as shown for tumor tissue in FIG. 1B.

### EXAMPLE 3

### Differentiation Between Colon Tumour and Healthy Colon Tissue

Differentiation of healthy samples and adenocarzinoma tumours. For tumour class prediction between healthy and tumor tissue we used a Support Vector Machine (SVM) on a set of selected CpG sites (F. Model, P. Adorjan, A. Olek, C. Piepenbrock, Feature selection for DNA methylation based cancer classification. Bioinformatics. 2001 June;17 Suppl 1:S157-64.). First we ranked the CpG sites for a given separation task by their significance of the difference between the two class means. The significance of each CpG was estimated by a two sample t-test (W, Mendenhall, T, Sincich, Statistics for engineering and the sciences (Prentice-Hall, New Jersey 1995).

In order to relate the methylation patterns to a adenocarcinoma tumour, it is initially required to comparatively analyze the DNA methylation patterns of healthy tissue and adenocarzinoma tumours tissue (FIGS. 2A and B). These analyses were carried out, analogously to Examples 1. The results obtained in this manner are stored in a database and the CpG dinucleotides which are methylated differently between the two groups are identified. This can be carried out by determining individual CpG methylation rates as can be done, for example, by sequencing, which is a relatively imprecise method of quantifying methylation at a specific CpG, or else, in a very precise manner, by a methylation-sensitive “primer extension reaction”. In a particularly preferred variant, as illustrated in the preceeding examples the methylation status of hundreds or thousands of CpGs may be analysed on an oligomer array. It is also possible for the patterns to be compared, for example, by clustering analyses which can be carried out, for example, by a computer.

A panel of genomic fragments of 11 different genes (listed in Table 1) were bisulphite treated and amplified by singleplex PCRs according to Example 1. However, as will be obvious to one skilled in the art, it is also possible to use other primers that amplify the genomic, bisulphite treated DNA in an adequate manner, and/or to carry out the PCRs in a multiplex format. However the primer oligonucleotide pairs as listed in Table 1 are particularly preferred. In order to differentiate adenocarzinoma tumour from healthy control samples optimal results were obtained by including at least 6 CpG dinucleotides, the most informative CpG positions for this discrimination being located within the p16, p53, CEA, c-erbB2 and estrogen receptor genes (cf. FIG. 2, Tab1). In addition, the majority of the analysed CpG dinucleotides of the panel showed different methylation patterns between the two phenotypes. The results prove that methylation fingerprints are capable of providing differential diagnosis of adenocarzinoma tumours and could therefore be applied in a large number clinical situations

For class prediction a SVM was trained on the most significant CpG positions, where the optimal number of CpG sites depends on the complexity of the separation task. Implementation of the SVM used the Sequential Minimal Optimization algorithm to find the 1-norm soft margin separating hyperplane (N. Christianini, J. Shawe-Taylor, An Introduction to Support Vector Machines, Cambridge University Press, Cambridge 2000). The box constraint was set to C=10. Generalization performance was estimated by averaging over 50 cross validation runs on randomly permutated samples partitioned into 8 groups.

### EXAMPLE 4

### Analysis of the Methylation Status of the most Informative CpG Positions of the Genes c-erbB2, p53, CEA, p16 and ER1

The methylation status of the most informative CpG positions of the gene fragments of genes c-erbB2, p53, CEA, p16 and ER1 are shown in this example. Corresponding to Example 2, where the methylation status is demonstrated by spots, Table 2 describes in a more detailed way the methylation status of different gene fragments of various patients by calculating the methylation status of colon tumour and healthy colon tissue. The first column indicates the specific gene fragment, the second column describes the investigated CpG Oligonukleotide, the third column depicts the diagnosis of the investigated tissue (T=tumor, H=healthy) and columns 4 to 17 show the logarithm of the ratio ofv the fluorescence signal of the CG oligonucleotide versus TG oligonucleotide of colon tumour and healthy colon tissue of 14 different patients. For example, a comparison of the methylation status of gene p16, patient 11, shows that the healthy tissue is less methylated compared to the tumour tissue for this sample. The opposite ratio can be observed, for example, for gene c-erbB2 for patient 11. In this case the tumour sample is more methylated than the healthy, sample. The analyzed CpG positions show that the genes p53, CEA, p16 and ER1 are hypermethylated, whereas c-erbB2 is hypomethylated in most of the tumour samples compared with the healthy controls.

### EXAMPLE 5

### Identification of the Methylation Status of CpG Sites of Genes CEA and p16 by Methylation Sensitive Restriction Enzyme Digest

In the CEA gene, a defined fragment having a length of 351 bp, which contains 7 CpG sites, is amplified with the specific primer oligonucleotides TGGTTAAATGTGTGGGAGAT (Sequence ID 524) and TCCTGAGTGATGTCTGTGTG (Sequence ID No. 525) and in the p16 gene, a defined fragment having a length of 391 bp, which contains 26 CpG sites, is amplified with the specific primer oligonucleotides ATGACACCAAACACCCCGAT (Sequence ID 526) and CTGTCCCTCAAATCCTCTG (Sequence ID No. 527). CGCG for gene CEA with Cytosins at positions 127 and 129 of the amplificate and CGCG for gene p16 with Cytosins at positions 362 and 364 of the amplificate, are located in a SacII restriction enzyme recognition sequence, CCGCGG. The cleavage of SacII is blocked by methylation of at least one of the two CpG dinucleotides.

The genomic DNA isolated from adenocarzinoma of colon tissue and from healthy colon tissue was hydrolysed by SacII as recommended by the manufacturer (New England Biolabs GmbH).

10 ng of the SacII restricted DNA was used as template for the amplification of the above indicated CEA and p16 gene fragments. The PCR reaction was performed using a thermocycler (Eppendorf GmbH) using 10 ng of DNA, 6 pmole of each primer, 200 μM of each dNTP, 1.5 mM MgCl2 and 1 U of HotstartTaq (Qiagen AG). The other conditions were as recommended by the Taq polymerase manufacturer. Using the above mentioned primers, gene fragments were amplified by PCR performing a first denaturation step for 14 min at 96° C., followed by 30-45 cycles (step 2:60 sec at 96° C., step 3:45 sec at 55° C., step 4: 75 sec at 72° C.) and a subsequent final elongation of 10 min at 72° C. The presence of PCR products was analysed by agrarose gel electrophoresis.

PCR products were detectable with SacII hydrolyzed DNA isolated from colon cancer tissue, when step 2 to step 4 of the cycle program were repeated 34, 37, 39, 42 and 45 fold. In contrast PCR products were only detectable with SacII hydrolyzed DNA isolated from healthy colon tissue when step 2 to step 4 of the cycle program were repeated 42 and 45 fold. These results indicate that at least one of CpG positions located within the SacII recognition sequence of the analysed CEA and the p16 gene fragment showed a higher methylation status in cancer samples compared to the healthy control.

### DESCRIPTION OF FIGURES

FIG. 1

FIG. 1 shows the hybridisation of fluorescent labelled amplificates to a surface bound olignonucleotide. Sample A being from healthy tissue and sample B being from colon adenocarzinoma tissue. Fluorescence at a spot, denoted by an arrow, indicates hybridisation of the amplificate against the olignonucleotide. Hybridisation to a CG olignonucleotide with the sequence TAAGTGTTCGGAGTTAAT (SEQ ID NO: 238) denotes methylation at the cytosine position being analysed, hybridisation to a TG olignonucleotide with the sequence TAAGTGTTTGGAGTTAAT (SEQ ID NO: 239) denotes no methylation at the cytosine position being analysed. It can be seen that sample A was umethylated for CG positions of the amplificate of gene p16 whereas in comparison sample B had a higher degree of methylation at the same position.

FIG. 2

Differentiation of colon tumour (A) from healthy colon tissue (B). High probability of methylation corresponds to red, uncertainty to black and low probability to green. The labels on the left side of the plot are gene (e.g. for the topmost: 2064) and CpG (e.g. for the topmost: 1485A) identifiers. The hybridisation was carreid out with Cy5 labelled amplificates generated by singlplex PCR reactions using primer oligonucleotides as shown in Table 1. The labels on the right side give the significance (p-value, T-test) of the difference between the means of the two groups. Each row corresponds to a single CpG and each column to the methylation levels of one sample. CpGs are ordered according to their contribution to the distinction to the differential diagnosis of the two lesions with increasing contribution from top to bottom.

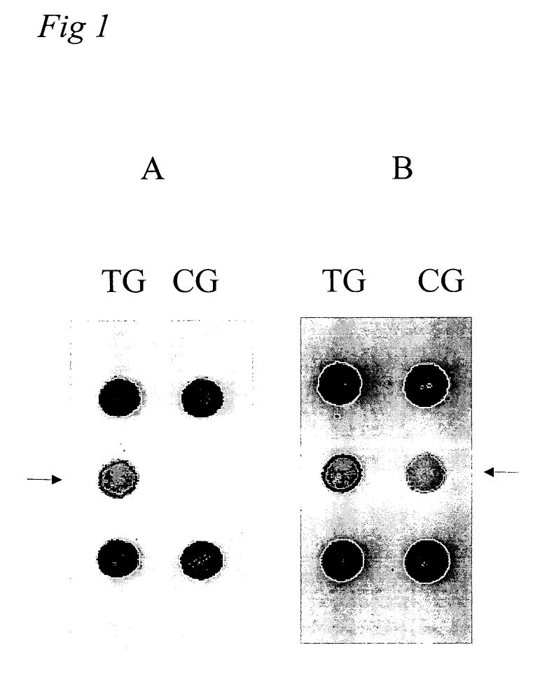

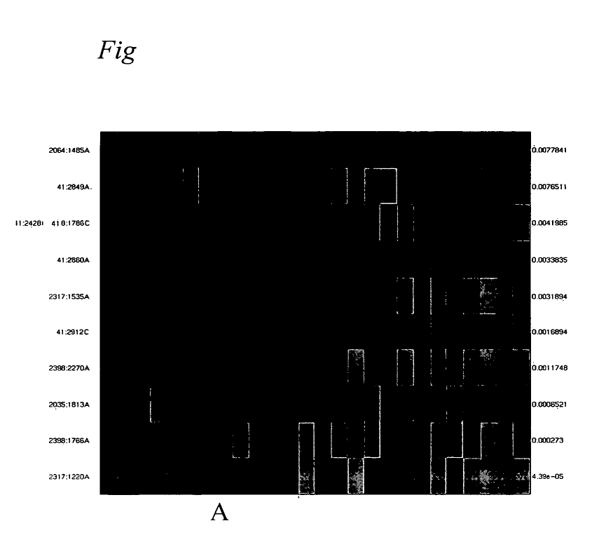

TABLE 1
List of genes, reference numbers (ID) according FIG. 2 and primer
oligonucleotides according to Example 2 and FIGS. 1 and 2.

| | | | | |
|---|---|---|---|---|
| | Gene | | | |
| Gene | Seq-ID | Accession no | Primer Seq-ID PCR primer | Primer Seq-ID PCR primer |

| | | | | | | |
|---|---|---|---|---|---|---|
| Estrogen- | 1 | NM_000125 | 76 | AGGAGGGGGAATTAAATAGA | 77 | ACAATAAAACCATCCCAAATAC |
| receptor | | | | | | |
| P21 | 2 | NM_000389 | 78 | GGATTAGTGGGAATAGAGGTG | 79 | AAACCCAAACTCCTAACTACC |
| P27 | 3 | NM_004064 | 80 | GTGGGGAGGTAGTTGAAGA | 81 | ATACACCCCTAACCCAAAAT |
| P16 | 4 | NM_000077 | 82 | TTGAAAATTAAGGGTTGAGG | 83 | CACCCTCTAATAACCAACCA |
| Progesteron- | 5 | NM_000926 | 84 | GAGGGGGTAGTGGAATTTAG | 85 | CCTTTACCTTCAACTCAATCA |
| receptor | | | | | | |
| Myoglobin | 6 | NM_005368 | 86 | GTTTTTGGTAAAGGGGTAGAA | 87 | CCTAAAATATCAACCTCCACCT |
| PCNA | 7 | NM_002592 | 88 | TTTTTAGGTTGTAAGGAGGTTTT | 89 | TAAATACCTCCAACACCTTTCT |
| CDC2 | 8 | NM_001786 | 90 | ATTAGAAGTGAAAGTAATGGAATTT | 91 | TCAATTTCCAAAAACCAAC |
| c-erbB2 | 9 | NM_004448 | 92 | GGAGGGGGTAGAGTTATTAGTT | 93 | TATACTTCCTCAAACAACCCTC |
| P53 | 10 | NM_000546 | 94 | GATTGGGTAAGTTTTTGATTGA | 95 | AAATCTCCCAACAATACAACTC |
| CEA | 11 | NM_004363 | 96 | GTTTAGGATGGGATTAAGTGTG | 97 | AATCAAATATCCCCAAATACAA |

TABLE 2

| | | | log (fluorescence OGoligo/fluorescence TGoligo) of matched pair of colon tumor (T) | | | | | | | | | | | | | |
|---|---|---|---|---|---|---|---|---|---|---|---|---|---|---|---|---|
| | | | and healthy colon tissue (H)^{a} | | | | | | | | | | | | | |
| gene | OpG | Diagnosis | 11 | 3 | 9 | 1 | 8 | 2 | 4 | 13 | 12 | 14 | 15 | 5 | 10 | 6 |
| c-erbB2 | 2064:148 | T | −1.07 | −1.72 | −1.11 | −1.53 | −1 | −1.3 | −1.63 | −1.01 | −1.64 | −1.22 | −1.33 | −0.88 | −1.57 | −1.22 |
| | | H | −0.82 | −1.09 | −1.34 | −1.17 | −0.75 | −1.09 | −1.36 | −0.89 | −0.88 | −0.72 | −1.06 | −0.93 | −1.43 | −0.7 |
| p53 | 2317:122 | T | −2.17 | −0.58 | −2.37 | −1.91 | −2.08 | −0.32 | −0.3 | −1.63 | −2.19 | −1.87 | −1.71 | −4.31 | −3.15 | −1 |
| | | H | −4.03 | −3.01 | −3.14 | −3.83 | −1.53 | −2.29 | −1.86 | −2.96 | −2.76 | −4.09 | −3.44 | −4.77 | −2.33 | −3.97 |
| p53 | 2317:153 | T | −2.38 | −2.84 | −2.77 | −2.57 | −2.93 | −2.44 | −2.89 | −3.12 | −2.77 | −2.32 | −2.7 | −2.89 | −2.13 | −2.29 |
| | | H | −3.36 | −3.36 | −3.17 | −3.32 | −3.15 | −3.47 | −2 | −2.94 | −3.8 | −3.77 | −3.67 | −4.12 | −2.2 | −3.12 |
| CEA | 2398:176 | T | −2.76 | −1.84 | −3.7 | −2.42 | −2.59 | −0.83 | −2.14 | −1.96 | −2.86 | −4.02 | −2.76 | −5.71 | −5.07 | −2.33 |
| | | H | −4.32 | −2.64 | −4.72 | −3.9 | −4.43 | −4.85 | −2.95 | −3 | −2.67 | −4.19 | −2.92 | −4.63 | −4.53 | −3.64 |
| CEA | 2398:227 | T | −3.35 | −2.15 | −3.83 | −3.88 | −4.02 | −2.95 | −2.81 | −3.98 | −3.9 | −4.01 | −4.75 | −4.64 | −4.34 | −3.15 |
| | | H | −4.7 | −4.37 | −5.1 | −5.77 | −4.7 | −4.64 | −3.33 | −3.65 | −5.48 | −5.2 | −5.32 | −5.75 | −3.84 | −5.4 |
| p16 | 2035:181 | T | −1.64 | −1.99 | −2.66 | −2.6 | −3.78 | −1.07 | −2.21 | −2.18 | −3.24 | −1.8 | −2.19 | −3.21 | −3.25 | −2.13 |
| | | H | −2.74 | −3.02 | −4.09 | −3.52 | −3.74 | −3.87 | −2.88 | −2.76 | −3.28 | −2.27 | −3.19 | −3.38 | −3.88 | −3.49 |
| ER1 | 41:2912 | T | −0.4 | −0.37 | −1.23 | −0.96 | −1.36 | −0.47 | 0.37 | −0.34 | −0.76 | −0.85 | −0.56 | −1.32 | −1.53 | −1.33 |
| | | H | −0.8 | −1.25 | −2.1 | −1.23 | −1.52 | −1.38 | −0.44 | −0.93 | −1.26 | −1.55 | −1.21 | −1.55 | −1 | −1.45 |
| ER1 | 41:2860 | T | −1.06 | −0.77 | −2.06 | −1.8 | −1.7 | −0.53 | −1.52 | −1.82 | −1.19 | −1.59 | −0.82 | −1.6 | −1.85 | −1.69 |
| | | H | −1.83 | −2.03 | −2.05 | −2.22 | −2.36 | −2.13 | −2.26 | −1.72 | −0.91 | −2.11 | −1.6 | −2.05 | −1.9 | −2.14 |
| ER1 | 41:2428 | T | 0.02 | 0.84 | −1.41 | −1.22 | −1.39 | 0.67 | −0.13 | −0.33 | −0.66 | −1.41 | 0.22 | −1.78 | −1.61 | −1 |
| | | H | −0.97 | −0.86 | −1.61 | −1.36 | −1.02 | −1.78 | −0.88 | −1.05 | −1.65 | −1.29 | −1.19 | −1.53 | −1.45 | −1.96 |
| ER1 | 41:2849 | T | −0.86 | −0.43 | −1.55 | −0.98 | −1.22 | −0.97 | −2.16 | −1.2 | −0.66 | −1.07 | −0.54 | −1.59 | −1.45 | −0.78 |
| | | H | −1.11 | −1.04 | −1.97 | −1.27 | −2.06 | −2.21 | −2.77 | −1.01 | −1.08 | −1.59 | −1.08 | −1.9 | −2.02 | −1.76 |

^{a}the values indicate the mean of at least 12 OG/TG oligo pairs analysed in 3 independent chip hybridisation

1. A method to determine the methylation status of CpG dinucleotides within one or more of the genes estrogen receptor, p21, p27, p 16, progesterone receptor, myoglobin, pcna, cdc2, c-erB2, p53 and CEA comprising contacting the target nucleic acid in a biological sample with at least one reagent or series of reagents wherein said reagent or series of reagents distinguishes between methylated and non methylated CpG dinucleotides within the target nucleic acid and concluding from the methylation status of one or more of said CpG positions on the presence or absence of a colon cell proliferative disorder.

2. A method according to claim 1 comprising the following steps:
obtaining a biological sample containing genomic DNA
extracting the genomic DNA
in the genomic DNA sample, cytosine bases which are unmethylated at the 5-position are converted, by treatment, to uracil or another base which is dissimilar to cytosine in terms of base pairing behavior;
fragments of the pretreated genomic DNA are amplified using sets of primer oligonucleotides according to Seq ID 76 to Seq ID 97 and a polymerase, the amplificates carrying a detectable label;
detection of the fragments
Identification of the methylation status of one or more cytosine positions

3. A method according to claim 2,
characterized in that the reagent is a solution of bisulfite, hydrogen sulfite or disulfite.

4. A method as recited in one of claims 2 and 3,
characterized in that the amplification is carried out by means of the polymerase chain reaction (PCR).

5. A method as recited in one of the claims 2 to 3,
characterized in that more than ten different fragments having a length of 100-2000 base pairs are amplified.

6. A method as recited in one of the claims 2 to 3,
characterized in that the amplification of several DNA segments is carried out in one reaction vessel.

7. A method as recited in one of the claims 2 to 3,
characterized in that the polymerase is a heat-resistant DNA polymerase.

8. A method as recited in one of the claims 2 to 3,
characterized in that the labels of the amplificates are fluorescence labels.

9. A method as recited in one of claims 2 to 3,
characterized in that the labels of the amplificates are radionuclides.

10. A method according to one of claims 2 to 3, characterized in that each amplificate is detected by hybridization to an oligonucleotide or peptide nucleic acid (PNA)-oligomer.

11. A method according to claim 10, characterized in that the olignonucleotide or peptide nucleic acid (PNA)-oligomer is taken from the group comprising Seq ID 98 to 523.

12. A method as recited in one of claims 2 to 3,
characterized in that the labels of the amplificates are detachable molecule fragments having a typical mass which are detected in a mass spectrometer.

13. A method as recited in one of claims 2 to 3,
characterized in that the amplificates or fragments of the amplificates are detected in the mass spectrometer.

14. A method as recited in claim 12, characterized in that the produced fragments have a single positive or negative net charge for better detectability in the mass spectrometer.

15. A method as recited in claim 12, characterized in that detection is carried out and visualized by means of matrix assisted laser desorption/ionization mass spectrometry (MALDI) or using electron spray mass spectrometry (ESI).

16. A method as recited in claim 2, characterized in that the amplification step preferentially amplifies DNA which is of particular interest in healthy and/or diseased colon tissues, based on the specific genomic methylation status of colon tissue, as opposed to background DNA.

17. A method according to claim 1 comprising the following steps;
a) obtaining a biological sample containing genomic DNA
b) extracting the genomic DNA,
c) digesting the target nucleic acids with one or more methylation sensitive restriction enzymes,
d) amplification of the DNA digest and
e) detection of the amplificates.

18. A method according to claim 17 wherein the target nucleic acids comprise one or more sequences taken from the group according to Seq ID 12 to Seq ID 31 or sequences hybridising thereto and fragments thereof.

19. A method as recited in one of claims 17 or 18, characterized in that the amplification is carried out by means of the polymerase chain reaction (PCR).

20. A method as recited in one of claims 17 to 18, characterized in that the amplification of several DNA segments is carried out in one reaction vessel.

21. A method as recited in one of claims 17 to 18, characterized in that the polymerase is a heat-resistant DNA polymerase.

22. An isolated nucleic acid of the pretreated genomic DNA according to one of the sequences taken from the group comprising Seq. ID No.32 to Seq. ID No.75 and sequences complementary thereto.

23. An oligomer, in particular an oligonucleotide or peptide nucleic acid (PNA)-oligomer, said oligomer comprising at least one base sequence of at least 10 nucleotides which hybridizes to or is identical to a pretreated genomic DNA according to one of the Seq. ID No. 32 to Seq. ID No 75 according to claim 22.

24. An oligomer or peptide nucleic acid (PNA)-oligomer as recited in claim 23, wherein the base sequence includes at least one CpG dinucleotide sequence.

25. An oligomer or peptide nucleic acid (PNA)-oligomer as recited in claim 23, characterized in that the cytosine of the at least one CpG dinucleotide is/are located approximately in the middle third of the oligomer.

26. An oligomer or peptide nucleic acid (PNA)-oligomer, in particular an oligonucleotide, according to one of the sequences taken from the group comprising Seq. ID No.98 to Seq. ID No. 523.

27. A set of oligomers or peptide nucleic acid (PNA)-oligomers, comprising at least two oligomers according to any of claims 22 to 26.

28. A set of oligomers or peptide nucleic acid (PNA)-oligomers as recited in claim 27, comprising oligomers for detecting the corresponding genomic methylation state of all CpG dinucleotides within one of the sequences according to Seq. ID Nos. 32 to 75, and sequences complementary thereto.

29. A set of at least two oligonucleotides or peptide nucleic acid (PNA)-oligomers as recited in claim 23, as primer oligonucleotides for the amplification of DNA sequences of one of Seq. ID 32 to Seq. ID 75 and/or sequences complementary thereto and segments thereof.

30. A set of oligonucleotides or peptide nucleic acid (PNA)-oligomers as recited in one of claims 22 and 23, characterized in that at least one oligonucleotide is bound to a solid phase.

31. Use of a set of oligomers or peptide nucleic acid (PNA)-oligomers according to one of the sequences taken from the group comprising Seq. ID No. 32 to Seq. ID No. 75 and sequences complementary thereto as probes for determining the cytosine methylation state and/or single nucleotide polymorphisms (SNPs) of a corresponding genomic DNA by analysis of a chemically pretreated genomic DNA according to claim 2.

32. Use of a pretreated genomic DNA according to claim 22 for the determination of the methylation status of a corresponding genomic DNA and/or detection of single nucleotide polymorphisms (SNPs).

33. A method for manufacturing an arrangement of different oligomers or peptide nucleic acid (PNA)-oligomers (array) for analyzing diseases associated with the corresponding genomic methylation status of the CpG dinucleotides within one of the Seq. ID 32 to Seq. ID 75 and sequences complementary thereto, wherein at least one oligomer according to any of the claims 22 to 26 is coupled to a solid phase.

34. An arrangement of different oligomers or peptide nucleic acid (PNA)-oligomers (array) obtainable according to claim 33.

35. An array of different oligonucleotide- and/or PNA-oligomer sequences as recited in claim 34, characterized in that these are arranged on a plane solid phase in the form of a rectangular or hexagonal lattice.

36. A DNA/PNA array for the analysis of prostate cell proliferative disorders associated with the methylation state of genes comprising at least one nucleic acid according to claim 22.

37. An array as recited in any of the claims 34 to 36, characterized in that the solid phase surface is composed of silicon, glass, polystyrene, aluminium, steel, iron, copper, nickel, silver, or gold.

38. Use of a method according to claim 1 for the characterisation, classification, diagnosis and differentiation of colon cell proliferative disorders.

39. A kit comprising a bisulfite (=disulfite, hydrogen sulfite) reagent as well as oligonucleotides and/or PNA-oligomers according to claim 22.

40. Use of a pretreated genomic DNA according to claim 22 for the characterisation, classification, diagnosis and differentiation of colon cell proliferative disorders.

41. A kit comprising a bisulfite (=disulfite, hydrogen sulfite) reagent as well as oligonucleotides and/or PNA-oligomers according to claim 26.

42. A DNA/PNA array for the analysis of prostate cell proliferative disorders associated with the methylation state of genes comprising at least one nucleic acid according to claim 26.

43. An array as recited in claim 42, characterized in that the solid phase surface is composed of silicon, glass, polystyrene, aluminium, steel, iron, copper, nickel, silver or gold.
